(19) United States
(12) Patent Application Publication
(10) Pub. No.: US 2025/0082801 A1
(43) Pub. Date: Mar. 13, 2025

(54) DECONTAMINATION OF GERMS ON HUMAN TOUCH POINTS

(71) Applicant: Microlumix LLC, Jacksonville, FL (US)

(72) Inventors: Christopher Eric Hickey, St. Augustine, FL (US); Debra Marie Vanderhoff, St. Augustine, FL (US)

(73) Assignee: Microlumix LLC, Jacksonville, FL (US)

(21) Appl. No.: 18/940,590

(22) Filed: Nov. 7, 2024

Related U.S. Application Data
(63) Continuation-in-part of application No. 17/694,599, filed on Mar. 14, 2022, now Pat. No. 12,151,044.
Application No. 17/694,599 is a continuation-in-part of application No. 17/467,208, filed on Sep. 4, 2021, now abandoned.
(60) Provisional application No. 63/075,040, filed on Sep. 4, 2020.

(51) Int. Cl. A61L 2/10; A61L 2/24

(52) U.S. Cl. CPC A61L 2/10; A61L 2/24; A61L 2202/11

(57) ABSTRACT

A germ decontamination apparatus includes an enclosure including an access door configurable to be in an open or closed position, an opening for positioning the enclosure over or around a fomite, opening means for opening the access door in response to a trigger or triggering event, one or more ultraviolet light sources disposed inside the enclosure and configured to decontaminate the fomite. The germ decontamination apparatus may include one or more sensors configured to detect a triggering event. The one or more sensors may include an obstruction sensor, a motion sensor or detector, a light sensor, a sound sensor, and/or a heat or infrared sensor. The access door may include one or more access panels. The one or more ultraviolet light sources may produce UV-C radiation with a wavelength in the range of 200-280 nm. Methods of use and systems comprising the germ decontamination apparatus are also provided.

RELATED APPLICATION DATA

This application is a continuation-in-part (CIP) application, pursuant to the provisions of 35 U.S.C. § 120, of prior U.S. patent application Ser. No. 17/694,599 (Attorney Docket No. MLXP001US) titled “DECONTAMINATION OF GERMS ON HUMAN TOUCH POINTS” by Hickey et al., filed on 2022 Mar. 14, the entirety of which is incorporated herein by reference for all purposes.

U.S. patent application Ser. No. 17/694,599 is a continuation-in-part (CIP) application, pursuant to the provisions of 35 U.S.C. § 120, of prior U.S. patent application Ser. No. 17/467,208, titled “DECONTAMINATION OF GERMS ON HUMAN TOUCH POINTS” by Hickey et al., filed on Sep. 4, 2021, the entirety of which is incorporated herein by reference for all purposes.

U.S. patent application Ser. No. 17/467,208 claims benefit, pursuant to the provisions of 35 U.S.C. § 119, of U.S. Provisional Application Ser. No. 63/075,040, titled “DECONTAMINATION OF GERMS ON HUMAN TOUCH POINTS”. naming Hickey et al. as inventors, and filed Sep. 4, 2020, the entirety of which is incorporated herein by reference for all purposes.

### FIELD OF THE INVENTION

The present invention is in the technical field of infectious disease prevention. More particularly, the present invention is in the technical field infectious disease prevention through decontamination of pathogenic microorganisms adjacent to human touch points using ultraviolet germicidal irradiation.

### BACKGROUND

Infectious diseases are caused by pathogenic microorganisms such as bacteria, viruses, fungi, and parasites which are often transmitted either directly or indirectly from person to person. Since germs thrive in a warm environment between 95-100°, the skin temperature of humans at 98.6° provides an optimal carrier platform for these microorganisms to survive and multiply. In fact, clinical studies have shown that some bacteria double every twenty minutes resulting in millions of bacteria forming in only eight hours.

While not all germs cause disease, all infectious disease is caused by germs. The four main classes of germs that can cause disease in humans include bacteria, viruses, fungi, and parasites. Studies have shown that 20% of people do not wash their hands after using the restroom and 30% of those that do wash their hands do not use soap. Overall, humans have between two and ten million germs between their fingertips and elbows at any given time. Each time an individual makes contact with a human touch point fomite (an inanimate object) such as, for example, a commercial door handle, a restroom stall latch, a credit card payment terminal, or a gas pump handle, the process of indirect germ transfer to the next user of the fomite is initiated.

Since 80% of infectious disease is transmitted by the hands, the rapid spread of pathogenic microorganisms through public touch points has been a major contributing factor in several global health pandemics including SARS and, most recently, COVID19. These events have crippled the world economy and led to the sickness and death of millions of people.

Current methods deployed to resolve this problem include manual cleaning, antimicrobial materials to manufacture and/or coat the fomites, automated and user-initiated mechanical sanitizing machines, and ultraviolet germicidal irradiation (UVGI). While each of these methods are helpful, their impact is relatively negligible in a high-traffic area due to issues such as rapid recontamination and a lengthy decontamination cycle.

Manual cleaning involves the use of sanitizers, disinfectants, and sterilizers to clean and disinfect surfaces of fomites, with each class of product designed to achieve a different result. Sanitizers prevent growth and/or kill bacteria, but not viruses, in 30 seconds to 5 minutes. Disinfectants serve as a microbicide on bacteria, some viruses, and fungi, achieving results in generally 10 minutes. Sterilizers are the most powerful cleaning agent and, when used properly, kill 100% of bacteria, viruses, fungi, and spores with the time to kill typically 10-15 minutes however this varies depending on the specific agent being used, the environment for which it is applied, and the composition of the material being sterilized.

In addition to the health risks to cleaning personnel and environmental hazards, the efficacy of cleaning agents is dependent on the application process and the surface material to which it is being applied. As noted previously, cleaning agents typically need to remain wet for 5-15 minutes to achieve a 100% reduction in pathogenic microorganisms. This time requirement is often neglected due to poor user training, worker productivity demands, and a desire to rapidly reinstate access to the fomite to users.

Additionally, cleaning personnel often use the same agents to clean all fomites regardless of whether they are constructed of porous or non-porous materials which reduces the sterilization efficacy since most cleaning agents are tailored toward specific types of surfaces. Hospital studies have also shown that some germs which have been “killed” through cleaning agents reproduce into living microorganisms in as little as two hours in a process known as photo reactivation. Lastly, even if the fomite has been properly sterilized, it only remains so until re-contaminated either by airborne bacteria or the next user interaction.

Antimicrobial materials have been used both as a material and as a surface coating for existing fomites. Most recently, copper and its alloys (brasses, bronzes, cupronickel, copper-nickel-zinc, etc.) have been shown to be natural anti-microbial materials with intrinsic properties to destroy a wide range of microorganisms. One detriment for the use of copper on public touch points is that studies have shown it takes two hours to kill 99.9% of bacteria and up to six hours to kill 99.9% of viruses when combined with a regular cleaning schedule.

Utilizing antimicrobial film and photodynamic polymer coatings have also been discussed as a potential solution. One of the problems with these solutions is the amount of time it takes for the material to be photosensitized. In the case of photodynamic polymers, which only require oxygen and natural light, this process takes sixty minutes to achieve a 1 log anti-microbial reduction.

There are three primary issues that prevent antimicrobial materials and coatings from being an adequate solution to prevent the transmission of infectious diseases from human touch point fomites. First, during the lengthy timeframe necessary to achieve a 99.9% inactivation of pathogenic microorganisms, millions of additional microorganisms have been placed on the fomite by new users making it unlikely that a fomite will be disinfected during a period of heavy use. Second, their efficacy varies depending on the germ it is combating. Some are only effective against bacteria or viruses but not both. Of those that have proven capable of killing both bacteria and viruses, many of those are unable to kill other classes of pathogenic microorganisms such as fungi, spores, and/or parasites. Meanwhile, none of these materials have shown to be equally effective against all microorganisms. Lastly, the cost and deployment time to replace and coat all public facing fomites makes this option both undesirable and unrealistic.

Mechanical sterilizing options of human touch point fomites include user-actuated and automated mechanical machines to kill pathogenic microorganisms, utilizing either chemicals in the form of sanitizers, disinfectants, or sterilizers or germicidal light performing ultraviolet germicidal irradiation (herein referred to as UVGI). Machines utilizing chemicals are typically mounted adjacent to a fomite and have a housing filled with cleaning product which is applied to the target surface either through a user actuated lever or the triggering of the action through an automated sensor. The user actuated model poses a problem at the point of initiation since germs are spread to the lever from each user's hand which accumulate each time the machine is used.

The automated sensor-actuated machine resolves the user interface problem, but other critical issues persist in the fight against human touch point germ contamination, especially in public spaces. First, chemicals typically need up to 15 minutes to achieve optimal efficacy in killing pathogenic microorganisms which is often not enough time for frequently accessed fomites to be contagion-free prior to the next user interaction. Second, even if effective in killing germs on the fomite, the chemical residue poses a new health risk as it is distributed to the hands of subsequent users. Lastly, the chemical residue surrounding the distribution area creates the potential for slip and fail injuries.

Ultraviolet germicidal irradiation (UVGI) has been validated as a method of sterilization in medical and surgical environments since the 1950's. The wavelength between 200-280 nm is classified as UV-C light and has the strongest germicidal effect. Through exposure to UV-C, the DNA of the pathogens is destroyed, rendering them incapable of replicating. Until relatively recently, the primary method of producing germicidal light was using mercury-filled tubes. Colloquially known as germicidal lamps, these are similar in appearance to a standard fluorescent light. Producing light at a peak of 253.7 nm, it is effective in killing pathogenic microorganisms but not optimal, since 265 nm has been proven to be the most effective wavelength against the broad spectrum of bacteria and viruses.

The use of UV-C light to eliminate pathogenic microorganisms is a globally accepted solution and is widely used in medical environments including sterilization of instruments, devices, operating and patient rooms, and within HVAC systems. It is also commonly used to treat air, water, and surfaces in various industries and sectors including but not limited to water purification plants, food production and packaging, and warehouses. In recent years, small user-actuated UV-C devices such as lamps and handheld wands have been made available to the consumer market for sterilization of surfaces such as sinks, toilets, toothbrushes, keys, and cell phones.

However, germicidal lamps have not proven to be a commercially viable solution for eradication of germs on public touch point fomites. The drawbacks of using germicidal lamps on high traffic surfaces such as door handles and elevator buttons, for example, include but are not limited to; the inability to perform rapid cycling, reduced total life expectancy when repeatedly cycled on and off, slow startup time to reach its peak wavelength, high heat production, additional equipment required to operate i.e. a ballast, and a danger to the general public if leaking mercury from defective or broken bulbs comes into contact with human skin or eyes.

Therefore, a need exists in the field for novel germ decontamination methods, devices, and apparatuses capable of rapid and efficient sterilization of human touch point fomites to prevent the spread of infectious disease and loss of millions of lives.

### DETAILED DESCRIPTION OF EXAMPLE EMBODIMENTS

### Overview

Various aspects described or referenced herein are directed to different techniques for facilitating germ decontamination of fomite regions of an object fixedly attached to a support structure.

One aspect disclosed herein is directed to a germ decontamination apparatus adapted to decontaminate at least one fomite region of an object fixedly attached to a support structure, the apparatus comprising: a housing defining an interior enclosure; the housing including a rear housing portion and a front housing portion; the rear housing portion including a rear opening disposed therein, the rear opening being configured or designed to provide access to the interior enclosure; the housing being configured or designed to be attachable to the support structure in a first configuration which permits the entirety of the at least one fomite region to be exposed to the interior enclosure; the front housing portion including a front cover plate and a front opening configured or designed to provide access to the interior enclosure; a movable access door movably attached to the housing for preventing access to the interior enclosure via the front opening, the access door being configurable in a closed configuration which prevents access to the interior enclosure, the access door being further configurable in an open configuration which permits access to the interior enclosure; wherein the access door comprises a first movable, stackable access panel, the first access panel being movable into a first stacked configuration with the front cover plate such that the first access panel is stacked either behind or in front of the front cover plate when the access door is configured in the open configuration; the first access panel including at least one first edge portion and a first body portion, the at least one first edge portion being different from the first body portion, the first body portion including a first curved body portion; a drive mechanism for causing the access door to open or close in response to at least one triggering event; one or more ultraviolet light sources disposed at the interior enclosure and configured to decontaminate the at least one fomite region; one or more sensors configured to detect the at least one triggering event; and a controller configured to control the one or more sensors, the drive mechanism, and/or the one or more light sources.

In at least one embodiment, a cross-section of the first access panel body is substantially U-shaped or substantially C-shaped.

In at least one embodiment, the access door further comprises a second movable access panel, the second access panel being movable into a second stacked configuration such that the second access panel is stacked either behind or in front of the first access panel when the access door is configured in the open configuration.

In at least one embodiment, the object is a door knob or door handle; the support structure is a door; the controller is configured or designed to execute instructions for: detecting a presence of a first person; configuring the access door in the open configuration to enable the detected first person to access the object in the interior enclosure; detecting an absence of the first person while the access door is configured in the open configuration; and delaying closing of the access door for at least 5 seconds after detecting the absence of the first person.

In at least one embodiment, the controller is configured or designed to execute instructions for: detecting a presence of a first person within a first predetermined distance from the apparatus; determining a current direction of motion of the first person; and configuring the access door in the open configuration in response to determining that: (i) the first person is within a first predetermined distance from the apparatus, and (ii) the current direction of movement of the first person is toward the apparatus.

In at least one embodiment, the controller is configured or designed to execute instructions for: detecting a presence of a first person within a first predetermined distance from the apparatus; determining a current direction of motion of the first person; configuring the access door in the open configuration in response to determining that: (i) the first person is within a first predetermined distance from the apparatus, and (ii) the current direction of movement of the first person is toward the apparatus; causing the access door to be or to remain in a closed configuration in response to determining that the first person is greater than the first predetermined distance from the apparatus; and causing the access door to be or to remain in a closed configuration in response to determining that the current direction of movement of the first person is not toward the apparatus.

In at least one embodiment, the object is a latch or locking mechanism for a door of a restroom stall; the controller is configured or designed to execute instructions for: detecting a presence of a first person in the restroom stall; configuring the access door in the open configuration while the first person is detected in the restroom stall; detecting an absence of the first person in the restroom stall; configuring the access door in the closed configuration in response to detecting the absence of the first person in the restroom stall; and initiating a first decontamination operation for decontaminating the at least one fomite region in response to: (i) detecting the absence of the first person in the restroom stall, and (ii) determining that the access door is in the closed configuration.

In at least one embodiment, the apparatus further comprises a first sensor configured or designed to monitor the interior enclosure and to detect a presence of an external object within the interior enclosure; wherein the controller is configured or designed to execute instructions for: configuring the access door in the open configuration; determining if the external object has been detected within the interior enclosure;

initiating a first decontamination operation for decontaminating the at least one fomite region in response to determining that the external object has been detected within the interior enclosure; and preventing initiation of the first decontamination operation in response to determining that the external object has not been detected within the interior enclosure.

In at least one embodiment, the apparatus further comprises a first sensor configured or designed to monitor the interior enclosure and to detect if an external object has made contact with the object within the interior enclosure; wherein the controller is configured or designed to execute instructions for: configuring the access door in the open configuration; determining if the external object has made contact with the object within the interior enclosure; initiating a first decontamination operation for decontaminating the at least one fomite region in response to determining that the external object made contact with the object; and preventing initiation of the first decontamination operation in response to determining that the external object did not make contact with the object.

In at least one embodiment, the object is an elevator button panel of an elevator cab; the controller is configured or designed to execute instructions for: detecting a presence of at least one person in the elevator cab; configuring the access door in the open configuration in response to detecting the presence of the at least one person in the elevator cab; detecting current vertical movement of the elevator cab; configuring the access door in the closed configuration in response to detecting the vertical movement of the elevator cab; and initiating a first decontamination operation for decontaminating the at least one fomite region in response to: (i) detecting the vertical movement of the elevator cab, and (ii) determining that the access door is in the closed configuration.

In at least one embodiment, the object is an elevator button panel of an elevator cab; the controller is configured or designed to execute instructions for: detecting a presence of at least one person in the elevator cab; detecting that the elevator cab is currently not moving; and configuring the access door in the open configuration in response to: (i) detecting the presence of the at least one person in the elevator cab, and (ii) detecting that the elevator cab is currently not moving; detecting current vertical movement of the elevator cab; configuring the access door in the closed configuration in response to detecting the vertical movement of the elevator cab; and initiating a first decontamination operation for decontaminating the at least one fomite region in response to: (i) detecting the vertical movement of the elevator cab, and (ii) determining that the access door is in the closed configuration.

In at least one embodiment, the first panel and the front cover plate are configured in the first stacked configuration while the access door is configured in the open configuration; and the first panel and the front cover plate are configured in a non-planar, stepped configuration while the access door is configured in the closed configuration.

In at least one embodiment, the housing is configured or designed to envelope the at least one fomite region while the housing is attached to the support structure according to the first configuration, and while the access door is configured in the closed configuration.

In at least one embodiment, the rear opening is configured or designed to enable the at least one fomite region to pass therethrough.

In at least one embodiment, the object is a fixture fixedly attached to the support structure, and the housing is configured or designed to be fixedly attachable to the support structure in a manner which causes the entirety of the at least one fomite region to be exposed to the interior enclosure via the rear opening.

In at least one embodiment, the apparatus further comprises at least one portable power source for providing power to at least one electronic component of the portable germ decontamination apparatus.

In at least one embodiment, the one or more sensors comprise an obstruction sensor, a motion sensor or detector, a light sensor, a sound sensor, and/or a heat or infrared sensor.

In at least one embodiment, the controller is configured or designed to execute a plurality of instructions for implementing the decontamination of the at least one fomite region in a manner which causes inactivation of germ populations of the at least one fomite region by at least 99%.

In at least one embodiment, the one or more ultraviolet light sources are configured or designed to produce UV-C radiation with a wavelength in the range of 200-280 nm.

In at least one embodiment, the housing is configured or designed to provide a substantially airtight interior enclosure while the housing is attached to the support structure via the first configuration, and while the access door is in the closed configuration.

In at least one embodiment, the apparatus is further configured or designed to decontaminate all exposed fomite regions of the object while the housing is attached to the support structure via the first configuration, and while the access door is in the closed configuration.

In at least one embodiment, the apparatus includes a wireless transceiver configured or designed to facilitate wireless communication with a second germ decontamination apparatus comprising a second movable access door; and the controller is configured or designed to execute a plurality of instructions for transmitting a first control signal to the second germ decontamination apparatus to cause the second germ decontamination apparatus to open the second access door.

In at least one embodiment, the apparatus includes a wireless transceiver configured or designed to facilitate wireless communication with a second germ decontamination apparatus comprising a second movable access door; and the controller is configured or designed to execute a plurality of instructions for transmitting a first set of control signals to the second germ decontamination apparatus for controlling movement of the second access door.

In at least one embodiment, the controller is configured or designed to execute instructions for: detecting a presence of at least one person; configuring the access door in the open configuration in response to detecting the presence of the at least one person; detecting movement of the apparatus; and configuring the access door in the closed configuration in response to detecting movement of the apparatus.

Another aspect disclosed herein is directed to a germ decontamination apparatus adapted to decontaminate at least one fomite region of an object fixedly attached to a support structure, the apparatus comprising: a housing defining an interior enclosure; the housing including a rear housing portion and a front housing portion; the rear housing portion including a rear opening disposed therein, the rear opening being configured or designed to provide access to the interior enclosure; the housing being configured or designed to be attachable to the support structure in a first configuration which permits the entirety of the at least one fomite region to be exposed to the interior enclosure; the front housing portion including a front cover plate and a front opening configured or designed to provide access to the interior enclosure; a movable access door movably attached to the housing for preventing access to the interior enclosure via the front opening, the access door being configurable in a closed configuration which prevents access to the interior enclosure, the access door being further configurable in an open configuration which permits access to the interior enclosure; wherein the access door comprises a first movable, stackable access panel, the first access panel being movable into a first stacked configuration with the front cover plate such that the first access panel is stacked either behind or in front of the front cover plate when the access door is configured in the open configuration; the first access panel including at least one first edge portion and a first body portion, the at least one first edge portion being different from the first body portion, the first body portion including a first curved body portion; a drive mechanism for causing the access door to open and close; one or more sensors; a first decontamination mechanism disposed at the interior enclosure and configured or designed to decontaminate the at least one fomite region; and a controller configured to control the one or more sensors, the drive mechanism, and/or first decontamination mechanism.

In at least one embodiment, a germ decontamination apparatus includes an enclosure including an access door configurable to be in an open or closed position, an opening for positioning the enclosure over or around a fomite, a drive assembly configured to open the access door in response to a trigger or triggering event, one or more ultraviolet light sources disposed inside the enclosure and configured to decontaminate the fomite. The germ decontamination apparatus may include one or more sensors configured to detect a triggering event. The one or more sensors may include a motion detector sensor and/or a Sight sensor. The access door may include one or more access panels. The one or more ultraviolet light sources may produce UV-C radiation with a wavelength in the range of 200-280 nm.

The disclosure is also directed to a germ decontamination method and apparatus which forms a chamber that affixes to and encloses human touch point fomites including but not limited to door handles, restroom stall latches, deadbolts, gas pump handles, retail point of-sale (POS) terminals, automatic teller machines, shopping cart handles, elevator control panels, public telephones, paper towel extraction levers, toilet handles and seats, etc. This apparatus automatically kills adjacent germs within seconds after each Interaction with a user through ultraviolet germicidal irradiation (herein referred to as “UVGI”). The UVGI dose is delivered through UV-C LED semiconductor chips (herein also referred to as “UV-C”) optimally mounted at a fixed or adjustable angle to the baseplate and/or upper housing to ensure proper coverage and the most effective placement. The chips are preferably delivering their dose at an optimal wavelength of 265 nm or alternatively through a multiwavelength UV-C LED array to specifically target different classes of germs. Internal components enveloping fomites within the chamber are layered with a UV-C reflective material such as aluminum foil, PTFE, UV-reflective paint, or any similar material proven to optimize reflectivity. Once the UVGI dose has been administered, the fomite remains sealed within the enclosure to prevent recontamination resulting from airborne pathogenic microorganisms. Upon detection of the presence of a subsequent user through sensor technology, the drive and pulley system retract the stacking access panels to allow germ-free touch point interaction with the fomite which, upon conclusion, triggers the closure of the access panels and the UVGI cycle to be repeated.

The disclosure is also directed to a germ decontamination system comprising a product comprising a fomite, wherein any of the germ decontamination apparatuses described herein are configured to be integrated into the product comprising a fomite. In certain embodiments, the product comprising a fomite is a door, a restroom stall, a deadbolt, a gas pump, a retail point of-sale (POS) terminal, an automatic teller machine, a shopping cart, an elevator, a public telephone, a paper towel dispenser, a computer keyboard, or a toilet.

Other features and aspects will be apparent from the following detailed description, the drawings, and the claims.

Various objects, features and advantages of the various aspects described or referenced herein will become apparent from the following descriptions of its example embodiments, which descriptions should be taken in conjunction with the accompanying drawings.

### BRIEF DESCRIPTION OF THE DRAWINGS

The following figures show various features and aspects of the present invention.

FIG. 1A presents an elevated front view of one example of a germ decontamination chamber for human touch point fomites according to various embodiments of the present invention.

FIG. 1B depicts an elevated side perspective view of the right side of a germ decontamination chamber comprising the battery components and a partially retracted drive panel.

FIG. 1C illustrates an elevated front perspective view of the front interior including access panel rails and obstruction sensors.

FIG. 1D shows an elevated rear perspective view of the baseplate mounted to a germ decontamination chamber.

FIG. 2 portrays an elevated rear exploded view of the components comprised within the upper housing assembly and baseplate assembly.

FIG. 3A shows a front view of the baseplate of a germ decontamination chamber comprising the microcontroller and the mounted UV-C source.

FIG. 3B portrays a front view of a baseplate cover.

FIG. 3C represents an exploded front view of the placement of the baseplate cover over the baseplate.

FIG. 3D exhibits a front view of the baseplate cover combined with the baseplate to form the baseplate assembly.

FIG. 4 exhibits an elevated front exploded view of the upper housing, baseplate cover, and baseplate.

FIG. 5 reveals an exploded top view of the adjustable UV-C mounting stand components.

FIG. 6A displays a side view of pivot angles ranging from 15°-90° created by the UV-C mounting stand.

FIG. 6B illustrates a side perspective view of the UV-C mounting stand tilted at a 30° forward angle.

FIG. 6C depicts a top view of the UV-C stand tilted at a 75° angle.

FIG. 6D shows a front perspective view of the UV-C mounting stand tilted at a 45° angle.

FIG. 6E portrays an elevated front view of the UV-C mounting stand tilted at a 15° angle.

FIG. 6F presents a top view of the swivel action created by the UV-C mounting stand.

FIG. 7 depicts a front closeup view of the access panel assembly.

FIG. 8A represents a top view of an access panel frame with embedded rails.

FIG. 8B exhibits a side closeup view of the nylon glide within a panel rail.

FIG. 9A reveals a side view of a drive clip.

FIG. 9B displays a top view of a drive clip.

FIG. 9C presents an outer side view of an access panel support arm.

FIG. 9D illustrates an inner side view of an access panel support arm.

FIG. 10A depicts a side sectional view of the left access panel frame, panel rails, and drive rail.

FIG. 10B shows the side sectional view depicted in 8A adding the drive clip, support arms, and an exploded view of the pulley and chain.

FIG. 10C portrays the side sectional view shown in 8B amended to show the pulley and chain in its operational position.

FIG. 11 represents a side sectional view of the access panels connected to their respective rails in the closed position and identifies the location of the panel bay when the access panels are retracted.

FIG. 12A exhibits a side sectional view of the access panel group in the closed position.

FIG. 12B exhibits a side sectional view of the access panel group 25% retracted.

FIG. 12C exhibits a side sectional view of the access panel group 50% retracted.

FIG. 12D exhibits a side sectional view of the access panel group 75% retracted.

FIG. 12E exhibits a side sectional view of the access panel group fully retracted and parked in the panel bay.

FIG. 13 shows a rear closeup view of the drive assembly.

FIG. 14 represents a rear closeup view of the upper housing assembly.

FIG. 15A reveals a front interior view of an open germ decontamination chamber adjacent to an elevator control panel.

FIG. 15B displays a front interior view of an open germ decontamination chamber adjacent to a door handle.

FIG. 15C presents a front interior view of an open germ decontamination chamber adjacent to a wall-mounted courtesy telephone.

FIG. 15D illustrates a front interior view of an open germ decontamination chamber adjacent to a restroom stall deadbolt.

FIG. 16 depicts a process flow diagram for the germ decontamination chamber.

FIG. 17A shows a front view of a germ decontamination chamber closed and sealed.

FIG. 17B shows a front view of a germ decontamination chamber with the access panels 25% retracted.

FIG. 17C shows a front view of a germ decontamination chamber with the access panels 50% retracted.

FIG. 17D shows a front view of a germ decontamination chamber with the access panels 75% retracted.

FIG. 17E shows a front view of a germ decontamination chamber with the access panels 100% retracted and parked in the panel bay revealing an elevator control panel.

18A displays a front view of a detached fitted door handle baseplate and commercial door handle and lock.

FIG. 18B presents a front view of a fitted door handle baseplate adjacent to a commercial door handle and lock.

FIG. 18C illustrates a front view of a detached door handle baseplate cover and fitted door handle baseplate adjacent to a commercial door handle and lock.

FIG. 18D depicts a front view of a fitted door handle baseplate assembly and commercial door handle and lock.

FIG. 19A shows a front perspective exploded view of the upper housing assembly projected into position adjacent to a door handle baseplate assembly and commercial door. FIG. 19B portrays a front view of a commercial door handle germ decontamination chamber with access panels retracted adjacent to a commercial door.

FIG. 20A represents a front view of a detached fitted gas pump baseplate with a microcontroller, UV-C, and gas pump handle.

FIG. 20B exhibits a front view of a fitted gas pump baseplate with microcontroller and UV-C adjacent to a gas pump handle.

FIG. 20C reveals a front view of a detached gas pump baseplate cover and fitted baseplate adjacent to a gas pump handle.

FIG. 20D displays a front view of a fitted gas pump baseplate assembly and gas pump handle.

FIG. 21A presents a front exploded perspective view of the upper housing assembly projected into position adjacent to a gas pump baseplate assembly and gas pump handle.

FIG. 21B illustrates a front view of a germ decontamination chamber with access panels retracted adjacent to a gas pump handle.

FIG. 21C depicts a front view of a gas pump service island with a gas pump handle germ decontamination chamber adjacent to a gas pump.

FIG. 22A shows a front view of a restroom stall latch germ decontamination chamber in the closed position.

FIG. 22B exhibits an elevated side perspective view of a restroom stall latch germ decontamination chamber and brush shield.

FIG. 22C portrays a front view of a restroom stall latch germ decontamination chamber in the open position adjacent to a stall latch.

FIG. 22D represents a top perspective view of a restroom stall latch germ decontamination chamber and battery access door.

FIG. 23A displays a front view of a detached fitted stall latch baseplate, microcontroller, and mounted UV-C.

FIG. 23B exhibits a front view of a fitted stall latch baseplate with microcontroller and UV-C adjacent to a restroom stall latch.

FIG. 23C reveals a front view of a detached stall latch baseplate cover and fitted baseplate adjacent to a restroom stall latch.

FIG. 23D displays a front view of a fitted stall latch baseplate assembly and restroom stall latch.

FIG. 24 shows a front closeup view of a restroom stall latch access panel assembly.

FIG. 25 portrays a rear closeup view of the restroom stall latch drive assembly.

FIG. 26 represents a rear exploded view of the components comprised within the restroom stall latch upper housing assembly.

FIG. 27 reveals a rear closeup view of the restroom stall latch upper housing assembly.

FIG. 28 displays a front exploded view of the restroom stall latch upper housing, baseplate cover, and baseplate.

FIG. 29 presents a front view of a restroom stall latch germ decontamination chamber in the open position adjacent to a restroom stall latch and door.

FIG. 30A illustrates a front view of a retail point-of-sale terminal (“POS”) germ decontamination chamber (herein referred to as “POS chamber”) and mounting stand.

FIG. 30B presents a front view of an open POS chamber and mounting stand.

FIG. 30C illustrates a rear perspective view of a POS chamber and mounting stand.

FIG. 31A depicts a front view of a POS baseplate with microcontroller and UV-C.

FIG. 31B shows a front view of a POS baseplate cover with UV-C cutouts.

FIG. 32A portrays a front exploded view of a POS baseplate cover being projected into position over and adjacent to a POS baseplate and mounting stand.

FIG. 32B represents a front view of a POS baseplate assembly and mounting stand.

FIG. 33A exhibits a front closeup view of a POS chamber access panel assembly.

FIG. 33B reveals a rear view of a POS chamber drive assembly.

FIG. 33C displays a rear view of a POS chamber upper housing assembly.

FIG. 34A exhibits a front exploded view of a POS chamber upper housing assembly projected into position upon the POS baseplate assembly and mounting stand.

FIG. 34B depicts a front view of a closed POS chamber.

FIG. 35A reveals a front view of a POS chamber in the closed position.

FIG. 35B reveals a front view of a POS chamber with one access panel retracted.

FIG. 35C reveals a front view of a POS chamber with two access panels retracted.

FIG. 35D reveals a front view of a POS chamber with three access panels.

FIG. 35E reveals a front view of a POS chamber with four access panels retracted.

FIG. 35F reveals a front view of a POS chamber with five access panels retracted.

FIG. 35G reveals a front view of a POS chamber with six access panels retracted.

FIG. 36 illustrates a front perspective view of a POS chamber mounted at a retail checkout counter.

FIG. 37A depicts a front view of a shopping cart cylindrical germ decontamination chamber (“herein also referred to as “SC chamber”) adjacent to a shopping cart.

FIG. 37B shows a front view closeup of an SC chamber, access sensor, and status lights.

FIG. 38A portrays a front view of a SC chamber baseplate.

FIG. 38B represents a top perspective view of a SC chamber baseplate and UV-C.

FIG. 39A exhibits an elevated side view of a SC chamber baseplate cover with UV-C cutouts.

FIG. 39B displays an elevated front perspective closeup exploded view of a SC chamber baseplate cover projected into position over the SC chamber baseplate collectively forming the undercarriage assembly.

FIG. 40A presents a front exploded view of the undercarriage assembly projected adjacent to a shopping cart handle and left and right housing of the SC chamber.

FIG. 40B depicts a side perspective view of an assembled left housing and battery access panel.

FIG. 41A illustrates an elevated side perspective exploded view of the components comprising the left housing of the SC chamber.

FIG. 41B exhibits a side closeup view of the left housing and battery access panel.

FIG. 42A shows a side perspective exploded view of the drive hub (herein also referred to as “hub”) projected adjacent to the driven drum (herein also referred to as “drum”) of the left housing.

FIG. 42B portrays a side closeup view of the left hub and drum assembly (herein also referred to as “H&D assembly”) in the closed position.

FIG. 43A depicts an elevated side perspective exploded view of the components comprising the right housing of the SC chamber.

FIG. 43B presents a side perspective view of the oppositely disposed and parallel left housing and right housing adjacent to a cyl drive panel (herein also referred to as “cyl panel #1”) in the closed position.

FIG. 44A illustrates a side closeup view of the left hub and drum assembly and cylindrical rail group (herein also referred to collectively as “cyl rails” or “rails”) in the closed position with projection lines detailing the rotation of the drive hub and drum components during the retraction process.

FIG. 44B depicts a side closeup view of the nylon glide.

FIG. 45A presents a top perspective view of a cylindrical access panel.

FIG. 45B illustrates a bottom perspective view of a cylindrical access panel.

FIG. 45C represents a bottom perspective exploded view projecting the interface of three access panels with cylindrical drive clips (herein also referred to as “cyl drive clips”) and cylindrical channel guides (herein also referred to as “cyl channel guides”).

FIG. 46A shows a side closeup view of the left hub and drum assembly in hub position “0” (closed).

FIG. 46B shows a side closeup view of the left hub and drum assembly in hub position “1” (one panel retracted).

FIG. 46C shows a side closeup view of the left hub and drum assembly in hub position “2” (two panels retracted).

FIG. 46D shows a side closeup view of the left hub and drum assembly in hub position “3” (three panels retracted).

FIG. 46E shows a side closeup view of the left hub and drum assembly in hub position “4” (access panels open).

FIG. 47A displays an elevated top view of the panels of an SC chamber in a closed state (hub position “0”).

FIG. 47B displays an elevated top view of the panels of an SC chamber with one access panel retracted (hub position “1”).

FIG. 47C displays an elevated top view of the panels of an SC chamber with two access panels retracted (hub position “2”).

FIG. 47D displays an elevated top view of the panels of an SC chamber with three access panels retracted (hub position “3”).

FIG. 47E displays an elevated top view of the panels of an SC chamber with all access panels retracted (hub position “4”) providing access to the sterilized shopping cart handle.

FIG. 48 shows a perspective view of an example embodiment of a Germpass Elevator Hall Call Unit configured in a closed configuration.

FIG. 49 shows a perspective view of an example embodiment of a Germpass Elevator Hall Call Unit configured in an open configuration.

FIG. 50 shows a front view of an example embodiment of a Germpass Elevator Hall Call Unit configured in a closed configuration.

FIG. 51 shows a perspective view of an example embodiment of a Germpass Elevator Hall Call Unit configured in a closed configuration.

FIG. 52 shows a left view of an example embodiment of a Germpass Elevator Hall Call Unit configured in a closed configuration.

FIG. 53 shows a right view of an example embodiment of a Germpass Elevator Hall Call Unit configured in a closed configuration.

FIG. 54 shows a top view of an example embodiment of a Germpass Elevator Hall Call Unit configured in a closed configuration.

FIG. 55 shows a bottom view of an example embodiment of a Germpass Elevator Hall Call Unit configured in a closed configuration.

FIG. 56 shows a rear view of an example embodiment of a Germpass Elevator Hall Call Unit configured in a closed configuration.

FIG. 57 shows a front view of an example embodiment of a Germpass Elevator Hall Call Unit configured in an open configuration.

FIG. 58 shows a perspective view of an example embodiment of a Germpass Elevator Hall Call Unit configured in an open configuration.

FIG. 59 shows a left view of an example embodiment of a Germpass Elevator Hall Call Unit configured in an open configuration.

FIG. 60 shows a right view of an example embodiment of a Germpass Elevator Hall Call Unit configured in an open configuration.

FIG. 61 shows a top view of an example embodiment of a Germpass Elevator Hall Call Unit configured in an open configuration.

FIG. 62 shows a bottom view of an example embodiment of a Germpass Elevator Hall Call Unit configured in an open configuration.

FIG. 63 shows a rear view of an example embodiment of a Germpass Elevator Hall Call Unit configured in an open configuration.

FIG. 64 shows a perspective view of an example embodiment of a Germpass Embedded Elevator Cab Unit configured in an open configuration.

FIG. 65 shows a front view of an example embodiment of a Germpass Embedded Elevator Cab Unit configured in a closed configuration.

FIG. 66 shows a perspective view of an example embodiment of a Germpass Embedded Elevator Cab Unit configured in a closed configuration.

FIG. 67 shows a left view of an example embodiment of a Germpass Embedded Elevator Cab Unit configured in a closed configuration.

FIG. 68 shows a right view of an example embodiment of a Germpass Embedded Elevator Cab Unit configured in a closed configuration.

FIG. 69 shows a top view of an example embodiment of a Germpass Embedded Elevator Cab Unit configured in a closed configuration.

FIG. 70 shows a bottom view of an example embodiment of a Germpass Embedded Elevator Cab Unit configured in a closed configuration.

FIG. 71 shows a rear view of an example embodiment of a Germpass Embedded Elevator Cab Unit configured in a closed configuration.

FIG. 72 shows a front view of an example embodiment of a Germpass Embedded Elevator Cab Unit configured in an open configuration.

FIG. 73 shows a perspective view of an example embodiment of a Germpass Embedded Elevator Cab Unit configured in an open configuration.

FIG. 74 shows a left view of an example embodiment of a Germpass Embedded Elevator Cab Unit configured in an open configuration.

FIG. 75 shows a right view of an example embodiment of a Germpass Embedded Elevator Cab Unit configured in an open configuration.

FIG. 76 shows a top view of an example embodiment of a Germpass Embedded Elevator Cab Unit configured in an open configuration.

FIG. 77 shows a bottom view of an example embodiment of a Germpass Embedded Elevator Cab Unit configured in an open configuration.

FIG. 78 shows a rear view of an example embodiment of a Germpass Embedded Elevator Cab Unit configured in an open configuration.

FIG. 79 shows a perspective view of an example embodiment of a Germpass Retrofit Elevator Cab Unit configured in a closed configuration.

FIG. 80 shows a perspective view of an example embodiment of a Germpass Retrofit Elevator Cab Unit configured in an open configuration.

FIG. 81 shows a front view of an example embodiment of a Germpass Retrofit Elevator Cab Unit configured in a closed configuration.

FIG. 82 shows a perspective view of an example embodiment of a Germpass Retrofit Elevator Cab Unit configured in a closed configuration.

FIG. 83 shows a left view of an example embodiment of a Germpass Retrofit Elevator Cab Unit configured in a closed configuration.

FIG. 84 shows a right view of an example embodiment of a Germpass Retrofit Elevator Cab Unit configured in a closed configuration.

FIG. 85 shows a top view of an example embodiment of a Germpass Retrofit Elevator Cab Unit configured in a closed configuration.

FIG. 86 shows a bottom view of an example embodiment of a Germpass Retrofit Elevator Cab Unit configured in a closed configuration.

FIG. 87 shows a rear view of an example embodiment of a Germpass Retrofit Elevator Cab Unit configured in a closed configuration.

FIG. 88 shows a front view of an example embodiment of a Germpass Retrofit Elevator Cab Unit configured in an open configuration.

FIG. 89 shows a perspective view of an example embodiment of a Germpass Retrofit Elevator Cab Unit configured in an open configuration.

FIG. 90 shows a left view of an example embodiment of a Germpass Retrofit Elevator Cab Unit configured in an open configuration.

FIG. 91 shows a right view of an example embodiment of a Germpass Retrofit Elevator Cab Unit configured in an open configuration.

FIG. 92 shows a top view of an example embodiment of a Germpass Retrofit Elevator Cab Unit configured in an open configuration.

FIG. 93 shows a bottom view of an example embodiment of a Germpass Retrofit Elevator Cab Unit configured in an open configuration.

FIG. 94 shows a rear view of an example embodiment of a Germpass Retrofit Elevator Cab Unit configured in an open configuration.

FIG. 95 shows a perspective view of an example embodiment of a Germpass Lever Handle Unit configured in a closed configuration.

FIG. 96 shows a perspective view of an example embodiment of a Germpass Lever Handle Unit configured in an open configuration.

FIG. 97 shows a front view of an example embodiment of a Germpass Lever Handle Unit configured in a closed configuration.

FIG. 98 shows a perspective view of an example embodiment of a Germpass Lever Handle Unit configured in a closed configuration.

FIG. 99 shows a left view of an example embodiment of a Germpass Lever Handle Unit configured in a closed configuration.

FIG. 100 shows a right view of an example embodiment of a Germpass Lever Handle Unit configured in a closed configuration.

FIG. 101 shows a top view of an example embodiment of a Germpass Lever Handle Unit configured in a closed configuration.

FIG. 102 shows a bottom view of an example embodiment of a Germpass Lever Handle Unit configured in a closed configuration.

FIG. 103 shows a rear view of an example embodiment of a Germpass Lever Handle Unit configured in a closed configuration.

FIG. 104 shows a front view of an example embodiment of a Germpass Lever Handle Unit configured in an open configuration.

FIG. 105 shows a perspective view of an example embodiment of a Germpass Lever Handle Unit configured in an open configuration.

FIG. 106 shows a left view of an example embodiment of a Germpass Lever Handle Unit configured in an open configuration.

FIG. 107 shows a right view of an example embodiment of a Germpass Lever Handle Unit configured in an open configuration.

FIG. 108 shows a top view of an example embodiment of a Germpass Lever Handle Unit configured in an open configuration.

FIG. 109 shows a bottom view of an example embodiment of a Germpass Lever Handle Unit configured in an open configuration.

FIG. 110 shows a rear view of an example embodiment of a Germpass Lever Handle Unit configured in an open configuration.

FIG. 111 shows a perspective view of an example embodiment of a Germpass Restroom Stall Latch Unit configured in a closed configuration.

FIG. 112 shows a perspective view of an example embodiment of a Germpass Restroom Stall Latch Unit configured in an open configuration.

FIG. 113 shows a front view of an example embodiment of a Germpass Restroom Stall Latch Unit configured in a closed configuration]

FIG. 114 shows a perspective view of an example embodiment of a Germpass Restroom Stall Latch Unit configured in a closed configuration.

FIG. 115 shows a left view of an example embodiment of a Germpass Restroom Stall Latch Unit configured in a closed configuration.

FIG. 116 shows a right view of an example embodiment of a Germpass Restroom Stall Latch Unit configured in a closed configuration.

FIG. 117 shows a top view of an example embodiment of a Germpass Restroom Stall Latch Unit configured in a closed configuration.

FIG. 118 shows a bottom view of an example embodiment of a Germpass Restroom Stall Latch Unit configured in a closed configuration.

FIG. 119 shows a rear view of an example embodiment of a Germpass Restroom Stall Latch Unit configured in a closed configuration.

FIG. 120 shows a front view of an example embodiment of a Germpass Restroom Stall Latch Unit configured in an open configuration.

FIG. 121 shows a perspective view of an example embodiment of a Germpass Restroom Stall Latch Unit configured in an open configuration.

FIG. 122 shows a left view of an example embodiment of a Germpass Restroom Stall Latch Unit configured in an open configuration.

FIG. 123 shows a right view of an example embodiment of a Germpass Restroom Stall Latch Unit configured in an open configuration.

FIG. 124 shows a top view of an example embodiment of a Germpass Restroom Stall Latch Unit configured in an open configuration.

FIG. 125 shows a bottom view of an example embodiment of a Germpass Restroom Stall Latch Unit configured in an open configuration.

FIG. 126 shows a rear view of an example embodiment of a Germpass Restroom Stall Latch Unit configured in an open configuration.

FIG. 127 shows a perspective view of an example embodiment of a Germpass Crashbar Unit configured in a closed configuration.

FIG. 128 shows a perspective view of an example embodiment of a Germpass Crashbar Unit configured in an open configuration.

FIG. 129 shows an example flow diagram of an Elevator Cab Button Panel Germpass Unit Decontamination Procedure 1200.

FIG. 130 shows an example flow diagram of a Germpass Unit User Intent Interpretation Procedure 1300.

FIG. 131 shows an example graph illustrating various interpretations of an object's movement(s) of an object as a function of distance versus time measurements.

FIG. 132 shows a first example graph of Germpass Unit sensor data measuring a person's movements as a function of distance vs time measurements, where the person is moving in a first direction which is relatively parallel to the Germpass Unit.

FIG. 133 shows a second example graph of Germpass Unit sensor data measuring a person's movements as a function of distance versus time measurements, where the person is standing still.

FIG. 134 shows a third example graph of Germpass Unit sensor data measuring a person's movements as a function of distance versus time measurements, where the person is moving in a third direction toward Germpass Unit.

FIG. 135 shows a fourth example graph of Germpass Unit sensor data measuring a person's movements as a function of distance versus time measurements, where the person is moving in a fourth direction away from the Germpass Unit.

FIGS. 136 and 137 show different perspective views of a Germpass Elevator Hall Call Unit movable access panel 4920 in accordance with at least one embodiment.

FIGS. 138 and 139 show different perspective views of a Germpass Retrofit Elevator Cab Unit movable intermediate access panel 7920 in accordance with at least one embodiment.

FIGS. 140 and 141 show different perspective views of a Germpass Retrofit Elevator Cab Unit movable end access panel 7922 in accordance with at least one embodiment.

FIGS. 142 and 143 show different perspective views of a Germpass Lever Handle Unit movable access panel 9520 in accordance with at least one embodiment.

Throughout the drawings and the detailed description, the same reference numerals can refer to the same elements. The drawings may not be to scale, and the relative size, proportions, and depiction of elements in the drawings may be exaggerated for clarity, illustration, and convenience.

### SPECIFIC EXAMPLE EMBODIMENTS

Various techniques will now be described in detail with reference to a few example embodiments thereof as illustrated in the accompanying drawings. In the following description, numerous specific details are set forth in order to provide a thorough understanding of one or more aspects and/or features described or reference herein. It will be apparent, however, to one skilled in the art, that one or more aspects and/or features described or referenced herein may be practiced without some or all of these specific details. In other instances, well known process steps and/or structures have not been described in detail in order to not obscure some of the aspects and/or features described or referenced herein.

One or more different inventions may be described in the present application. Further, for one or more of the invention(s) described herein, numerous embodiments may be described in this patent application, and are presented for illustrative purposes only. The described embodiments are not intended to be limiting in any sense. One or more of the invention(s) may be widely applicable to numerous embodiments, as is readily apparent from the disclosure. These embodiments are described in sufficient detail to enable those skilled in the art to practice one or more of the invention(s), and it is to be understood that other embodiments may be utilized and that structural, logical, software, electrical and other changes may be made without departing from the scope of the one or more of the invention(s). Accordingly, those skilled in the art will recognize that the one or more of the invention(s) may be practiced with various modifications and alterations. Particular features of one or more of the invention(s) may be described with reference to one or more particular embodiments or figures that form a part of the present disclosure, and in which are shown, by way of illustration, specific embodiments of one or more of the invention(s). It should be understood, however, that such features are not limited to usage in the one or more particular embodiments or figures with reference to which they are described. The present disclosure is neither a literal description of all embodiments of one or more of the invention(s) nor a listing of features of one or more of the invention(s) that must be present in all embodiments.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. To the contrary, a variety of optional components are described to illustrate the wide variety of possible embodiments of one or more of the invention(s).

Further, although process steps, method steps, algorithms or the like may be described in a sequential order, such processes, methods and algorithms may be configured to work in alternate orders. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the invention(s), and does not imply that the illustrated process is preferred.

When a single device or article is described, it will be readily apparent that more than one device/article (whether or not they cooperate) may be used in place of a single device/article. Similarly, where more than one device or article is described (whether or not they cooperate), it will be readily apparent that a single device/article may be used in place of the more than one device or article.

The functionality and/or the features of a device may be alternatively embodied by one or more other devices that are not explicitly described as having such functionality/features. Thus, other embodiments of one or more of the invention(s) need not include the device itself.

Techniques and mechanisms described or referenced herein will sometimes be described in singular form for clarity. However, it should be noted that particular embodiments include multiple iterations of a technique or multiple instantiations of a mechanism unless noted otherwise.

The terminology used herein is for the purpose of describing particular embodiments only and is not intended to be limiting of the invention. As used herein, the term “and/or” includes all combinations of one or more of the associated listed items. As used herein, the singular forms “a,” “an,” and “the” are intended to include the plural forms as well as the singular forms unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification should be read with the understanding that such combinations are entirely within the scope of the invention.

New methods and devices to decontaminate germs from human touch point inanimate objects, hereafter referred to as “fomites”, and to seal the fomite from airborne pathogens between use to prevent the spread of infectious diseases are discussed herein. For the purpose of the present invention, examples of fomites include but are not limited to door handles, restroom stall latches, deadbolts, gas pump handles, retail point-of-sale (POS) terminals, automatic teller machines, shopping cart handles, elevator control panels, public telephones, paper towel extraction levers, toilet handles and seats, and the like. In the following description, for the purposes of explanation, numerous specific details are set forth to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or descriptions below.

In accordance with an embodiment, a method of sterilizing and sealing a human touch point fomite is provided. A device comprising a solid exterior shell is adjacent with or attaches to the fomite and forms a chamber to seal the fomite from airborne pathogens. The shell comprises a cutout in the front and an open rear. The front cutout is optimally located in front of the touch point and is sealed with one or more retractable panels which, when retracted, provide a user access to the fomite. The rear of the exterior shell is scaled either by a baseplate or directly to the structure to which the fomite is attached. After each use, a germicidal process is completed to kill/inactivate the microorganisms; subsequently, the device remains scaled, only opened upon user-detection through sensor technology, to prevent airborne pathogens from attaching to the fomite between use.

In accordance with an embodiment, an apparatus (also referred to as “device” or “chamber”) configured to decontaminate and seal human touch point fomites from germs is provided. The device comprises a solid exterior front shell which is positioned over the fomite and secured either adjacent with or attached to the fomite to form a sealed chamber. The exterior shell features an opening in front of the fomite, which is scaled by one or more retractable panels, and another in the rear which is fully or partially surrounded by a baseplate. The interior of the chamber is coated with a UV-C reflective material such as aluminum foil. PTFE, UV-reflective paint, or any similar substance proven to maximize UV reflectivity. The chamber interior also comprises one or a plurality of ultraviolet-C wavelength LED semiconductor chips (hereafter referred to as “UV-C”, “UV-C source”, or “chips”), optimally mounted at a fixed or adjustable angle to the baseplate, and/or upper housing assembly including the retractable panels to ensure proper coverage and the most effective placement, surround the fomite to kill adjacent germs within seconds after each interaction with a user through ultraviolet germicidal irradiation (herein also referred to as “UVGI”). In this embodiment, the UV-C is/are preferably delivering their dose at an optimal wavelength of 265 nm. The device remains sealed after the UVGI cycle to prevent airborne pathogens from contaminating the fomite between users. Upon detection of a user through sensor technology, the access panels retract to provide unimpeded access to the germ-free fomite, then are closed after use to perform the UVGI cycle and seal the fomite from airborne pathogens once again.

In accordance with certain embodiments, the device may be comprised of a single alternative UV-C wavelength in place of 265 nm, such as far UV-C ranging between 207-222 nm, to target a specific germ or germs which may be optimally inactivated at alternative wavelengths. In accordance with certain embodiments, the device may be comprised of a multi-wavelength UV-C array within the chamber to target varying classes of germs which are optimally inactivated at alternative wavelengths. For example, some protein-based germs are optimally killed at 220 nm instead of 265 nm while others may be more susceptible to wavelengths of 280 nm. In accordance with certain embodiments, the chamber of the device may be comprised of ozone-producing UV operating at a wavelength of 185 nm which can be deployed in conjunction with non-ozone-producing UV-C or as a stand-alone germicidal solution. In accordance with certain embodiments, the UV light source within the chamber could be LED, pulsed-xenon, low-pressure mercury, or any other suitable UV light delivery format. In accordance with certain embodiments, the device may be comprised with a single self-contained housing without a rear baseplate.

In accordance with certain embodiments, a germ decontamination system is also provided comprising any of the germ decontamination apparatuses described herein configured to be integrated into an apparatus or product comprising a fomite. For the purpose of the present invention, examples of an apparatus or product comprising a fomite include but are not limited to a door, a restroom stall, a deadbolt, a gas pump, a retail point of-sale (POS) terminal, an automatic teller machine, a shopping cart, an elevator, a public telephone, a paper towel dispenser, a computer keyboard, a toilet, and the like.

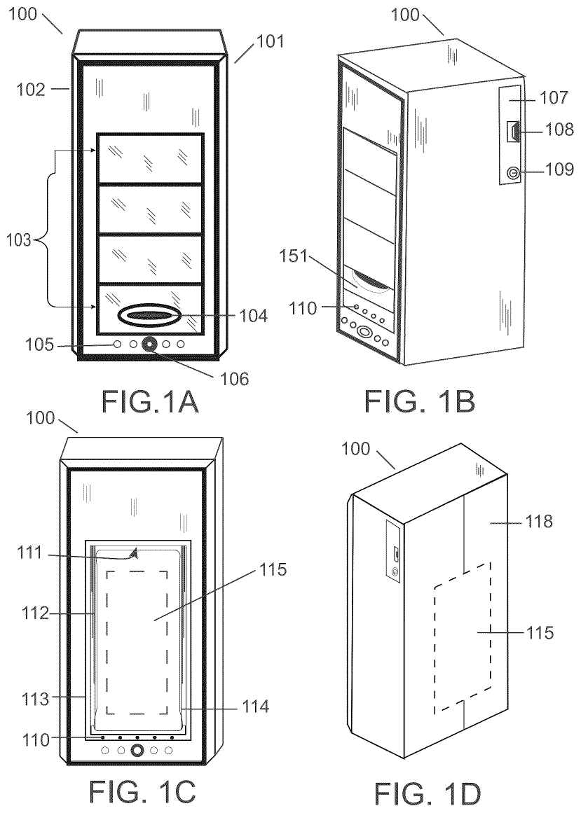

The present invention will now be described by referencing the appended figures
representing preferred embodiments. FIGS. 1A-17E describe an embodiment of a germ decontamination chamber for use with a broad spectrum of human touch point fomites.

FIGS. 1A-1D present a front view, side perspective view, front perspective view, and elevated rear perspective view, respectively, of the germ decontamination chamber (herein also referred to as “chamber” or “device”) 100 for human touch point fomites 115. FIG. 1A presents a front view of the exterior of a germ decontamination chamber 100 and identifies the exterior elements of the upper housing assembly 101 (herein also referred to as “UHA”). The chassis 102 is the outer shell of the chamber 100 which comprises a center cutout to provide frontal access to the fomite 115 and an open rear area to allow it to be positioned over the fomite 115 (FIG. 1D) and adjacent to the baseplate assembly 118 at the rear of the chamber 100 (FIG. 1D). The chassis 102 can be constructed of plastic, aluminum, carbon fiber, fiberglass, or any other suitable material. Behind the cutout, the access panel group 103 comprising an aggregation of four access panels for the device 100 (herein also referred to as “access panels” or “panels”) is positioned to seal the front of the chamber 100 during the ultraviolet germicidal irradiation (herein referred to as “UVGI” or “UVGI cycle”) cycle and when access is not required to prevent re-contamination through airborne microorganisms.

FIG. 1A also depicts an embedded emergency handle 104 located in proximity to the bottom of the access panels 103 to raise and lower them in the event of power loss or mechanical malfunction. An access sensor 106 is located below the access panels 103 to identify the presence of a user and trigger the opening of the access panels 103. Two chamber status lights 105 are located on each side of the access sensor to visually report system readiness, i.e., power on, UVGI progress,
malfunction, and battery status.

FIG. 1B, portrays a side perspective view of the chamber 100 with access panel #4 151 (herein also referred to as the “drive panel”) partially retracted to reveal the obstruction sensors 110 as are also exhibited in FIG. 1C. The right side of the chamber is comprised of the battery access door 107, battery release latch 108 (herein also referred to as “battery latch”). and battery lock 109. In preferred embodiments, the battery 126 can be lithium nickel manganese cobalt oxide (Li-NMC), lithium ion (“Li-ION), or any other long-lasting type which will optimize the performance of the chamber. In certain embodiments, the device can be powered by AC connection, wireless, solar, or any other means which will provide sufficient power for which it to operate.

FIG. 1C depicts a front view of the chamber 100 with the access panels 103 raised into the panel bay 111 (not visible) revealing the peripheral components of the access panel assembly 139 (herein also referred to as “AP assembly”) comprising the access panel frame 113 (herein also referred to as “AP frame”), embedded panel rails 112 (herein also referred to as “rails” or individually as “rail”), support bridge 114, and the fomite 115 as indicated by the rectangular broken lines. FIG. 1C additionally identifies the location of the obstruction sensors 110 which detect the presence of a user or foreign object during the closing of the access panels 103, prompting the chamber 100 to reverse the closing procedure and retract the access panels 103 into the panel bay 111. The rear of the chamber 100 is presented in the elevated view of FIG. 1D comprising the baseplate assembly 118 and an example of a fomite 115 demarcated by the rectangular broken lines.

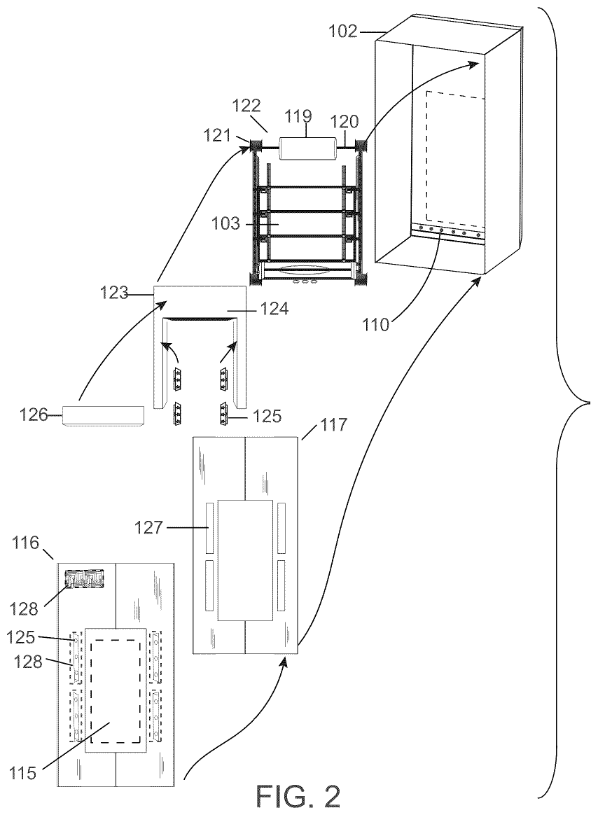

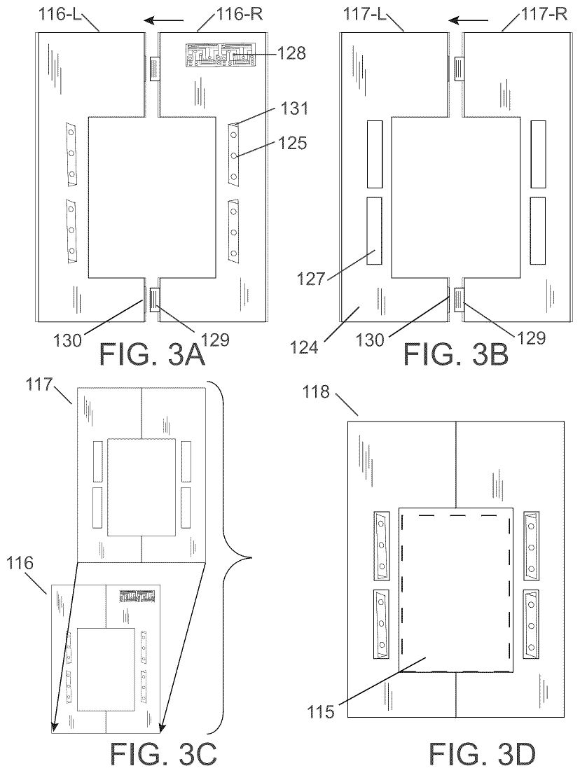

Now referring to FIG. 2, an elevated rear perspective exploded view exhibits the chamber's 100 main components comprised within the upper housing assembly 101 and the baseplate assembly 118. Viewing from the upper right diagonally to the lower left, a rear view of the chassis 102 is exemplified. A drive assembly 122 comprising the drive motor 119, drive shaft 120 (herein also referred to as “shaft”), pulley 121, and access panels 103 is mounted to align the access panels 103 to the front center opening of the chassis 102. The u-shaped shroud 123 comprising a UV-reflective coating 124, is secured over the drive motor 119 and the shroud's legs 123 extend to cover the sides of the drive assembly 122. UV-C 125 is mounted adjacent to the vertical arms of the shroud, the position of which delivers a direct UVGI dose to the front and/or sides of the fomite 115. In certain embodiments, UV-C 125 may be mounted in alternate locations within the upper housing assembly 101 including on the rear of the access panels 103 facing the fomite 115 to deliver the optimal UVGI dose. The battery 126 is secured behind the top of the shroud 123 to complete the major components of the upper housing assembly 101. The baseplate cover 117 comprised of UV-C cutouts 127 attaches to the rear of the upper housing assembly 101 followed by the baseplate 116, comprised of a microcontroller 128 and UV-C 125, which is positioned adjacent to the fomite 115. The combined baseplate cover 117 and baseplate 116 form the baseplate assembly 118 as shown in FIG. 3D.

Now referring to FIGS. 3A-D. FIG. 3A and 3B depict a front view of the baseplate 116 and baseplate cover 117, respectively, each separated into two sides, L&R. The right side of the baseplate 116-R and baseplate cover 117-R each comprise top and bottom interlocking male tabs 129 which connect to the female tab receivers 130 on the left side (collectively referred to herein as “interlocking tabs”) of the baseplate 116-L and baseplate cover 117-L. This allows the baseplate 116 and baseplate cover 117 to be mounted adjacent to the base of the fomite 115 as a single combined unit to form the baseplate assembly 118 as shown in FIG. 3D.

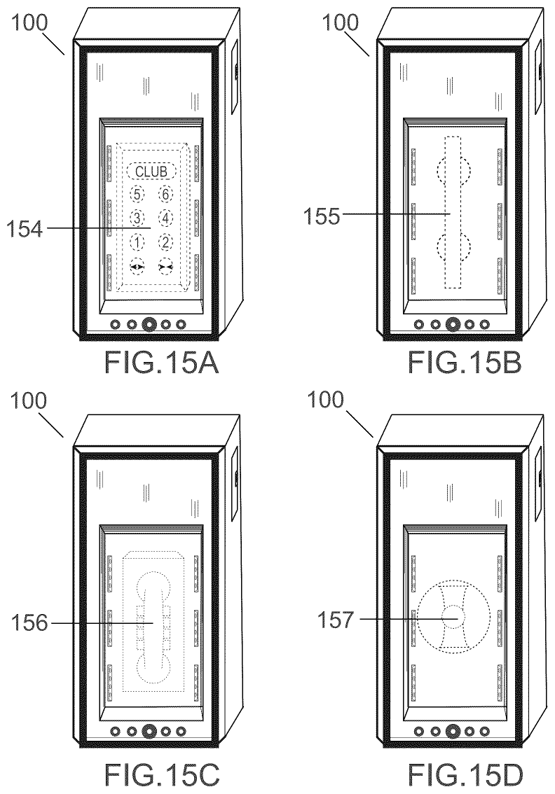

Returning to FIG. 3A, the baseplate 116 comprises a microcontroller 128 to manage the power, sensors, mechanical, and all programmatic functions of the chamber 100. In the preferred embodiment, the baseplate 116 also comprises one or more UV-C LED chip(s) 125 (herein also referred to as “UV-C”, “UV-C source”, or “chip(s)”) which is/are embedded within or affixed to an adhesive strip which is secured to the adjustable UV-C mounting stand 131. The UV-C mounting stand 131 is then attached adjacent to the baseplate 116. The UV-C 125 affixed to the baseplate allows the wavelength to be directed toward the rear and sides of those fomites 115 which receive some or all human contact in those areas instead of the front, such as door handles 155 (FIG. 15B) which receive a small percentage of contact to the front or face. In an alternative embodiment, the UV-C mounting stand 131 is eliminated allowing the UV-C 125 to be affixed directly to the baseplate 116.

Continuing to reference the UV-C 125 in FIG. 3A, the preferred embodiment is for the UV-C LED's 125 to perform precisely at 265 nm, universally recognized as the optimal wavelength for ultraviolet sterilization. Although virtually all germs are proven to be inactivated by UV-C 125 at a wavelength of 265 nm, the optimal wavelength for some protein-based germs is 220 nm while others are most quickly inactivated closer to 280 nm. Therefore, an alternative embodiment calls for a multi-wavelength or multimodal UV-C 125 array deployed throughout the inside of the chamber 100 and delivered in a pulse-format to specifically target certain classes of germs.

As depicted in FIG. 3B, the baseplate cover 117 is coated with a UV-reflective material or substance 124 such as PTFE reflectors, paint, aluminum foil or any other material or coating proven to enhance UV reflectivity. The baseplate cover 117 has UV-C cutouts 127 which are positioned directly above the UV-C 125 on the baseplate 116. In the preferred embodiment, the UV-C cutouts 127 are not covered, however, in certain embodiments they may be covered with a suitable translucent material to
hermetically seal the UV-C 125 as required by the application.

The baseplate cover 117 overlays the baseplate 116, as shown in the front exploded projection view of FIG. 3C, and they collectively form the baseplate assembly 118, as shown in FIG. 3D. The baseplate 116 and baseplate cover 117 can be constructed of plastic, metal, or any other suitable material. While this is the preferred embodiment, alternative embodiments could be deployed to achieve the desired result such as a one-piece baseplate 116 and cover with a hollow core to allow placement through the fomite 115, a baseplate 116 without a cover, a single integrated baseplate 116 and cover, or other embodiments not mentioned herein.

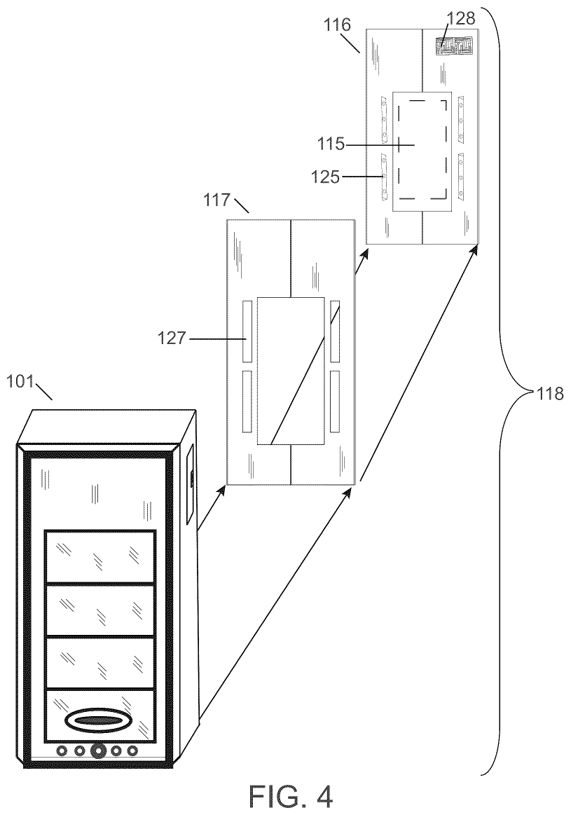

Now referring to FIG. 4, an exploded front view of the upper housing assembly 101 (“UHA ”) projected upon the baseplate cover 117 and baseplate 116 is further detailed. The baseplate 116 with UV-C 125 and microcontroller 128 are mounted adjacent to a fomite 115 and the baseplate cover 117 with UV-C cutouts 127 attach to the baseplate 116 which collectively form the baseplate assembly 118 as depicted in FIG. 3D. The upper housing assembly 101 is then placed over the fomite 115 and secured to the baseplate assembly 118 to operationalize the chamber 100. In alternative embodiments, the upper housing 101 and baseplate assembly 118 are pre-assembled allowing the chamber 100 to be attached to the fomite 115 in one piece.

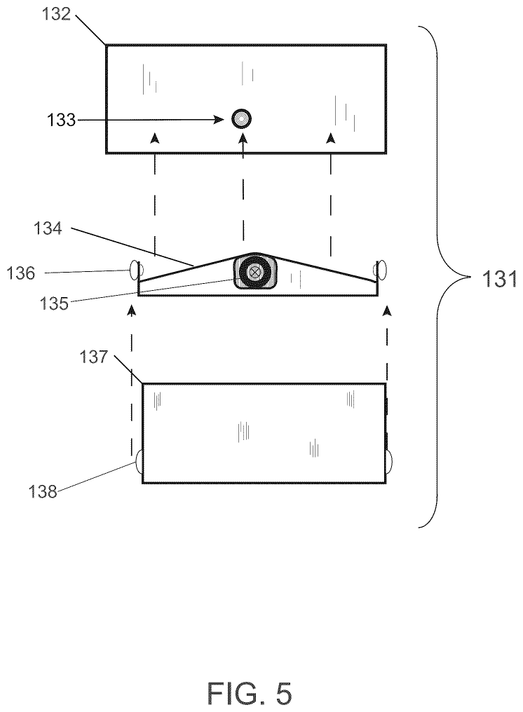

FIG. 5 exhibits a top exploded view of the UV-C mounting stand 131 comprising the mounting base 132, pivot plate 134, and the UV-C mounting tray 137 (herein also referred to as “UV-C tray”, “tray”, or “mounting tray”). As indicated by the broken line projection arrows, the pivot plate 134 connects to the mounting base 132 with the pivot plate screw and washer 135 being placed through the center of the pivot plate and into the threaded screw receiver 133 in the mounting base 132 allowing the pivot plate 134 to swivel horizontally. The UV-C tray 137 attaches to parallel and oppositely disposed pivot plate hinges 136 on each side of the pivot plate 134 using the mounting tray hinge screws 138 allowing the UV-C tray 137 to pivot forward and backward. With the UV-C mounting stand 131 affixed to the baseplate 116, the UV-C 125 can be positioned to deliver the UV-C dose at the optimal direction and angle to most efficiently perform its UVGI function.

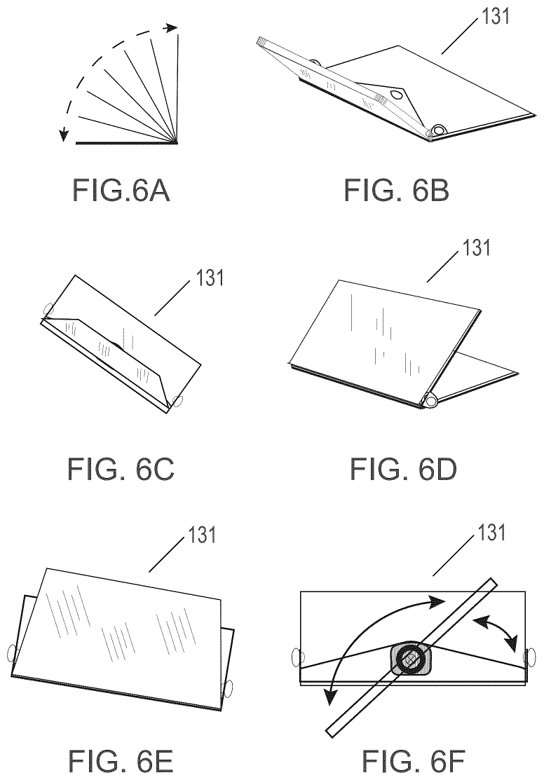

FIGS. 6A-F show the directional and angular flexibility provided by the UV-C mounting stand 131. FIG. 6A reveals a side view of pivot angles ranging from 15°-90° in 15° increments. FIG. 6B shows a front perspective view of a 30° forward angle, FIG. 6C displays a top view of a 75° tilt angle, FIG. 6D presents a front perspective view of a 45° tilt angle, FIG. 6E illustrates a front perspective view of a 15° tilt angle, and FIG. 6F depicts a top view of the swivel range of the mounting stand 131.

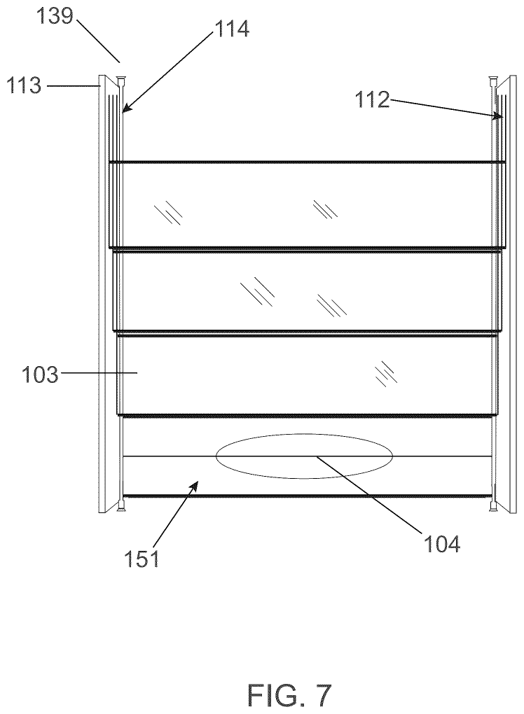

Now referring to FIG. 7, a front closeup view of the access panel assembly 139 (herein
also referred to as “AP assembly”) is depicted. The AP assembly 139 comprises the access panel group (herein also referred to as “access panels”) 103 adjacent to the access panel frame 113 (herein also referred to as “AP frame”), panel rails 112, and support bridge 114 positioned parallel and oppositely disposed to form a left and right side of the AP assembly 139.

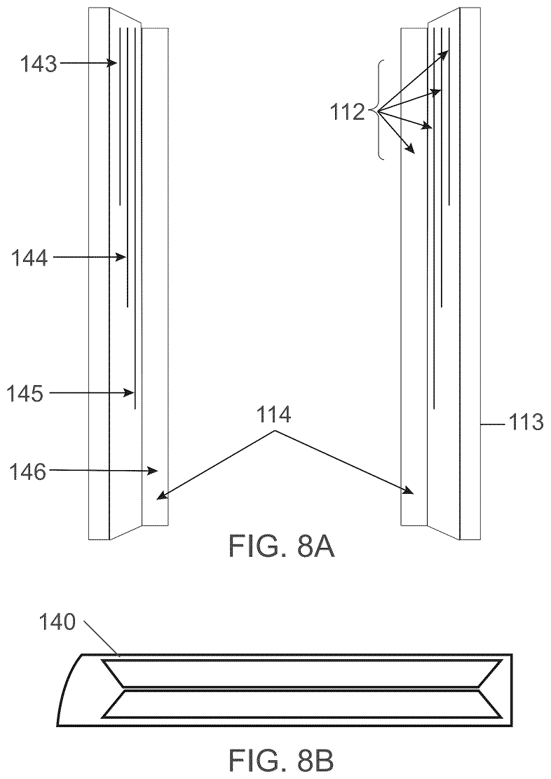

FIG. 8A shows a top view of the parallel and oppositely disposed access panel frame 113, panel rails 112, and support bridge 114 on the left and right side. Referring to the access panel frame 113, there are 3 embedded panel rails 112 exhibited, each lined with a nylon glide 140 as illustrated in FIG. 8B to improve the sliding action and reduce friction during movement of the access panels 103. The 4th rail in this four-panel embodiment is the opening created between the base of the access panel frame 113 identified as the support bridge 114 and the bottom edge of the 3rd embedded panel rail.

In addition to the panel rails 112 being identified as a component group, each individual panel rail is identified in FIG. 8A individually ranging from rail #1-4 in this four-panel embodiment. Reviewing the left side of the illustration from the top rail to the bottom (illustrated as left to right), rail #1 143 in this four-panel configuration travels from its panel bay 111 position to shield the first 25% of the chamber 100 opening. Rail #2 144 shields the 25-50% portion of the chamber 100 opening. Rail #3 145 shields the 50- 75% portion of the opening. Rail #4 145 (herein also referred to as the “drive rail”) is the opening created between the base of the access panel frame 113 identified as the support bridge 114 and the bottom edge of panel rail #3 145 and shields the 75-100% (bottom) of the chamber 100 opening to finalize the closure and scaling of the chamber 100.

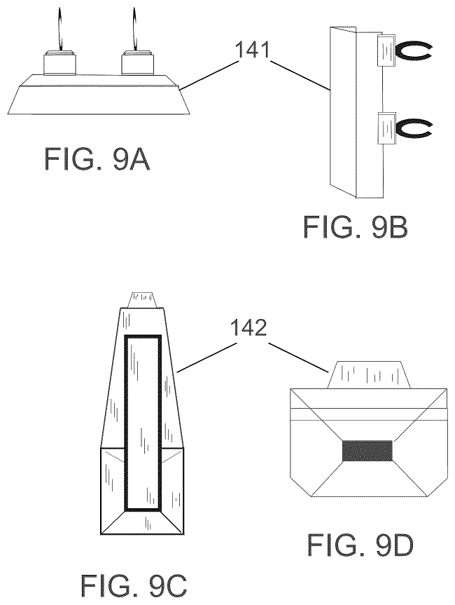

FIGS. 9A and 9B show a top and side view respectively of the drive clip 141. A drive clip 141 attaches to (or optionally is molded within) the outer right and left portion of the drive panel 151 and its circular forks attach to the drive chain 147 which moves the drive panel 151 in each direction. The flat base of the drive clip 141 travels on the protruding edge of the AP frame 113 referred to herein as the support bridge 114, abutting against the panel support arms 142 during retraction to insure each of the panels 103 maintain synchronization and stability.

FIGS. 9C and 9D present an elevated side view and side closeup view respectively of the support arm 142 which attaches to (or optionally molded within) the outer left and right sides of each individual access panel within the AP group 103. The base of the support arm 142 moves laterally along the support bridge 114 to stabilize and maintain synchronization of the access panels 103. During retraction, the support arms 142 arc pushed by the drive clip as they are moved, stacked, and parked in the panel bay 111.

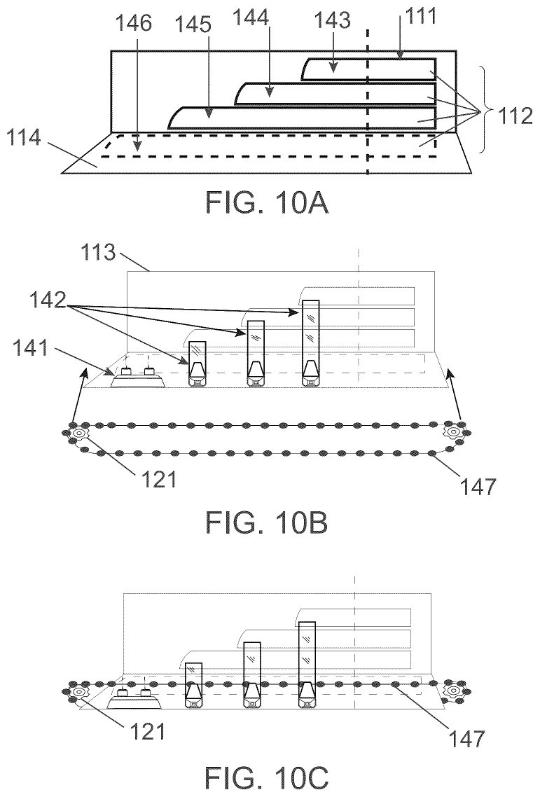

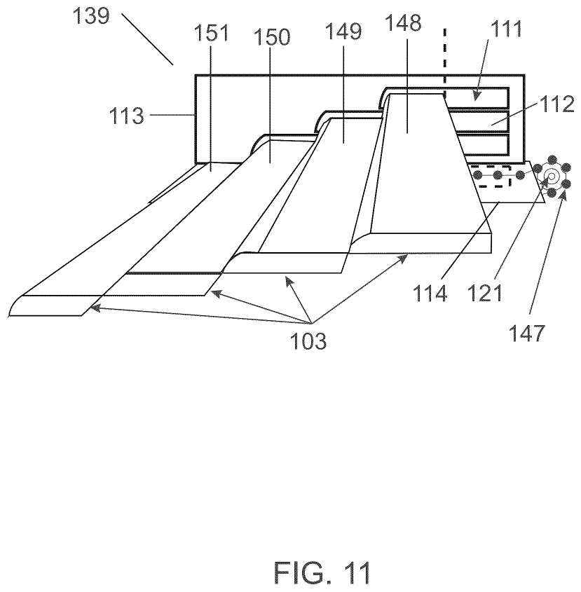

FIGS. 10A-10C exhibit a side sectional view of the left side access panel frame 113, panel rails 112, and support bridge 114 (supported by a depiction of the attached panels 103 in FIG. 11). FIG. 10A, as also referenced in the top view from FIG. 8A, identifies rail #1 143 as the top rail and serves as the side support for access panel #1 148 (FIG. 11) which is responsible for sealing the top 25% of the chamber 100 when the panels 103 are fully closed. Rail #2 144 is adjacent to access panel #2 149 (FIG. 11) and seals from 25-50%, rail #3 145 is adjacent to access panel #3 150 (FIG. 11) and seals from 50-75%, and rail #4 146 (the drive rail) is adjacent to access panel #4 151 (herein also referred to as the “drive panel”) (FIG. 11) seals from 75-100% of the chamber 100 opening. FIG. 10B extends the detail from 10A by adding a transparent view of the support arms 142 and drive clip 141 extending from their respective panel to the support bridge 114. Additionally, FIG. 10B shows an exploded projection of the pulley 121 and drive chain position 147 relative to the access panel frame 113. FIG. 10C finalizes this view by placing the pulley 121 and drive chain 147 into position for this embodiment. This view is mirrored on the right side of the chamber.

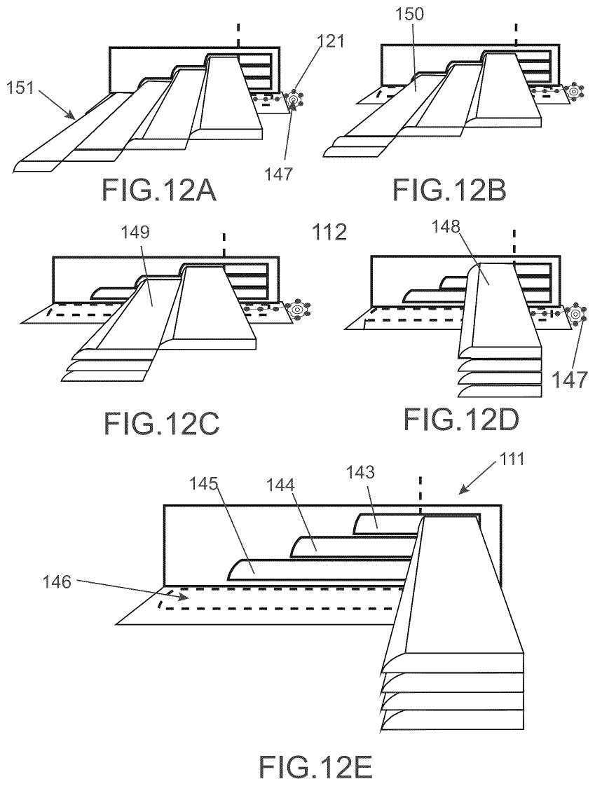

FIG. 11 depicts a side sectional closeup view of the access panel assembly 139 comprising the AP frame 113, support bridge 114, rails 112, and access panels 103 with the access panels 103 in the closed position and each panel connected to their individually dedicated panel rails 112 (previously revealed in FIG. 10A). Viewing the illustration of FIG. 11 from right to left, panel #1 148 is scaling the top 25% of the front of the chamber 100 followed by panel #2 149, panel #3 150, and panel #4 151 (herein also referred to as “drive panel”) which seal the remainder of the chamber 100 in 25% increments in this four-panel embodiment. When retracted, the panels 103 are stacked upon each other and “parked” in the panel bay 111 to reduce the footprint of the chamber 100 outside of the fomite 115 coverage area as clearly illustrated in FIG. 12E. In this embodiment, the pulley 121 is located at each end of the AP frame 113 and the drive chain 147 is looped around the top and bottom of the support bridge 114.

Now referring to the operation of the AP assembly 139 in more detail, FIGS. 12A-E reveals side sectional closeup views of the 5-stages of panel retraction in a four-panel embodiment. FIG. 12A depicts the access panels 103 in the closed position. Panel #4 151 (the drive panel) is positioned at the bottom of the access panels 103 and as it is retracted, it begins to push access panel #3 150 as depicted in FIG. 12B. As panel #3 150 continues to be retracted by the drive panel 151, it captures panel #2 149 as presented in FIG. 12C. The drive panel 151, access panel #3 150, and access panel #2 149 continue to retract in synchronization as they interface with panel #1 148, shown in FIG. 12D where all access panels 103 are stacked upon each other. The chain 147 illustrated in FIG. 12D continues to retract the drive panel 151, causing panel #2 149 to capture panel #1 148 until they are all seated within their respective rail 143, 144, 145, 146 in the panel bay 111 (the panel bay area is demarcated by the broken vertical line in FIGS. 12A-E) as shown in FIG. 12E.

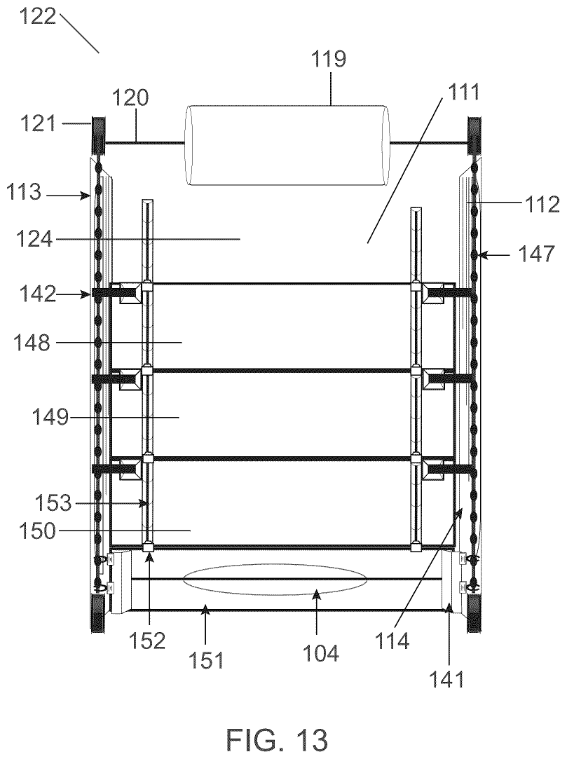

FIG. 13 presents a rear closeup view of the drive assembly 122 comprising the drive motor 119, drive shaft 120, pulley 121, chain 147, access panel frame 113, rails 112, support bridge 114, access panels 103 comprising AP #1-4 148, 149, 150, 151 (referred to individually in this depiction), support arms 142, drive panel 151, drive clip 141, channel guide 153, guide clip 152, panel bay 111, and UV-reflective coating 124. Upon actuation of the drive motor 119, the drive shaft 120 and pulley 121 begin to move the chain 147 and attached drive clip 141 which in turn begins movement of the drive panel 151. Two oppositely disposed guide clips 152 are attached (or embedded into) to the horizontal leading edge of the drive panel 151 and each subsequent access panel 103 with the protruding front edge of the guide clips 152 fitted into the adjacent channel guide 153. During retraction, the two guide clips 152 on the drive panel 151 move vertically within the channel guides 153 of access panel #3 150 and begin to push it toward access panel #2 149. The guide clip 152 on access panel #3 150 and each subsequent access panel travel within their adjacent channel guide 153 to push the adjacent panel in the appropriate direction until the access panels 103 are parked within the panel bay 111. The access panels 103 are stabilized and synchronized during movement by the support arms 142 and base of the drive clip 141 which slide along and are buttressed by the support bridge 114. In alternative embodiments, the drive assembly 122 could be comprised of any mechanism capable of raising and lowering the access panels including but not limited to belts, springs, magnets, and hydraulic, pneumatic, or electrical linear actuators.

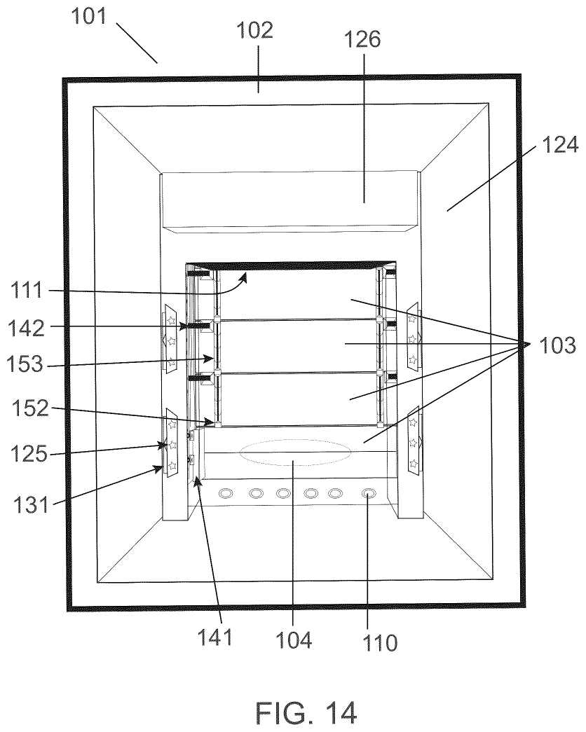

FIG. 14 reveals a rear closeup view of the interior of the upper housing assembly 101. Illustrated components in this view comprise the chassis 102, battery 126, panel bay 111, UV-C 125, UV-C mounting stand 131, support arm(s) 142, channel guide 153, guide clip 152, drive clip 141, emergency handle 104, obstruction sensors 110, access panels 103, and UV-reflective surface 124 (not visible).

FIGS. 15A-D represent a front perspective view of a germ decontamination chamber 100 with the access panels 103 retracted revealing examples showing the positioning of different fomites 115 within the chamber. FIG. 15A depicts a germ decontamination chamber 100 adjacent to an elevator control panel 154. FIG. 15B exhibits a chamber 100 adjacent to an interior vertical bar-style door handle 155 such as those used in theaters and auditoriums. FIG. 15C shows a chamber 100 adjacent to a wall mounted courtesy telephone 156 such as those at airports and hotels. FIG. 15D presents a chamber 100 adjacent to a restroom stall deadbolt handle 157.

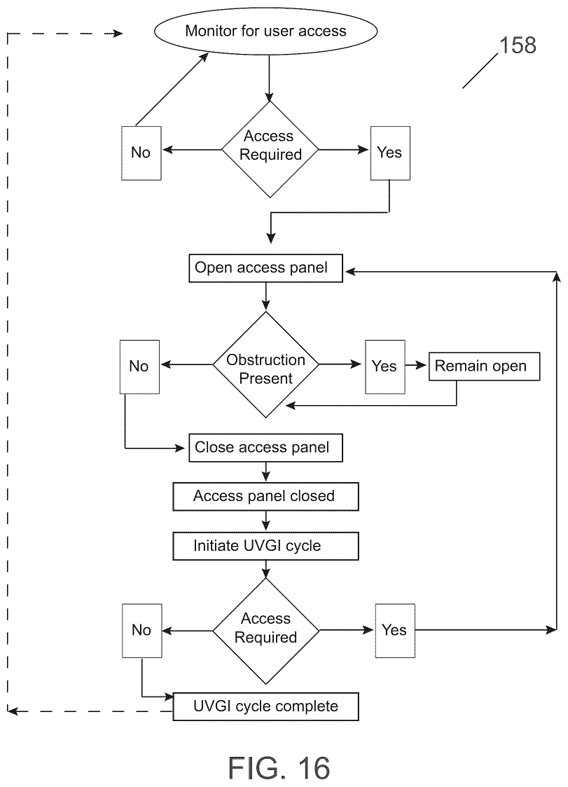

FIG. 16 reveals a flow diagram 158 illustrating one version of the operation of the germ decontamination chamber 100 for use with human touch point fomites 115. In standby mode, an access sensor 106 monitors for the presence of a user, the definition of which varies depending on the application. In certain embodiments, a user may be defined as any person within a defined distance of the chamber 100 i.e. six feet, while in certain other embodiments a user may be defined as someone who has placed their hand within a defined range of the sensor 106 i.e. six inches, while in yet even other embodiments, a user may be defined as someone within range of the chamber 100 possessing a mobile app, key fob, or other similar method or apparatus which is defined as an authorized user by the chamber 100. When a user has been detected, the access panels 103 are retracted and remain open either for a programmed period, until the sensor 106 no longer detects any obstructions, or a combination of both. When the closing criteria has been met, the access panels 103 begin to close and are only stopped in the event of an obstruction or newly defined user in which case the access panels 103 will begin to retract once again. After the device 100 is scaled, a UVGI cycle is initiated. If a user is detected while a cycle is in progress, the cycle stops and the access panels 103 are opened. Once the UVGI cycle is completed, the access panels 103 remain closed to prevent recontamination by airborne pathogens and the device 100 remains in a standby state until the presence of a user is detected.

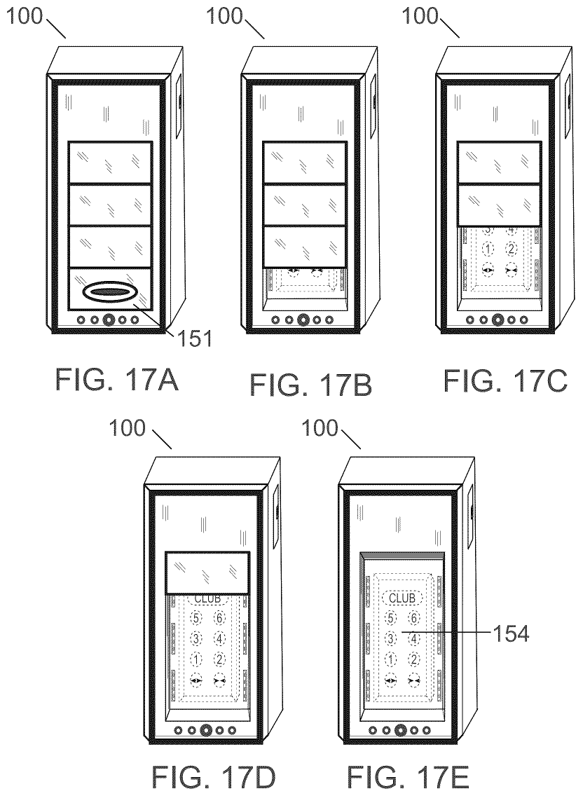

FIGS. 17A-E reveal a front perspective view of a germ decontamination chamber 100 illustrating the 5-stages of panel retraction using an elevator control panel 154 (shown in FIG. 17E) as the fomite 115 example. FIG. 17A shows a closed and scaled chamber 100, FIG. 17B presents a chamber 100 with panel #4 151 (drive panel) retracted behind access panel #3 150, FIG. 17C portrays a chamber 100 50% open, FIG. 17D represents a chamber 100 75% open, and FIG. 17E presents an open chamber 100 revealing a sterilized and germ-free elevator control panel 154.

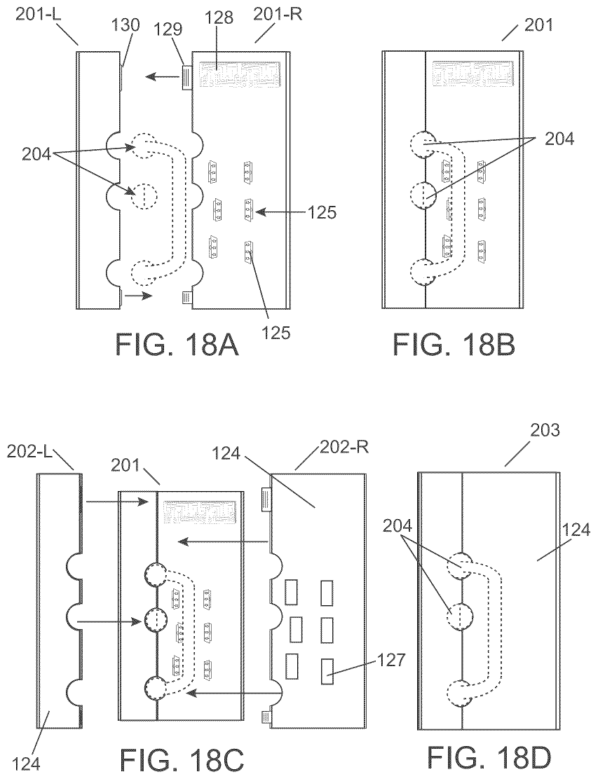

Referring now to an alternative embodiment, FIGS. 18A-20B illustrate a commercial door handle and lock germ decontamination chamber 200 (herein also referred to as “DHL chamber”). This embodiment is unchanged from the invention of FIG. 1A (the germ decontamination chamber) apart from the configuration of the baseplate 116 and baseplate cover 117; therefore, the present depiction is limited to the changes and the resulting embodiment of the invention.

As depicted in the front views of FIGS. 18A-D, the door handle and lock baseplate assembly 203 (herein also referred to as “DHL baseplate assembly”) in this embodiment has form fitted cutouts to conform to the contours of the fomite 115, in this case the commercial door handle and lock 204. FIG. 18A depicts the left and right sides of the two-piece door handle and lock baseplate 201_L, 201-R (herein also referred to as “DHL baseplate”) comprising the microcontroller 128, UV-C 125, and UV-C mounting stand 131 preparing to be fitted adjacent to the base of the handle & lock 203 using the male 129 and female 130 interlocking tabs. The resulting one-piece DHL baseplate 201 is shown in FIG. 18B. FIG. 18C shows the detached left and right sides of the fitted door handle and lock baseplate cover 202-L, 202-R (herein also referred to as “DHL baseplate cover”) comprising a UV-reflective coating 124 and UV-C cutouts 127 preparing to be fitted over the DHL baseplate 201, and FIG. 18D presents the resulting DHL baseplate assembly 203 with UV-reflective coating 124 fitted adjacent to the door handle and lock 204.

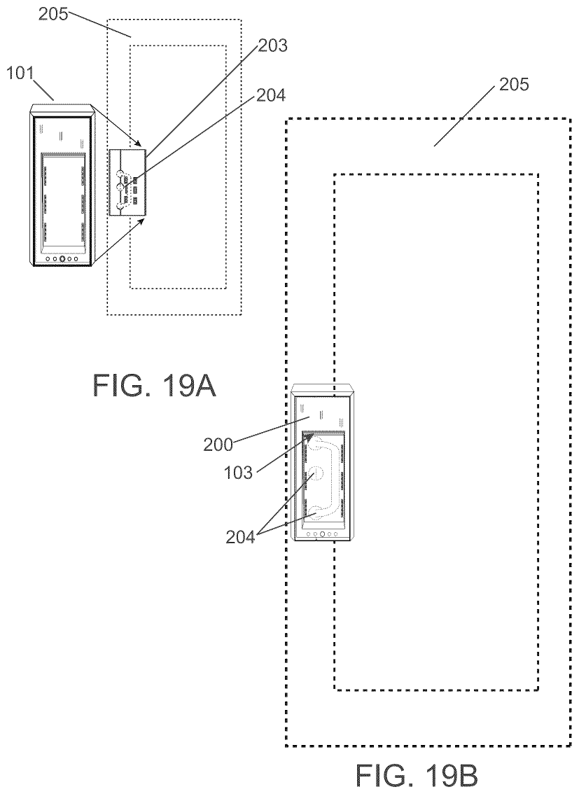

FIG. 19A represents a distant front perspective view of a commercial door 205, handle, and lock 204 with the adjacent fitted DHL baseplate assembly 203 illustrated from FIG. 18D along with an exploded front view of the upper housing assembly 101 projected into position adjacent to the DHL baseplate assembly 203. FIG. 19B presents a front perspective view of the DHL germ decontamination chamber 200 mounted to the commercial door 205 with access panels 103 open revealing the adjacent door handle and lock 204.

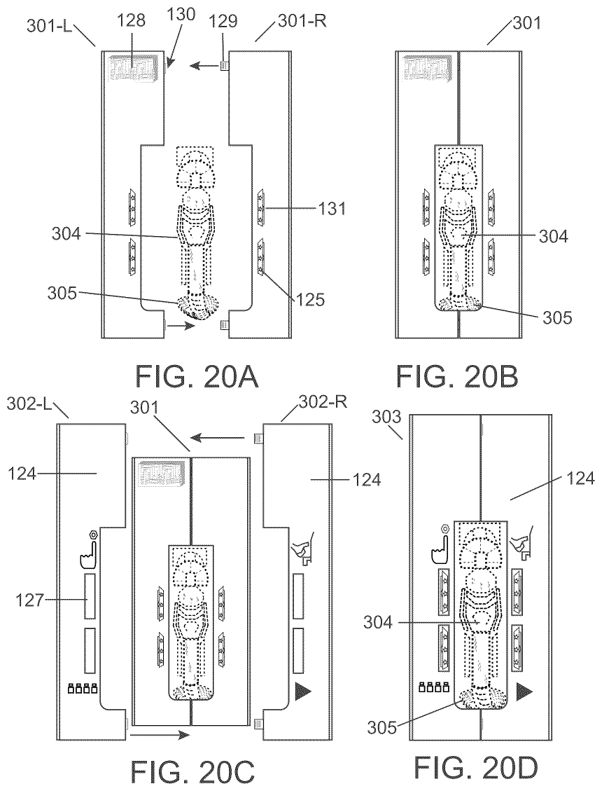

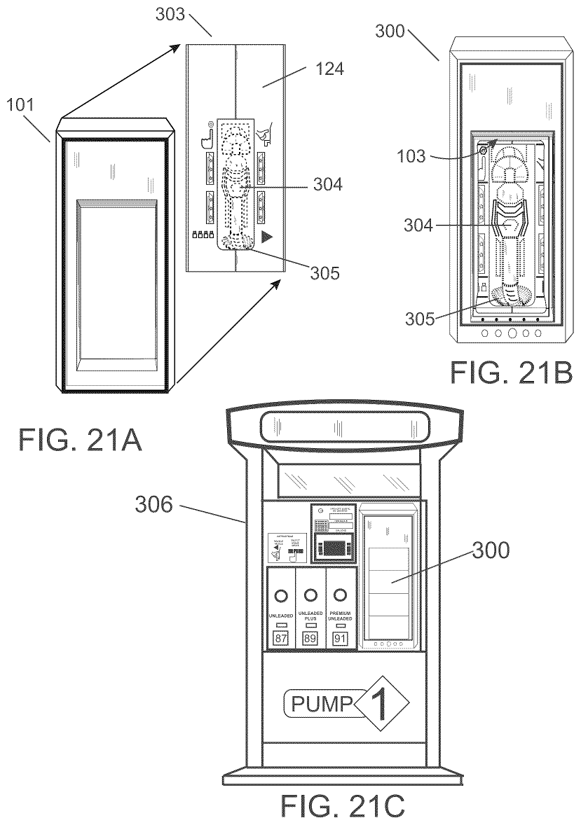

Referring now to another embodiment, FIGS. 20A-21C illustrate a gas pump handle germ decontamination chamber (herein also referred to as “GP chamber”). This embodiment is unchanged from the embodiment of the invention of FIG. 1A (the germ decontamination chamber) apart from the configuration of the baseplate 116 and baseplate cover 117, therefore this depiction is limited to the changes and an illustration of the resulting embodiment of the invention.

As depicted in the front views of FIGS. 20A-D, the gas pump baseplate assembly 303 (herein also referred to as “GP baseplate assembly”) in this embodiment has form fitted cutouts to conform to the contours of the fomite 115, in this case the gas pump handle 304 (herein also referred to as “GP handle” or “gas pump”). FIG. 20A depicts the left and right sides of the two-piece gas pump baseplate 301_L, 301-R (herein also referred to as “GP baseplate”) comprising the microcontroller 128, UV-C 125, and UV-C mounting stand 131 preparing to be fitted adjacent to the base of the gas pump handle 304 and retractable hose well 305 using the male 129 and female 130 interlocking tabs. The resulting one-piece GP baseplate 301 is shown in FIG. 20B adjacent to the GP handle 304 and retractable hose well 305. FIG. 20C shows the detached left and right sides of the fitted gas pump baseplate cover 302-L, 302-R (herein also referred to as “GP baseplate cover”) comprising a UV-reflective coating 124 and UV-C cutouts 127 preparing to be fitted over the GP baseplate 301, and FIG. 20D presents the resulting GP baseplate assembly 303 with UV-reflective coating 124 fitted adjacent to the GP handle 304 and retractable hose well 305.

FIG. 21A represents a distant front perspective view of a gas pump handle 304 and
retractable hose well 305 with the adjacent fitted GP baseplate assembly 303 with UV-reflective coating 124 illustrated from FIG. 20D along with an exploded front view of the upper housing assembly 101 projected into position adjacent to the gas pump handle 304. FIG. 21B presents a front perspective view of the GP germ decontamination chamber 300 with panels 103 open revealing the adjacent gas pump handle 304 and retractable hose well 305. FIG. 21C presents an example of a gas pump handle germ decontamination chamber 300 in the closed position adjacent to a gas pump handle 304 within the context of a gas station service island 306.

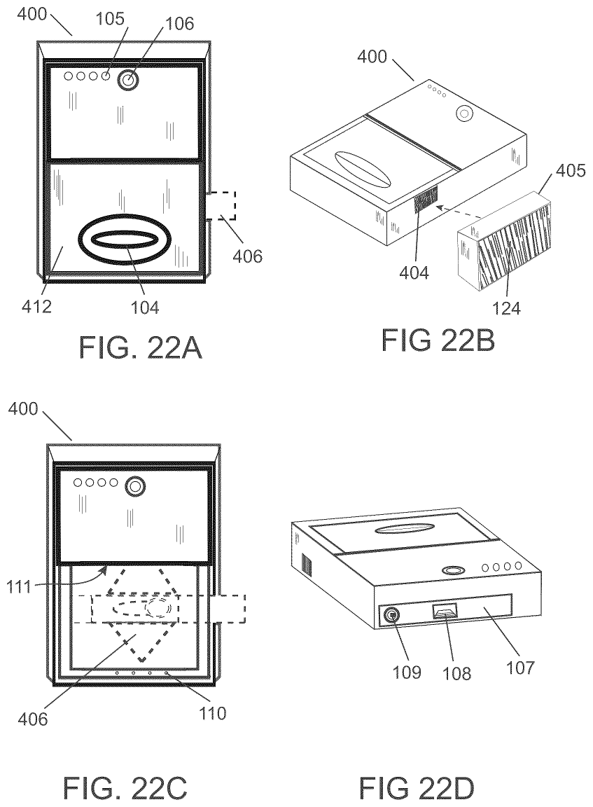

Referring now to another embodiment of the invention, FIG. 22A-D presents a front closed, side perspective, front open, and top perspective view, respectively, of a single-panel germ decontamination chamber in the embodiment of a restroom stall latch germ decontamination chamber 400 (herein also referred to as “RS chamber”). As shown in FIG. 22A, the front of the RS chamber comprises a restroom stall drive panel 412 (access panel) (herein also referred to as “RS drive panel”), an emergency handle 104, access sensor 106, and four system status lights 105 to the left side of the access sensor 106 with the stall latch 406 protruding from the side.

As depicted in FIG. 22B, the elevated side perspective view shows the latch gateway 404 on the side of the RS chamber 400 along with an exploded view of the brush shield 405 being projected into position adjacent to the latch gateway 404. The brush shield 405 frames the latch gateway 404 and is comprised of multiple layers of dense yet flexible fiber to seal the latch gateway 404 while allowing the stall latch 406 (herein also referred to as “latch”) to have freedom of lateral movement. The interior-facing fibers of the brush shield 405 are layered with a UV-reflective coating 124 to promote UV reflectivity while the outer-facing layers are dense enough to prevent light from escaping the interior of the RS chamber 400. In certain other embodiments, the brush shield 405 could be constructed with any material or substance that allows the latch 406 to be laterally moved while continuing to seal the latch gateway 404. In certain other embodiments, the RS chamber 400 might not include a brush shield 405.

FIG. 22C portrays a front view of the RS chamber 400 with the drive panel 412 retracted into the panel bay 111 to provide user access to the latch 406 and identifies the obstruction sensors 110 located in proximity to the bottom of the RS chamber 400. An elevated top perspective view of the RS chamber 400 is seen in FIG. 22D displaying the battery access door 107, battery release latch 108 (herein also referred to as “battery latch”), and battery lock 109 (herein also referred to as “security lock”).

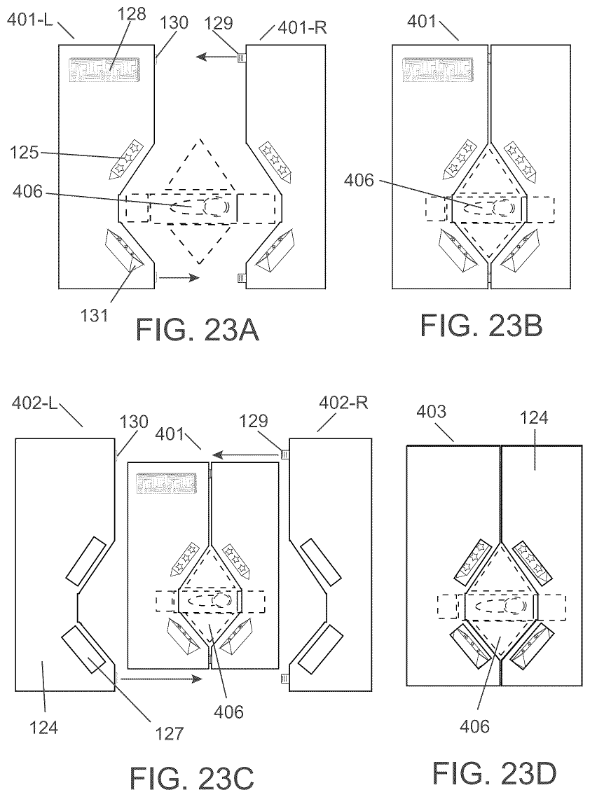

As depicted in the front views of FIGS. 23A-D, the restroom stall latch baseplate assembly 403 (herein also referred to as “RS baseplate assembly”) has form fitted cutouts to conform to the contours of the fomite 115, in this case the restroom stall latch 406 (herein also referred to as “stall latch”). FIG. 23A depicts the left and right sides of the two-piece restroom stall latch baseplate 401 (herein also referred to as “RS baseplate”) comprised of microcontroller 128, UV-C 125, and UV-C mounting stand 131 preparing to be fitted adjacent to the base of the stall latch 406 using the male 129 and female 130 interlocking tabs. The resulting one-piece RS baseplate 401 is shown in FIG. 23B. FIG. 23C presents the left and right sides of the two-piece restroom stall latch baseplate cover 402 (herein also referred to as “RS baseplate cover”) comprising UV-C cutouts 127 and UV-reflective coating 124 projecting to be fitted over the RS baseplate 401 and adjacent to the stall latch 406, and FIG. 23D presents the resulting RS baseplate assembly 403 with UV-reflective coating 124 fitted to the stall latch 406.

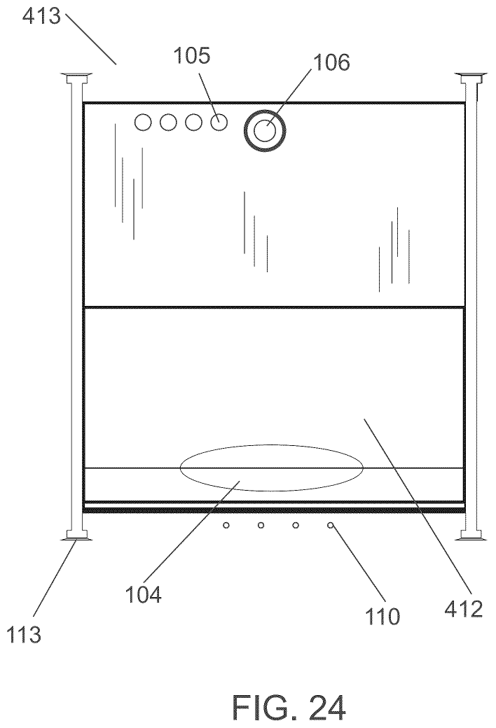

Now referring to FIG. 24, a front closeup view of the restroom stall latch access panel assembly 413 (herein also referred to as “RS access panel assembly”) is exhibited. The RS access panel assembly 413 comprises an access panel frame 113, support bridge 114, and RS drive panel 412 as contrasted with previous multi-panel embodiments which also include rails for the secondary panels. The functionality remains the same as in previous embodiments. The access sensor 106, status lights 105, obstruction sensors 110, and emergency handle 104 are also illustrated.

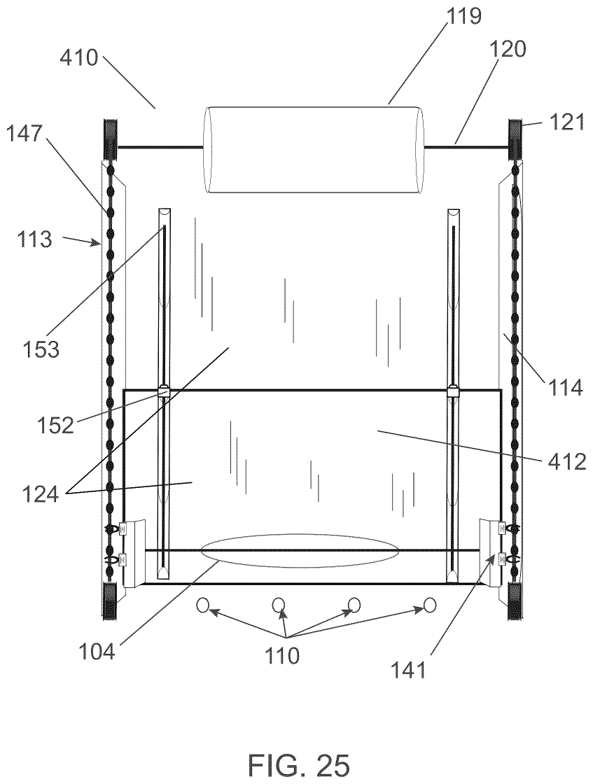

FIG. 25 shows a rear closeup view of the restroom stall latch drive assembly 410 (herein also referred to as “RS drive assembly”) comprising the drive motor 119, drive shaft 120, pulley 121, drive chain 147, access panel frame 113, support bridge 114, drive clip 141, channel guide 153, guide clip 152, obstruction sensors 110, RS drive panel 412, and emergency handle 104. The functionality is the same as the invention of 1A.

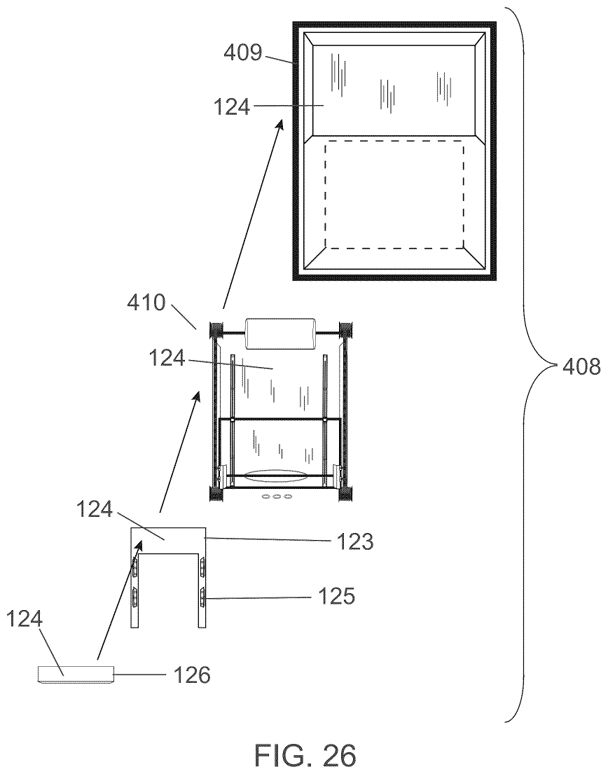

FIG. 26 depicts an exploded rear perspective view of the restroom stall latch upper housing assembly 408 (herein also referred to as “RS UHA”) comprising the restroom stall chassis 409 (herein also referred to as “RS chassis”) comprising UV reflective coating 124, RS drive assembly 410 comprising UV-reflective coating 124, shroud 123 comprising UV-reflective coating, UV-C 125, and battery 126 comprising UV-reflective coating 124. The functionality remains congruent with the invention of FIG. 1A; however, the physical structure differs due to being a single panel embodiment which eliminates the additional panels, panel rails, and related supporting components.

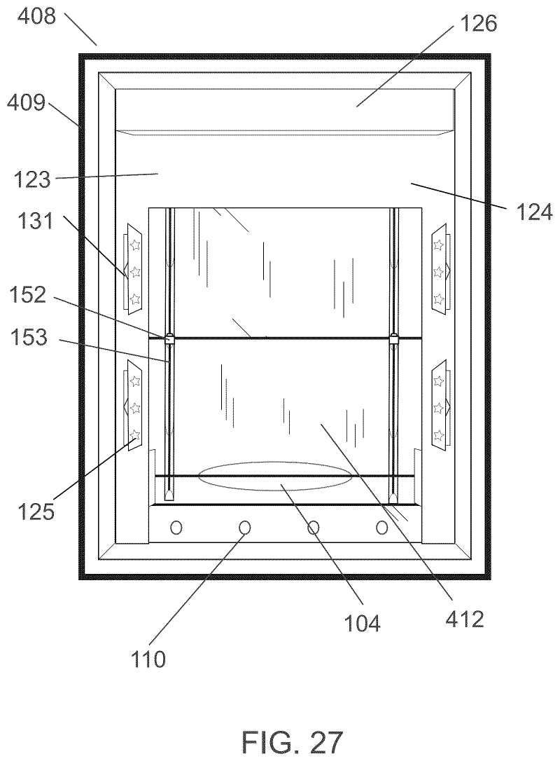

FIG. 27 presents a rear view of an assembled RS UHA 408 with visible components comprising the RS chassis 409, shroud 123, battery 126, UV-C 125, UV reflective coating 124, RS drive panel 412, channel guides 153, guide clips 152, obstruction sensors 110, and emergency handle 104.

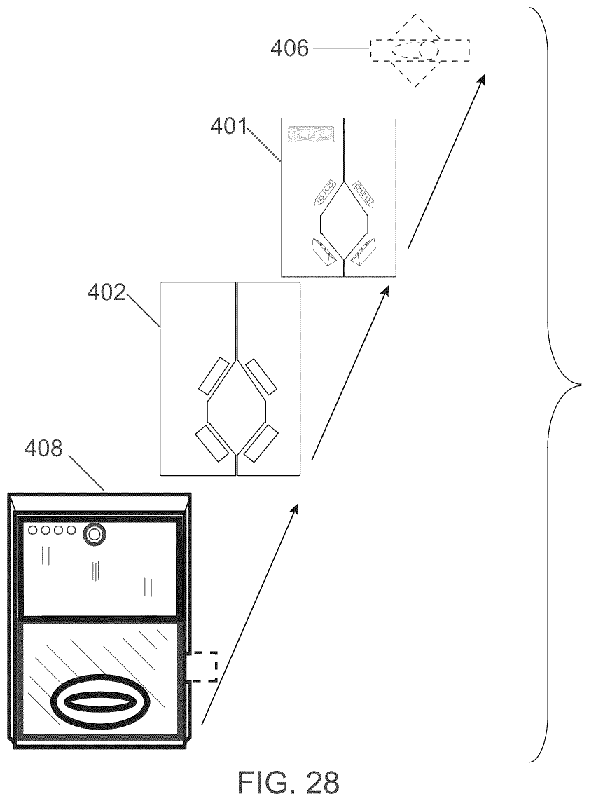

FIG. 28 reveals a front exploded front view of the RS baseplate 401, RS baseplate cover 402, and RS UHA 408 projected into position adjacent to a restroom stall latch 406.

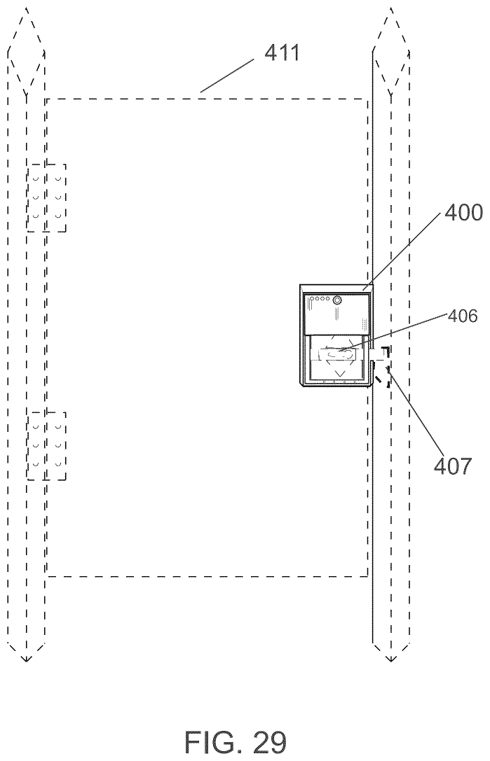

FIG. 29 presents a front perspective view of a restroom stall door 411, stall latch 406, and stall latch receiver 407 with an adjacent RS chamber 400 with the RS drive panel 412 open to allow access to the latch 406.

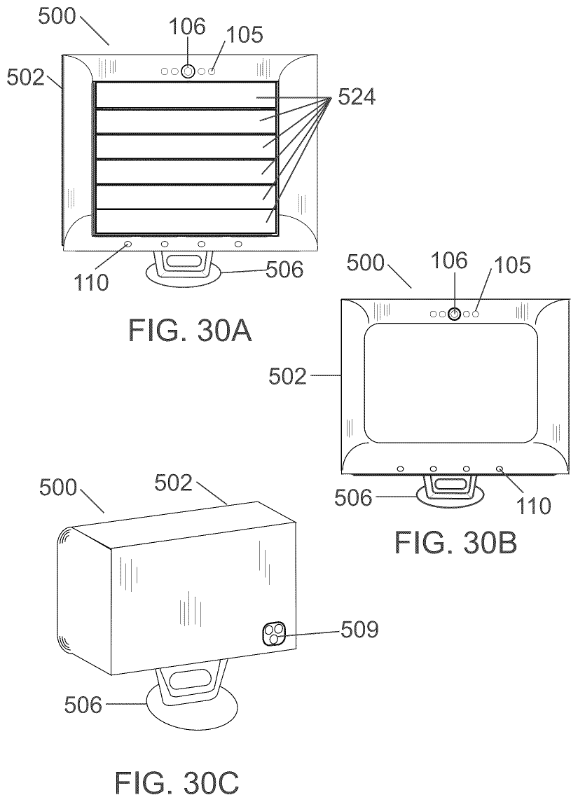

Now referring to an additional embodiment of the invention, FIGS. 30A-36 present a free-standing point-of-sale (POS) terminal germ decontamination chamber 500 (herein also referred to as “POS chamber”) for use at retail checkout counters 508 and the like.

FIGS. 30A-C portray a front closed view, front open view, and rear perspective view, respectively of a POS chamber adjacent to a POS mounting stand. The stand can be permanently affixed to a fixture such as a table or counter through fasteners or adhesives or alternatively can be unmounted and moved when necessary depending on the application and environment. In this embodiment, the front of the POS chamber comprises the chassis 502, access sensor 106, status lights 105, obstruction sensors 110, a POS stand 506, and a six-panel access panel group (herein also referred to as the “access panels”) 524 to minimize the vertical footprint of the device as illustrated in FIG. 30A-B. The rear perspective view of FIG. 30C reveals the direct AC electrical power connection 509 for the POS chamber. Alternative embodiments could comprise a battery 126 power source for environments where an AC connection is unavailable. The POS chamber can be constructed of plastic, metal, or any other suitable material.

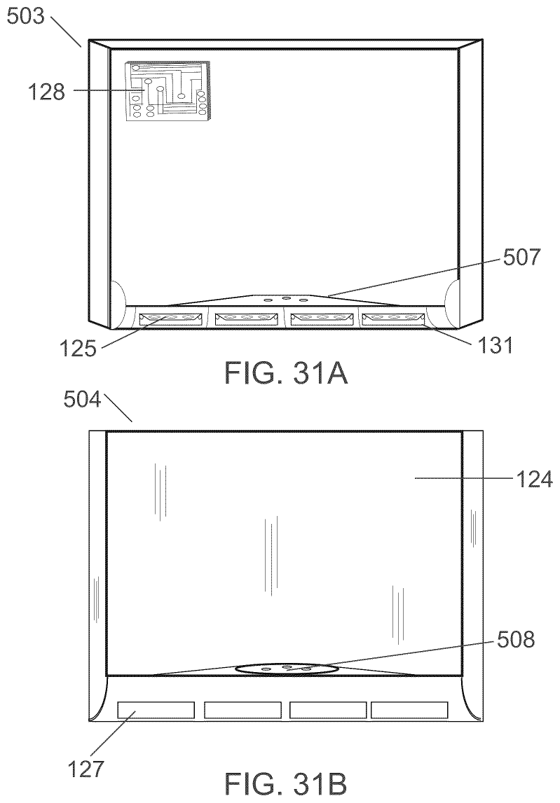

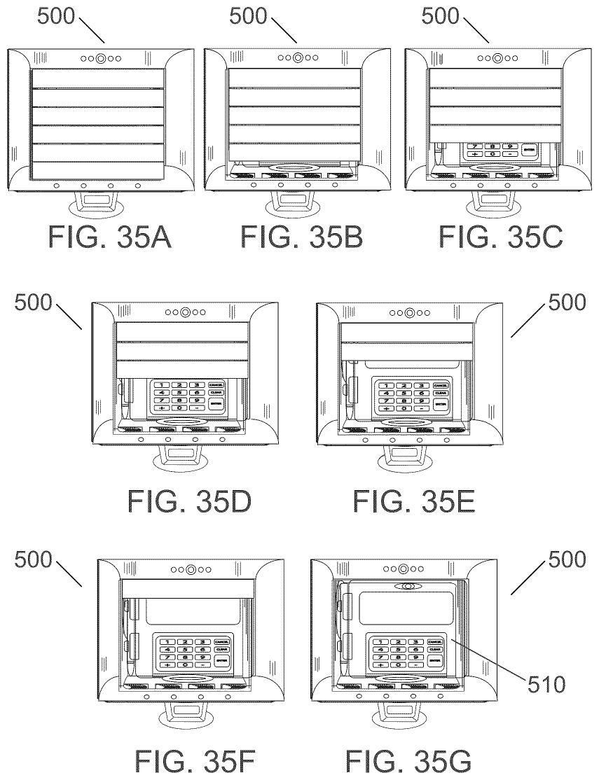

Now referring to FIGS. 31A-B, FIG. 31A reveals a front view of the POS baseplate 503 comprising the baseplate 503, microcontroller 128, embedded POS mounting plate surface 507, UV-C 125, and UV-C mounting stand 131. The depth of the POS baseplate 503 allows placement of the POS terminal 510 (FIG. 35G) and an upwardly sloped lower edge with UV-C 125 and UV-C mounting stand 131 mounted to its surface allowing the light to project to the face of the POS terminal 510 (FIG. 35G). In an alternative embodiment, the UV-C 125 can be placed directly on the surface of the baseplate 503 without the UV-C mounting stand 131. In another alternative embodiment, the UV-C 125 can be positioned on the rear of the access panels 524. FIG. 31B displays a front view of the POS baseplate cover 504 comprising UV-C cutouts 127, UV-reflective surface 124, and a POS stand mounting plate 508.

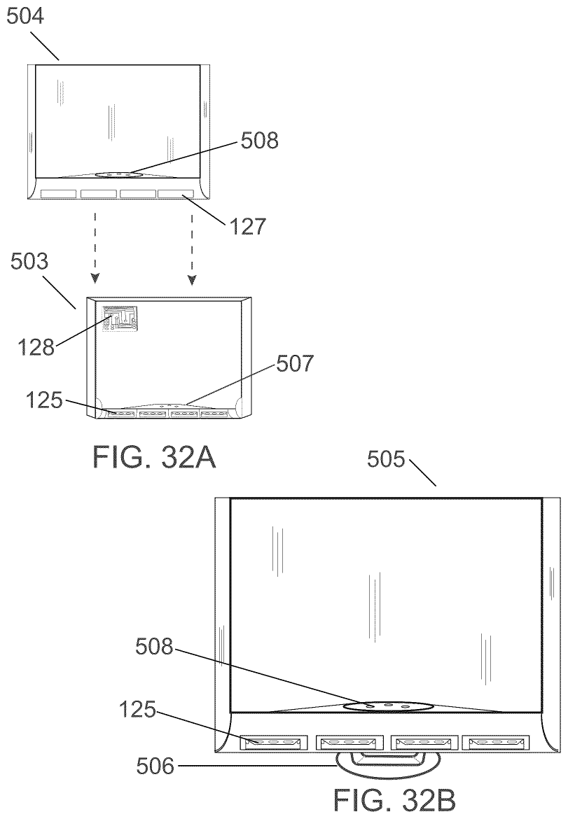

FIG. 32A depicts a front exploded view of the POS baseplate cover 504 comprising UV-C cutouts 127 and mounting stand plate 508 being projected into position on top of and adjacent to the POS baseplate 503 comprising microcontroller 128, UV-C 125, and embedded POS mounting plate surface 507. As illustrated in FIG. 32B, the combined POS baseplate 503 and POS baseplate cover 504 form the POS baseplate assembly 505. The POS mounting stand plate 508 attaches to the POS stand 506 as revealed in FIG. 32B. In an alternative embodiment, the POS baseplate assembly 505 can function as a stand-alone assembly without the use of a POS stand 506 or external mounting apparatus.

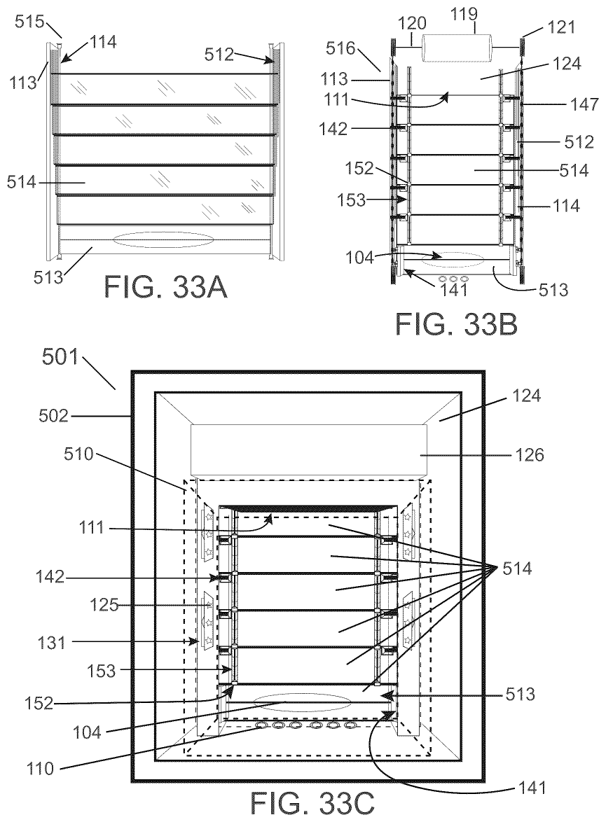

FIG. 33A reveals a front view of the POS access panel assembly 515 (herein also referred to as “POS AP assembly”) comprising parallel and oppositely disposed access panel frames 113 (herein also referred to as “AP frame(s)”), POS panel rails 512 (herein also referred to as “POS rails”), and support bridges 114 to form a left and right side of the POS AP assembly 515 and framing the POS access panel group 514 (herein also referred to as “POS access panels” or “access panels”) which comprises all access panels including the individually defined POS drive panel 513. The POS AP assembly 515 comprises relatively equivalent components and shares the same functional operation as the invention of FIG. 1A and the description provided for the access panel assembly 139 illustrated in FIG. 7 apart from the number of access panels 514 (six versus four) and the number of rails 515 to support the access panels 514 (five versus three).

FIG. 33B shows a rear closeup view of the POS drive assembly 516 comprising the drive motor 119, drive shaft 120, pulley 121, chain 147, access panel frame 113, POS rails 512, support bridge 114, POS access panels 514, support arms 142, POS drive panel 513, drive clip 141, channel guide 153, guide clip 152, panel bay 111, emergency handle 104, and UV-reflective coating 124.

Still referring to the POS drive assembly 516 presented in FIG. 33B, upon actuation of the drive motor 119, the drive shaft 120 and pulley 121 begin to move the chain 147 and attached drive clip 141 which in turn begins movement of the POS drive panel 513. Two oppositely disposed guide clips 152 are attached (or embedded into) to the horizontal leading edge of the POS drive panel 513 and each subsequent POS access panel 514 with the protruding front edge of the guide clips 152 fitted into the adjacent channel guide 153. During retraction, the two guide clips 152 on the drive panel 513 move vertically within the channel guides 153 of the adjacent POS access panel 514 and begin to push it toward the next adjacent POS access panel 514. The guide clip 152 on each POS access panel 514 travel within their adjacent channel guide 153 to push the adjacent access panel 514 in the appropriate direction until the POS access panels 514 are parked within the panel bay 111. The POS access panels 514 are stabilized and synchronized during movement by the support arms 142 and base of the drive clips 141 which slide along and are buttressed by the support bridge 114. The POS drive assembly 516 illustrated in FIG. 33B comprises relatively equivalent components and shares the same functional operation as the invention of FIG. 1A and detailed description of FIG. 13 aside from the number of access panels 514, rails 512, and their supporting components.

FIG. 33C displays a rear closeup view of the POS upper housing assembly 501 (herein also referred to as “POS UHA”) including the adjacent POS terminal 510 as indicated by the rectangular broken lines. Illustrated components in this view comprise the POS chassis 502, optional battery 126, panel bay 111, UV-C 125, UV-C mounting stand 131, support arm(s) 142, channel guide 153, guide clip 152, drive clip 141, emergency handle 104, obstruction sensors 110, POS access panels 514 (including the drive panel 513), and UV-reflective surface 124 (not visible). The POS UHA 501 illustrated in FIG. 33C comprises relatively equivalent components and shares the same functional operation as the invention of FIG. 1A and detailed description of FIG. 14 aside from the number of access panels 514, rails 512, and their supporting components.

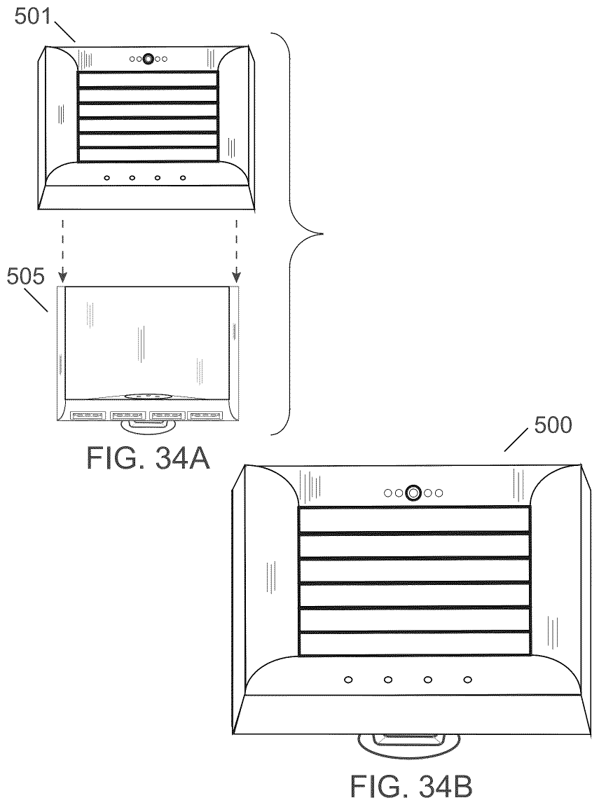

FIG. 34A illustrates a front exploded view of the POS UHA 501 being projected into position on top of and adjacent to the POS baseplate assembly 505 with the resulting front perspective view of the POS chamber 500 being presented in FIG. 34B.

FIGS. 35A-G present a front view of the seven access panel positions of the POS chamber 500 beginning with being closed and sealed in FIG. 35A and concluding with the POS terminal 510 being fully accessible in FIG. 35G.

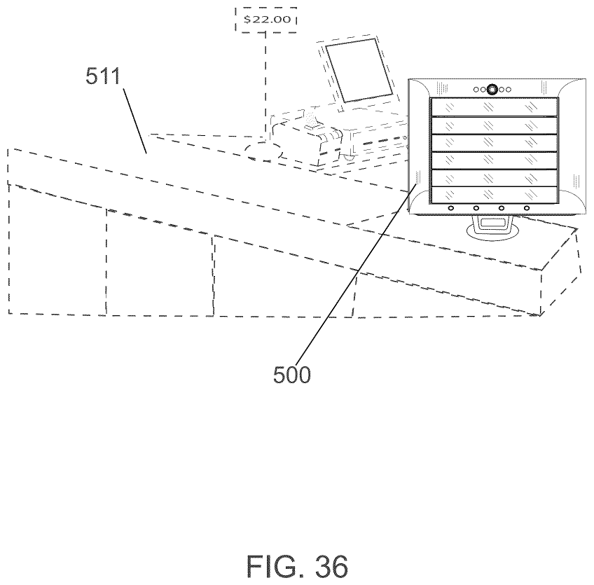

FIG. 36 reveals a front perspective example of the POS chamber 500 adjacent to a retail checkout counter 511.

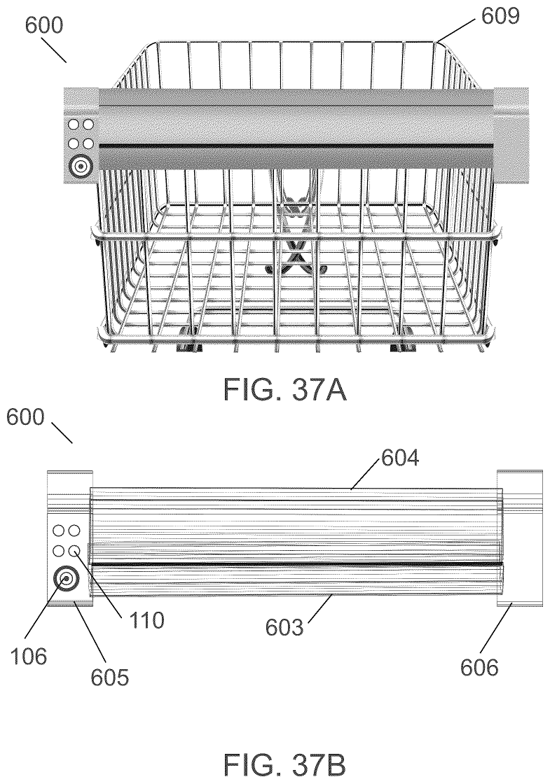

Now referring to an additional embodiment of the invention, FIGS. 37A-47E show a cylindrical germ eradication chamber 600. In the preferred embodiment, the cylindrical chamber 600 is used to decontaminate germs from elongated and horizontally displaced fomites 115 i.e., push-style door handles (ex: panic bars, crash bars, horizontal push bars), shopping cart handles and the like. In alternative embodiments, the cylindrical germ decontamination chamber 600 could be vertically or diagonally oriented on fomites 115 better served by a cylindrical chamber 600 than a linear chamber.

Referring now to the invention illustrated in FIGS. 37A-47E, a cylindrical germ decontamination chamber for shopping cart handles 600 (herein also referred to as “SC chamber”) is presented. FIG. 37A provides a front view example of the SC chamber 600 adjacent to a shopping cart 609. FIG. 37B shows a front view of the SC chamber 600 detached from the shopping cart 609 with front facing components comprising the cylindrical access panel group 604 (herein also referred to as “cyl panels” or “access panels”), left housing 605, right housing 606, access sensor 106, status lights 105, and the undercarriage assembly 603.

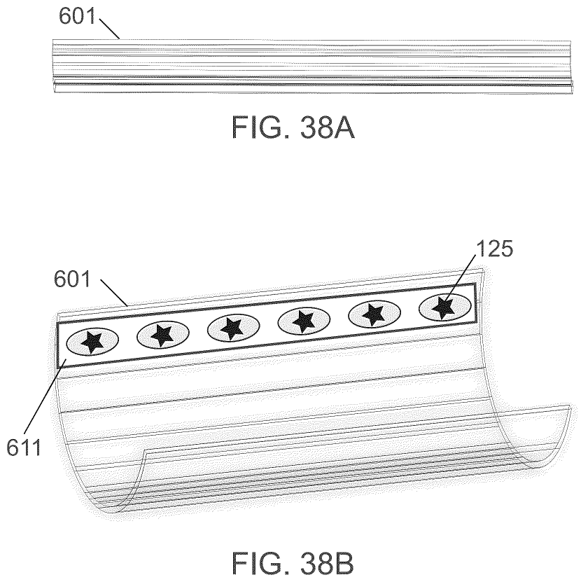

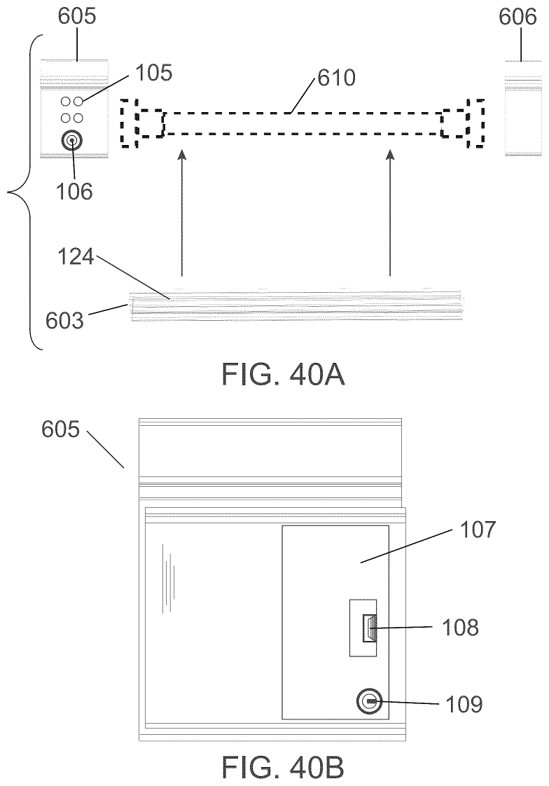

FIGS. 38A-B show a front view and an elevated front perspective view, respectively, of the cylindrical baseplate 601 (herein also referred to as “cyl baseplate”) of the SC chamber 600. As illustrated in FIG. 38B, the cyl baseplate 601 comprises UVC 125 positioned in proximity to the upper rear of the slope to deliver UVGI directly toward the shopping cart handle 610 (FIG. 40A). In the preferred embodiment, the UV-C 125 is embedded or affixed to UV adhesive strips 611 which are pre-wired to deliver power to the UV-C 125. Alternative embodiments include but are not limited to the UVC 125 could be embedded directly into the surface during the manufacturing process, adhered to the surface directly, mounted to the cyl baseplate 601 with the UV-C mounting stand 131, or through any other suitable method. In another alternative embodiment, the UV-C 125 could be affixed directly to one or more of the cyl panels 604.

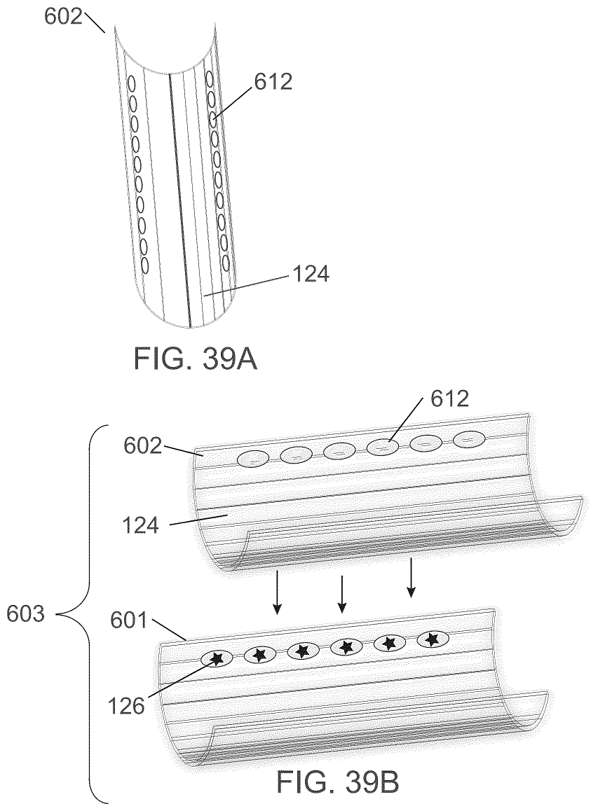

FIG. 39A depicts a top perspective view of the cylindrical baseplate cover 602 (herein also referred to as “cyl baseplate cover”) comprising a UV-reflective surface 124 comprised of UV-reflective paint, TPFE, aluminum foil, or any other substance/material proven to optimize ultraviolet reflectivity. The cyl baseplate cover 602 also comprises UV-C cutouts 612 which overlay the UV-C 125 from the cyl baseplate 601 to allow the light to be delivered to the shopping cart handle 610 (FIG. 40A) while preventing tampering by users.

FIG. 39B displays an elevated front perspective exploded view of the cyl baseplate cover 602 projected and seated upon the cyl baseplate 601 to form the undercarriage assembly 603 of the SC chamber 600. The cyl baseplate 601 and cyl baseplate cover 602 can be manufactured of metal, plastic, or any other suitable material.

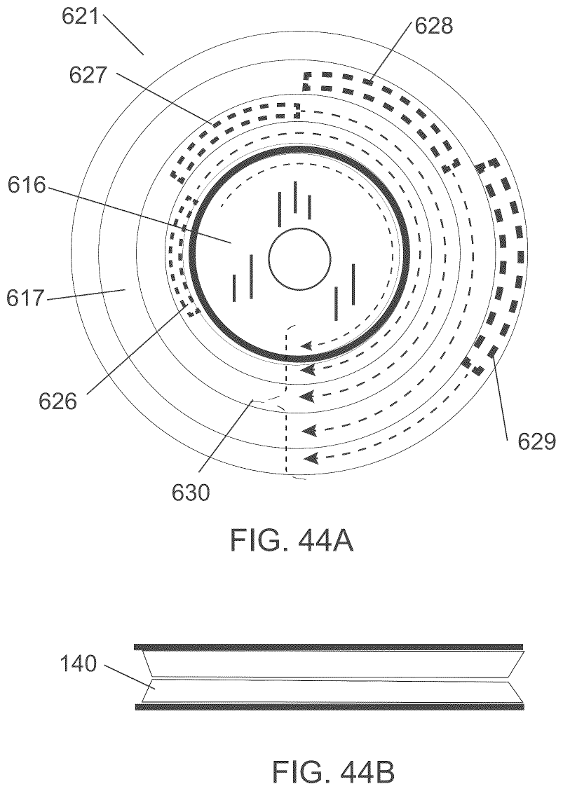

The shopping cart handle 610 is flanked on each side by parallel containers identified as the left housing 605 and the right housing 606 as shown in the exploded front view of FIG. 40A. The left housing 605 (herein also referred to as the “drive housing”) contains the functional power, electrical, and motorized components of the SC chamber 600 including the access sensor 106 and status lights 105. The right housing 606 (herein also referred to as the “driven housing”) functions as a receiver for the right side of the cyl access panels 604 (FIG. 44B). Still referring to FIG. 40A, an exploded view illustrates the undercarriage assembly 603 being projected toward its position on the chamber 600 centered between and adjacent to the left 605 and right housings 606 and underneath the shopping cart handle 610.

FIG. 40B shows a side view of the left housing 605 comprising the battery access door 107, battery latch 108, and security lock 109. Both the left 605 and right housing 606 can be constructed of metal, plastic, or any other suitable material which can provide the necessary strength, rigidity, and durability to optimize the performance of the chamber 600.

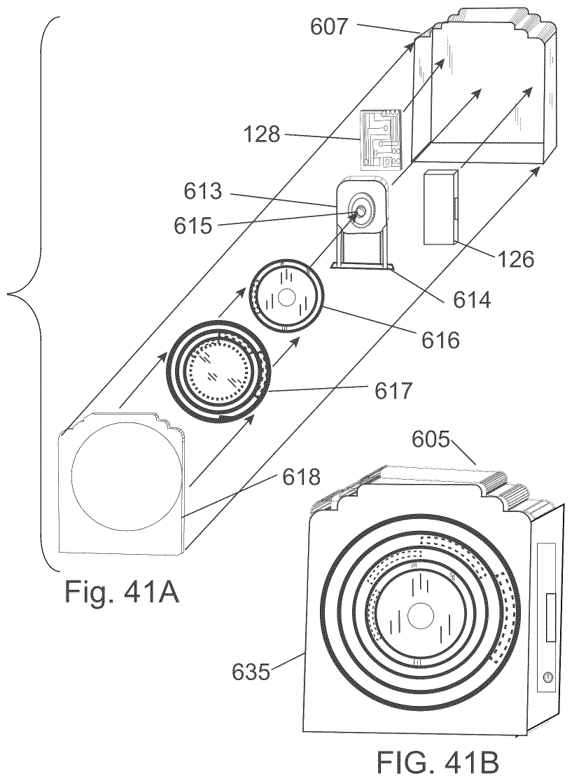

FIG. 41A illustrates a side perspective exploded view of the components of the left
housing 605 comprising the left housing chassis 607, microcontroller 128, battery 126, cylindrical drive motor 613 (herein also referred to as “cyl motor” or “motor”), drive shaft 615, motor support 614, drive hub 616 (herein also referred to as “hub”), driven drum 617 (herein also referred to as “drum”), and the end cap 618. The hub 616 connects directly to the cyl motor 613 and drive shaft 615 as depicted in FIG. 41A, rotating clockwise to retract the cyl access panels 604 in a stacked array providing access to the shopping cart handle 610 and counterclockwise to close and seal the chamber 600. The drum 617, by contrast, functions as a stationary component and as such does not rotate.

The open elliptical center of the end cap 618 is placed around the edges of the drum 617, securing it in place, and then attached to the left chassis 607 to seal the left housing 605. An assembled view of the left housing 605 in hub position “0” (closed) 635 is depicted in FIG. 41B.

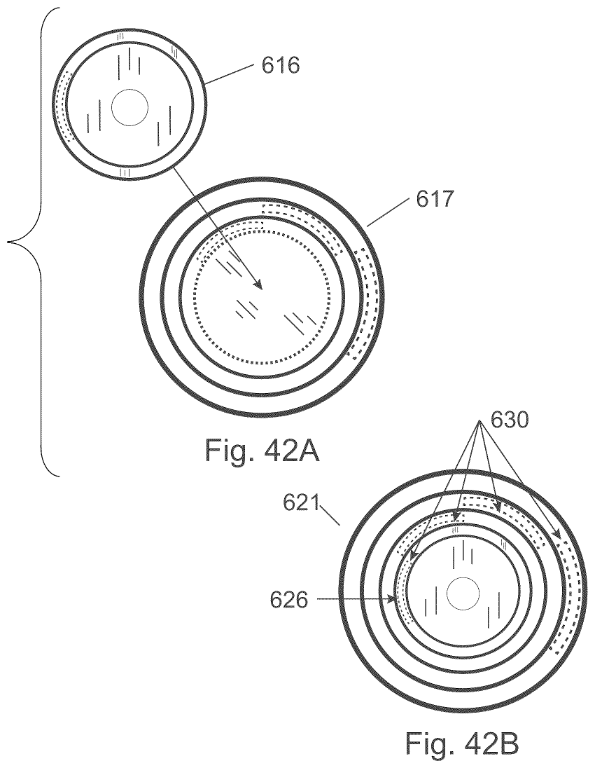

FIG. 42A exhibits a front closeup exploded view of the left hub 616 projected into its position within the center of the left drum 617. The two integrated components form the left hub & drum assembly 621 (herein also referred to as “left H&D assembly”) as illustrated in FIG. 42B. The cyl rails 630 which support the cyl panels 604 are also depicted in FIG. 42B. Additional detail regarding the interface between the hub 616, drum 617, and cyl access panels 604 is provided in FIGS. 44A-48E.

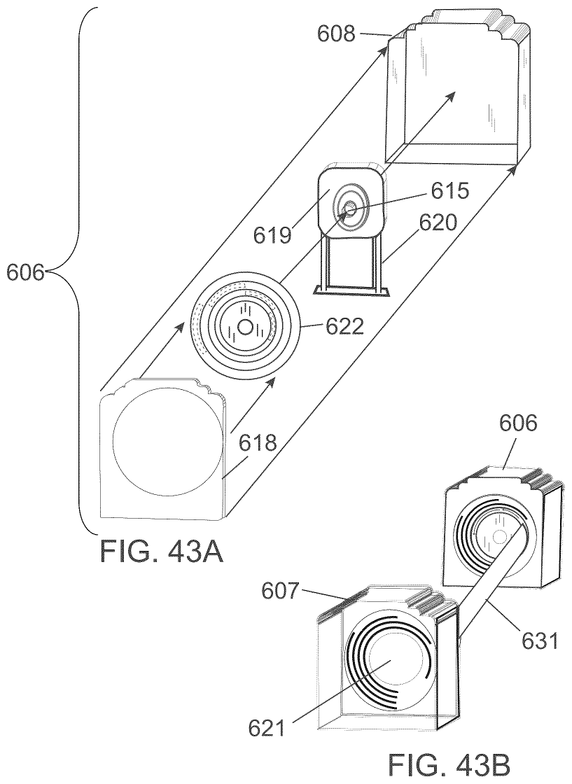

In more detail, still referring to the invention of FIG. 37A, a side perspective exploded view of the right housing 606 is depicted in FIG. 43A comprising the right chassis 608, free spinning hub 619, drive shaft 615, free spinning hub support 620, right H&D assembly 622, and end cap 618. The right housing 606 is a “driven housing” as previously mentioned; subordinated to the left housing 605 in that it has no power or control functions within the SC chamber 600. The right chassis 608 is additionally distinguished from the left chassis 607 by having less internal area due to the absence of a microcontroller 128 and battery 126 from the chassis 608 as previously depicted in FIG. 40A. Alternative embodiments could include, but are not limited to, a motorized H&D assembly in both the left and right housing 621, 622 through a single or plurality of cyl drive motors 613 and powered by a single or plurality of batteries 126.

Still referring to FIG. 43A in more detail, the right housing 606 comprises a free spinning hub 619 and a connected drive shaft 615 which is actuated through the movement of the components within the opposing left housing 605. The drum 617 overlays the free spinning hub 619 within the right chassis 608 and is secured in a stationary position when the end cap 618 is inserted into the chassis 608 to close the right housing assembly 606.

FIG. 43B represents a side perspective view of the left chassis 607 (illustrated with the side removed) and the left H&D assembly 621 (other internal components removed for visibility) connected to cyl panel #1 631 (herein also referred to as “the drive panel”) which is connected to the right housing 606 in the closed position.

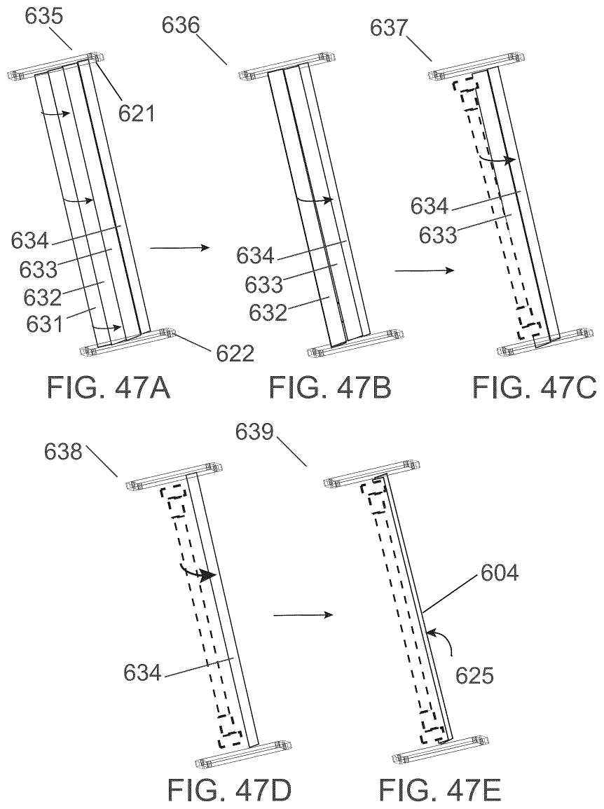

FIG. 44A presents a side closeup view of the left H&D assembly 621 in hub position “0” 635 (closed). The left hub 616, when viewed from the right side (inside) of the left chassis 608, rotates in a clockwise direction to retract the cyl access panels 604 upon each other in a stacking form as indicated by the directional arrows within the left H&D assembly 621 in FIG. 44A. In hub
position “zero” 635 (closed position) cyl panel #1 631 (the drive panel as exhibited in FIG. 47A) is affixed to cyl rail #1 626 (herein also referred to as the “drive rail”) in the 8-10 o'clock slot on the hub 616 as indicated in FIG. 44A. Each embedded rail within the drum 617 comprises an embedded nylon glide 140, illustrated in FIG. 44B, to promote freedom of movement and prevent friction during cyl panel 604 movement. Alternative embodiments include, but are not limited to, the cyl rails 630 comprising ball bearings or similar fittings, surface coatings, materials, or any other suitable solution that promotes freedom of movement and reduces friction for the cyl panels 604. In another alternative embodiment, the cyl panels 604 could be constructed of any material that promotes freedom of movement and reduces friction between the cyl rails 630 without the use of additional components.

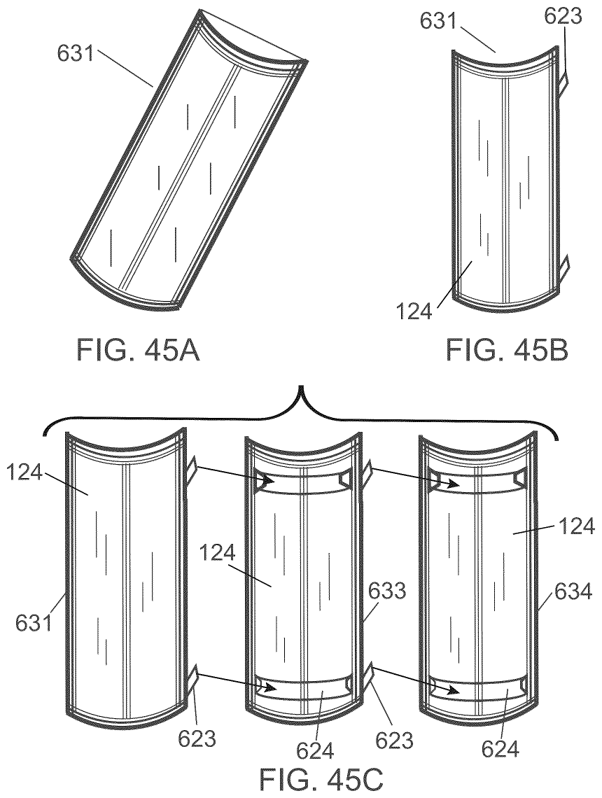

FIGS. 45A-B show a top and bottom perspective view, respectively, of the cylindrical access panels 604 using cyl panel #1 631 in FIGS. 45A-45B and the left illustration in 45C. The bottom view of FIG. 45B reveals the cylindrical drive clips 623 (herein also referred to as “cyl drive clips” or “drive clips”) and a UV-reflective coating 124 such as aluminum foil, UV-reflective paint, TPFE, or any other substance/material that optimizes reflectivity of UV-C light within the SC chamber 600. All interior areas within the undercarriage assembly 603 and access panels 604 of the SC chamber 600 are coated with a UV-reflective material/substance 124 as are all fomite-facing components within the various embodiments of this invention. FIG. 45C exhibits a bottom exploded view of a three-access panel 604 example of their interface with projection arrows indicating the placement of each access panel 604 within the array. Describing FIG. 45C in more detail as shown from left to right, the left panel is an example of cyl panel #1 631 (the drive panel) depicting a panel with cyl drive clips 623 but absent of channel guides 624. The cyl drive clips 623, as shown in the top and bottom of the illustration, are vertically oriented allowing them to fit within the recessed barrel of the channel guides 624 in adjacent cyl panel #3 633 (center) as illustrated by the arrows. The channel guides 624 have a solid edge at each end which causes the panel to be pushed or pulled, depending on the direction of the panel movement, by the cyl drive clips 623 attached to the adjacent panel. Cyl panel #3 633 (center) is comprised of channel guides 624 and cyl drive clips 623. The cyl drive clips 623 from the center panel are fitted within the parallel and oppositely disposed edges of the channel guides 624 on the panel on the right side of FIG. 45C referenced as cyl panel #4 634 (the exit panel). The right panel is characterized as an exit panel as evidenced by being comprised of channel guides 624 to allow it to be pushed and pulled during retraction and closing through the action of the preceding panel's cyl drive clips 623; however, it is not comprised of its own drive clips 623 since, as the final panel in the array, it is itself moved but does not otherwise move any other panels 604.

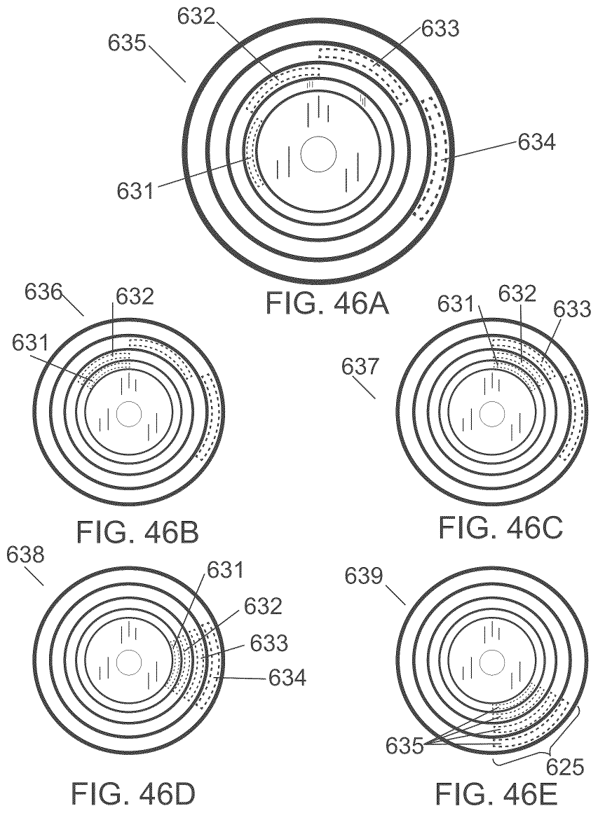

FIGS. 46A-E and FIGS. 47A-E further detail the operation of the cyl access panels 604 (previously illustrated in FIG. 37B) and their interface with the H&D assembly 621 (FIG. 47A). FIGS. 46A-E depict a side closeup view of the left H&D assembly 621 illustrating the five stages of panel retraction and FIGS. 47A-E exhibit a top perspective view of the corresponding cyl panels 604 (FIG. 47A) being retracted throughout the five-stage retraction process in the four-panel SC chamber 600 embodiment.

Referring to FIG. 46A, the hub 616 and drum 617 slots are in hub position “0” 635 (closed) within the left housing 605 (shown in FIG. 41B) which is mirrored within the opposite side right housing 606 (presented in FIG. 43B). Defining the cyl rails 630 by number, cyl rail #1 626 is the drive rail for which cyl panel #1 631, depicted in FIG. 47A, is affixed to the location on the drive hub 616 identified by the broken lines between 8-10 o'clock. Cyl rail #1 626 comprises a fixed width slot to which cyl panel #1 631 (drive panel shown in FIG. 47A) connects and rotates in synchronization with the hub 616. Continuing in a clockwise direction, the fixed-mount drum 617 comprises cyl rail #2 627, cyl rail #3 628, and cyl rail #4 629 as identified in FIG. 46A with the closed panel position for each respective rail indicated by broken lines. Each of the three rails on the drum 617 in this embodiment comprise an embedded rail with a nylon glide 149 (FIG. 44B) which continues throughout the rotation area terminating in the cyl panel bay 625 depicted in FIG. 46E. A corresponding view of the SC chamber 600 in this position with the cyl panels attached is shown in FIG. 47A.

FIG. 46B shows hub position #1 636 whereby the hub 616 and cyl rail #1 626 has rotated clockwise into a position underneath cyl rail #2 627 as indicated by the alignment of the broken lines. The corresponding position of the cyl panels 604 within the SC chamber 600 in this position is portrayed in FIG. 47B.

FIG. 46C represents hub position #2 637 whereby the hub 616 and cyl rail #1 626 has rotated into a position underneath cyl rail #3 628 and brought with it the cyl rail #2 627 creating three stacked panels as depicted by the alignment of the broken lines in FIG. 46C. The corresponding position of the cyl panels 604 within the SC chamber 600 in this position is portrayed in FIG. 47C.

FIG. 46D reveals hub position #3 638 whereby the hub 616 and cyl rail #1 626 has
rotated into a position underneath cyl rail #4 629, moving the panels within cyl rail #2 627 and #3 628 with it thereby creating four stacked panels. The corresponding position of the cyl panels 604 within the SC chamber 600 in this position is represented in FIG. 47D illustrating the cyl panels 604 as being 75% open. Hub position #4 639 is the final stage in the panel retraction process as is presented in FIG. 46E. In this stage, the hub 616 and cyl rail #1 626 (drive rail) has rotated into the innermost position within the cyl panel bay 625 and brought with it the cyl panels 604 (FIG. 47E) attached to cyl rail #2 627, #3 628, and #4 629 so the cyl rail group 630 is aligned upon each other. The corresponding position of the cyl panels 604 within the SC chamber 600 in this position is represented in FIG. 47E revealing the cyl access panels 604 100% retracted and stacked upon each other within the cyl panel bay 625 as illustrated in FIG. 47E.

Referring to FIGS. 47A-E in more detail, these present a top perspective view of the five stages of panel retraction for the SC chamber 600. FIG. 47A illustrates a fully closed and scaled SC chamber 600 and identifies the individual cyl panels 604. Viewing FIG. 47A from left to right, cyl panel #1 631 serves as the drive panel which is connected to the hub 616 as illustrated in FIG. 46A; followed by cyl panel #2 632, cyl panel #3 633, and cyl panel #4 634 in order from left to right which are attached to their dedicated rail on the drum 617 as illustrated in FIG. 46A.

FIG. 47B shows cyl panel #1 631 retracted underneath cyl panel #2 632 which would reveal 25% of the shopping cart handle 610 (not shown). With cyl panel #1 631 drive now positioned beneath cyl panel #2 632, the continued rotation of cyl panel #1 631 pushes cyl panel #2 632 underneath cyl panel #3 633 to reveal 50% of the shopping cart handle 610 as depicted in FIG. 47C. Cyl panel #1 631 continues to rotate and push cyl panel #2 632 and cyl panel #3 633 to stack underneath cyl panel #4 634 revealing 75% of the shopping cart handle 610 as represented in FIG. 47D. In the final stage of retraction, as cyl panel #1 631 is pushing cyl panel #2 632, cyl panel #3 633, and cyl panel #4 634 into the cylindrical panel bay 625 where all four panels are stacked upon each other as revealed in FIG. 47E. To close the panels and seal the SC chamber 600 (as depicted in FIG. 37A), the process is repeated led by cyl panel #1.

As used herein, the term enclosure generally is as described above and can generally include or be a chamber or a chassis with one or more sides. An enclosure can be in a variety of geometric shapes and will generally completely enclose a fomite with the exceptions of a door or access panels and an opening to accommodate the fomite when the fomite is attached to something else. For example, a door handle connected to a door, or a gas pump handle connected to a gas pump, etc. In embodiments, an enclosure can be three dimensional rectangular in shape with six sides. In embodiments, the enclosure can be cube, rectangular prism, sphere, cone and/or cylindrical in shape but is not limited thereto. An enclosure may be airtight and/or watertight when the door or access panels are closed.

In embodiments, a sensor as used herein, may include an obstruction sensor, a motion sensor or detector, a light sensor, a sound sensor, and/or a heat or infrared sensor. As discussed above, in embodiments, a sensor can detect the presence of a user and then automatically trigger the opening of the door or access panels of the enclosure or chamber. Such a system allows a user to access the fomite without touching the door or access panel.

A trigger or a trigger event is an event or trigger which opens the access door and is generally detected by a sensor. That is, a user approaching a fomite may trigger a sensor causing the door or access panel of the enclosure to open allowing access to the fomite. Thus, a trigger event may be an event detectable by a sensor as described above. For example, in a restroom environment, a motion sensor or light sensor can be used to detect a trigger event and the presence of a user (as is routinely done at restroom stalls to trigger the flushing of the toilet, or turning on or off, of a water faucet). For a door handle, a trigger event could be a user approaching the door, or being near to the door or access panels, as detected by a motion sensor or light sensor. Nevertheless, the disclosure is not limited to the use of sensors and a trigger could also be produced by mechanical means, for example, a foot pedal.

An access door is generally a door or panel incorporated in, or integral to, the enclosure which can be opened to allow access to the interior of the enclosure. A door can be opened by any conventional means, for example, by swinging open, sliding open, an accordion-type access door or panel opening, etc. The size of the door or panel will necessarily vary in accordance with the size of the fomite and the access necessary to utilize the fomite. For example, for a door handle the opening will necessarily be large enough to accommodate the door handle and a hand of a user opening the door. For a point-of-sale terminal, an opening large enough to allow a user to use the point-of-sale terminal will necessarily be required. In embodiments therefore the size of the door or access panels will be at least large enough to accommodate a user's hand.

A door in an open position is any position which is not fully closed. A door in a closed position generally means the door is fully closed scaling or protecting the fomite from the outside environment. In embodiments, the door may be airtight, watertight, may include a clear or see-through material, for example a plastic polycarbonate, glass, or any other see-through material. In other embodiments the door or access panels may include metal or plastic or composite and may be light blocking.

The enclosure surrounding a fomite generally means that the enclosure or chamber completely encloses the fomite. In embodiments, the enclosure surrounds the fomite and provides an airtight or semi-airtight enclosure where air flow cannot easily pass from outside to the inside of the enclosure.

A UV light source is described above and can be any UV light source that can operate in the UV-C range. The UV light source will generally be able to produce a UV light intensity or power sufficient to kill germs, bacteria, virus, or other pathogens. The UV light power may range between 2,000 and 8,000 μW·s/cm². See Ultraviolet germicidal irradiation, Wikipedia (en.wikipedia.org/wiki/Ultraviolet_germicidal_irradiation), Date of last revision: 20 Feb. 2021, herein incorporated by reference.

As mentioned above, the UV light source may preferably be an LED array capable of providing UV light in one or more frequency ranges optimized to kill germs, bacteria, viruses, and other pathogens. For example, a UV array may produce light at 265 nm, 220 nm, and/or 280 nm. In other embodiments, a UV array may produce light at 220 nm, 225 nm, 230 nm, 235 nm, 240 nm, 245 nm, 250 nm, 255 nm, 260 nm, 265 nm, 270 nm, 275 nm, and/or 280 nm.

Decontaminate as the term is used herein generally means the destruction or neutralization of bacteria or virus. In embodiments, 99% reduction of bacterial or virus is achieved in 5 seconds or less. In embodiments 99% reduction of bacteria or virus is achieved in 3 seconds or less. In embodiments 99% reduction of bacteria or virus is achieved in 1 second. In embodiments, 99.9% reduction of bacterial or virus is achieved in 5 seconds or less. In embodiments 99.9% reduction of bacteria or virus is achieved in 3 seconds or less. In embodiments 99.9% reduction of bacteria or virus is achieved in 1 second. In embodiments the virus is SARS-COV or SARS-COV-1 or variants including the alpha or delta variants. In embodiments 99.9% reduction of SARS-COV or SARS-COV-1 or variants including the alpha or delta variants is achieved in 1 second.

The decontamination can be of any relevant germ, bacteria, or virus, but is preferably a pathogen such as a virus, bacterium, protozoan, prion, viroid, or fungus that can produce disease in a mammal including a human. In one preferred embodiment the pathogen may be SARS-COV or SARS-COV-1 or variants including the alpha or delta variants. See e.g., Pathogen, Wikipedia (en.wikipedia.org/wiki/Pathogen), last edited 8 Jul. 2021, herein incorporated by reference.

Mounting stands are generally used to mount the UV light sources inside the enclosure or chamber. The mounting stands may be movable and may be capable of directing the dosage of UV light in different directions or at different angles inside the enclosure. Attaching or connecting the UV light sources to the mounting stands may be done by any conventional means including mechanical connections, screws, tacks, etc., or using adhesives.

Microprocessors as used herein can generally include any computer processor where data processing logic and control is included on a single integrated circuit, or a small number of integrated circuits. A microprocessor is generally a multipurpose, clock-driven, register-based, digital integrated circuit that accepts binary data as input, processes it according to instructions stored in its memory, and then provides results as output. Microprocessors as contemplated herein, will be capable of managing the sensors, drive system for opening the door or access panels, the UV light sources, as well as managing power sources including battery power.

While this disclosure includes specific examples, it will be apparent after an understanding of the disclosure of this application has been attained that various changes in form and details may be made in these examples without departing from the spirit and scope of the claims and their equivalents.

### EXAMPLES

### Example 1—COVID-19 Experiment

SARS-COV-2 is the virus that causes COVID-19. To date, the current COVID-19 pandemic is responsible for more than 4.55 million deaths globally, of which over 645,000 were in the United States.

Crystal IS (Green Island, NY) is an ISO 9001:2015 certified company that makes Klaran UVC LEDS and systems. IS initiated research with Boston University's National Emerging Infectious Diseases Laboratories (NEIDL) to understand how SARS-COV-2 responds to ultraviolet light across the emission range of Klaran UVC LEDs (260 nm to 270 nm) and at different doses. Experiments were conducted using arrays of Klaran WD Series UVC LEDs at a distance of 7 cm from the test surface.

An array of Klaran UVC LEDs was used to irradiate a dried plastic surface containing SARS-COV-2 at a distance of 7 cm.

Results show log reduction achieved from exposing the virus to a UVC intensity of 1.25 mW/cm² at different time intervals. A UVC dose of 6.25 mJ/cm² resulted in a 99.9% reduction of the virus (Table 1, below).

TABLE 1
Log Reduction as a function of Dose and LED Peak Wavelength.

| | UVC LED | | | |
|---|---|---|---|---|
| | 1.25 mJ/cm² | 2.5 mJ/cm² | 3.75 mJ/cm² | 5 mJ/cm² |
| Wavelength | 1 sec. | 2 sec. | 3 sec. | 4 sec. |
| 260 nm | | | | 2.6 |
| 268 nm | 0.7 | 1.2 | 1.5 | 2.8 |
| 270 nm | | | | 2.8 |

The test was repeated using a dose of 5 mJ/cm² from LEDs of different peak wavelengths, representing both ends of the Klaran LED wavelength specification (260 nm and 270 nm). Results indicate similar efficacy across the tested wavelength range (Table 2, below). Comparing these results against published results from the University of Miyazaki (which used UVC LEDs emitting at 280 nm) highlights a marked drop in efficacy beyond 270 nm wavelength (see Inagaki et al. (2020) Rapid inactivation of SARS-COV-2 with deep-UV LED irradiation, Emerging Microbes & Infections, 9(1): 1744-1747).

TABLE 2
Impact of Wavelength on Log Reduction.

| | UVC LED | | |
|---|---|---|---|
| Wavelength | 5 mJ/cm² | 6.25 mJ/cm² | 37 mJ/cm² |
| 268 nm | 2.8 | >3 | |
| 280 nm¹ | 0.9¹ | | 3.1¹ |

### Conclusions

SARS-COV-2 is a relatively weak virus that can be inactivated by low doses of UVC light. SARS-CoV-2 can be effectively inactivated in a matter of seconds through exposure to low doses of UVC light in the key germicidal range. Furthermore, UVC wavelength matters. Published results from the University of Miyazaki (which used UVC LEDs emitting at 280 nm) imply a marked drop in efficacy beyond 270 nm wavelength. Klaran UVC LEDs emit UVC light in the 260 nm to 270 nm wavelength range, which is the wavelength range that can achieve complete virus inactivation in a matter of seconds.

### Example 2—MicroLumix Product Analysis and COVID-19

A simulation was conducted by Crystal IS of the efficacy of a germ decontamination apparatus according to an embodiment of the present invention against SARS-COV-2. For a door handle, the minimum average intensity on all surfaces, including the back of the handle, was >6.25 mW/cm². Per the results of Example 1, this allows for a 99.9% SARS-COV-2 reduction in 1 second.

### Germpass Elevator Hall Call Unit Example Embodiments

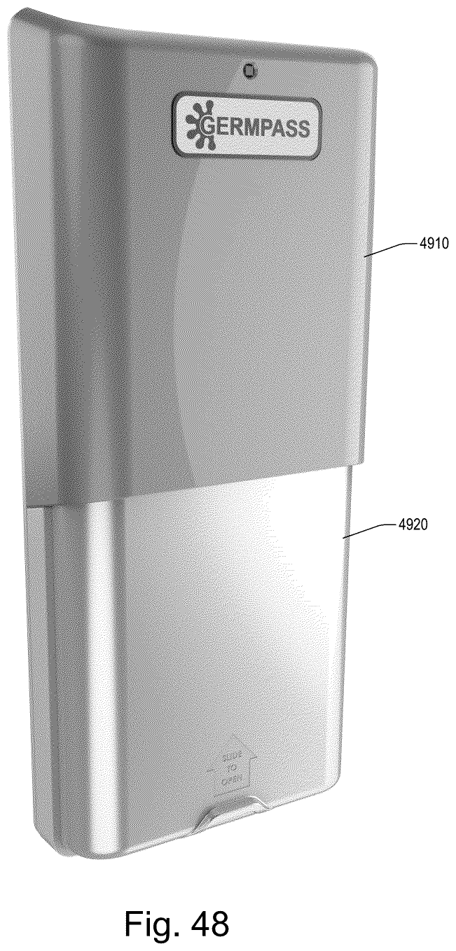

FIGS. 48-63 illustrate different views and features of a Germpass Elevator Hall Call Unit according to at least one embodiment.

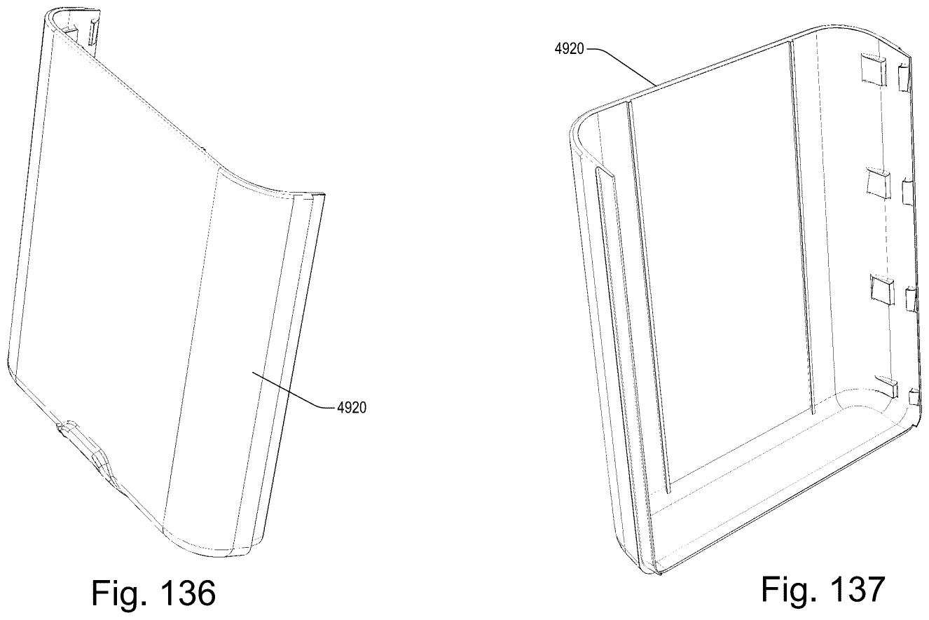

FIGS. 136 and 137 show different perspective views of a Germpass Elevator Hall Call Unit movable access panel 4920 in accordance with at least one embodiment.

FIG. 48 shows a perspective view of an example embodiment of a Germpass Elevator Hall Call Unit configured in a closed configuration.

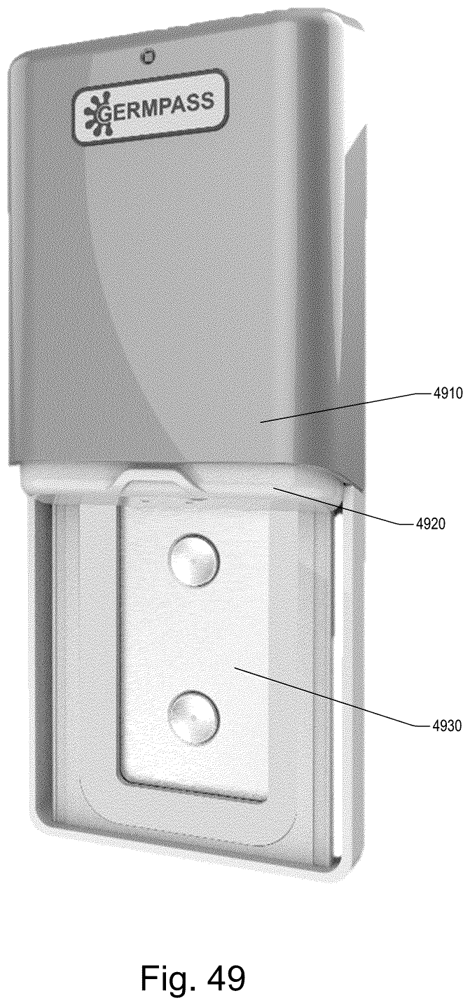

FIG. 49 shows a perspective view of an example embodiment of a Germpass Elevator Hall Call Unit configured in an open configuration.

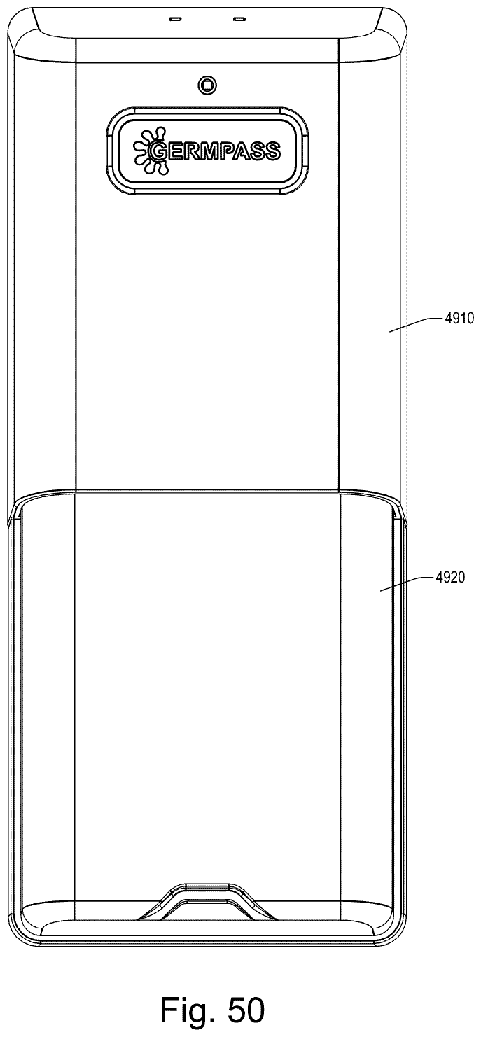

FIG. 50 shows a front view of an example embodiment of a Germpass Elevator Hall Call Unit configured in a closed configuration.

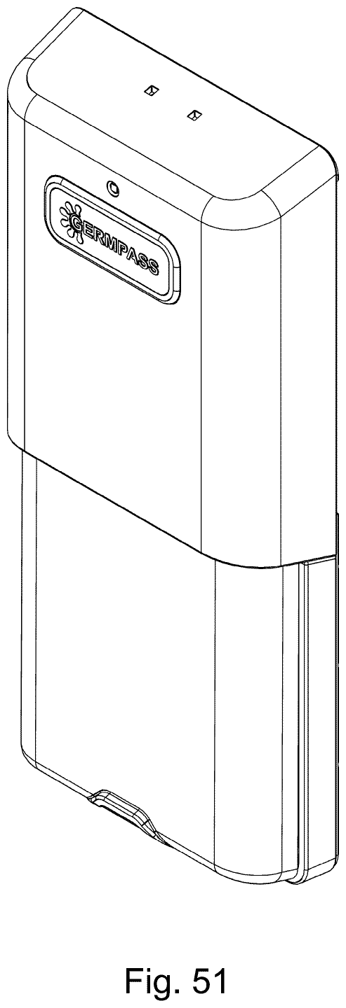

FIG. 51 shows a perspective view of an example embodiment of a Germpass Elevator Hall Call Unit configured in a closed configuration.

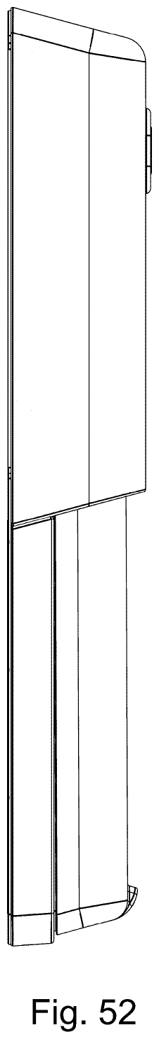

FIG. 52 shows a left view of an example embodiment of a Germpass Elevator Hall Call Unit configured in a closed configuration.

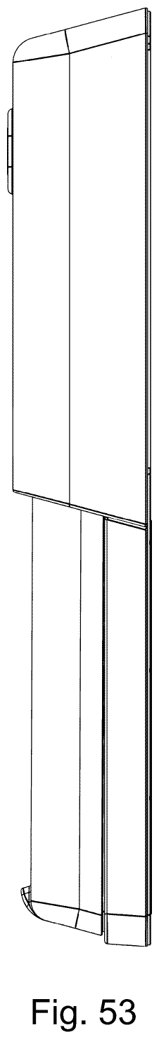

FIG. 53 shows a right view of an example embodiment of a Germpass Elevator Hall Call Unit configured in a closed configuration.

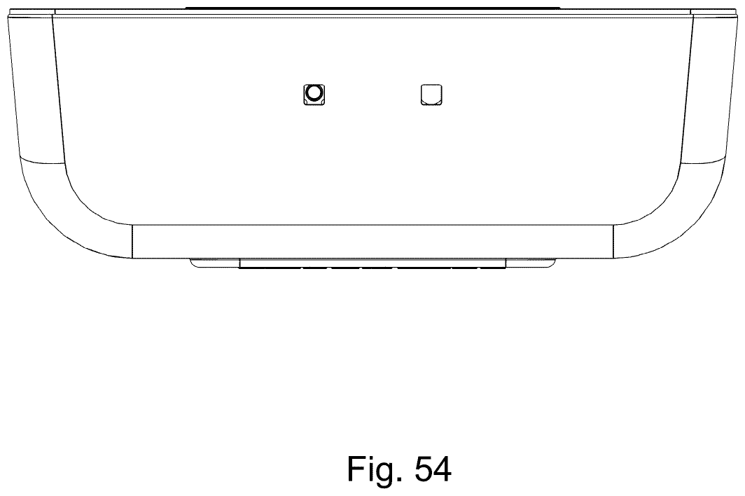

FIG. 54 shows a top view of an example embodiment of a Germpass Elevator Hall Call Unit configured in a closed configuration.

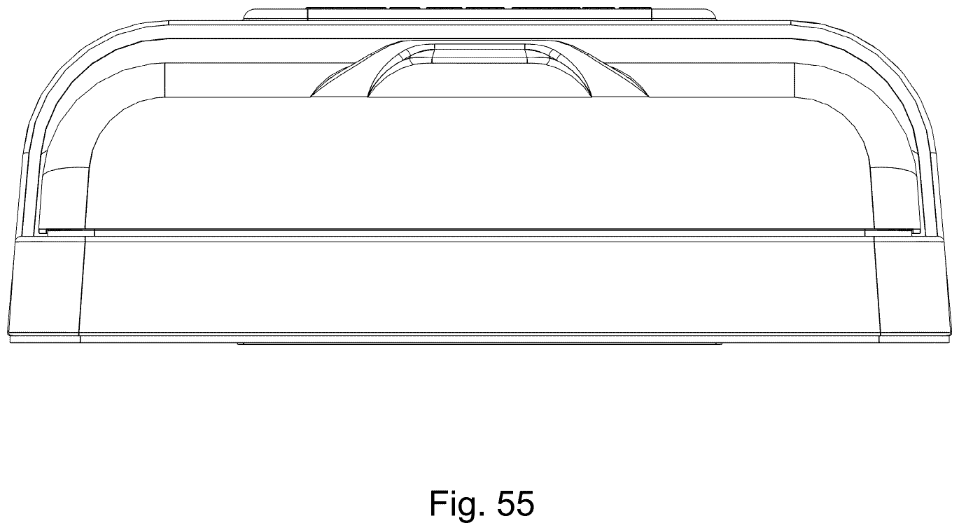

FIG. 55 shows a bottom view of an example embodiment of a Germpass Elevator Hall Call Unit configured in a closed configuration.

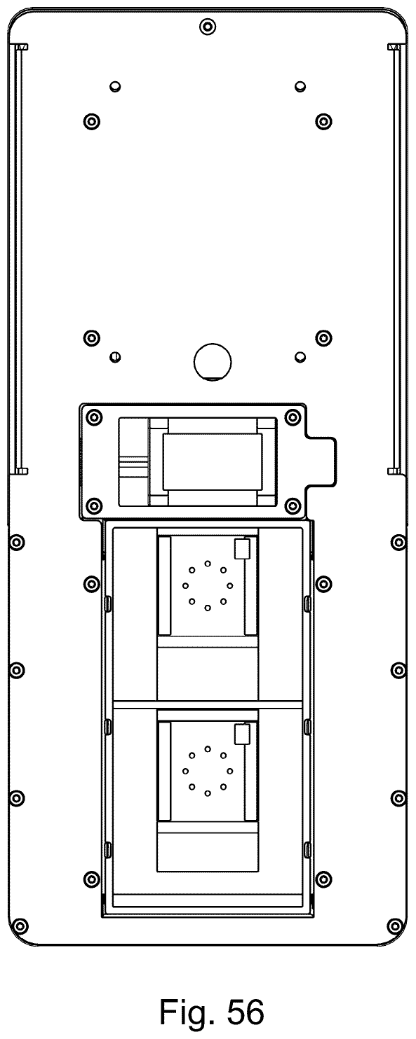

FIG. 56 shows a rear view of an example embodiment of a Germpass Elevator Hall Call Unit configured in a closed configuration.

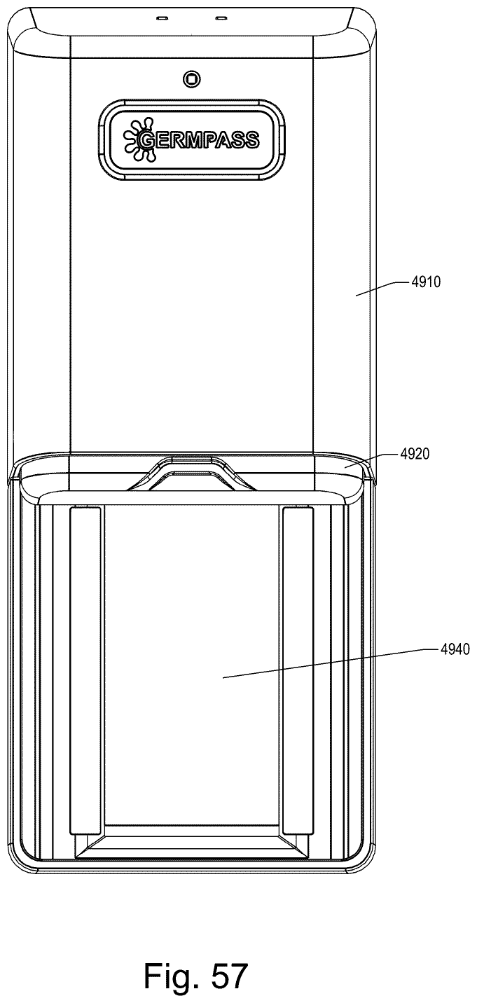

FIG. 57 shows a front view of an example embodiment of a Germpass Elevator Hall Call Unit configured in an open configuration.

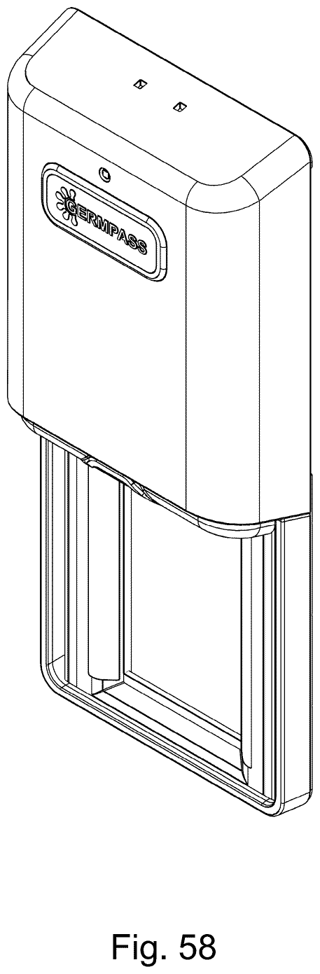

FIG. 58 shows a perspective view of an example embodiment of a Germpass Elevator Hall Call Unit configured in an open configuration.

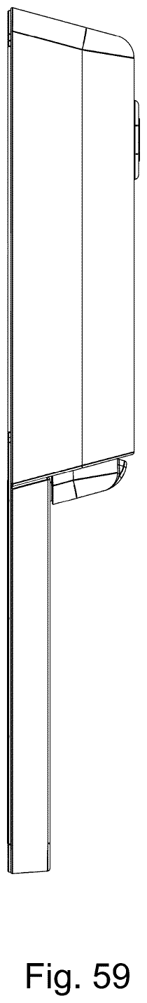

FIG. 59 shows a left view of an example embodiment of a Germpass Elevator Hall Call Unit configured in an open configuration.

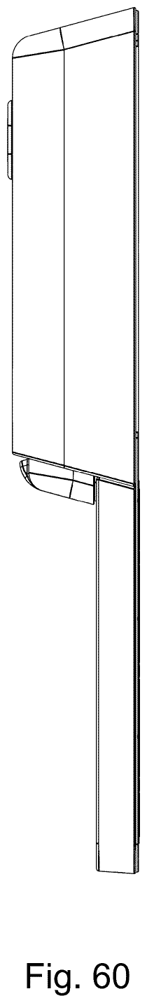

FIG. 60 shows a right view of an example embodiment of a Germpass Elevator Hall Call Unit configured in an open configuration.

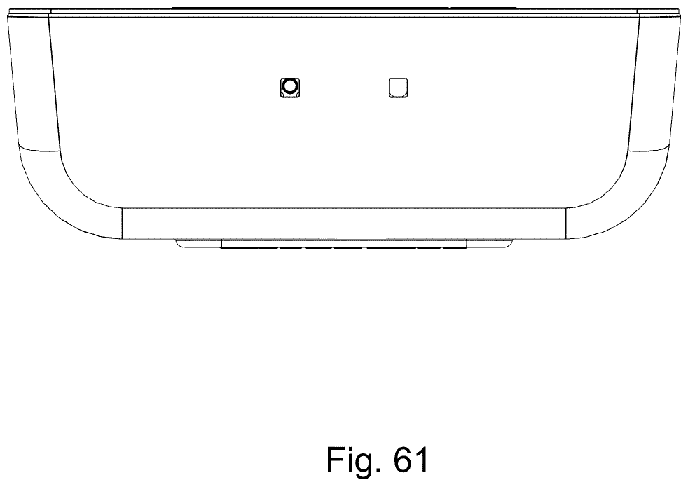

FIG. 61 shows a top view of an example embodiment of a Germpass Elevator Hall Call Unit configured in an open configuration.

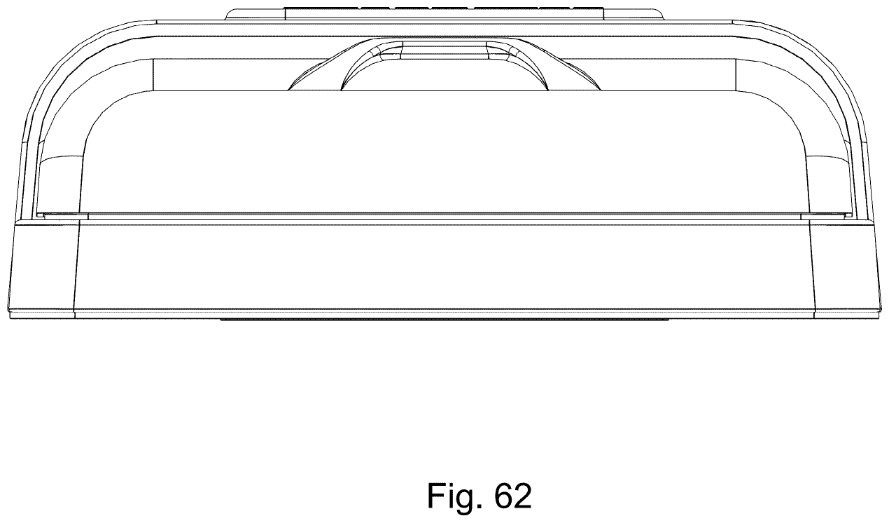

FIG. 62 shows a bottom view of an example embodiment of a Germpass Elevator Hall Call Unit configured in an open configuration.

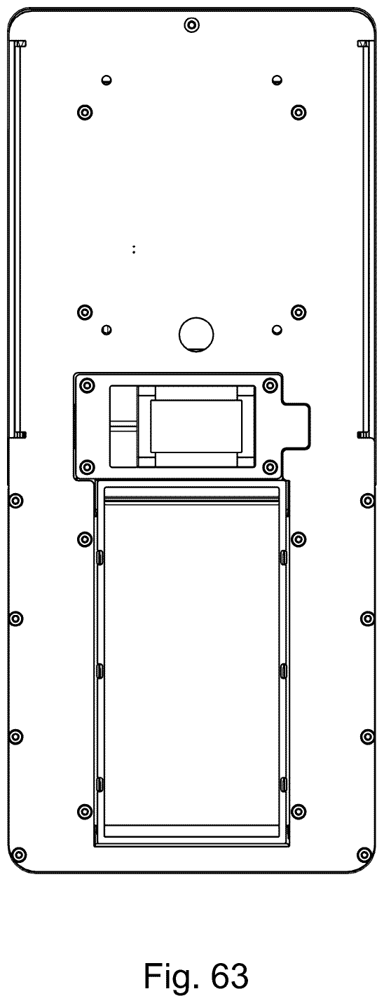

FIG. 63 shows a rear view of an example embodiment of a Germpass Elevator Hall Call Unit configured in an open configuration.

The Germpass Elevator Hall Call Unit is a specialized decontamination system designed to minimize the transmission of pathogenic microorganisms in public settings, particularly around elevator buttons, which are high-contact surfaces. This unit uses ultraviolet germicidal irradiation (UVGI) to sterilize the elevator call buttons after each user interaction, thus reducing contamination and the spread of infectious diseases. The unit operates in both a closed configuration, where the buttons are covered and protected, and an open configuration, where the buttons are exposed for user interaction.

In a closed configuration, as depicted, for example, in FIG. 48 through FIG. 56, the Germpass Elevator Hall Call Unit completely encloses the elevator call buttons to shield them from environmental contamination and airborne pathogens when not in use. This state is maintained until a user approaches, and only after confirming user intent, the unit transitions to the open configuration, allowing interaction.

The front cover plate (4910) is designed to accommodate the movement of the access panel(s) into a stacked configuration behind it when the access panel(s) are in the open state. This cover plate, which remains visible in the closed state, functions as a durable external shield, protecting the elevator call button panel (4930) from potential contaminants. Its robust construction, which may be high-impact plastic or coated metal, ensures resilience in high-traffic areas, with the potential for antimicrobial coatings to further reduce surface contamination. The size and shape of the front cover plate are optimized to facilitate the smooth retraction of the access panels behind it, maintaining the protective function of the unit while allowing seamless access to the buttons.

In at least one embodiment, the Germpass Unit may include one or more movable access panels (4920). These panels may be designed to move into a stacked configuration behind the front cover plate when they are in the open position. In some embodiments, the movable access panel may have a U-shaped cross-section, which aids in its guided movement during retraction and ensures a secure fit when closed. While in the closed position, the panel fully extends to cover the elevator call button panel (4930), preventing contamination. Upon detecting user presence, the panel retracts smoothly, exposing the buttons for interaction. The panel's movement is automated via precision motors and a drive assembly, allowing for seamless operation and swift user access.

The elevator call button panel (4930) contains the buttons used to call the elevator. This panel is recessed behind the access panel (4920) and remains protected during the closed state. The buttons are exposed only when a user is detected, which reduces the risk of contamination from multiple users touching the same surface.

The rear opening (4940), as depicted in FIG. 56 and FIG. 63, facilitates the retrofit installation of the Germpass Unit by allowing it to be positioned directly over the fomite region of an existing fixture without necessitating the removal of that fixture. This design enables seamless integration of the unit with the pre-existing infrastructure while still providing access to internal components, such as the electronic and mechanical systems that control the movement of the access panel and UV-C light sources. The rear opening also serves as a conduit for wiring or mounting hardware, simplifying installation in various settings.

In the open configuration, as illustrated, for example, in FIG. 57 through FIG. 63, the movable access panel (4920) retracts behind cover plate 4910, revealing the elevator call button panel (4930) and allowing users to press the buttons. Once the user interaction is complete, the panel closes, and a UVGI decontamination cycle begins to sterilize the buttons before the next user interaction.

When the system detects a user through motion or proximity sensors, the movable access panel (4920) is retracted upward or sideways, depending on the design, exposing the elevator call button panel (4930) for user interaction. The smooth and swift retraction of the panel ensures minimal delay in accessing the buttons. The movement of the panel is driven by an integrated motor and drive assembly, which may include sensors to monitor the position of the panel and ensure accurate opening and closing cycles. This automated process minimizes the risk of manual tampering or accidental obstruction during the panel's movement.

During execution of a decontamination cycle, a UV-C light source inside the Unit's enclosure is activated, emitting germicidal radiation at approximately 265 nm, which is highly effective in destroying the DNA and RNA of pathogenic microorganisms. The internal surfaces of the unit, including the rear of the movable access panel (4920) and surrounding areas, may be coated with UV-reflective materials to ensure complete coverage and maximize the efficacy of the decontamination cycle. This ensures that every part of the button panel is exposed to sufficient UV-C light for thorough sterilization. The UVGI system is designed to be energy-efficient, with LEDs or other UV-C sources that provide a high dose of irradiation in a short period.

The UV-C light system is contained within the closed unit to prevent accidental exposure to users. Multiple safety mechanisms, such as interlocks and sensors, ensure that the UVGI system operates only when the panel is securely closed. The system may also incorporate monitoring sensors that track the UV-C dosage to ensure consistent sterilization. The decontamination cycle is completed quickly, typically within a few seconds, after which the unit resets to detect the next user.

### Germpass Embedded Elevator Cab Unit Example Embodiments

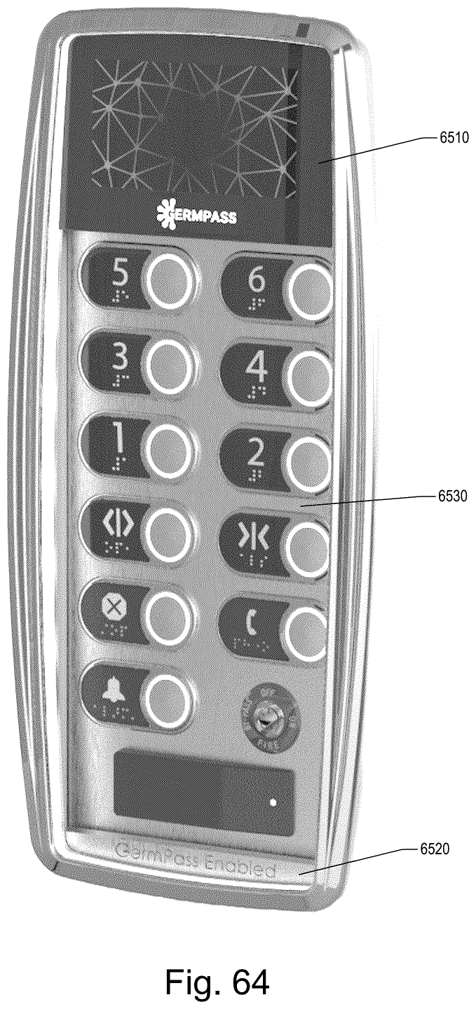

FIGS. 64-78 illustrate different views and features of a Germpass Embedded Elevator Cab Unit according to at least one embodiment.

FIG. 64 shows a perspective view of an example embodiment of a Germpass Embedded Elevator Cab Unit configured in an open configuration.

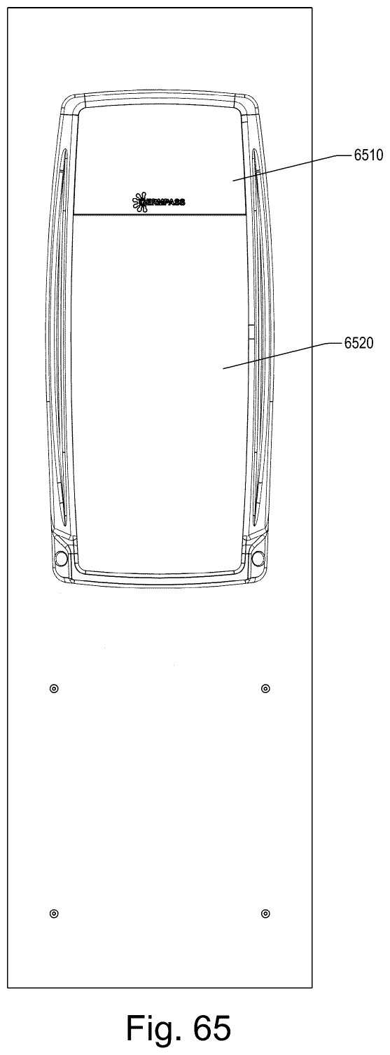

FIG. 65 shows a front view of an example embodiment of a Germpass Embedded Elevator Cab Unit configured in a closed configuration.

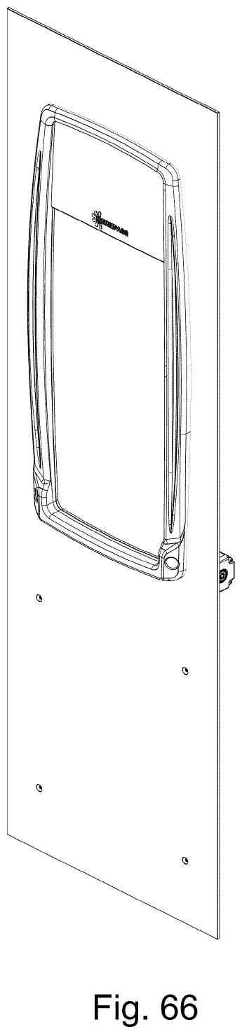

FIG. 66 shows a perspective view of an example embodiment of a Germpass Embedded Elevator Cab Unit configured in a closed configuration.

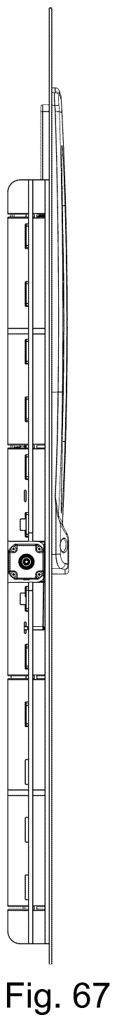

FIG. 67 shows a left view of an example embodiment of a Germpass Embedded Elevator Cab Unit configured in a closed configuration.

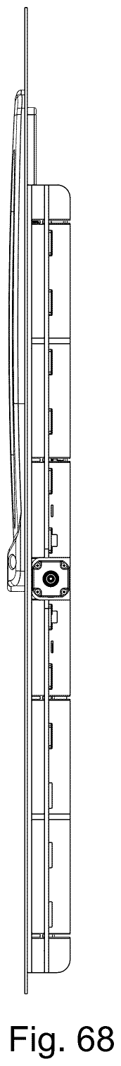

FIG. 68 shows a right view of an example embodiment of a Germpass Embedded Elevator Cab Unit configured in a closed configuration.

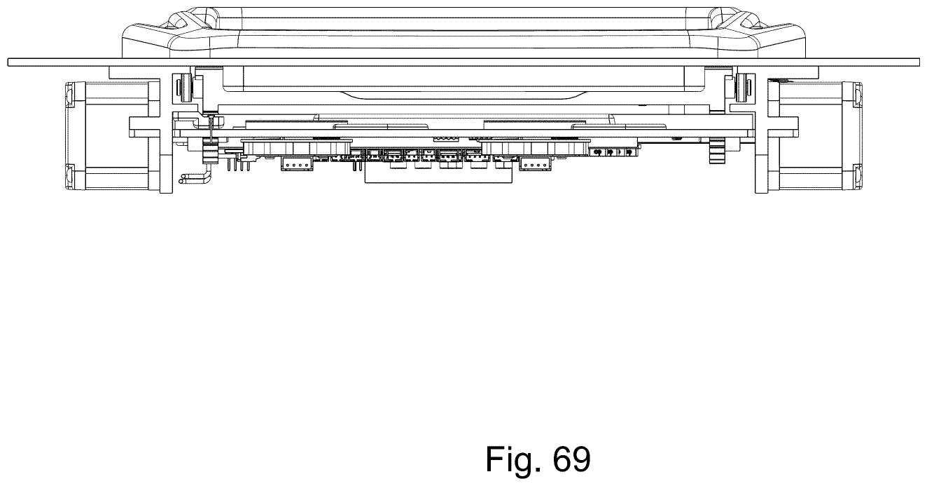

FIG. 69 shows a top view of an example embodiment of a Germpass Embedded Elevator Cab Unit configured in a closed configuration.

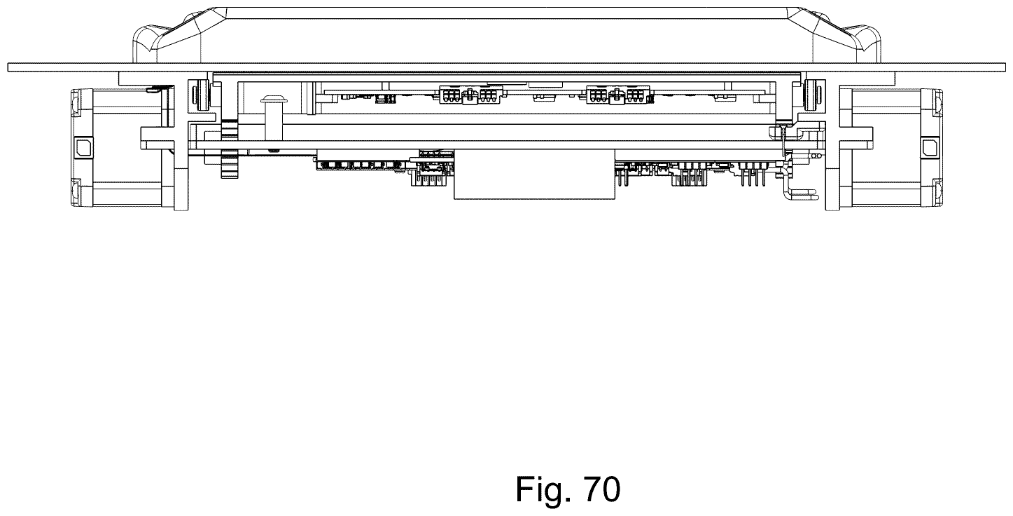

FIG. 70 shows a bottom view of an example embodiment of a Germpass Embedded Elevator Cab Unit configured in a closed configuration.

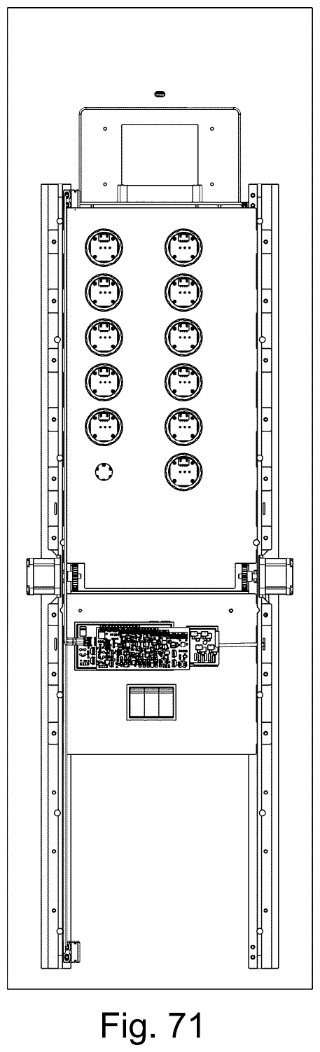

FIG. 71 shows a rear view of an example embodiment of a Germpass Embedded Elevator Cab Unit configured in a closed configuration.

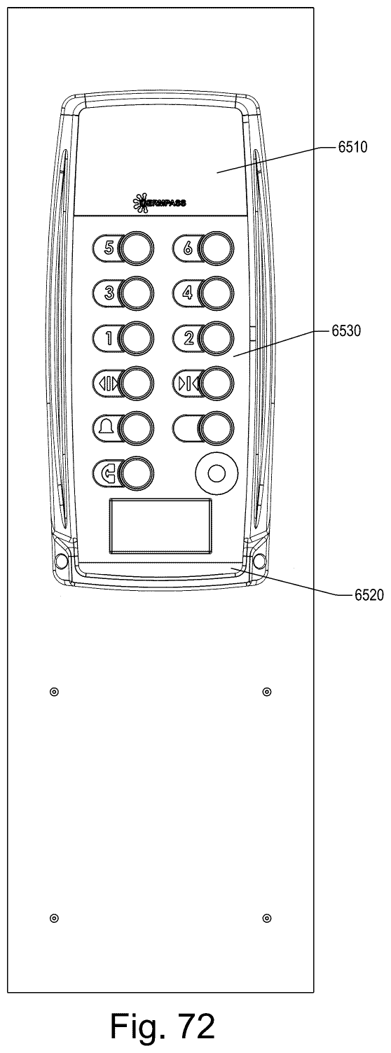

FIG. 72 shows a front view of an example embodiment of a Germpass Embedded Elevator Cab Unit configured in an open configuration.

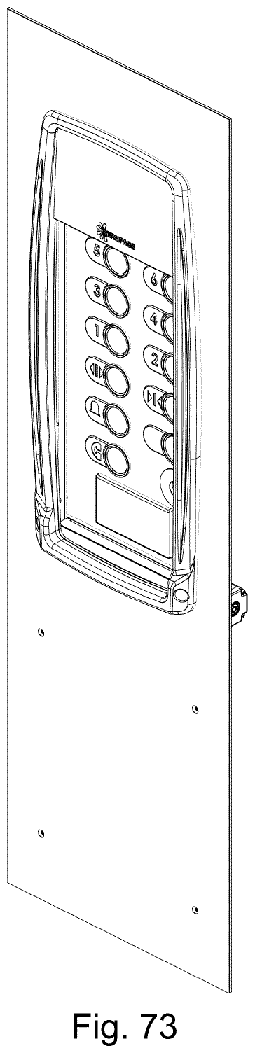

FIG. 73 shows a perspective view of an example embodiment of a Germpass Embedded Elevator Cab Unit configured in an open configuration.

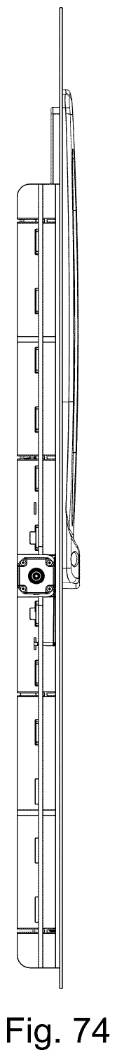

FIG. 74 shows a left view of an example embodiment of a Germpass Embedded Elevator Cab Unit configured in an open configuration.

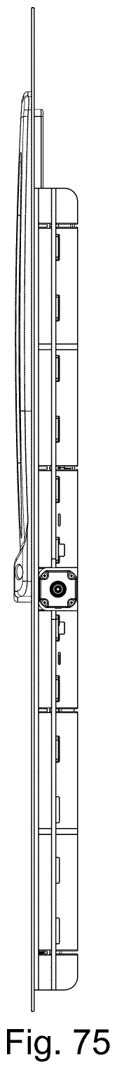

FIG. 75 shows a right view of an example embodiment of a Germpass Embedded Elevator Cab Unit configured in an open configuration.

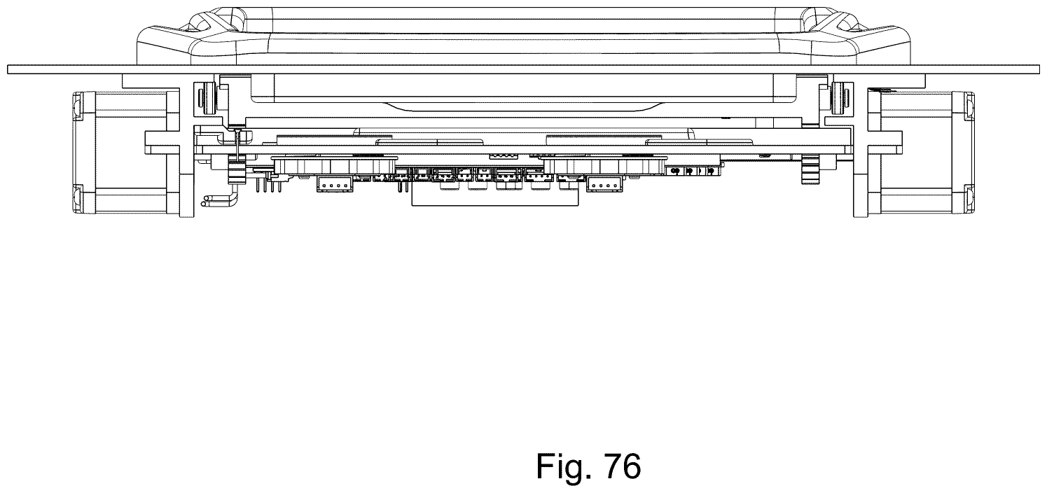

FIG. 76 shows a top view of an example embodiment of a Germpass Embedded Elevator Cab Unit configured in an open configuration.

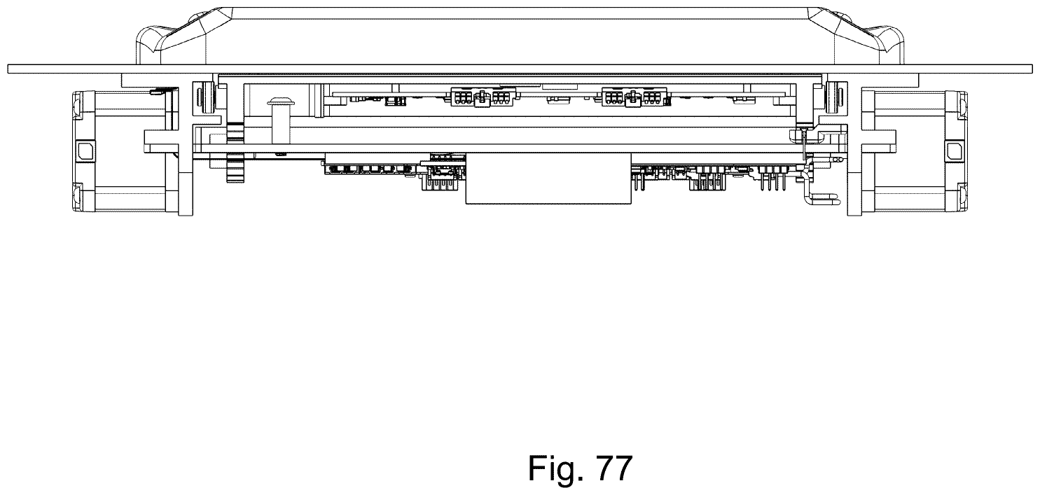

FIG. 77 shows a bottom view of an example embodiment of a Germpass Embedded Elevator Cab Unit configured in an open configuration.

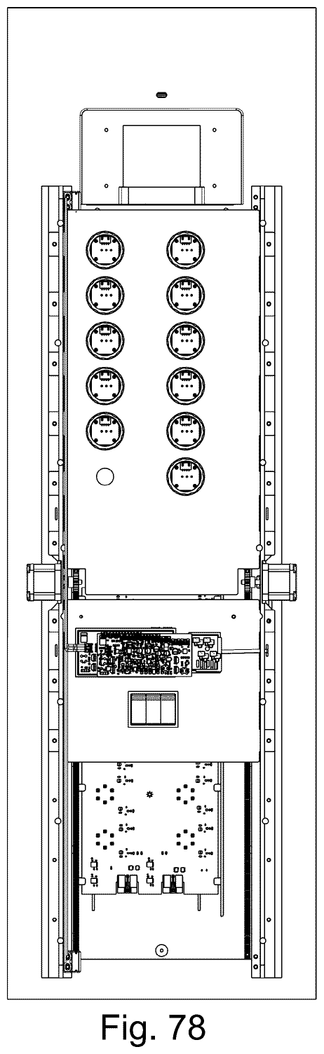

FIG. 78 shows a rear view of an example embodiment of a Germpass Embedded Elevator Cab Unit configured in an open configuration.

The Germpass Embedded Elevator Cab Unit is designed to rapidly and effectively sanitize elevator control panels using ultraviolet germicidal irradiation (UVGI). The following sections provide an in-depth description of its components and how each functions within the system.

The front cover plate (6510) is specifically designed to accommodate the movement of the access panel(s) into a stacked configuration behind it when the access panel(s) are in the open position. In its closed position (as shown in FIGS. 65-71), the cover plate acts as a protective barrier, fully shielding the elevator buttons from airborne pathogens, dust, and environmental contaminants. This design ensures that the buttons remain uncontaminated during periods of inactivity. The cover plate is constructed from durable materials and features reflective interior surfaces to optimize UV-C light reflection during the decontamination cycle, ensuring comprehensive irradiation of the button panel. Additionally, UV-resistant materials are incorporated to prevent degradation from prolonged exposure to ultraviolet light.

The Germpass Unit may include one or more movable access panels (6520), which are designed to move into a stacked configuration behind the front cover plate when in the open configuration, as depicted in FIGS. 64, 72-78. This panel allows users to access the control panel while facilitating smooth transitions between standby, access, and decontamination modes. Mounted on precision rails and guided by a motorized drive assembly, the panel's movement is tracked by position sensors for accurate operation. During decontamination, the access panel remains closed, fully enclosing the elevator buttons to enable a controlled UVGI cycle.

The access panel's design ensures that it remains securely locked during the decontamination process to prevent accidental exposure to UV-C light, which may harm users. Additionally, it is programmed to open as soon as a user approaches, as determined by motion and proximity sensors embedded within the unit. Upon completion of the user interaction, the panel automatically closes, initiating the next decontamination cycle.

The drive assembly is responsible for the movement of the access panel (6520). It consists of a motor, gears, and pulleys designed to smoothly retract or extend the panel as required. The assembly is controlled by a microcontroller, which coordinates with various sensors (e.g., proximity, position, and motion sensors) to ensure the precise and safe operation of the system. The drive assembly is also fitted with obstruction detection sensors, which immediately halt the panel's movement if any resistance or blockage is detected, ensuring the safety of passengers.

The rotary encoders in the drive assembly track the real-time position of the panel, enabling the system to identify any abnormal configurations, such as partial openings or unexpected stops. This helps maintain system integrity and ensures the panel always functions as intended.

The UVGI system embedded in the elevator cab unit includes multiple UV-C LEDs strategically positioned within the enclosure. These LEDs emit UV-C radiation at a wavelength of approximately 265 nm, which is optimal for destroying the DNA of pathogenic microorganisms, rendering them inactive. The UVGI system is activated immediately after the access panel (6520) closes, initiating a decontamination cycle that lasts for a preset duration, typically a few seconds, depending on the environmental settings and traffic in the elevator.

In some embodiments, a reflective coating inside the enclosure, especially on the front cover plate (6510), enhances the efficiency of the UV-C radiation by ensuring that the light reaches all exposed surfaces of the control panel. The UVGI system is also monitored by the microcontroller, which tracks the status of the UV-C LEDs and ensures that the required dosage of ultraviolet light is delivered to guarantee effective decontamination before the next user interaction.

In at least some embodiments, a Germpass Unit may include a comprehensive array of functional sensors strategically placed within the unit. The functional sensors may include one or more of the following types (or combinations thereof):
Motion Sensors: These detect the approach of passengers, triggering the opening of the access panel (6520).
Proximity Sensors: These help identify the precise moment when a user intends to interact with the elevator buttons, further optimizing panel operation.
Position Sensors: These monitor the location of the movable access panel (6520) along its track, ensuring correct panel positioning during opening and closing cycles

Each of these functional sensor types may be implemented using one or more underlying sensor technologies, including but not limited to:
Ultrasonic Sensors utilizing high-frequency sound wave reflection.
Millimeter Wave Sensors employing high-frequency radio wave detection
Infrared Sensors, including:
   Passive Infrared (PIR) sensors for thermal detection
   Active Infrared (AIR) sensors using beam interruption
Other suitable sensor technologies that enable the required detection capabilities

The functional sensors are placed strategically within the unit and may be configured or designed to detect human presence and motion, facilitating real-time adjustments to the Germpass Unit's configuration and initiating the UVGI cycle when appropriate. The system may utilize multiple underlying sensor technologies to implement each functional sensor type, providing redundancy and enhanced detection reliability.

For example, a motion sensor function may be implemented using a combination of ultrasonic and PIR technologies, while proximity sensing may utilize millimeter wave and AIR technologies. This multi-technology approach enables robust detection capabilities across varying environmental conditions while maintaining precise control over the unit's operations.

A microcontroller is the central processing unit that governs all operations within the Germpass Unit. It collects data from the sensor array, processes user interactions, and controls the UVGI cycle. The microcontroller operates under a real-time operating system (RTOS), which allows it to handle multitasking, such as sensor monitoring, panel control, and safety checks, without lag or errors. It ensures the unit operates efficiently in both low-and high-traffic environments by adjusting the sensitivity of the sensors and the timing of the decontamination cycles.

In addition to handling normal operations, the microcontroller also manages safety protocols, such as ensuring the UV-C light only activates when the access panel is closed and locked, and halting panel movement if an obstruction is detected.

Safety is an important consideration in the Germpass Embedded Elevator Cab Unit. The UV-C system is enclosed within the unit and cannot be activated unless the access panel is fully closed. Obstruction sensors prevent the panel from closing on a user's hand or any other object, ensuring passenger safety during operation. The microcontroller continuously monitors these safety systems and may automatically shut down the unit or notify maintenance personnel if any fault is detected. In the event of a malfunction, the unit is designed to open upon detection of failure.

### Germpass Retrofit Elevator Cab Unit Example Embodiments

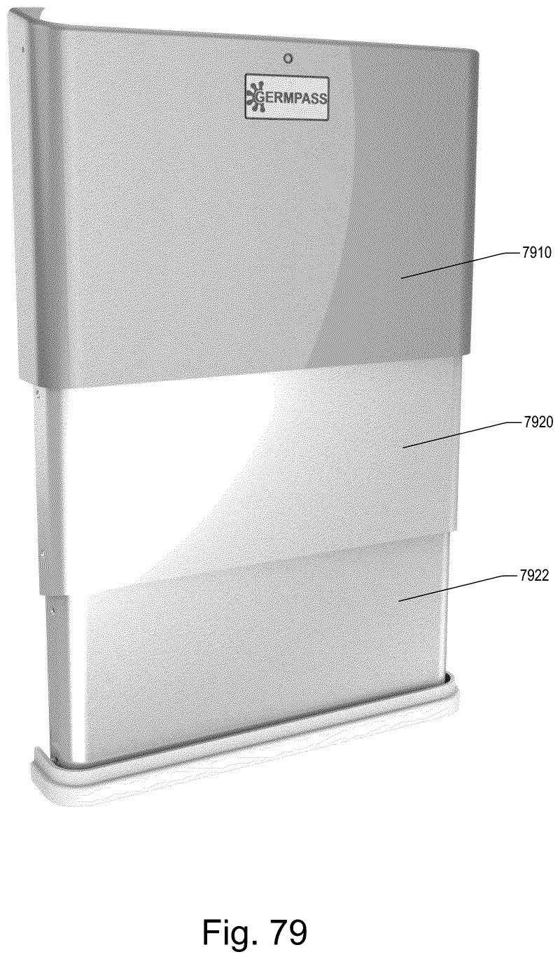

FIGS. 79-94 illustrate different views and features of a Germpass Retrofit Elevator Cab Unit according to at least one embodiment.

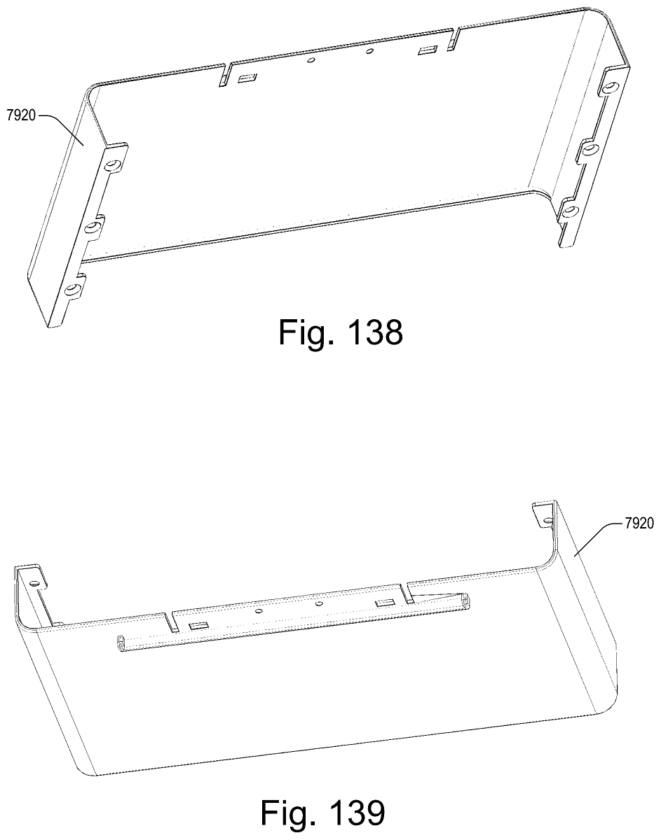

FIGS. 138 and 139 show different perspective views of a Germpass Retrofit Elevator Cab Unit movable intermediate access panel 7920 in accordance with at least one embodiment.

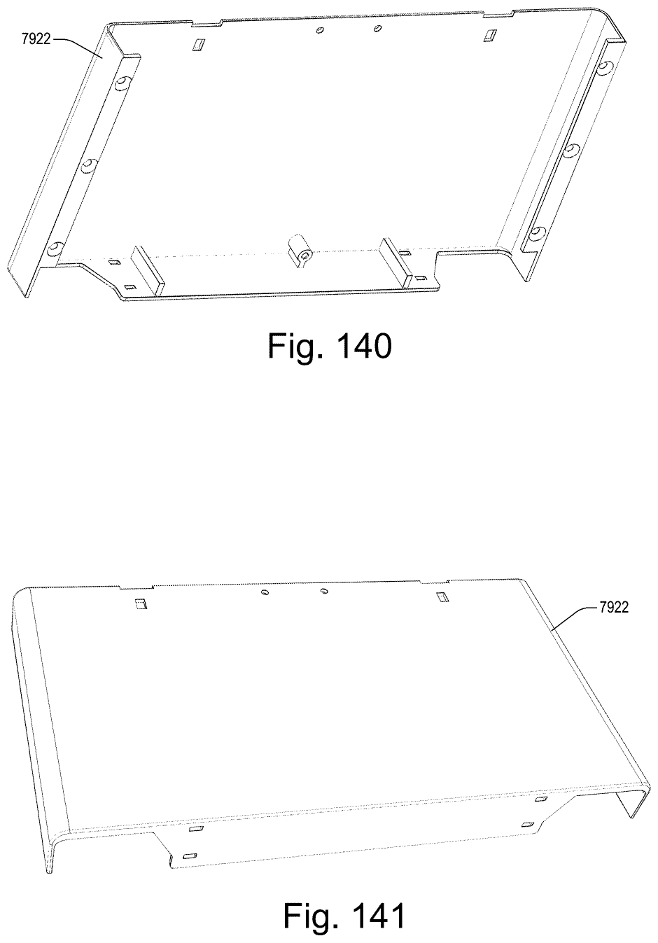

FIGS. 140 and 141 show different perspective views of a Germpass Retrofit Elevator Cab Unit movable end access panel 7922 in accordance with at least one embodiment.

FIG. 79 shows a perspective view of an example embodiment of a Germpass Retrofit Elevator Cab Unit configured in a closed configuration.

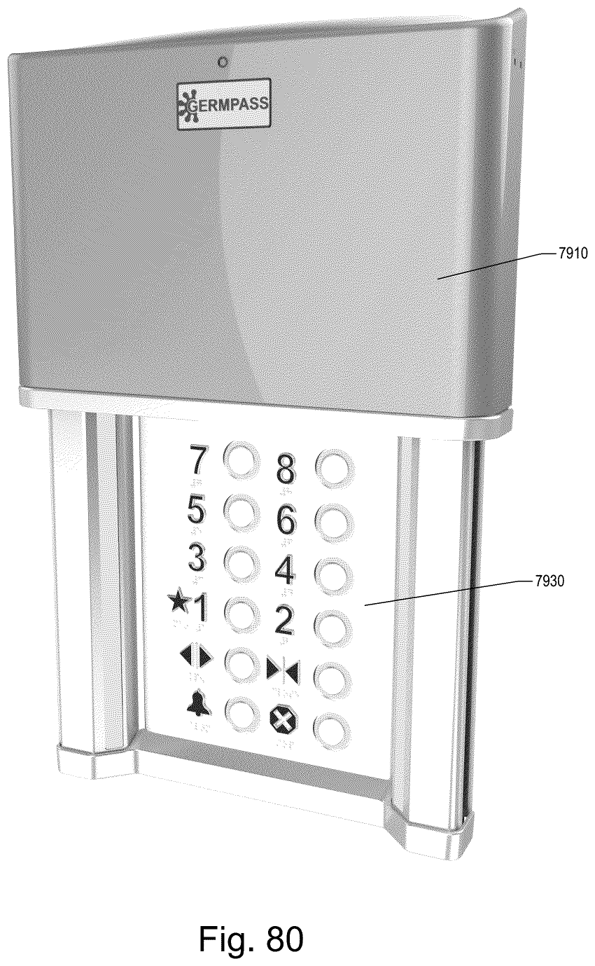

FIG. 80 shows a perspective view of an example embodiment of a Germpass Retrofit Elevator Cab Unit configured in an open configuration.

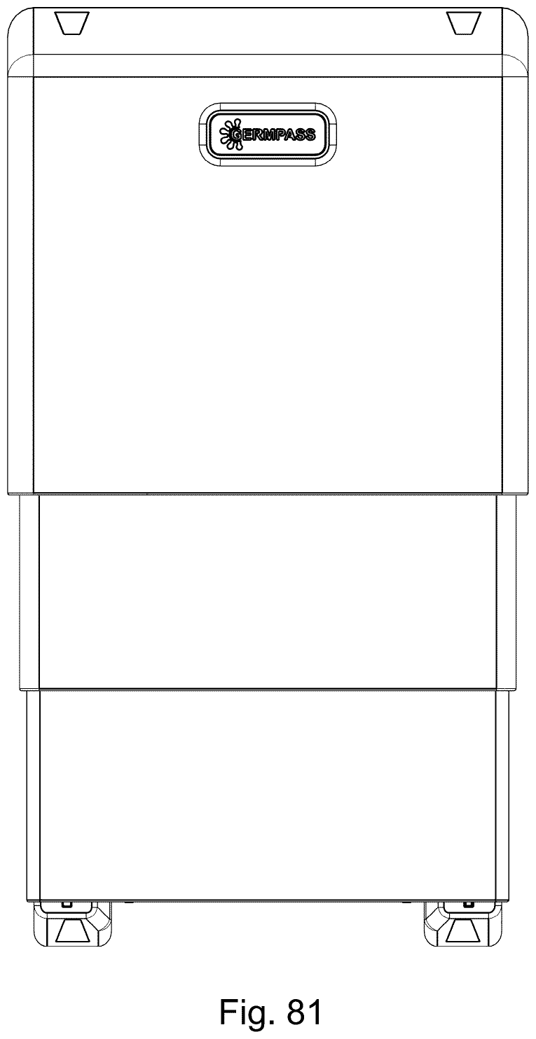

FIG. 81 shows a front view of an example embodiment of a Germpass Retrofit Elevator Cab Unit configured in a closed configuration.

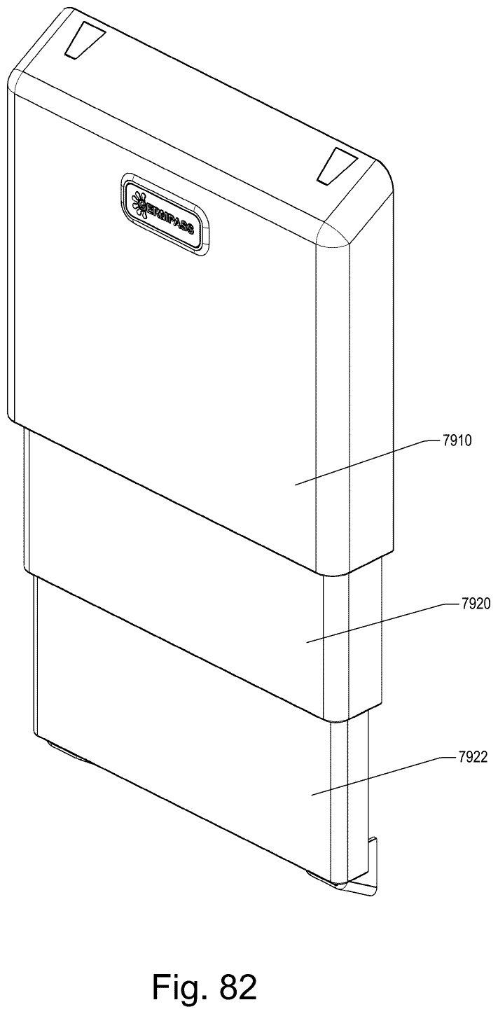

FIG. 82 shows a perspective view of an example embodiment of a Germpass Retrofit Elevator Cab Unit configured in a closed configuration.

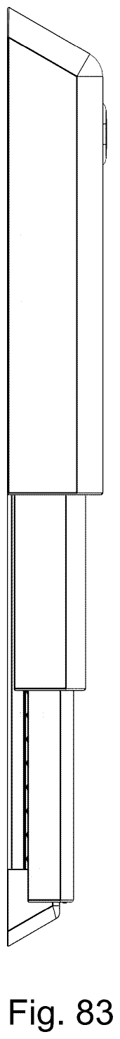

FIG. 83 shows a left view of an example embodiment of a Germpass Retrofit Elevator Cab Unit configured in a closed configuration.

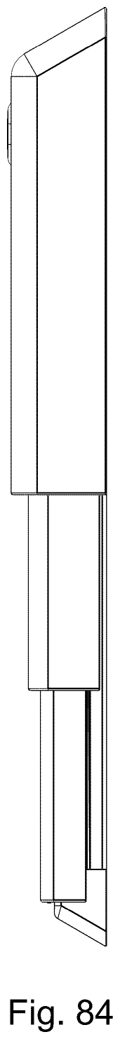

FIG. 84 shows a right view of an example embodiment of a Germpass Retrofit Elevator Cab Unit configured in a closed configuration.

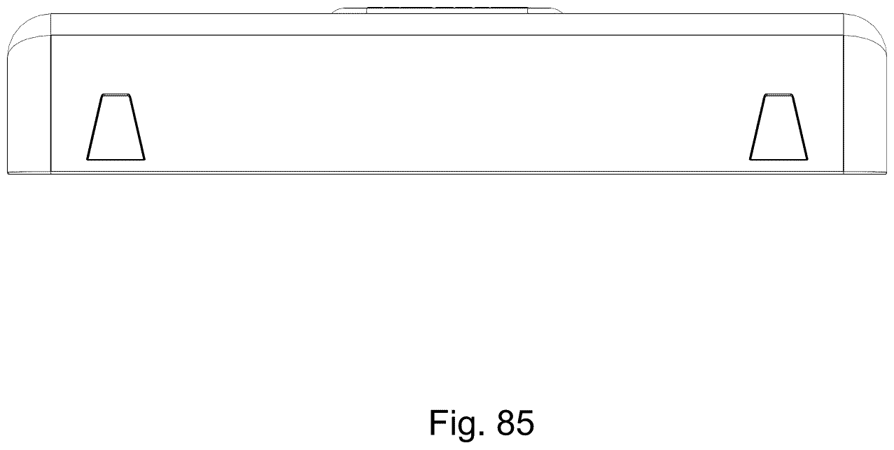

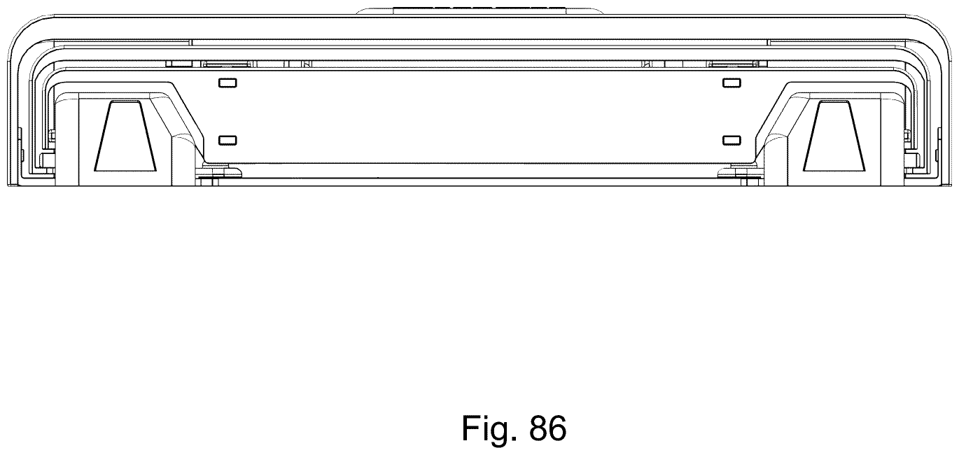

FIG. 85 shows a top view of an example embodiment of a Germpass Retrofit Elevator Cab Unit configured in a closed configuration.

FIG. 86 shows a bottom view of an example embodiment of a Germpass Retrofit Elevator Cab Unit configured in a closed configuration.

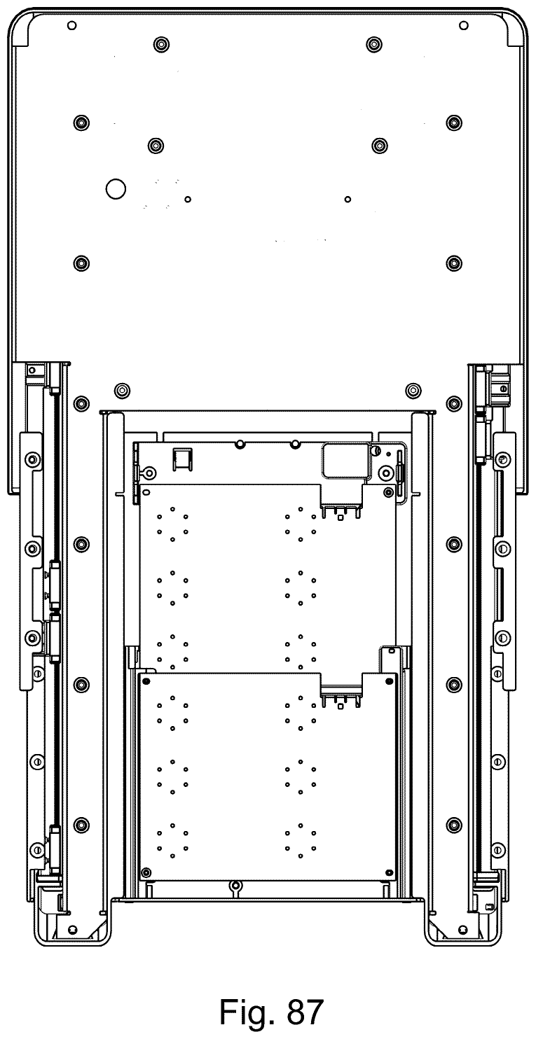

FIG. 87 shows a rear view of an example embodiment of a Germpass Retrofit Elevator Cab Unit configured in a closed configuration.

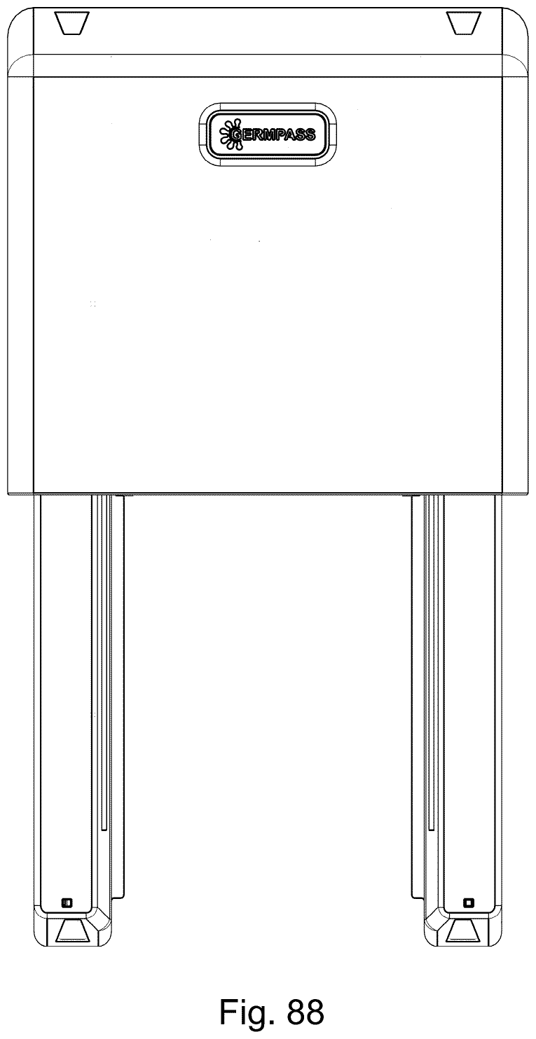

FIG. 88 shows a front view of an example embodiment of a Germpass Retrofit Elevator Cab Unit configured in an open configuration.

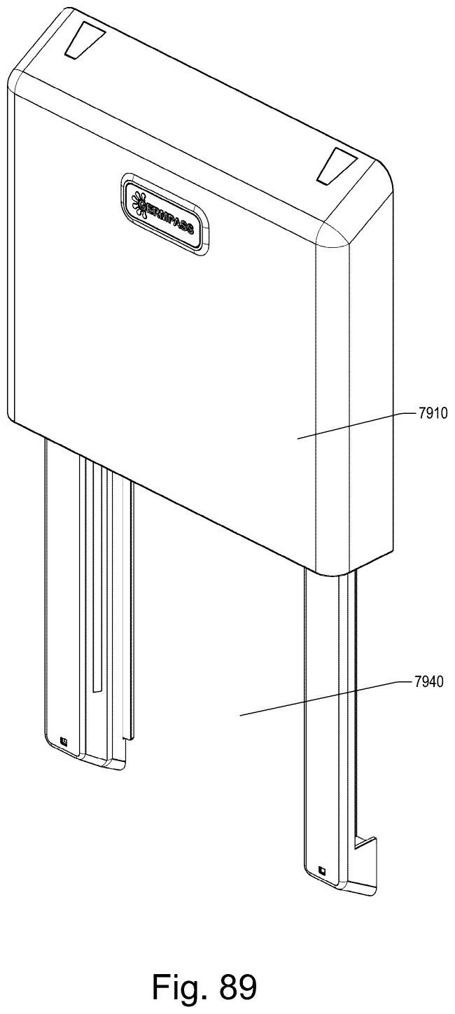

FIG. 89 shows a perspective view of an example embodiment of a Germpass Retrofit Elevator Cab Unit configured in an open configuration.

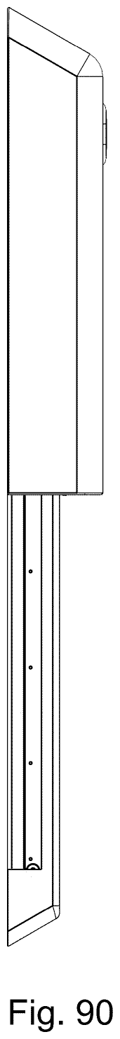

FIG. 90 shows a left view of an example embodiment of a Germpass Retrofit Elevator Cab Unit configured in an open configuration.

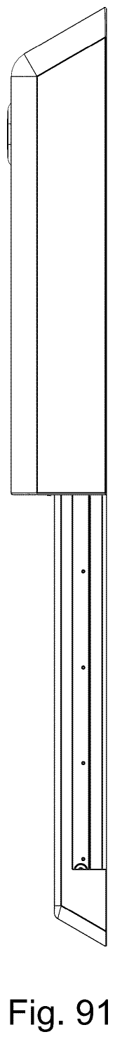

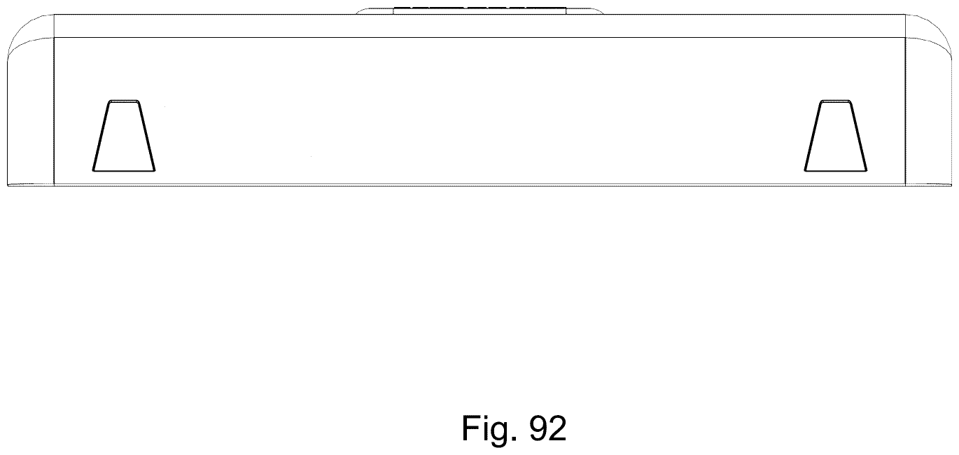

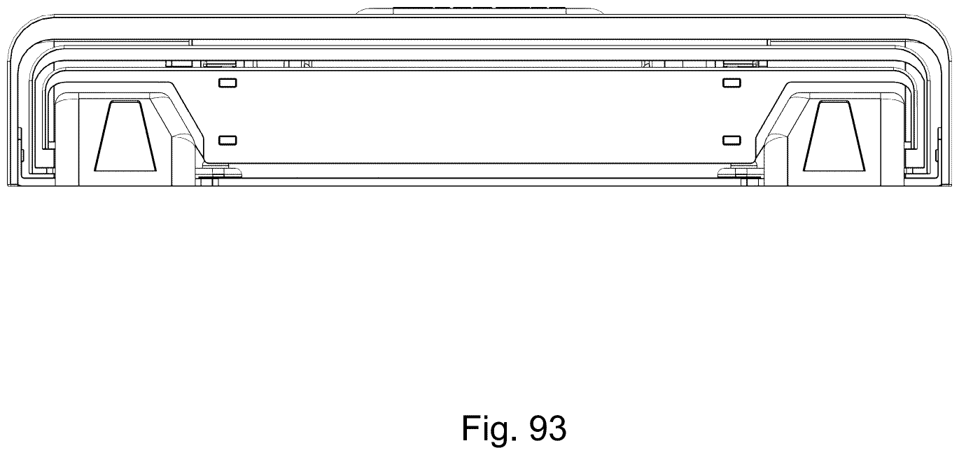

FIG. 91 shows a right view of an example embodiment of a Germpass Retrofit Elevator Cab Unit configured in an open configuration.

FIG. 92 shows a top view of an example embodiment of a Germpass Retrofit Elevator Cab Unit configured in an open configuration.

FIG. 93 shows a bottom view of an example embodiment of a Germpass Retrofit Elevator Cab Unit configured in an open configuration.

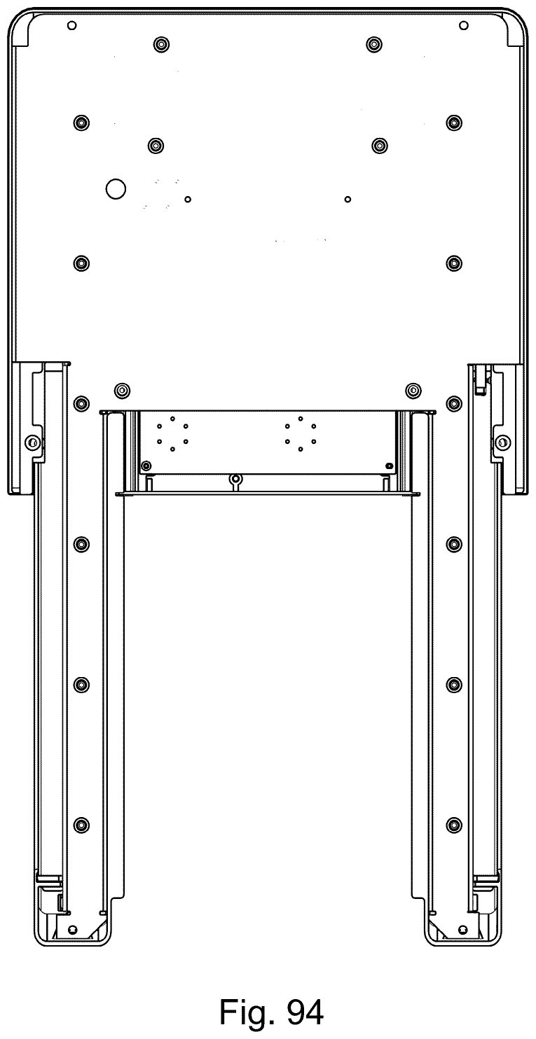

FIG. 94 shows a rear view of an example embodiment of a Germpass Retrofit Elevator Cab Unit configured in an open configuration.

The Germpass Retrofit Elevator Cab Unit represents a specialized decontamination system configured to protect elevator control panels from contamination by utilizing ultraviolet germicidal irradiation (UVGI). Below is an expanded description of the individual components of this system, with associated reference numbers based on the provided figures.

The front cover plate (7910) is specifically designed to allow the movable access panel(s) to be retracted and stacked behind it when the panel(s) are in the open configuration. In the closed configuration, the cover plate forms part of the protective barrier over the elevator control panel, preventing contamination by blocking airborne pathogens and direct contact with the buttons. Its size and shape are optimized to accommodate the access panel(s) as they move seamlessly into a stacked position behind the cover, ensuring unobstructed access to the buttons when triggered by user detection or a pre-determined event. The front cover plate may be constructed from durable materials that not only provide structural integrity but are also optimized for UV-C light reflection, enhancing the effectiveness of the germicidal decontamination process during the UV cycle.

In at least one embodiment, the Germpass Retrofit Elevator Cab Unit may include one or more movable access panels (7920) designed to retract into a stacked configuration behind the front cover plate when in the open configuration. These intermediate panels, which may have a U-shaped cross-section, facilitate smooth access to the elevator buttons by retracting partially when user interaction is detected. The U-shaped design ensures a secure fit over the button area, helping to maintain a controlled environment during decontamination. The panels retract smoothly via a motorized drive system engineered for minimal friction and wear, promoting long-term reliability in high-traffic settings. Additionally, the panels may be constructed from UV-C optimized materials to enhance the efficiency of germicidal light reflection during the decontamination cycle.

An end movable access panel (7922), also designed with a substantially U-shaped cross-section, complements the intermediate panel by covering the remaining portion of the button panel. Like the intermediate panel, the end panel retracts when triggered to grant user access to the control panel. It is constructed from similar UV-C optimized materials to enhance the decontamination process. The combination of the intermediate and end movable panels ensures that the entire button panel is exposed only when necessary, protecting it from contamination during idle periods. Both panels are integrated into the system's drive mechanism, which coordinates their retraction and extension based on sensor input.

The elevator cab button panel (7930) is the fomite that the Germpass Retrofit Elevator Cab Unit is specifically designed to protect. This panel typically contains the elevator buttons that passengers interact with to select floors and operate the elevator. The button panel is exposed to potential contamination from every user interaction, making it a notable area for decontamination. The Germpass system shields this panel when not in use and initiates a UV-C decontamination cycle to disinfect the surface after each use. The unit's sensors and automated panels ensure that the button panel is only exposed during user interaction, and it remains shielded and disinfected during idle periods.

The rear opening (7940) is specifically designed to facilitate the retrofit installation of the Germpass Retrofit Elevator Cab Unit over the fomite region of an existing fixture without requiring removal of the fixture. This opening allows the unit to be seamlessly installed while providing access to its internal components for maintenance, such as replacing UV-C LEDs, checking sensors, or servicing the drive mechanism. It remains securely closed during normal operation to protect internal electronics and mechanical systems, and may include dust-resistant or air-tight seals to prevent contaminants from entering. ensuring the reliability of the decontamination process.

When in operation, the Germpass Retrofit Elevator Cab Unit utilizes sensors to detect when a user approaches the elevator. The system then retracts the intermediate and end movable access panels, allowing the user to interact with the buttons. After the interaction, the panels close, and a UV-C decontamination cycle is initiated to disinfect the button panel. This process ensures that the elevator buttons are protected from contamination between uses, thereby reducing the transmission of germs in public spaces.

The combination of advanced sensor technologies, UV-C light optimization, and a robust motorized drive system makes this unit highly efficient in high-traffic environments such as office buildings, hospitals, and public transportation hubs, where the risk of germ transmission is particularly high.

The UV-C decontamination system integrated into the Germpass Unit is designed to emit ultraviolet light at a wavelength of 265 nm, which is highly effective in inactivating a wide range of pathogens, including viruses and bacteria. The reflective materials used in the front cover plate, intermediate, and end panels enhance the efficiency of this germicidal process by ensuring that the UV-C light is evenly distributed across the button panel surface. After the decontamination cycle is completed, the system resets to its standby mode, awaiting the next user interaction.

This comprehensive approach to elevator button decontamination, combined with advanced automation, ensures the continuous protection of public touch points, making the Germpass Retrofit Elevator Cab Unit a significant innovation in the field of germ prevention and public health technology.

### Germpass Lever Handle Unit Example Embodiments

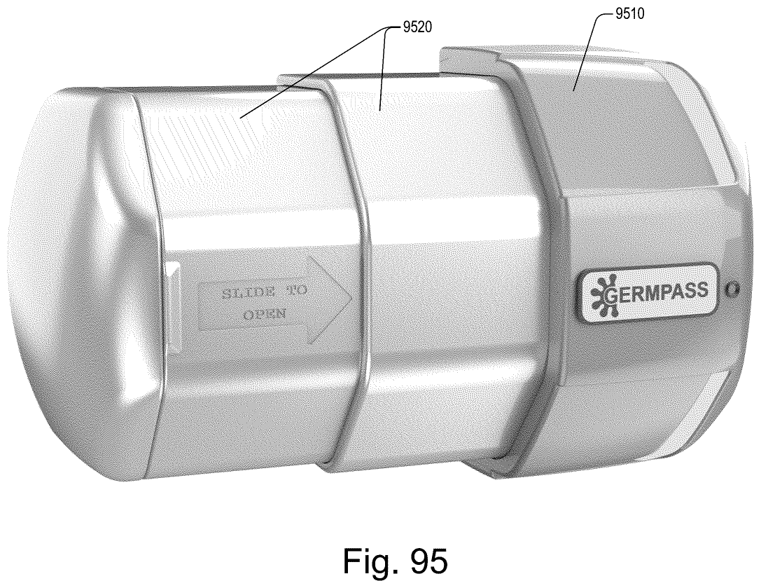

FIGS. 95-110 illustrate different views and features of a Germpass Lever Handle Unit according to at least one embodiment.

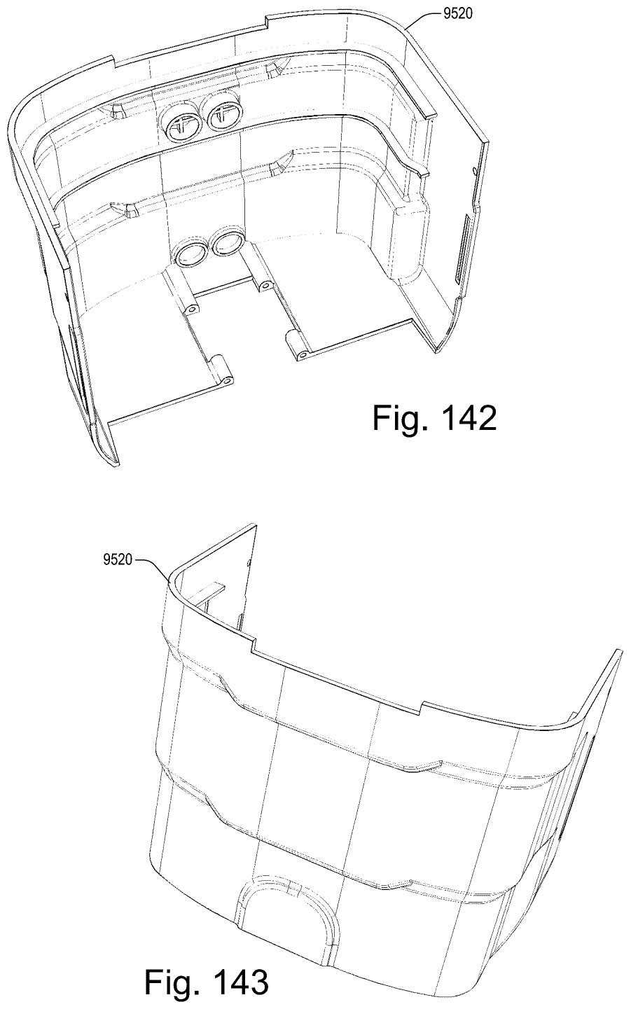

FIGS. 142 and 143 show different perspective views of a Germpass Lever Handle Unit movable access panel 9520 in accordance with at least one embodiment.

FIG. 95 shows a perspective view of an example embodiment of a Germpass Lever Handle Unit configured in a closed configuration.

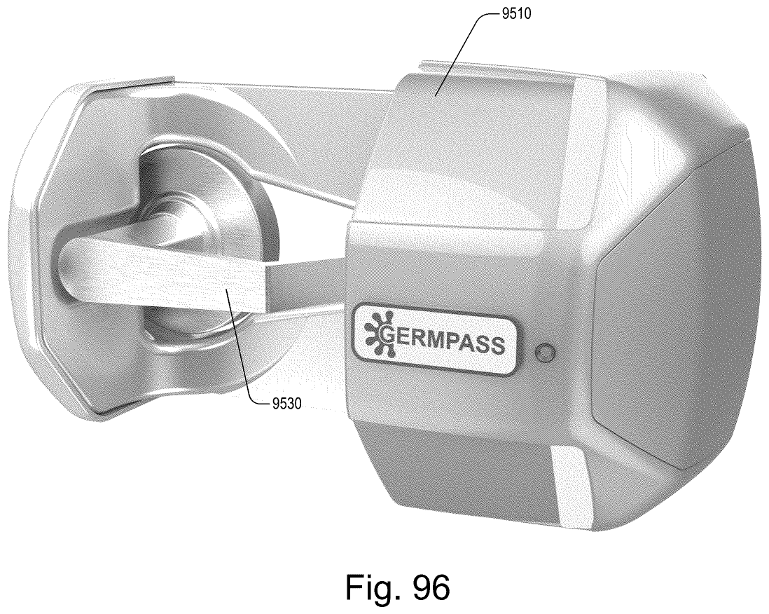

FIG. 96 shows a perspective view of an example embodiment of a Germpass Lever Handle Unit configured in an open configuration.

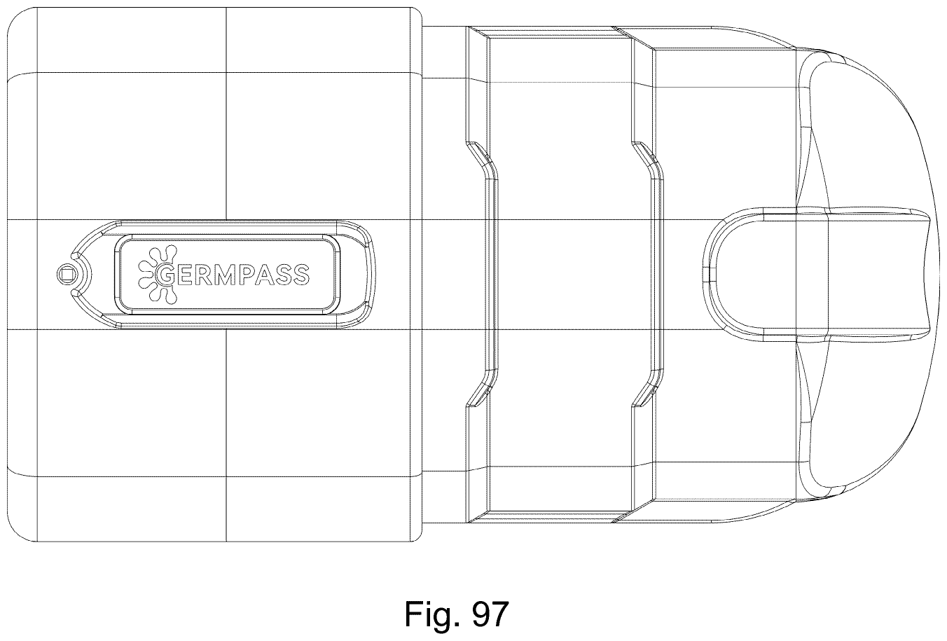

FIG. 97 shows a front view of an example embodiment of a Germpass Lever Handle Unit configured in a closed configuration.

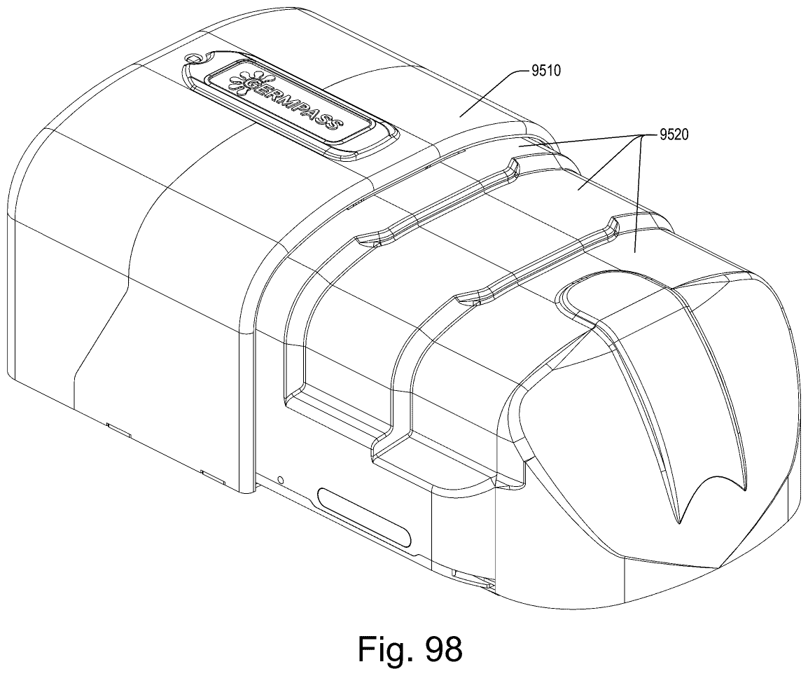

FIG. 98 shows a perspective view of an example embodiment of a Germpass Lever Handle Unit configured in a closed configuration.

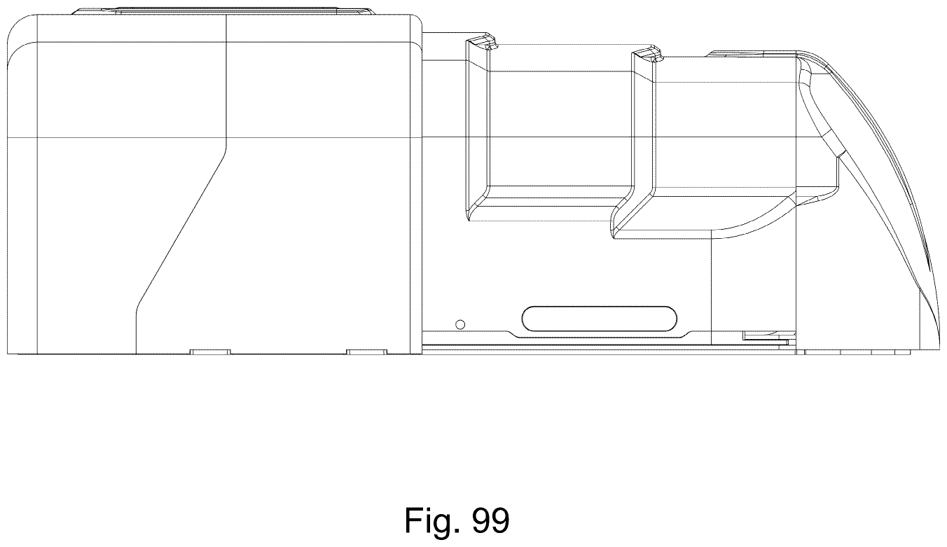

FIG. 99 shows a left view of an example embodiment of a Germpass Lever Handle Unit configured in a closed configuration.

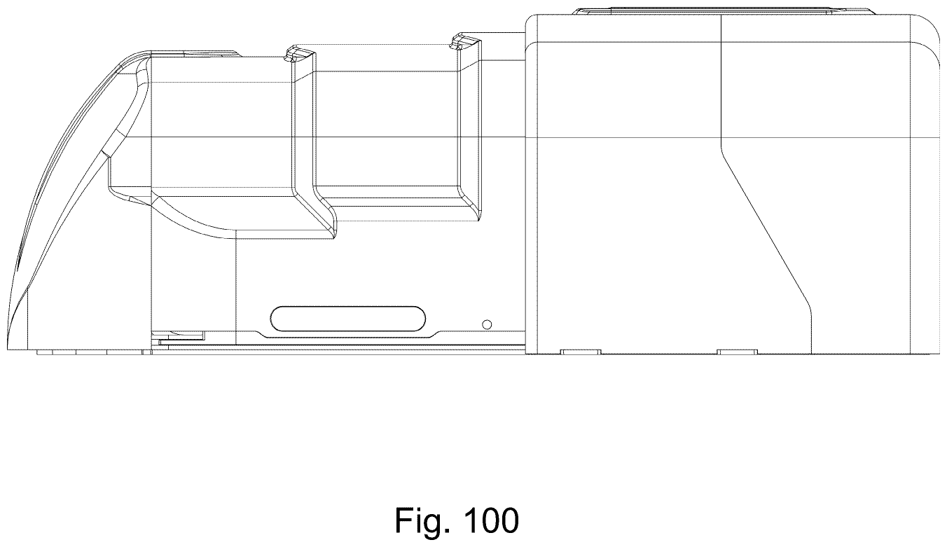

FIG. 100 shows a right view of an example embodiment of a Germpass Lever Handle Unit configured in a closed configuration.

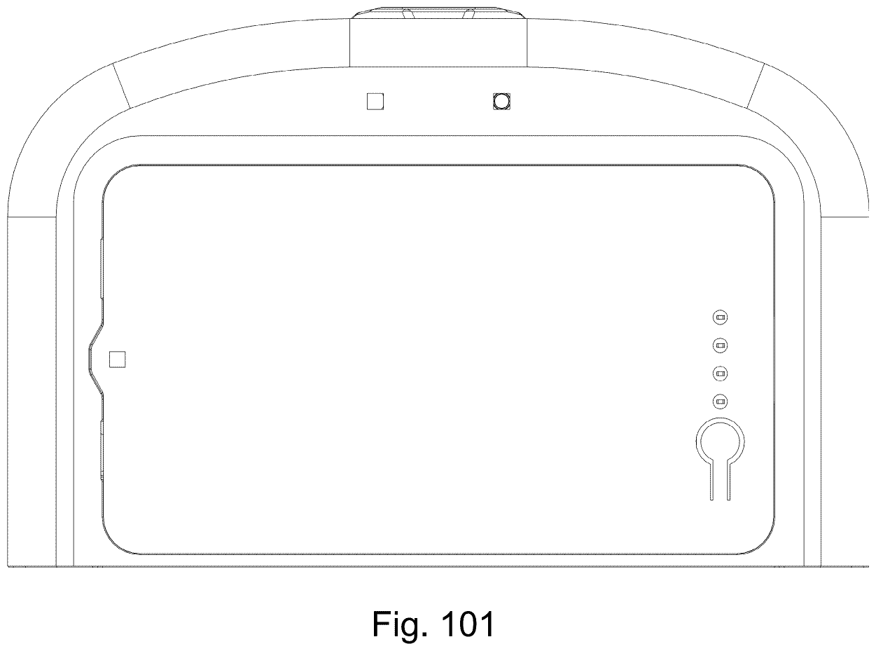

FIG. 101 shows a top view of an example embodiment of a Germpass Lever Handle Unit configured in a closed configuration.

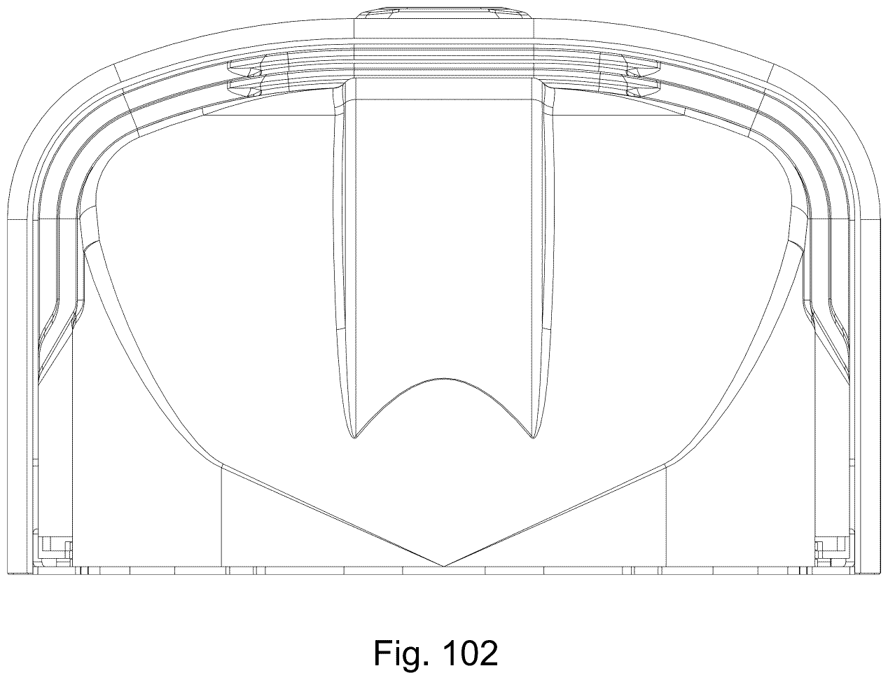

FIG. 102 shows a bottom view of an example embodiment of a Germpass Lever Handle Unit configured in a closed configuration.

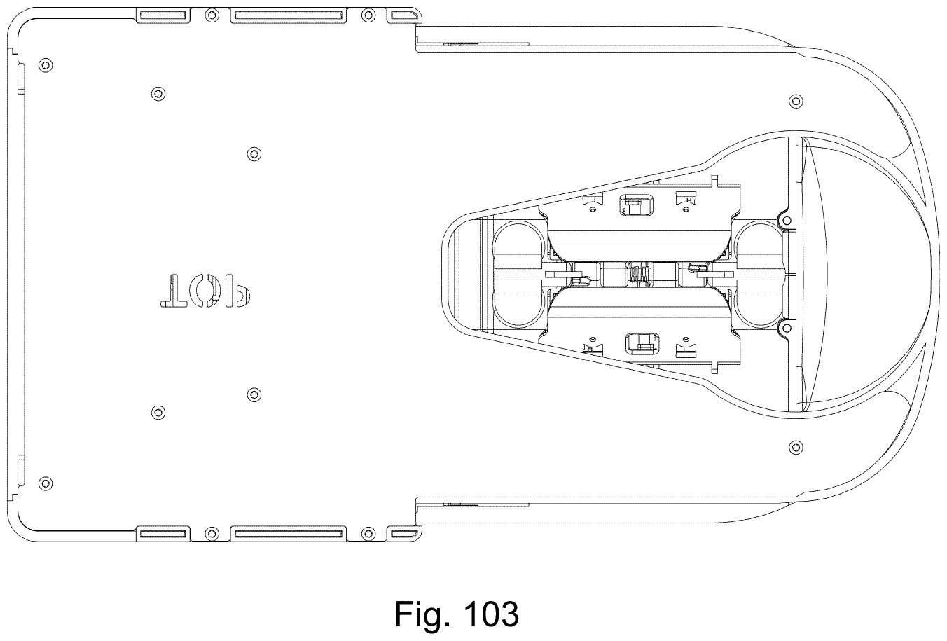

FIG. 103 shows a rear view of an example embodiment of a Germpass Lever Handle Unit configured in a closed configuration.

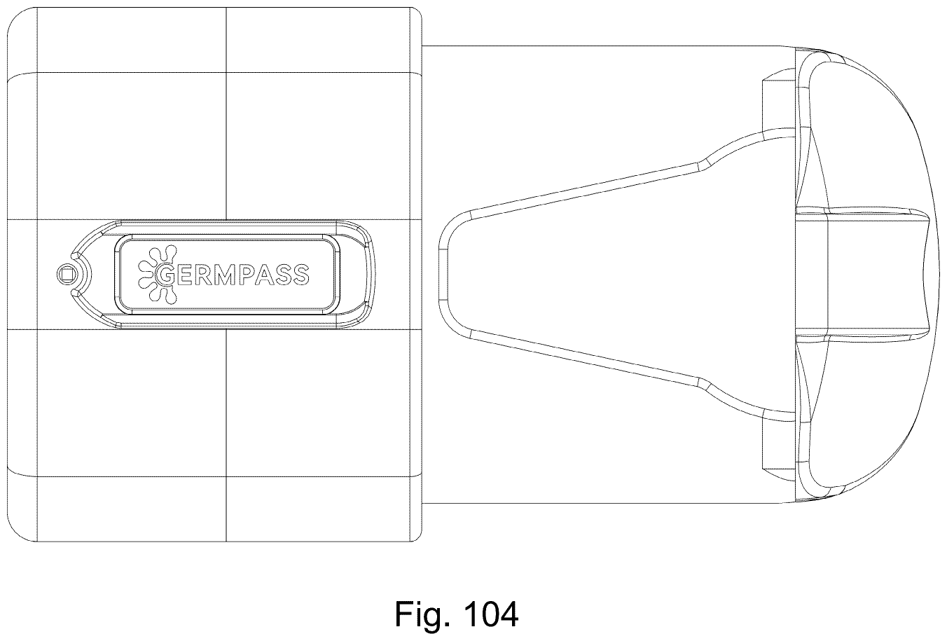

FIG. 104 shows a front view of an example embodiment of a Germpass Lever Handle Unit configured in an open configuration.

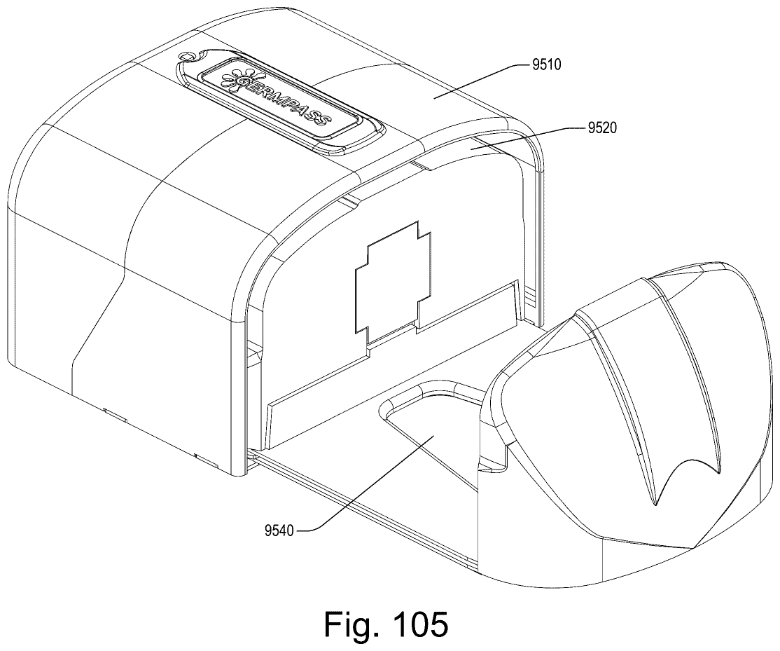

FIG. 105 shows a perspective view of an example embodiment of a Germpass Lever Handle Unit configured in an open configuration.

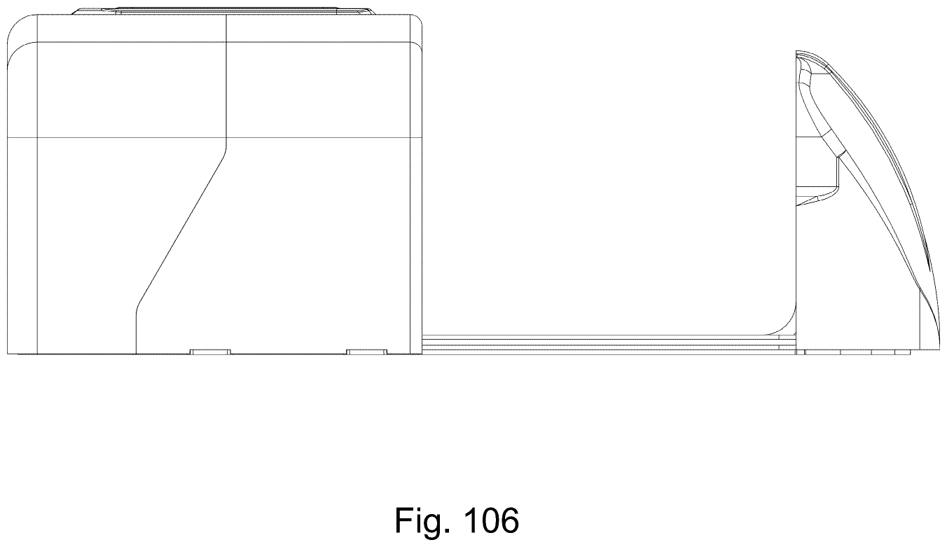

FIG. 106 shows a left view of an example embodiment of a Germpass Lever Handle Unit configured in an open configuration.

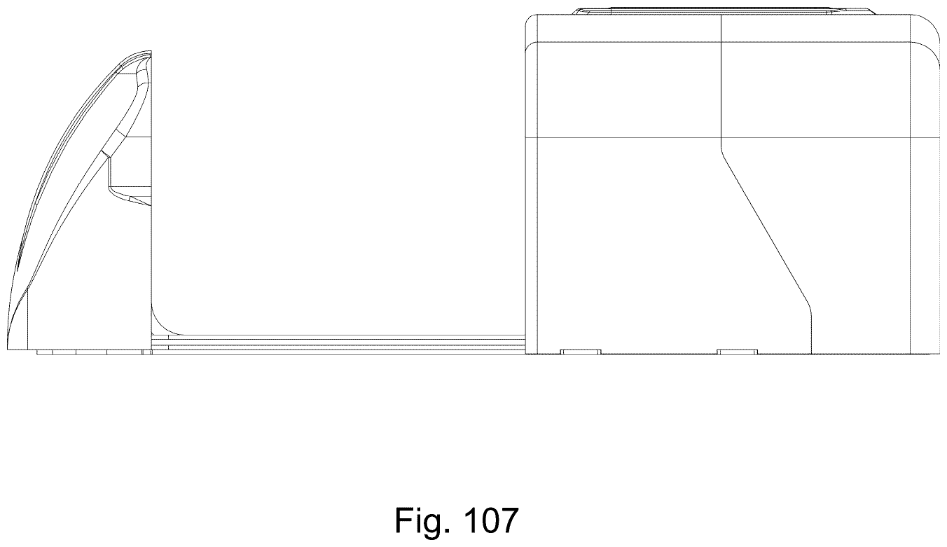

FIG. 107 shows a right view of an example embodiment of a Germpass Lever Handle Unit configured in an open configuration.

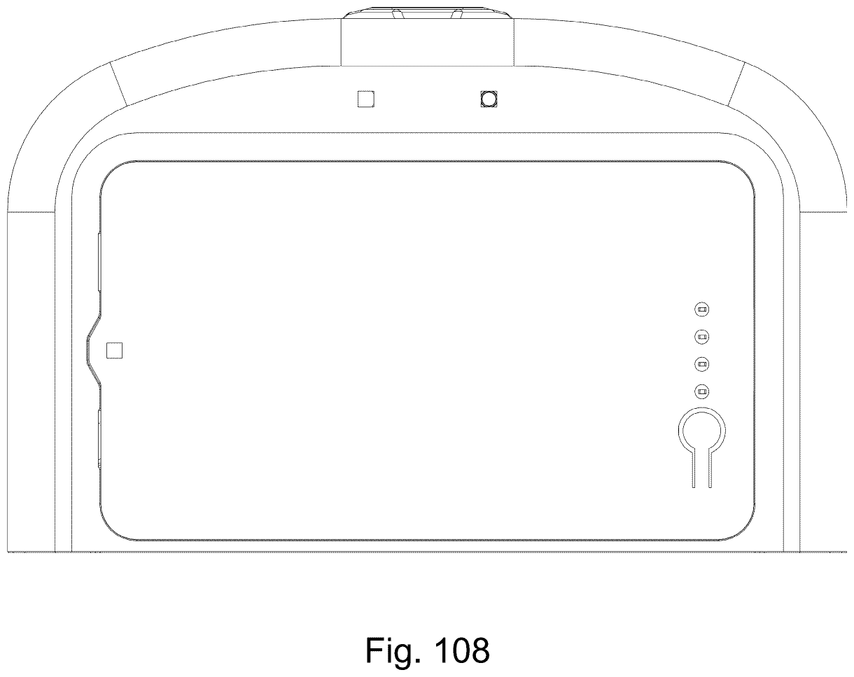

FIG. 108 shows a top view of an example embodiment of a Germpass Lever Handle Unit configured in an open configuration.

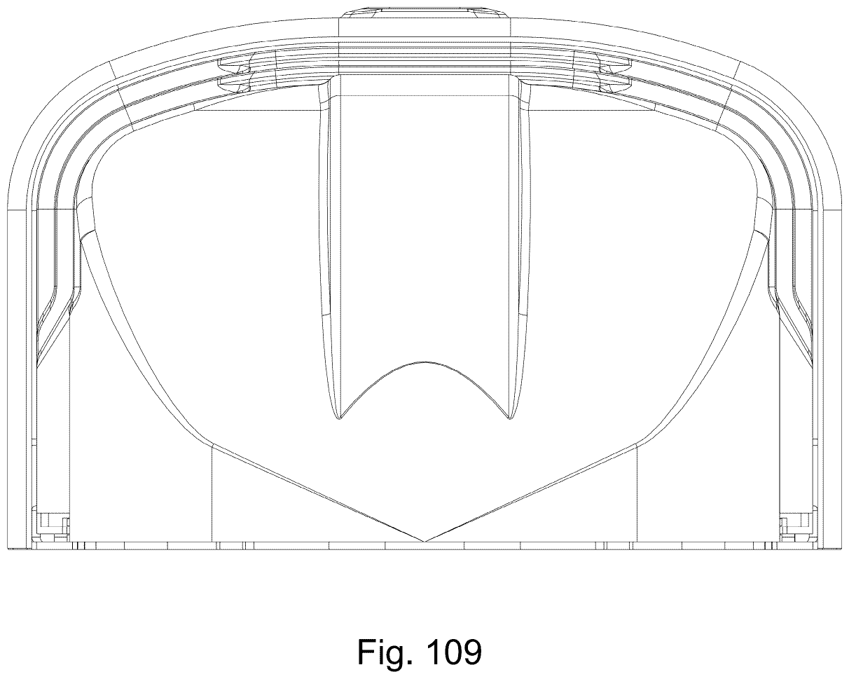

FIG. 109 shows a bottom view of an example embodiment of a Germpass Lever Handle Unit configured in an open configuration.

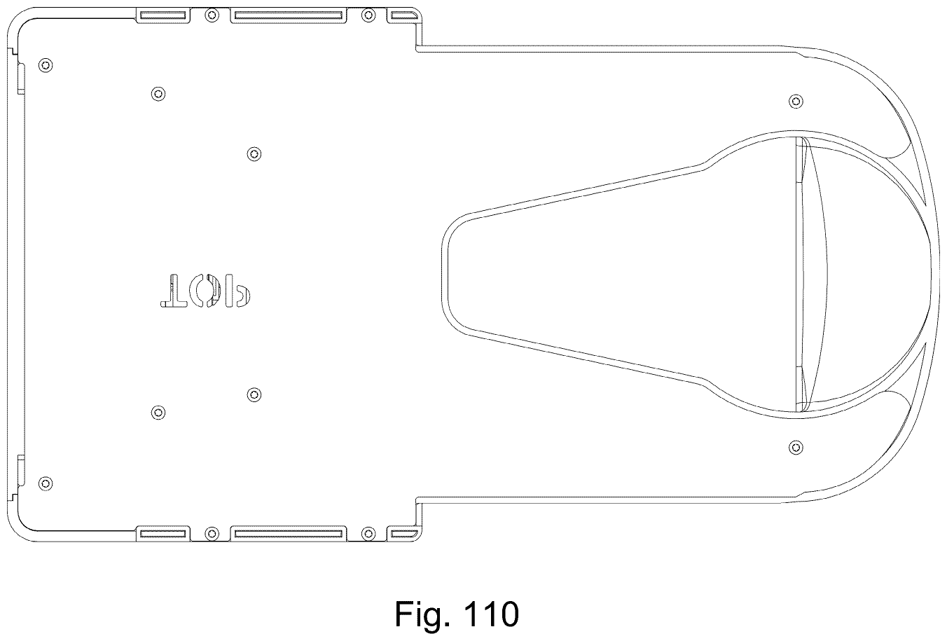

FIG. 110 shows a rear view of an example embodiment of a Germpass Lever Handle Unit configured in an open configuration.

The Germpass Lever Handle Unit is an automated germicidal decontamination system designed for public door lever handles. It utilizes ultraviolet germicidal irradiation (UVGI) to sterilize the lever handle after each user interaction, ensuring minimal contamination risk between uses. This system involves multiple mechanical components that work together to enclose, expose, and decontaminate the lever handle efficiently. The following sections provide an in-depth description of each component of the Germpass Lever Handle Unit as illustrated in FIGS. 95-110.

The front cover plate (9510) is specifically designed to accommodate the retraction of the movable access panel(s) into a stacked configuration behind it when the panel(s) are in the open position. As the exterior housing of the Germpass Lever Handle Unit, the front cover forms a protective barrier around the door handle, preventing contamination from the environment. Its size and shape are optimized to allow the access panel(s) to move smoothly into a stacked position behind the cover when the system transitions to the open configuration. Constructed from durable, germ-resistant materials, the front cover may also feature a UV-reflective interior surface to enhance the UVGI process. In the closed configuration, as shown in FIGS. 95, 98, and 101, it fully conceals the lever handle, maintaining a hygienic environment and protecting the handle from external contaminants between user interactions.

The movable access panel (9520), shown in FIGS. 95, 98, 105, and 142, is designed to move into a stacked configuration behind the front cover plate when in the open configuration, allowing user access to the lever handle. Featuring a U-shaped cross-section, the panel encloses the lever handle in the closed configuration and retracts to expose it when user presence is detected, as seen in FIGS. 104 and 105. The panel operates via a motorized drive system, which responds to proximity sensors, and retracts along guided rails for smooth transitions that minimize environmental exposure to the handle. After user interaction, the panel closes, scaling the handle within the sterilization chamber for UVGI treatment.

The Door Handle Lever (9530) is the fomite itself-the physical component that users interact with to open or close a door. Shown prominently in FIGS. 96 and 104, this lever handle is enclosed and exposed by the access panel during the system's operation. After a user touches the handle, it is immediately sealed off by the access panel and subjected to UV-C light for rapid decontamination. The door handle lever may also feature a specialized surface coating designed to be compatible with the UVGI process, ensuring maximum germicidal efficiency during each sterilization cycle.

The rear opening (9540), shown in FIG. 105, is designed to facilitate the retrofit installation of the Germpass Unit over the fomite region of an existing lever handle without requiring the removal of the door handle. This opening enables the unit to be securely mounted around the existing handle while maintaining full door functionality. It also provides access for notable components, such as the drive mechanisms and sensors, to interface with the door handle, ensuring seamless operation of the Germpass Unit without altering the original fixture.

In one embodiment, the Germpass Lever Handle Unit operates in both closed and open configurations. When no user is present, the unit remains sealed, with the access panel closed around the lever handle. This protects the handle from airborne pathogens or environmental contamination. Upon detecting an approaching user through its integrated sensors, the unit opens the access panel to allow interaction. After use, the access panel re-seals the handle and activates the UV-C LEDs to begin the germicidal irradiation process. This ensures that the handle is sterilized before the next user interaction.

In at least some embodiments, the Germpass system may include a sophisticated communication feature which enables two Germpass Lever Handle Units installed on opposite sides of a single door to work in synchrony. For example, in one embodiment, each unit is installed over the lever-style handle on a respective side of the door-one for the interior and one for the exterior. This synchronized setup is particularly useful for environments where hygiene is critical, as it minimizes unnecessary contact with door surfaces while ensuring smooth access. Lever handles on doors are typically interlocked, meaning that turning the handle on one side rotates the handle on the opposite side simultaneously. This inherent interdependency creates a need for both Germpass Units to operate in close coordination; otherwise, one unit's closed access panel would prevent the other side's handle from turning, hindering entry or exit.

In at least one embodiment, each Germpass Unit integrates a wireless transceiver module operating on designated frequencies to enable real-time communication with its paired unit. The wireless system utilizes a low-latency protocol specifically optimized for rapid signal transmission between paired units. When the sensors of a first unit detect an approaching user, the system initiates a precisely timed sequence of events. The detecting unit transmits (e.g., in real-time) an activation signal to its paired counterpart via the wireless link. This signal contains encoded instructions that trigger the synchronized opening of both units' access panels, ensuring unobstructed operation of the mechanically coupled door handles.

In practice, when a person approaches a door equipped with Germpass Lever Handle Units, sensors within the first unit detect the approaching individual and a wireless signal is transmitted to the paired unit on the opposite side of the door, prompting both access doors to automatically open simultaneously to expose the lever handle. This communication ensures that both units allow simultaneous access to the lever handle, thus facilitating seamless door operation from either side. This wireless communication is engineered to achieve minimal latency, with both units synchronizing their actions within approximately 80-90 milliseconds, creating a virtually instantaneous dual response. Such rapid response is important to maintaining a smooth and uninterrupted experience, especially in high-traffic or healthcare settings where delay could impact both functionality and user satisfaction.

The communication protocol between units may leverage secure, low-power wireless technology such as Bluetooth Low Energy (BLE) or a custom radio frequency (RF) link. This technology provides reliable signal transmission even in environments with high interference, ensuring robust performance and dependable timing. This synchronized interaction between the Germpass Units enables each unit to respond as a cohesive system, significantly improving the usability of dual-handle doors equipped with germicidal covers.

In some embodiments, the system also maintains a continuous heartbeat connection between paired units to verify operational status and trigger appropriate fail-safe modes if communication is interrupted. The system also shares operational data between paired units, including battery levels, UV-C component status, and usage patterns. This data sharing enables predictive maintenance scheduling and ensures consistent performance across both units.

The synchronized operation provides several unique advantages. By coordinating access panel movements, the system prevents mechanical binding that would occur if one handle were blocked while the other was actuated. This coordination reduces stress on both the door handle mechanism and Germpass Unit components, extending operational life. The wireless interface additionally supports remote configuration updates and system monitoring, enabling facility managers to maintain optimal operation of paired units throughout their installation environment. The communication system may also integrate with building management systems to provide usage analytics and maintenance alerts.

In the absence of this synchronized communication, several operational issues could arise. For instance, if a person tries to turn a lever handle while the opposite unit's access panel is closed, the handle will be blocked, causing frustration and possibly damaging the Germpass hardware if forced. The communication between units prevents such issues by coordinating the units operation, thereby optimizing the user's experience and preserving the hardware. This feature enhances the system's functionality in critical areas such as hospitals, laboratories, or public buildings, where user-friendly design and effective germ prevention are paramount. The synchronization not only contributes to hygiene by automating the germicidal process but also eliminates the need for users to pause or adjust their actions, making the door system both intuitive and efficient.

### Germpass Restroom Stall Latch Unit Example Embodiments

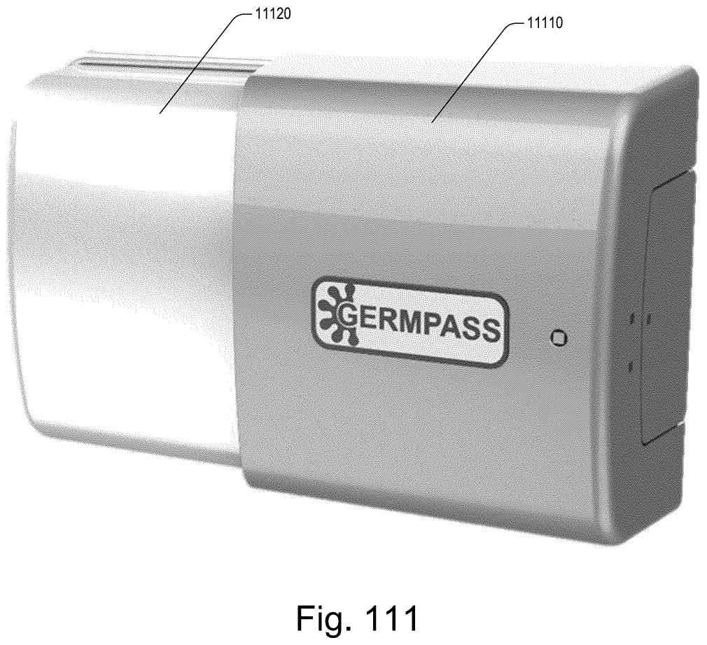

FIGS. 111-126 illustrate different views and features of a Germpass Restroom Stall Latch Unit according to at least one embodiment.

FIG. 111 shows a perspective view of an example embodiment of a Germpass Restroom Stall Latch Unit configured in a closed configuration.

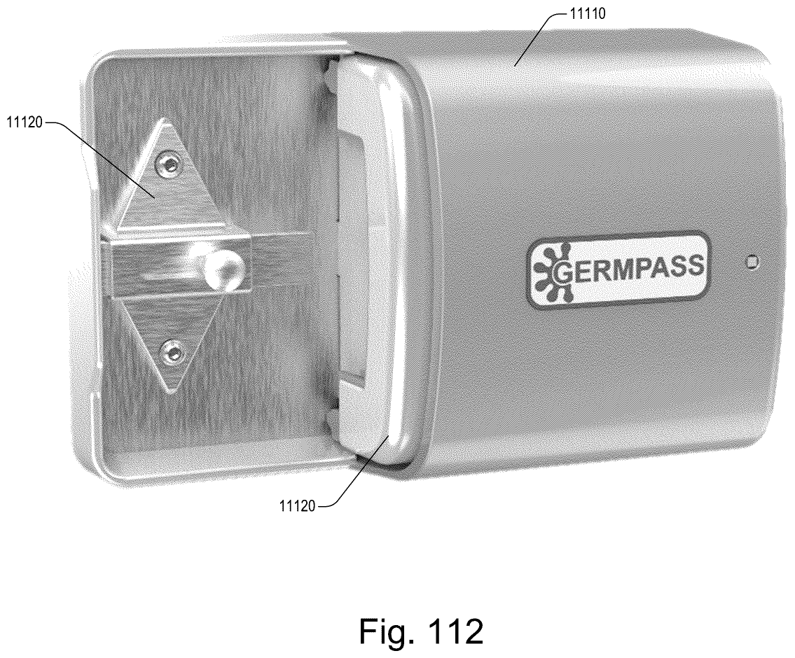

FIG. 112 shows a perspective view of an example embodiment of a Germpass Restroom Stall Latch Unit configured in an open configuration.

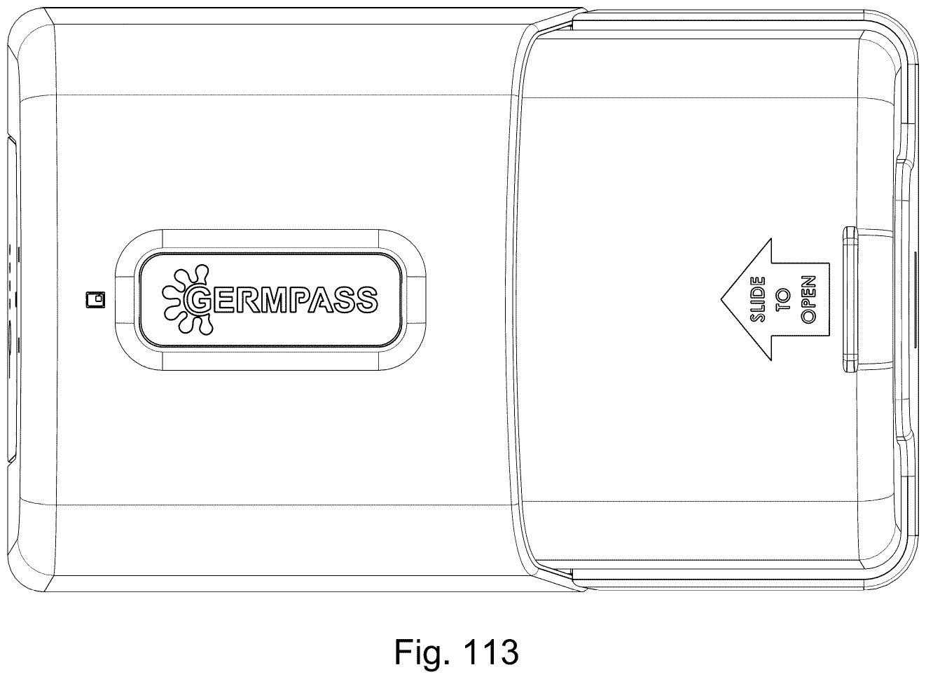

FIG. 113 shows a front view of an example embodiment of a Germpass Restroom Stall Latch Unit configured in a closed configuration]

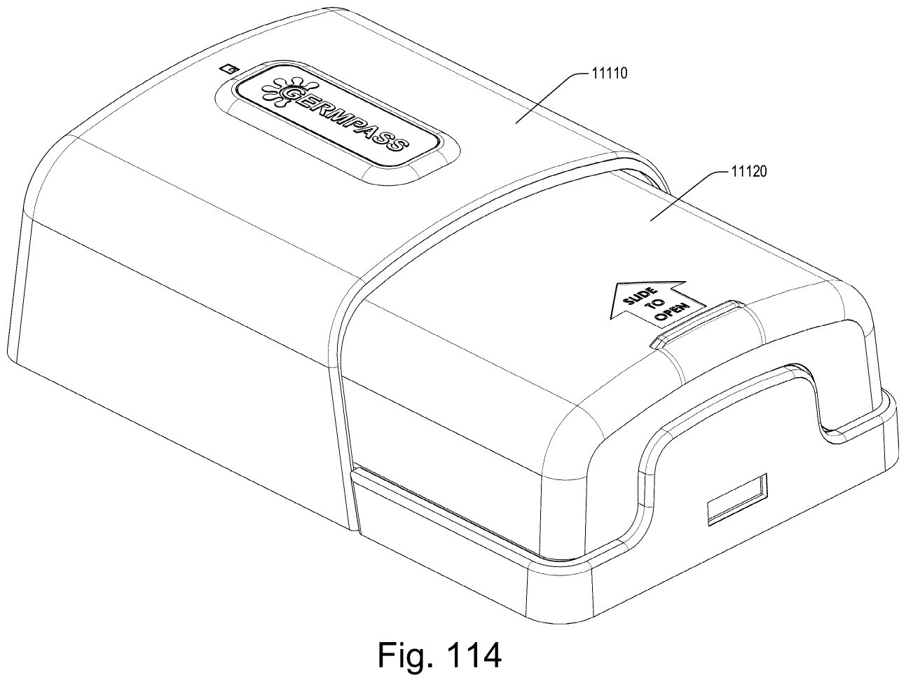

FIG. 114 shows a perspective view of an example embodiment of a Germpass Restroom Stall Latch Unit configured in a closed configuration.

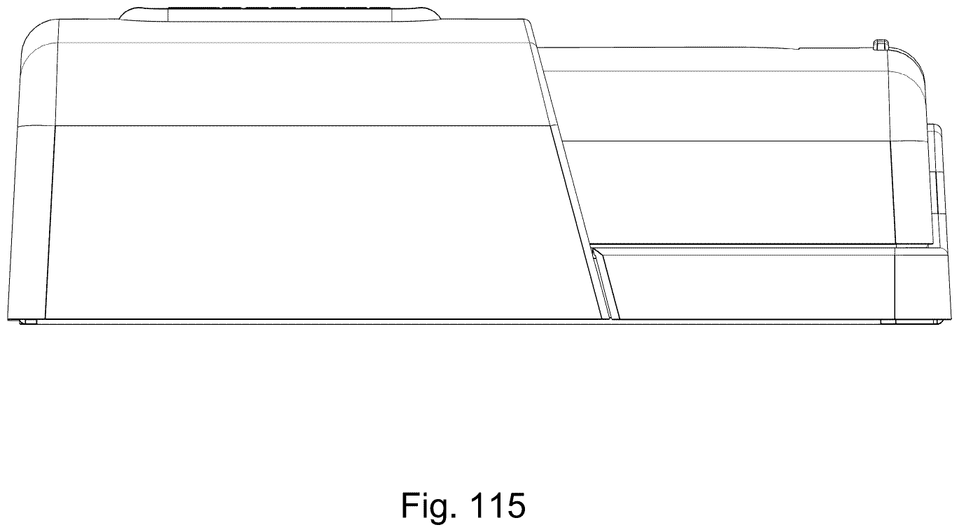

FIG. 115 shows a left view of an example embodiment of a Germpass Restroom Stall Latch Unit configured in a closed configuration.

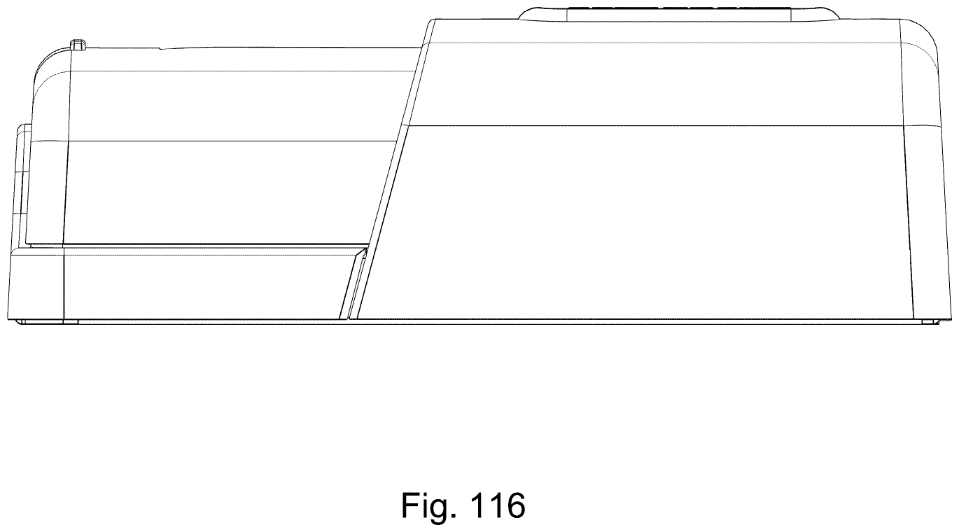

FIG. 116 shows a right view of an example embodiment of a Germpass Restroom Stall Latch Unit configured in a closed configuration.

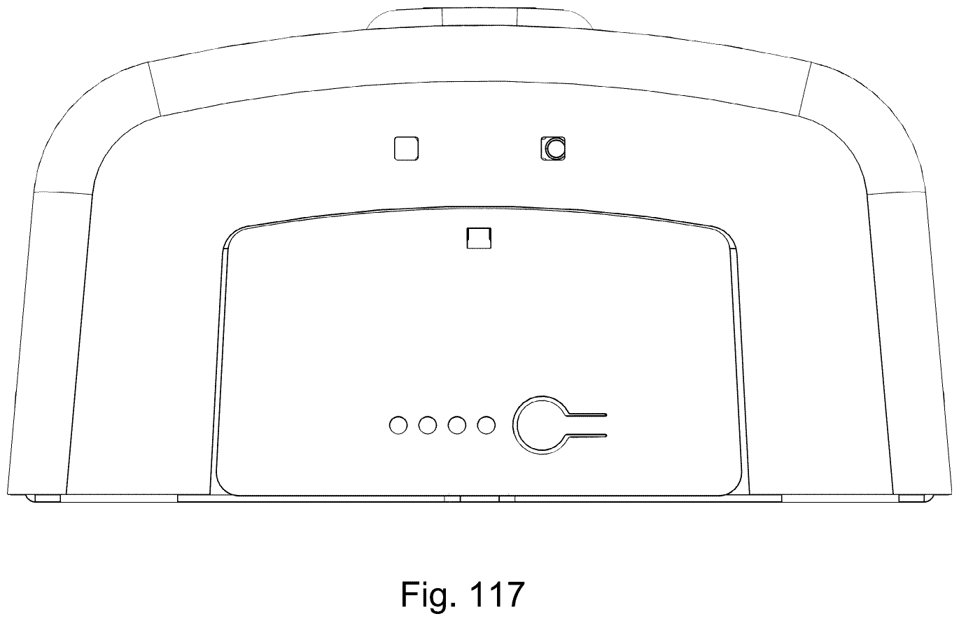

FIG. 117 shows a top view of an example embodiment of a Germpass Restroom Stall Latch Unit configured in a closed configuration.

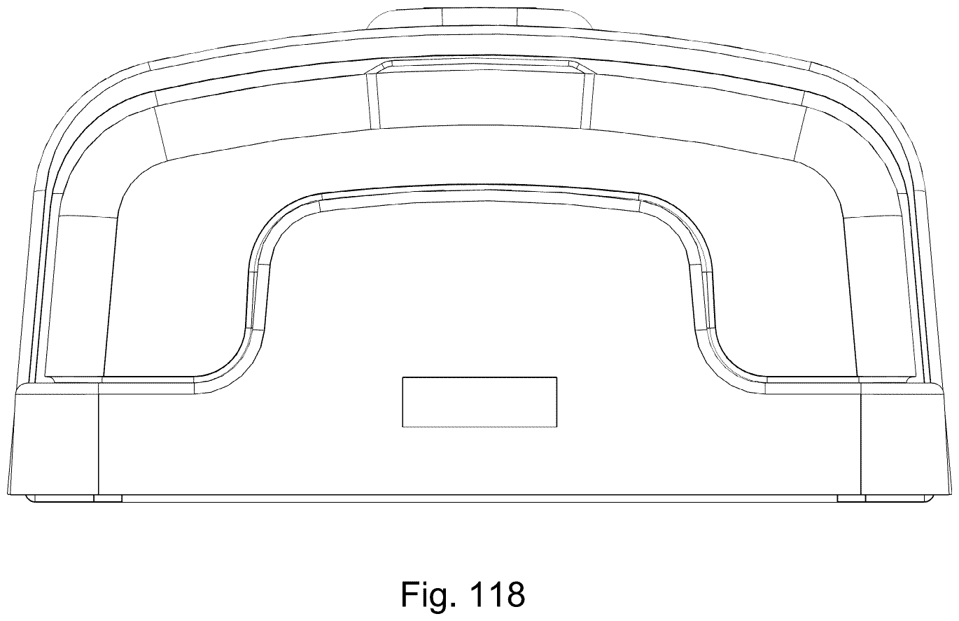

FIG. 118 shows a bottom view of an example embodiment of a Germpass Restroom Stall Latch Unit configured in a closed configuration.

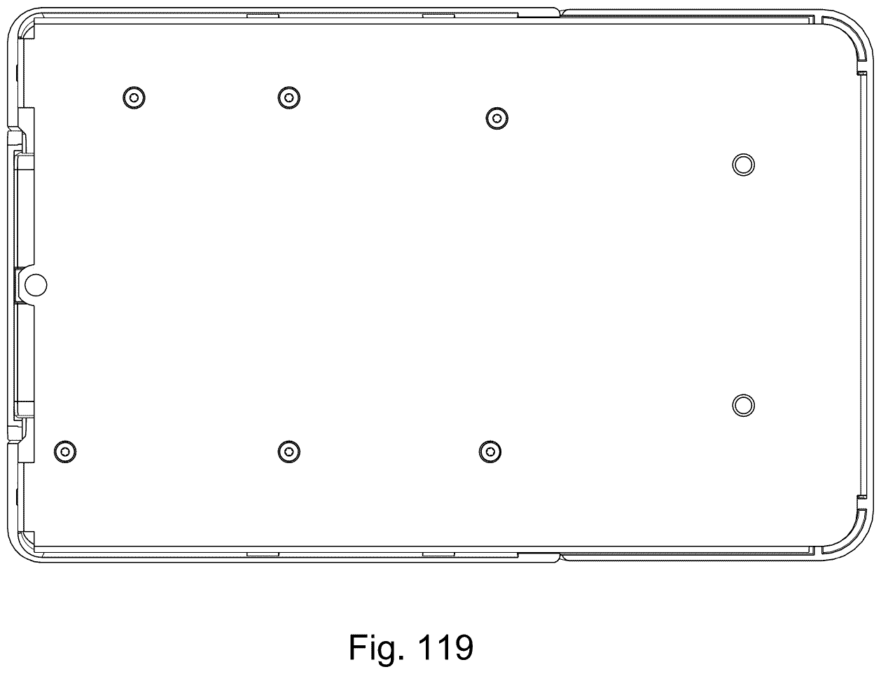

FIG. 119 shows a rear view of an example embodiment of a Germpass Restroom Stall Latch Unit configured in a closed configuration.

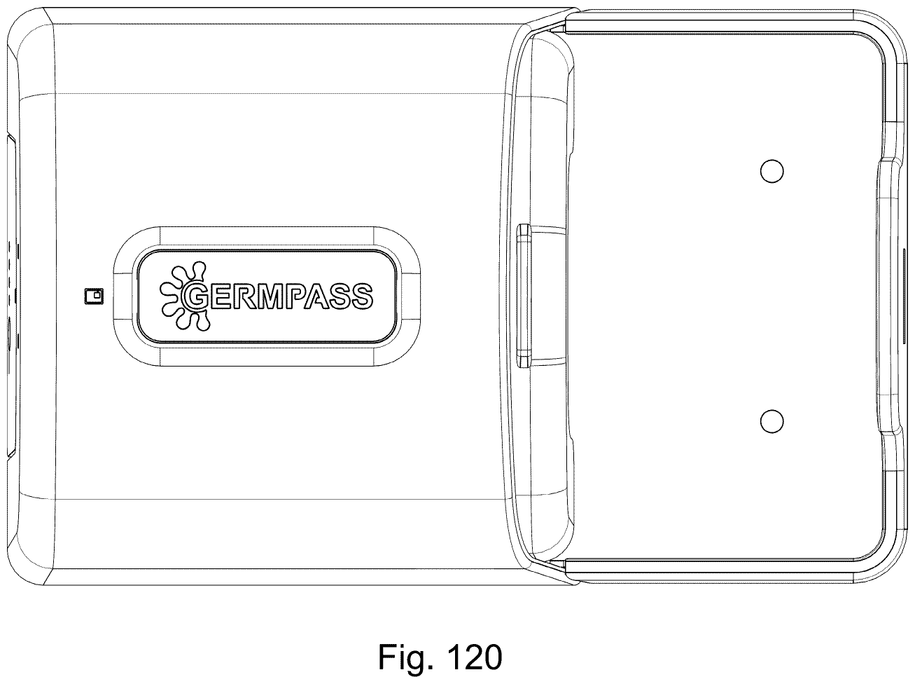

FIG. 120 shows a front view of an example embodiment of a Germpass Restroom Stall Latch Unit configured in an open configuration.

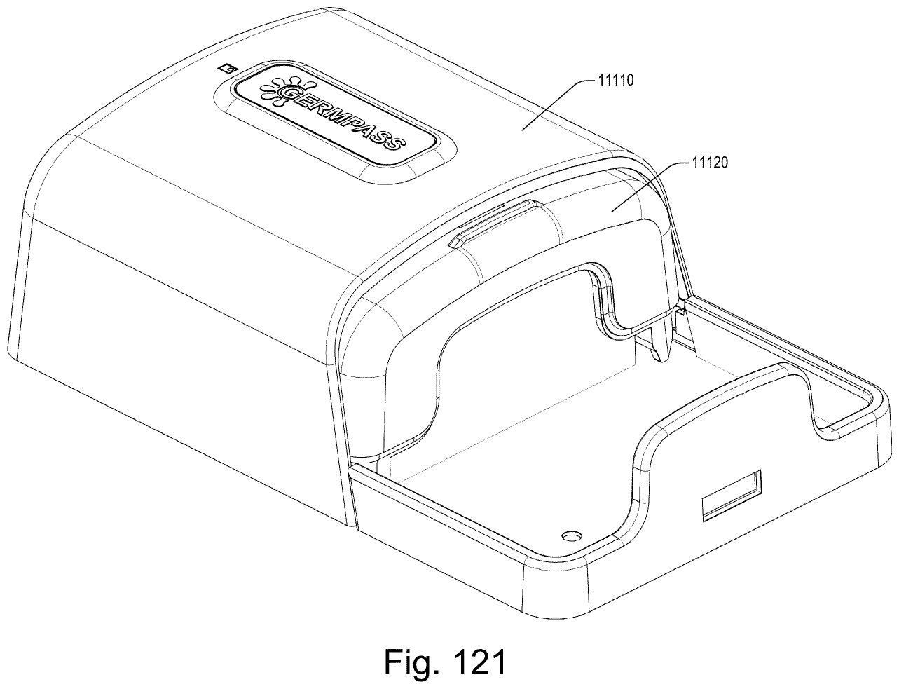

FIG. 121 shows a perspective view of an example embodiment of a Germpass Restroom Stall Latch Unit configured in an open configuration.

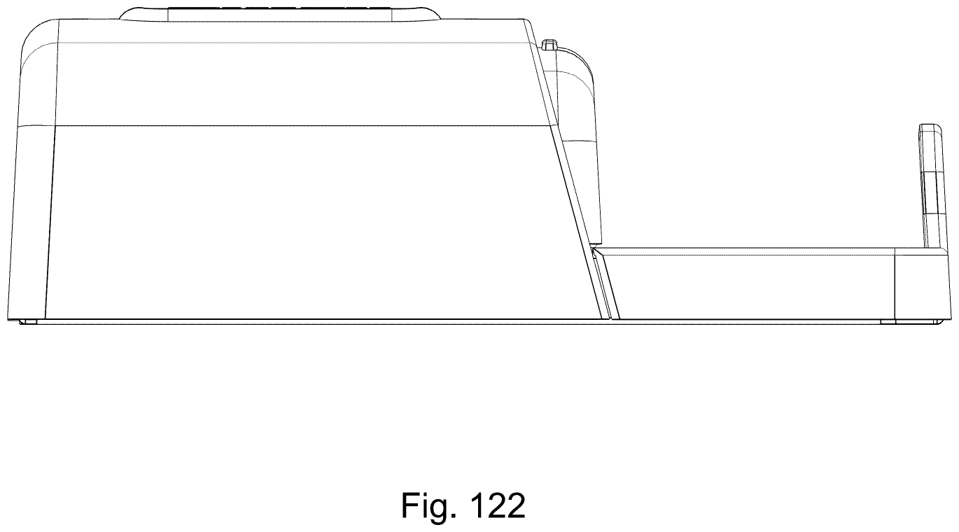

FIG. 122 shows a left view of an example embodiment of a Germpass Restroom Stall Latch Unit configured in an open configuration.

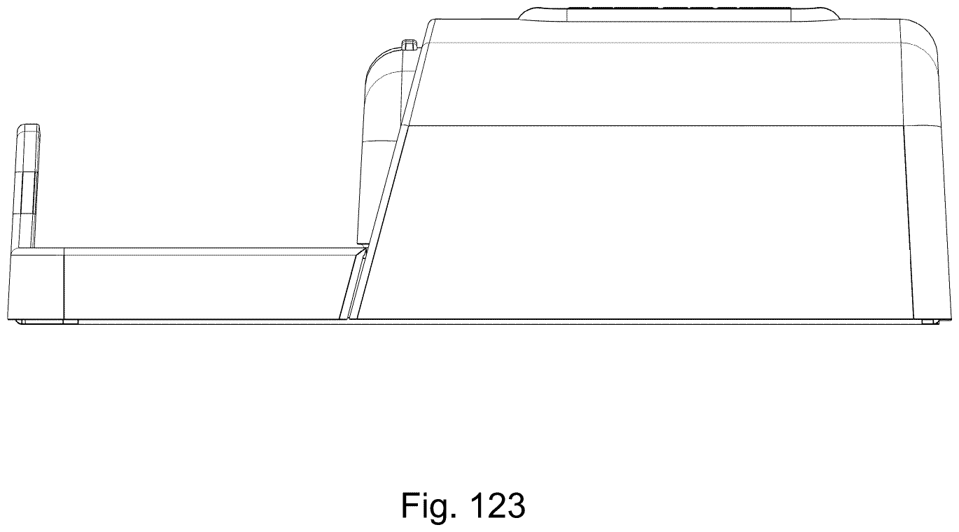

FIG. 123 shows a right view of an example embodiment of a Germpass Restroom Stall Latch Unit configured in an open configuration.

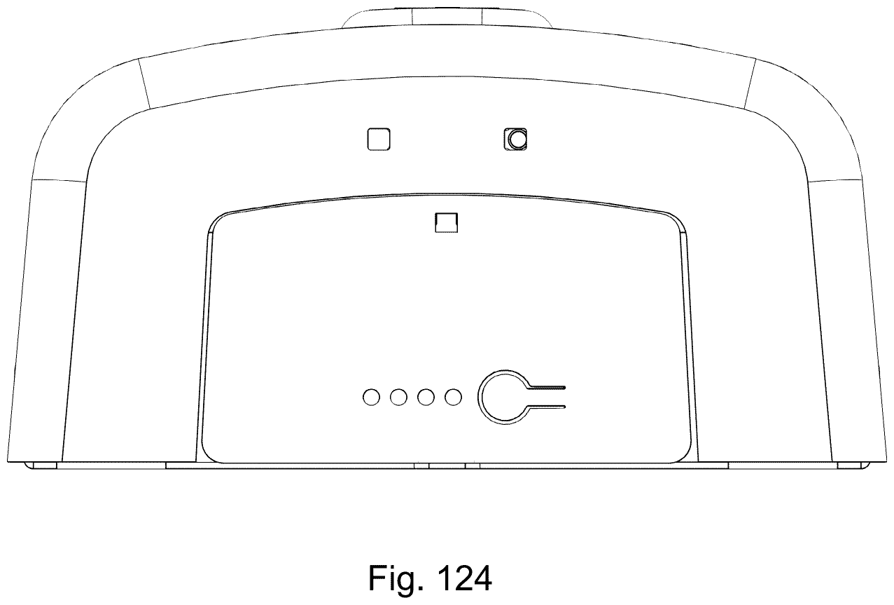

FIG. 124 shows a top view of an example embodiment of a Germpass Restroom Stall Latch Unit configured in an open configuration.

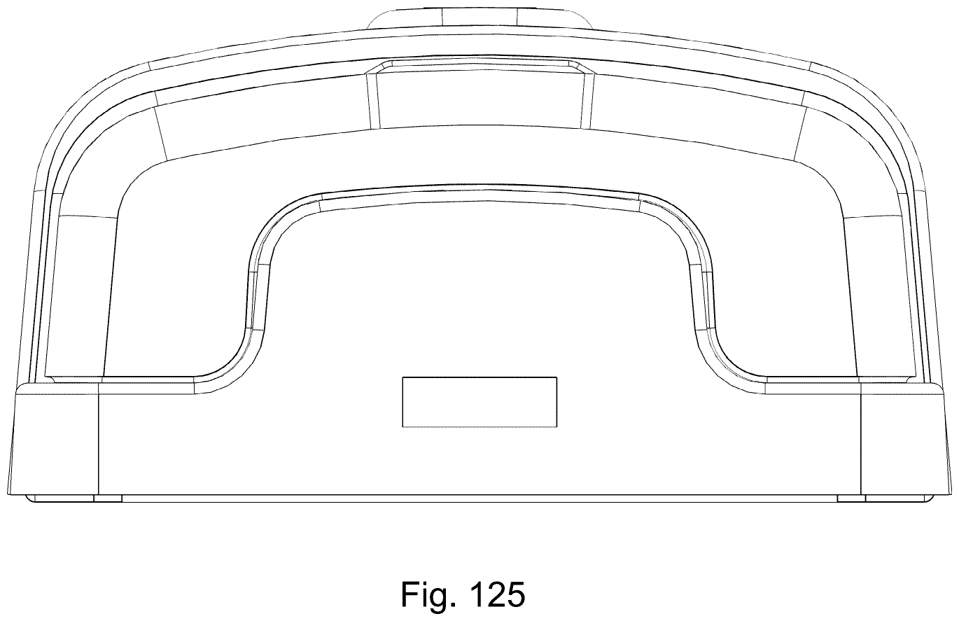

FIG. 125 shows a bottom view of an example embodiment of a Germpass Restroom Stall Latch Unit configured in an open configuration.

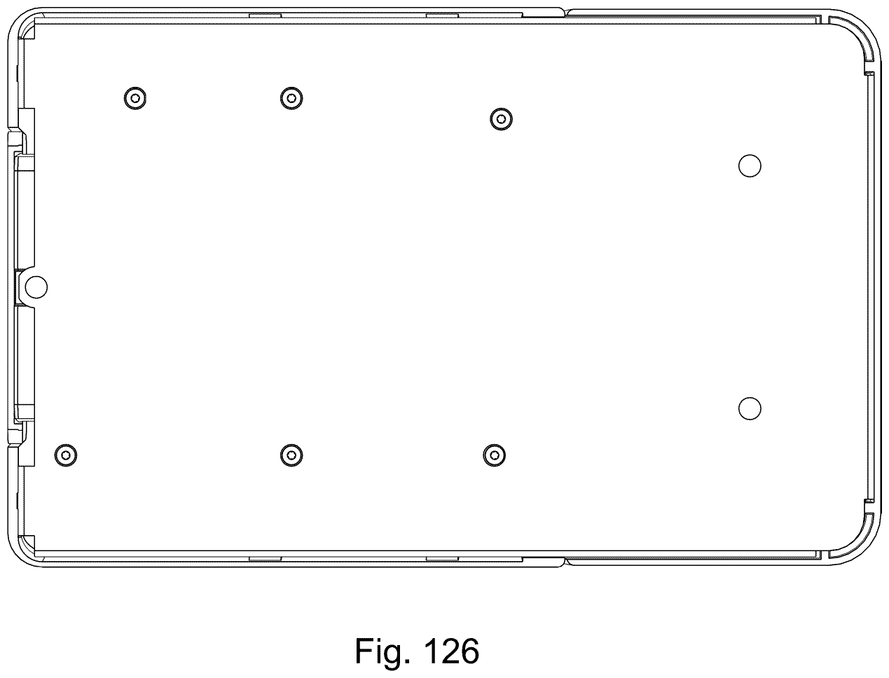

FIG. 126 shows a rear view of an example embodiment of a Germpass Restroom Stall Latch Unit configured in an open configuration.

The Germpass Restroom Stall Latch Unit is a specialized germ decontamination device designed to sanitize restroom stall latches through the use of ultraviolet germicidal irradiation (UVGI). This system is engineered to minimize the transmission of pathogenic microorganisms by decontaminating the latch between user interactions. The unit incorporates an enclosure that shields the latch when not in use and automatically exposes it when a user approaches. This section will provide an expanded description of each of the Germpass Restroom Stall Latch Unit components with associated reference numbers, based on the figures provided.

In its default closed configuration (e.g., as shown in FIGS. 111-119), the Germpass Restroom Stall Latch Unit forms a protective barrier over the latch, preventing any contamination by external factors such as airborne pathogens or accidental touches. The unit is triggered by user proximity, detected through integrated sensors, which activates a motorized access panel to reveal the latch for user interaction. After use, the access panel closes, and the unit initiates a UV-C light-based decontamination cycle.

The front cover plate (11110) is designed with a specific size and shape to allow the movable access panel(s) to retract and stack behind it when configured in the open position. As the outermost surface of the unit, the front cover plate encloses the internal components and serves as a protective shield over the latch. Fabricated from durable, non-porous materials, it prevents external contamination and is easy to clean. The cover plate is seamlessly integrated with the stall door and latch, ensuring a secure and sealed environment for the decontamination process. Additionally, it may feature indicators to display the unit's status, such as when it is in use or undergoing decontamination.

The movable access panel (11120) is designed to move into a stacked configuration behind the front cover plate when in the open configuration. This panel, which may feature a U-shaped cross-section, is motorized and slides open or closed in response to sensor inputs detecting user presence. The panel retracts smoothly to expose the latch when needed and ensures the latch remains protected from contamination when not in use. A motorized assembly, connected to proximity sensors, controls the panel's movement, allowing it to open only when a user is sufficiently close to interact with the latch. The access panel is constructed from lightweight materials with UV-reflective properties to enhance the efficiency of the UVGI system during decontamination. Additionally, the panel's interior is lined with materials that maximize UV-C light reflection, ensuring thorough coverage and disinfection of the latch once the access panel closes after user interaction.

The Restroom Door Latch (11130) is the functional component that users interact with when entering or exiting a restroom stall. The latch itself is housed within the Germpass Unit and protected by the access panel when not in use. The unit may accommodate various latch designs, including standard slide latches or deadbolt-style latches, depending on the specific configuration of the restroom stall.

When a user interacts with the latch, the system continues to monitor their presence and adjusts the decontamination cycle timing accordingly. After the user leaves and the access panel closes, the latch is automatically exposed to a UV-C light cycle that destroys any pathogens left on its surface. The design of the latch ensures that it is fully covered during decontamination, preventing any recontamination until the next user interaction.

The Germpass Restroom Stall Latch Unit operates automatically, utilizing a series of sensors to detect when a user approaches and interacts with the stall latch. When a user comes within a certain range, the unit opens the access panel, exposing the latch for use. After the user engages the latch and exits the stall, the panel closes, and a UV-C decontamination cycle is initiated. The UV-C light is emitted from strategically placed UV-C LEDs, which are configured to ensure complete coverage of the latch surface.

The system integrates intelligent control algorithms to minimize power consumption by initiating decontamination cycles only when necessary. Additionally, it features safety mechanisms such as sensors that prevent accidental exposure to UV-C light while users are nearby, ensuring the decontamination process does not interfere with normal stall use.

In conclusion, the Germpass Restroom Stall Latch Unit is designed to enhance public health safety in high-traffic areas by providing rapid and effective germicidal decontamination of restroom stall latches. Its motorized access panel, UV-C lighting system, and sensor-driven operation ensure that the latch remains sanitary and ready for use, reducing the risk of disease transmission in public restrooms.

### Germpass Germpass Crashbar Unit Example Embodiments

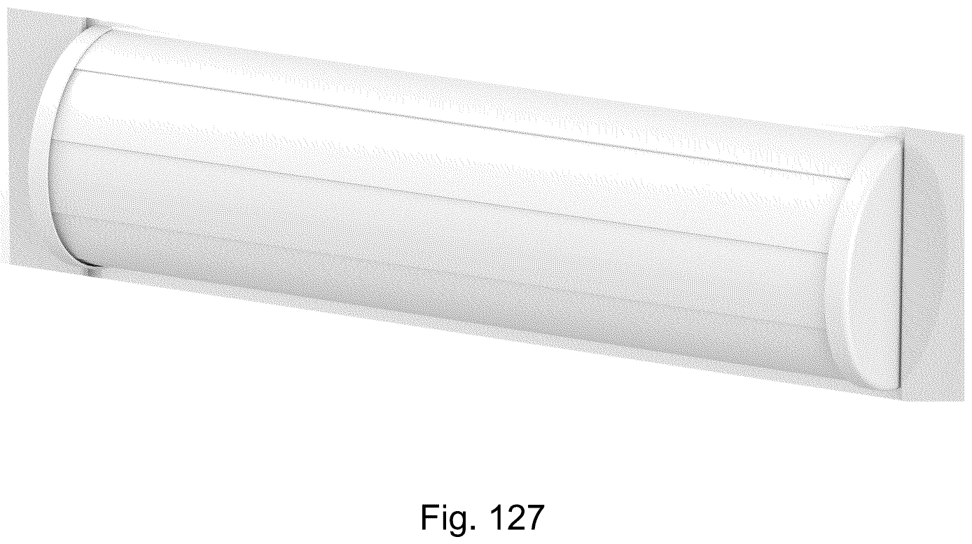

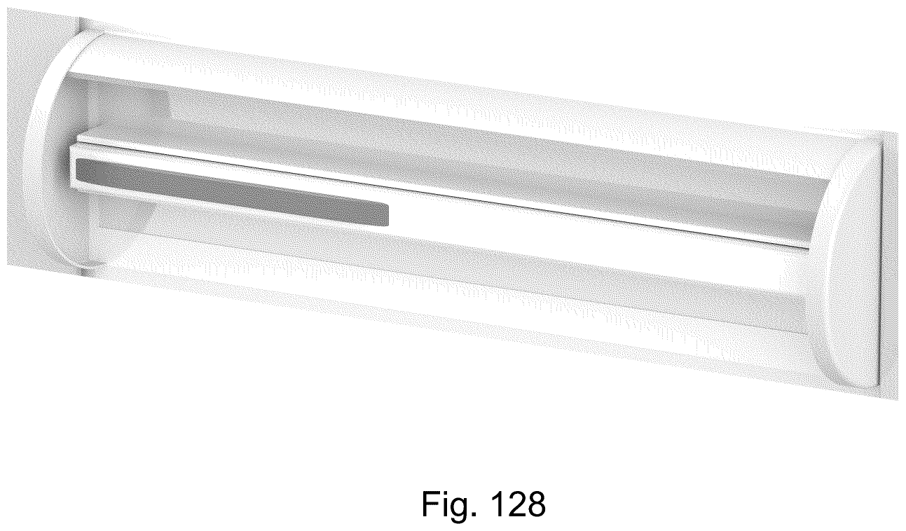

FIGS. 127-128 illustrate different views and features of a GermPass Crashbar Unit according to at least one embodiment.

FIG. 127 shows a perspective view of an example embodiment of a Germpass GermPass Crashbar Unit configured in a closed configuration.

FIG. 128 shows a perspective view of an example embodiment of a Germpass GermPass Crashbar Unit configured in an open configuration.

The Germpass GermPass Crashbar Unit is a sophisticated germ decontamination system specifically designed to protect crash bars (typically found on doors) from contamination in high-traffic environments. This unit utilizes a combination of sensor technology and ultraviolet germicidal irradiation (UVGI) to ensure that the crash bar is sterile before and after each user interaction. The system is equipped with an enclosure that opens and closes automatically in response to user proximity and intent, limiting the crash bar's exposure to contaminants while maintaining operational efficiency and safety.

In the closed configuration (e.g., FIG. 127), the GermPass Crashbar Unit is designed to encapsulate the crash bar, creating a protective barrier around the fomite. This setup shields the crash bar from environmental contamination and airborne particles, ensuring that it remains untouched by germs during idle periods. The system remains in standby mode, with all internal components, such as the UVGI system and motion sensors, primed to detect the approach of a user.

In this configuration, the access panels of the unit are fully closed. These panels are driven by a motorized assembly that ensures smooth, controlled movement. The closed state of the unit plays a notable role in conserving energy, as the system does not engage its decontamination or panel movement functions until user presence is detected.

Upon detecting a user approaching the crash bar, the GermPass Crashbar Unit transitions from a closed to an open configuration (e.g., FIG. 128). The opening process is facilitated by a motorized drive system that retracts the access panels to expose the crash bar for user interaction. This motion is carefully controlled by rotary encoders and sensors that ensure the panels are aligned correctly and moved smoothly into the retracted position.

While the panels are open, the unit continues to monitor the user's interaction with the crash bar. Sensors detect when the user has completed their action, and after a brief delay to account for any stragglers, the system re-closes the access panels to initiate the next decontamination cycle.

### Example Germpass Crashbar Unit Components

Enclosure and Access Panels: The enclosure forms the protective shell of the system, housing the crash bar within a sealed environment during idle periods. The access panels are integral to the system's functionality, as they open to allow user interaction and close to protect the bar when not in use. These panels are driven by a stepper motor that ensures precise control over their movement.
Motorized Drive System: The drive system is responsible for the movement of the access panels. It consists of a motor and drive chain connected to the access panels via support arms. The motor's operations are carefully monitored by encoders that provide real-time feedback on panel position, ensuring that the panels open and close smoothly.
UVGI System: The Ultraviolet Germicidal Irradiation (UVGI) system is central to the decontamination process. It employs UV-C LEDs that emit light in the 265 nm wavelength, which is highly effective at destroying a wide range of pathogens, including bacteria and viruses. The UVGI system is activated once the user has completed their interaction with the crash bar, delivering a dose of UV-C light to sanitize the surface. The system is designed to minimize power usage by operating only when necessary, based on input from proximity and motion sensors.
Sensor Array: The sensor system enables the unit to detect user presence and movement. It includes infrared sensors, ultrasonic sensors, and other proximity detectors that track the user's approach and determine their intent to interact with the crash bar. Once the user is detected, the system calculates the velocity and direction of their movement to anticipate the interaction. Upon completion of the interaction, the sensors continue to monitor for the user's departure, allowing the system to close the access panels and initiate the UVGI cycle.
Microcontroller and Software: The microcontroller acts as the brain of the unit, coordinating the actions of the sensors, drive system, and UVGI lights. It processes sensor data in real-time, using filtering algorithms to reduce noise and ensure accurate detection of user presence and motion. The software is designed to manage multitasking efficiently, ensuring that the unit responds promptly to user interactions while managing energy consumption and optimizing decontamination cycles.
Reflective Materials: Inside the enclosure, reflective materials such as aluminum foil or PTFE are used to enhance the effectiveness of the UVGI system. These materials reflect UV-C light onto all surfaces of the crash bar, ensuring comprehensive exposure and more effective sterilization.
Safety and Obstruction Detection: To prevent accidents, the system includes obstruction sensors that halt panel movement if any foreign object or user hand is detected in the path of the closing panels. This feature enhances the safety of the unit, particularly in high-traffic areas where users may inadvertently place their hands near the panels during closure.

The decontamination process begins once the system detects that the user has moved away from the crash bar. The access panels close to create a sealed environment, and the UVGI system activates, delivering a dose of UV-C light that effectively kills 99.9% of germs on the crash bar. The UVGI process is completed in a matter of seconds, after which the system resets to standby mode, ready for the next user interaction.

The decontamination cycle is calibrated to deliver just enough UV-C light to ensure thorough sterilization without over-consuming power. The system monitors the performance of the UV-C LEDs to ensure that they are operating at optimal levels, and the software adjusts the decontamination time based on real-time environmental conditions, such as ambient light and temperature.

### Elevator Cab Button Panel Germpass Unit Decontamination Procedure

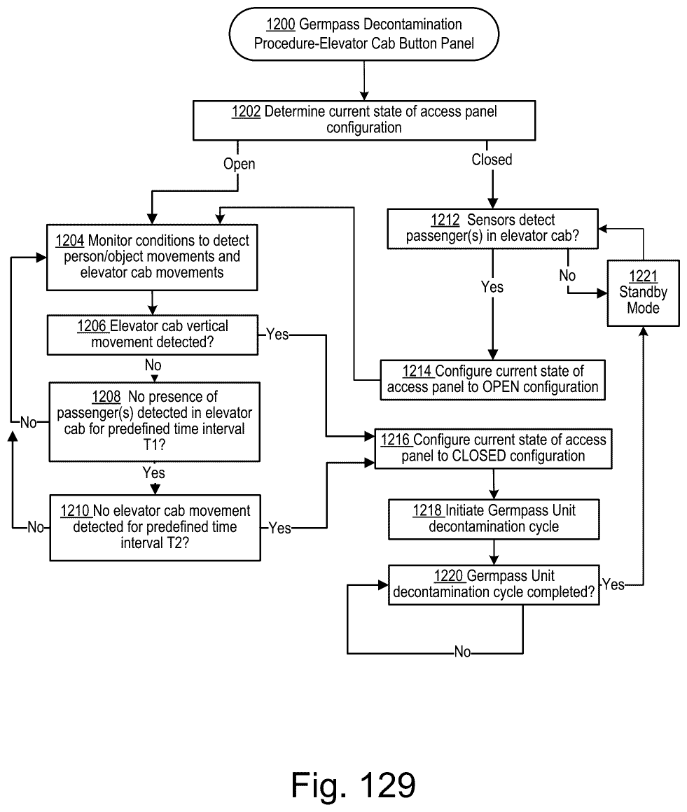

FIG. 129 shows and example flow diagram of an Elevator Cab Button Panel Germpass Unit Decontamination Procedure 1200.

The Germpass Decontamination Procedure implemented within the elevator cab setting, as illustrated in FIG. 129, is a multi-step process designed to ensure that the elevator panel buttons are rapidly and efficiently sanitized using ultraviolet germicidal irradiation (UVGI). This process begins with determining the current state of the Germpass Unit's access panel, followed by monitoring passenger presence and elevator cab movements. Based on these conditions, the system may initiate a decontamination cycle, during which UV-C light is deployed to sanitize the elevator panel. The system's ability to differentiate between open and closed states, as well as its adaptive response to sensor inputs, ensures rapid decontamination cycles without impeding user access.

Step 1202: Determine Current State of Access Panel Configuration: In one embodiment, as shown at 1202, the Germpass Unit may determine whether the access panel of the Germpass Unit is in an open or closed state. The microcontroller of the Germpass Unit continuously monitors the state of the access panel using multiple sensor inputs to ensure accurate state determination. Sensors integrated into the access panel—such as contact sensors, proximity sensors, position sensors, optical sensors, or magnetic reed switches—provide real-time feedback regarding the panel's configuration.

Position sensors mounted at various points along the panel track offer precise location data, while optical sensors or magnetic reed switches at the fully open and fully closed positions confirm the panel's state with redundant verification. The drive assembly incorporates rotary encoders that track the precise position of the panel during movement, enabling the system to detect any partial opening or abnormal positioning.

The microcontroller maintains a state machine that records the current panel configuration and validates it against the sensor inputs to ensure system integrity. This multi-sensor approach enables reliable state determination even in cases of power interruption or system reset. The configuration state directly influences subsequent system behavior. An open state indicates that passenger interaction may be in progress and protects against interrupted access, while a closed state allows the system to prepare for potential decontamination cycles.

Step 1204: Monitor Conditions to Detect Person/Object Movements and Elevator Cab Movements: In this step, the Germpass Unit implements comprehensive environmental monitoring through an array of specialized sensors. Various sensors such as motion detectors, occupancy sensors, and a Micro-Electro-Mechanical System (MEMS) accelerometer monitor both the presence of passengers and the vertical motion of the elevator cab.

A MEMS accelerometer, typically mounted in a vibration-isolated housing within the unit, provides high-precision detection of vertical acceleration and movement. The accelerometer samples at rates of 100 Hz. or higher, ensuring rapid detection and high-precision monitoring. The microcontroller processes the accelerometer data stream, implementing sophisticated signal processing and digital filters—such as low-pass filters to eliminate high-frequency noise and Kalman filters for predictive analysis—to remove noise and differentiate actual cab movement from vibration or other environmental factors.

Multiple motion and occupancy sensors are strategically positioned to provide overlapping coverage of the cab interior. These may include passive infrared (PIR) sensors for heat signature detection, ultrasonic sensors for movement detection, and time-of-flight sensors for precise distance measurement. The sensor array operates continuously, with the microcontroller processing and filtering the inputs using advanced sensor fusion algorithms. These algorithms combine data from multiple sensors to improve detection reliability and reduce false positives, ensuring accurate monitoring of passenger presence and elevator movement.

The microcontroller employs a real-time operating system (RTOS) to manage multitasking and real-time data processing requirements. The software architecture is designed for efficient handling of sensor data streams, prioritizing tasks such as sensor fusion, movement detection, and state machine updates. Interrupt-driven routines are utilized for high-priority events, ensuring immediate responsiveness to sensor inputs.

The system maintains separate monitoring threads for passenger presence and cab movement, enabling independent response to cither condition. This comprehensive monitoring allows the Germpass Unit to determine whether conditions are met to initiate a decontamination cycle or to continue waiting.

Step 1206: Detect Vertical Movement of Elevator Cab: The Germpass Unit includes a MEMS accelerometer as a sensor to detect vertical movement of the elevator cab. This component measures the acceleration forces acting on the elevator in real time. By analyzing the data from the MEMS accelerometer, the system may detect whether the elevator is stationary or in motion.

The accelerometer continuously monitors vertical acceleration, sampling at rates of 100 Hz or higher, providing high-precision detection of vertical movement. The system detects elevator movement within 0.1 seconds of onset, highlighting the responsiveness of the accelerometer and microcontroller. Acceleration thresholds are calibrated to the specific elevator installation, ensuring accurate detection of genuine vertical movement. Calibration involves adjusting sensitivity settings to account for the elevator's typical acceleration profile and ambient vibrations within the building.

The accelerometer data is processed through digital filters, including low-pass filters to remove high-frequency noise and Kalman filters for predictive smoothing, enhancing the accuracy of motion detection. The movement detection algorithm includes hysteresis to prevent false triggering from minor vibrations, ensuring reliable detection of actual cab movement. The system maintains movement history logs to support intelligent decision-making about decontamination timing and to refine detection algorithms over time.

In at least some embodiments, the Germpass Unit may be configured or designed to utilize a high-precision digital barometric pressure sensor to detect vertical movement of the Elevator Cab through changes in atmospheric pressure. For example, as the elevator cab moves vertically, the barometric sensor measures minute variations in air pressure-pressure decreases as the cab ascends and increases as it descends. The barometric sensor provides readings at millisecond intervals with resolution down to 0.013 hPa (approximately 11 cm of altitude change).

The system processes both accelerometer and barometric data streams through sensor fusion algorithms to achieve highly accurate movement detection. Cross-referencing data from these complementary sensor types enables the system to filter out environmental noise and confirm genuine vertical motion. The barometric sensing provides an independent verification of movement detected by the accelerometer.

The microcontroller implements real-time digital filtering and analysis of the sensor data. For barometric detection, the system:
Establishes a baseline pressure reading when the elevator is stationary
Calculates the rate of pressure change to determine direction and speed
Applies threshold detection to identify start/stop of movement
Compensates for atmospheric pressure variations using long-term trending
Correlates pressure changes with accelerometer data for movement validation

This dual-sensor approach provides redundancy and enhanced reliability compared to single-sensor implementations. If one sensor type experiences interference or degraded performance, the system maintains movement detection capability through the other sensor. The combination of accelerometer and barometric sensing ensures accurate triggering of panel closure and decontamination cycles based on verified cab movement.

The barometric detection feature is particularly valuable for low-speed elevator movement where acceleration forces may be subtle. The pressure sensor reliably detects the altitude changes even during gradual elevator motion. This comprehensive sensing strategy optimizes the system's ability to coordinate UV-C decontamination with elevator operation while maintaining safety and efficiency.

Action in Response to Vertical Movement: When vertical movement is detected, indicating that the elevator has started moving between floors, the system automatically proceeds to Step 1216, where the panel is closed and a decontamination cycle is initiated. Initiating the decontamination process during vertical movement takes advantage of a period when the elevator buttons are least to be in use, ensuring effective cleaning without impeding user access.

This automatic and hands-free operation is a significant improvement over manual cleaning systems that rely on human operators to initiate sanitation. By closing the access panel and initiating decontamination only when the elevator is in motion, the system ensures that the elevator panel remains sanitary without exposing users to UV-C light or interrupting normal operation.

The system's software architecture supports real-time responsiveness, with priority given to safety-related tasks such as panel closure during elevator movement. The firmware includes safeguards to prevent unintended behavior, such as closing the panel while passengers are interacting with it.

Step 1208: Detect Presence of Passengers Within Predefined Time Interval T1: In the event that no vertical movement is detected, the system remains in its monitoring state and proceeds to determine if there are passengers in the cab. The system checks whether passengers have been detected within a predefined time interval T1 (e.g., 25 seconds). The Germpass Unit employs a sophisticated passenger detection system utilizing multiple sensor technologies operating in concert.

The primary detection array includes both PIR sensors for heat signature detection and ultrasonic sensors for movement detection. These sensors are positioned at optimal angles to provide complete coverage of the elevator cab interior while minimizing blind spots. The microcontroller implements a rolling time window tracker that maintains occupancy status over the predefined T1 interval.

The detection algorithm may require multiple confirmed sensor triggers within specified time windows to validate passenger presence, reducing false positives from environmental factors. Time-stamped sensor data is stored in a circular buffer, allowing the system to accurately track occupancy duration. Detection threshold parameters are configurable through the system firmware, allowing adjustments based on specific installation requirements and usage patterns. This configurability ensures that the system may adapt to different elevator environments and traffic levels.

The system also considers accessibility requirements to ensure that all users, including those with disabilities, are detected reliably. Sensor placement and sensitivity settings are designed to accommodate various passenger behaviors and mobility devices, complying with accessibility standards to provide equitable access.

If no passenger presence is detected for the full Tl interval, the system advances to evaluate elevator movement status in Step 1210. If passengers are detected, it loops back to continue monitoring the situation, ensuring that the panel remains accessible when needed.

Step 1210: Detect Elevator Movement Within Predefined Time Interval T2: This step checks for elevator movement within a second predefined time interval T2 (e.g., 30 seconds). The system maintains a separate monitoring thread for elevator movement status using data from the MEMS accelerometer. A dedicated timer tracks the duration of movement inactivity, with the T2 interval providing a buffer to confirm sustained dormancy.

The movement detection algorithm implements adaptive thresholding that accounts for building vibration and environmental factors while maintaining reliable movement detection. The microcontroller adjusts sensitivity thresholds dynamically based on historical data and environmental conditions.

The microcontroller maintains separate counters for T1 (passenger detection) and T2 (movement detection) intervals, enabling independent tracking of both conditions. When both timers exceed their respective thresholds—indicating sustained vacancy and inactivity—the system prepares for a decontamination cycle by closing the panel in Step 1216.

Step 1216: Close the Access Panel: Once conditions are met for a decontamination cycle, the Germpass Unit closes the access panel. Upon meeting the conditions for panel closure, the microcontroller activates the panel drive assembly through a controlled sequence. The closure is achieved using a drive assembly that activates based on sensor feedback.

The drive system employs a precision stepper motor or servo mechanism to ensure smooth, consistent panel movement. Position encoders and rotary encoders provide real-time feedback during closure, enabling the system to detect and respond to any obstruction or mechanical resistance. The closure sequence includes intermediate position verification to ensure proper panel alignment throughout the movement.

The drive system implements soft-start and soft-stop acceleration profiles, controlled by the microcontroller, to minimize mechanical stress and ensure quiet operation. This is particularly important in environments where noise reduction is desired. Multiple safety interlocks and obstacle detection mechanisms prevent panel closure if any sensor detects potential passenger interference. The system monitors all sensor inputs during panel movement, and if any obstruction is detected, the panel movement is halted immediately to prevent any potential harm.

The microcontroller's state machine validates the panel configuration against sensor inputs to ensure system integrity, especially after power interruptions or resets. The system includes error detection and fault tolerance features, such as redundancy in components and fail-safe modes, enhancing reliability.

Steps 1218-1220: Initiate and Complete the Germpass Unit Decontamination Cycle: After the access panel is securely closed, the system initiates a UV-C decontamination cycle. The decontamination sequence begins with system verification of panel closure and safety interlock status. The UV-C LED arrays within the Germpass enclosure are activated, emitting UV-C light at an optimal wavelength of 265 nm to rapidly destroy any microorganisms on the elevator panel buttons.

The UV-C light sources inside the unit are positioned to target the entire surface area of the elevator control panel. Internal reflective surfaces, coated with UV-optimized materials such as aluminum or specialized coatings, maximize irradiation efficiency by increasing the light exposure on all areas of the panel. This design ensures uniform UV-C dosage across the panel, enhancing the effectiveness of the decontamination process.

The microcontroller monitors UV-C LED performance through integrated photodiodes and current sensors, adjusting output levels to maintain optimal dosage and consistent UV-C output throughout the cycle. The firmware includes routines for real-time power monitoring and control, compensating for any variations in LED performance or power supply fluctuations.

The decontamination cycle duration is precisely controlled based on the required UV-C dosage to achieve effective germicidal action while minimizing cycle time and energy consumption. The system adheres to relevant safety standards and regulations regarding UV-C exposure limits, such as those established by the International Commission on Non-Ionizing Radiation Protection (ICNIRP), ensuring that no harmful UV-C light escapes the enclosure and that the system operates within safe parameters.

During this cycle, the UVGI process destroys the DNA and RNA of pathogenic microorganisms, rendering them incapable of replication or infection. Upon cycle completion, the system performs a verification sequence to confirm successful decontamination before transitioning to standby mode. The verification sequence includes checks of UV-C LED performance data, confirmation that the required UV-C dosage was delivered, and system self-tests for any anomalies.

The system's firmware logs decontamination cycles and performance data, supporting maintenance protocols and enabling predictive maintenance. Maintenance protocols specify intervals for inspecting and replacing UV-C LEDs, sensors, and other components to ensure ongoing reliability and effectiveness. The system may alert maintenance personnel when component performance degrades below acceptable thresholds.

Steps 1212-1214: Monitor Sensors and Open the Access Panel When Passengers Are Detected: Once the decontamination cycle is complete, the system remains in standby mode, continuously monitoring for passenger presence. In standby mode, the microcontroller continues high-frequency sampling of all sensor inputs while maintaining the panel in a closed position. The passenger detection algorithm remains active, processing sensor data through noise rejection and validation filters.

If passengers are detected, the access panel opens automatically, allowing users to interact with the buttons. When passenger presence is confirmed, the system immediately initiates the panel opening sequence. The drive assembly executes a rapid but controlled opening movement, with position feedback ensuring proper operation. The opening sequence includes obstacle detection to prevent any potential interference.

Once fully open, the panel position is maintained through active holding torque provided by the motor or mechanical detents to ensure stability while passengers interact with the elevator buttons. The system continues monitoring all sensors to manage subsequent state transitions based on passenger activity or elevator movement. High-frequency sampling ensures immediate responsiveness to passenger presence or elevator movement.

User interface elements, such as indicator lights or display panels, inform passengers about the system's status. For example, a green light may indicate that the panel is accessible, while a blue light signifies that the decontamination process is in progress. These visual cues enhance user experience by keeping passengers informed without requiring interaction.

The system ensures accessibility for all users, including those with disabilities. Sensor placements and algorithms are designed to detect users with mobility aids or those who may interact with the panel differently. The system complies with accessibility standards and guidelines, such as the Americans with Disabilities Act (ADA), to provide equitable access.

The Germpass Decontamination Procedure leverages advanced sensing technologies, sophisticated signal processing, and automated control mechanisms to ensure that elevator button panels are efficiently sanitized without impeding user access. By intelligently monitoring passenger presence and elevator movements, the system optimizes the timing of decontamination cycles, enhancing hygiene in high-traffic environments such as elevator cabs. The system's comprehensive monitoring, precise mechanical operations, and robust safety features ensure reliable and effective decontamination. Configurable parameters and calibration to specific elevator installations allow for adaptability to various environments, maximizing the system's effectiveness in maintaining elevator hygiene.

Maintenance protocols and reliability measures, such as error detection, fault tolerance, and redundancy, contribute to the system's long-term performance. Regular maintenance schedules include calibration of sensors, inspection of mechanical components, and replacement of UV-C LEDs as needed to ensure consistent operation. The system may also include remote monitoring capabilities for maintenance personnel to track system status and performance.

The Germpass Unit adheres to relevant safety standards and regulations, including UV-C exposure limits, electrical safety codes, and elevator safety standards such as those outlined by the American Society of Mechanical Engineers (ASME) and European Committee for Standardization (CEN). The system is designed to prevent any exposure of passengers to UV-C light and to operate safely within the mechanical and electrical constraints of elevator installations.

Energy efficiency is considered in the system's design, with features such as precise control of UV-C LED activation duration and power levels, as well as energy-saving modes during standby periods. Using UV-C LEDs offers advantages over traditional sanitization methods, such as chemical cleaners or mercury-based UV lamps, by reducing environmental impact, eliminating the need for consumables, and minimizing hazardous waste.

Through the integration of high-frequency sensor sampling, advanced signal processing, real-time responsiveness, and adherence to safety and accessibility standards, the Germpass Unit provides a seamless, safe, and hygienic experience for elevator users. The system represents a significant advancement in automated sanitation technology, contributing to improved public health and user confidence in shared spaces.

In the retrofit embodiment, existing elevator cab button panels may be retrofitted with a Germpass Unit attached externally. Sensors such as infrared (IR) and ultrasonic sensors monitor the elevator environment for passenger presence and cab movement. For embedded embodiments, the Germpass system is integrated directly into new elevator control panels, allowing for more seamless sensor integration, such as direct MEMS accelerometer mounting and enhanced sensor coverage.

Both embodiments utilize a drive system (e.g., pulley and motor assemblies) to control the opening and closing of the access panels, while the decontamination cycle is managed by a UV-C LED array. In terms of software, a microcontroller operates using inputs from multiple sensors, such as MEMS accelerometers, motion detectors, and ultrasonic sensors, processing the inputs to decide when to open or close the panel and when to initiate the decontamination process.

### Example Walk-Through Scenarios

The Germpass Unit is designed to sanitize elevator button panels using ultraviolet germicidal irradiation (UVGI) while ensuring immediate passenger access when needed. The system employs advanced sensors—including passive infrared and ultrasonic detection systems—microcontroller logic, and precise mechanical movements to achieve optimal sanitization without disrupting the passenger experience. Throughout all scenarios, the system maintains consistent monitoring of environmental conditions at millisecond intervals, managing state transitions smoothly and safely. The procedural flow ensures optimal sanitization opportunities by utilizing transit times and idle periods for decontamination cycles, all while prioritizing immediate passenger access and adhering to strict safety protocols.

### Example Scenario 1: Empty Elevator Cab

Initially, the elevator cab is unoccupied, and the Germpass Unit maintains its access panel in a closed position while the cab responds to a call from a specific floor, such as the 15th floor. The microcontroller actively monitors inputs from its motion and occupancy sensors, ready to detect any passenger presence.

Step 1212: Passenger Detection: As the elevator arrives at the 15th floor and opens its doors, several waiting passengers are present. The occupancy sensors—which include both passive infrared and ultrasonic detection systems—immediately register the presence of the new occupants. The microcontroller processes these sensor inputs and executes Step 1212 of the procedural flow, confirming passenger detection. This rapid detection occurs in under one second, ensuring immediate system responsiveness.

If, alternatively, no passengers are detected when the doors open, the Germpass Unit remains in standby mode with the access panel closed, as the system detects no need for interaction. The microcontroller continues to monitor the sensors at millisecond intervals for any change in occupancy.

Step 1214: Access Panel Opening: Upon confirming passenger detection in Step 1212, the microcontroller initiates Step 1214. The drive assembly activates to smoothly open the access panel, revealing the elevator button panel. This action ensures that passengers experience no delay in accessing the elevator controls. The panel remains open, allowing uninterrupted interaction until further system input dictates a state change.

### Example Scenario 2: Elevator Starts Moving

With passengers present and the access panel open, the elevator doors close, and the elevator cab begins vertical movement to the selected floors.

Step 1206: Vertical Movement Detection: The MEMS accelerometer within the Germpass Unit detects the onset of vertical acceleration, signaling Step 1206 of the procedural flow. This immediate detection allows the system to respond promptly to the change in elevator state.

Step 1216: Access Panel Closure: Following the detection in Step 1206, the microcontroller initiates Step 1216. The drive assembly activates to close the access panel smoothly and securely. This action seals off the elevator button panel, preparing it for the decontamination process while preventing any passenger interaction during movement.

Step 1218: Decontamination Cycle Initiation: With the access panel closed in Step 1216, the system begins the decontamination cycle in Step 1218. The UV-C LED array is activated to deliver a precise 265 nm wavelength dose to the button surfaces. This specific wavelength is chosen for its optimal germicidal effectiveness, as it effectively disrupts the DNA and RNA of microorganisms, leading to their inactivation.

The decontamination cycle is timed to align with the elevator's transit duration, maximizing sanitization effectiveness during movement.

Step 1220: Decontamination Cycle Completion: As the elevator approaches its destination floor, the decontamination cycle completes in Step 1220. The system ensures that sufficient UV-C exposure has been applied to achieve high levels of sanitization. The access panel remains closed until further input is received from the sensors or operational changes occur.

Throughout the decontamination process, the microcontroller continues to monitor sensor inputs at millisecond intervals. The occupancy sensors remain vigilant for any passenger movement, and the MEMS accelerometer tracks changes in vertical acceleration, ensuring the system may respond immediately to any state changes.

In some embodiments, the Elevator Cab Germpass Unit may incorporate an intelligent access management system that prioritizes both sanitation and passenger convenience through its dynamic reopening functionality. After passengers enter the elevator and select their desired floors, the system detects the initiation of cab movement through its integrated MEMS accelerometer and temporarily closes the access door to perform a rapid UV-C decontamination cycle. This cycle effectively sanitizes the button panel surface following the initial passenger interactions. However, unlike conventional systems that maintain panel closure throughout elevator movement, the Germpass Unit implements a sophisticated reopening protocol. Upon completion of the decontamination cycle, the system's occupancy sensors continue to monitor the presence of passengers within the cab. If passengers are detected, the access door automatically reopens to provide continued access to the button panel, even while the elevator remains in motion.

This dynamic reopening feature addresses an important passenger experience consideration in elevator operations. By reopening the panel after decontamination while the elevator is still moving, the system ensures passengers maintain access to the button panel throughout their journey. This functionality proves particularly valuable in scenarios where passengers may need to modify their destination selections or activate additional elevator features during transit. The system achieves this enhanced accessibility while still maintaining its primary sanitation function through the brief initial decontamination cycle performed at the start of elevator movement.

The integration of occupancy detection with the reopening mechanism creates a responsive system that adapts to real-world usage patterns. The continuous monitoring of passenger presence ensures the access panel remains open only when needed, while the rapid initial decontamination cycle ensures optimal sanitization is achieved without causing significant user inconvenience. This balanced approach represents a significant advancement over traditional elevator sanitation systems that may either leave panels continuously exposed or maintain extended closure periods during cab movement.

### Example Scenario 3: Intermediate Floor Stop Operations

During vertical transit with a closed access panel and active decontamination cycle, the elevator may stop at an intermediate floor due to a passenger request or hall call.

Step 1206: Detection of Vertical Movement Cessation: The MEMS accelerometer detects the cessation of vertical movement, triggering Step 1206 again. This detection signals that the elevator has stopped moving vertically, requiring a reassessment of system operations.

Immediate Interrupt Sequence: Simultaneously, the occupancy sensors detect passenger presence-cither existing passengers wishing to select new floors or new passengers entering the cab. Recognizing the need for immediate passenger interaction, the microcontroller executes an immediate interrupt sequence. The UV-C LED operation is halted mid-cycle to prevent any potential exposure to passengers, adhering to strict safety protocols.

Step 1214: Access Panel Reopening: Following the interrupt, the microcontroller initiates Step 1214 to open the access panel. The drive assembly activates to smoothly reveal the elevator button panel. This rapid transition ensures that passengers experience no perceptible delay in button availability, maintaining a seamless user experience even during unexpected stops.

### Example Scenario 4: Idle Elevator at Lobby

Upon arrival at the lobby floor, the Germpass Unit detects both the stop in vertical movement and the presence of existing passengers.

Step 1214: Access Panel Opening for Passenger Exit: The access panel remains open or opens automatically via Step 1214 to facilitate passenger exit. The occupancy sensors monitor the departure of passengers from the cab, ensuring accurate detection of the cab's occupancy state.

Vacancy Detection and Timer Initiation: After the last passenger departs, the occupancy sensors register the vacant state. The microcontroller initiates two timers to confirm sustained vacancy and cab dormancy:
T1 Timer (e.g., 25 seconds): This timer measures 25 seconds of continuous vacancy, ensuring that no passengers are present during this period.
T2 Timer (e.g., 30 seconds): This timer measures 30 seconds of no vertical movement, confirming that the elevator is idle.

These timers run concurrently, and both conditions must be satisfied before proceeding to the next steps. Step 1216: Access Panel Closure Post Timers Expiration: When both the T1 and T2 timers expire—confirming more than 25 seconds of vacancy and more than 30 seconds of no movement—the microcontroller executes Step 1216 to close the access panel. The drive assembly securely seals the elevator button panel, preparing it for sanitization without impacting any potential passenger use.

Step 1218: Decontamination Cycle Initiation: Following the closure in Step 1216, the system begins a complete decontamination cycle in Step 1218. The UV-C LED array activates, delivering the 265 nm wavelength dose to the button surfaces. Utilizing the idle period for decontamination maximizes sanitization opportunities without interfering with elevator operations.

Step 1220: Decontamination Cycle Completion: Upon completion of the decontamination cycle in Step 1220, the system ensures the elevator button panel is thoroughly sanitized. The access panel remains closed to protect the sanitized surfaces from airborne contamination, maintaining hygiene until the next passenger interaction.

Step 1221: Standby Mode Entry: The Germpass Unit enters standby mode in Step 1221. In this state, the microcontroller continues to monitor all sensor inputs at millisecond intervals, ready to detect and respond to new passenger arrivals. The system remains vigilant, ensuring immediate access panel opening upon passenger detection.

Passenger safety is paramount in all operations. The system is designed to prevent any exposure to UV-C light. If passengers are detected during an active decontamination cycle—such as in Scenario 3—the UV-C LEDs are immediately deactivated. The access panel opens without delay, ensuring passengers have instant access to the elevator controls. The use of safety interlocks and immediate interrupt sequences guarantees compliance with safety standards.

The microcontroller's continuous monitoring at millisecond intervals allows for rapid detection and response to any changes in the elevator's operational state. This high-frequency monitoring ensures that the system may swiftly transition between modes—whether opening the access panel for passenger access or initiating decontamination cycles during appropriate times—without causing perceptible delays.

The combination of passive infrared and ultrasonic detection systems enhances the accuracy and reliability of passenger detection. Passive infrared sensors detect the heat emitted by passengers, while ultrasonic sensors use sound waves to detect movement. Together, they ensure accurate detection regardless of environmental conditions, contributing to the system's responsiveness and reliability.

The UV-C LED array emits light at a wavelength of 265 nm, which is within the optimal range for germicidal effectiveness. This wavelength effectively inactivates a wide range of microorganisms by damaging their nucleic acids, preventing replication and ensuring high levels of sanitization on the elevator button surfaces. The system calculates the necessary exposure time based on scientific data to achieve effective decontamination during each cycle.

In at least one embodiment, the T1 and T2 timers help facilitate optimizing decontamination opportunities without disrupting passenger service. The T1 Timer ensures that the cab has been vacant for a minimum of T1 seconds (e.g., T1=25 sec, T1=30 sec, etc.), reducing the likelihood of interrupting a decontamination cycle due to late passenger detection. The T2 Timer confirms that the elevator has been idle—no vertical movement—for a minimum of T2 seconds (e.g., T2=25 sec, T2=30 sec, etc.), indicating an opportune moment for sanitization. Only when both timers have expired does the system proceed with closing the access panel and initiating the decontamination cycle, ensuring efficiency and passenger convenience.

Each operational phase is clearly defined and executed based on precise sensor inputs and procedural steps:
Standby Mode: The system awaits passenger detection with the access panel closed.
Passenger Detection (Step 1212): Occupancy sensors detect passengers, triggering the access panel to open.
Access Panel Opening (Step 1214): Drive assembly opens the panel, providing immediate access to controls.
Vertical Movement Detection (Step 1206): Accelerometer detects movement, prompting panel closure.
Access Panel Closure (Step 1216): Panel closes in preparation for decontamination.
Decontamination Cycle Initiation (Step 1218): UV-C LEDs activate for sanitization.
Decontamination Cycle Completion (Step 1220): Cycle ends, and system awaits next input.
Standby Mode Entry (Step 1221): System returns to standby after idle decontamination.

Throughout all scenarios, the Germpass Elevator Cab Unit demonstrates seamless integration of advanced technologies to maintain hygiene without compromising passenger convenience or safety. The system strategically utilizes elevator transit times and idle periods to perform effective decontamination cycles, ensuring that elevator button panels remain sanitized. By separately detailing each procedural step and emphasizing the interaction of sensors, microcontroller logic, and mechanical components, the operational flow is transparent and comprehensible. The meticulous design and execution of the Germpass Unit highlight its capability to enhance public health measures in shared spaces while delivering an uninterrupted and safe passenger experience.

Users interact with the Germpass system passively. When they enter the elevator, the access panel automatically opens to allow button interaction. No direct input from users is required to trigger decontamination; the system responds entirely to sensor data and environmental conditions.

### Noteworthy Aspects and Features

1. Automatic Transition Between Open and Closed Access Panel States Based on Environmental Inputs: The Germpass Unit automatically transitions between open and closed states of the access panel, based on sensor inputs such as motion and presence detection. This automatic operation ensures the panel remains closed during decontamination, reducing potential contamination risks. Unlike manual or user-triggered systems, the Germpass Unit responds entirely autonomously to real-time conditions, minimizing human interaction and improving sanitation efficiency.
2. Vertical Movement Detection to Trigger Decontamination Cycle: One of the novel aspects of the Germpass Unit is its ability to detect vertical movement of the elevator cab using a MEMS accelerometer. When vertical movement is detected (Step 1206), the system assumes the elevator doors have closed and automatically initiates a decontamination cycle. This feature optimizes the timing of the cleaning process, as decontamination occurs when the elevator is in motion and the panel is not in use, reducing the likelihood of recontamination. This contrasts with prior art techniques where cleaning is either manual or initiated at fixed intervals, regardless of elevator activity. The integration of vertical movement detection allows for a more dynamic and efficient cleaning process.
3. UV-C LED Decontamination During Transit: The Germpass Unit's UV-C LED array is activated during elevator transit, ensuring rapid decontamination of the control panel while the elevator is moving between floors. This cycle is initiated immediately after the access panel is closed, and the UV-C LEDs emit light at the optimal wavelength of 265 nm to destroy pathogens on the panel surface. The strategic use of UVGI during transit, when user interaction is minimal, differentiates this system from slower chemical-based sanitation methods that often leave residues or may require manual application. The reflective internal surfaces within the unit maximize UV-C exposure, ensuring effective germicidal action across the entire surface area.

These noteworthy aspects, including automatic panel state transition, vertical movement detection using MEMS accelerometers, and UV-C LED decontamination, work together to create an optimized, efficient, and fully automated germicidal process. Unlike traditional cleaning methods, which rely on fixed schedules or manual initiation, the Germpass system dynamically responds to real-world elevator conditions, providing effective sanitization without disrupting user experience.

### Germpass Unit User Intent Interpretation Procedure

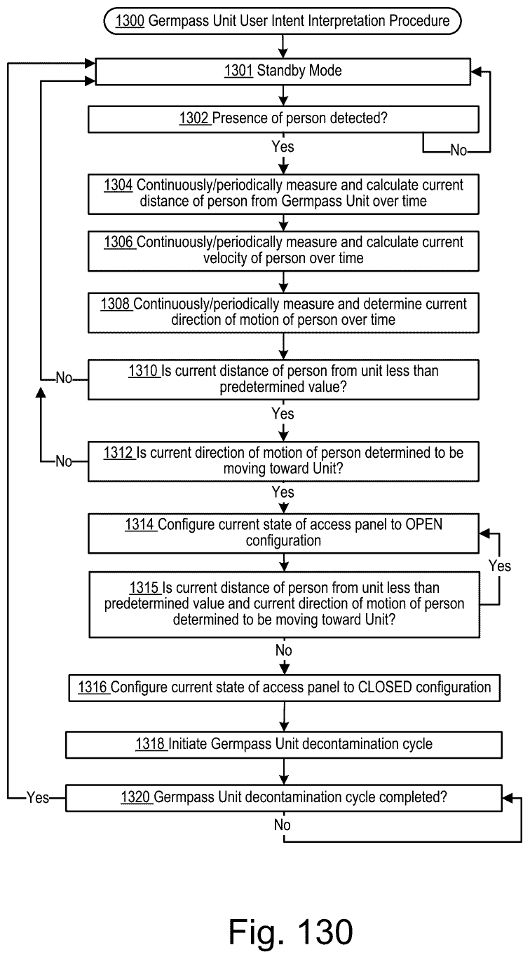

FIG. 130 shows and example flow diagram of an Germpass Unit User Intent Interpretation Procedure 1300.

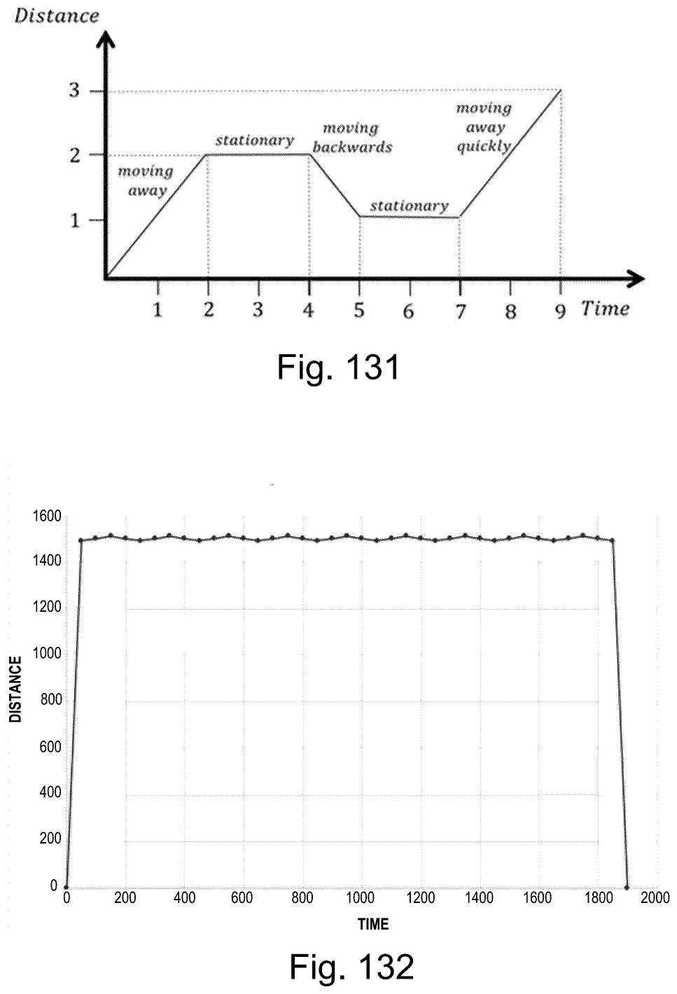

FIG. 131 shows an example graph illustrating various interpretations of an object's movement(s) of an object as a function of distance versus time measurements.

FIG. 132 shows a first example graph of Germpass Unit sensor data measuring a person's movements as a function of distance vs time measurements, where the person is moving in a first direction which is relatively parallel to the Germpass Unit.

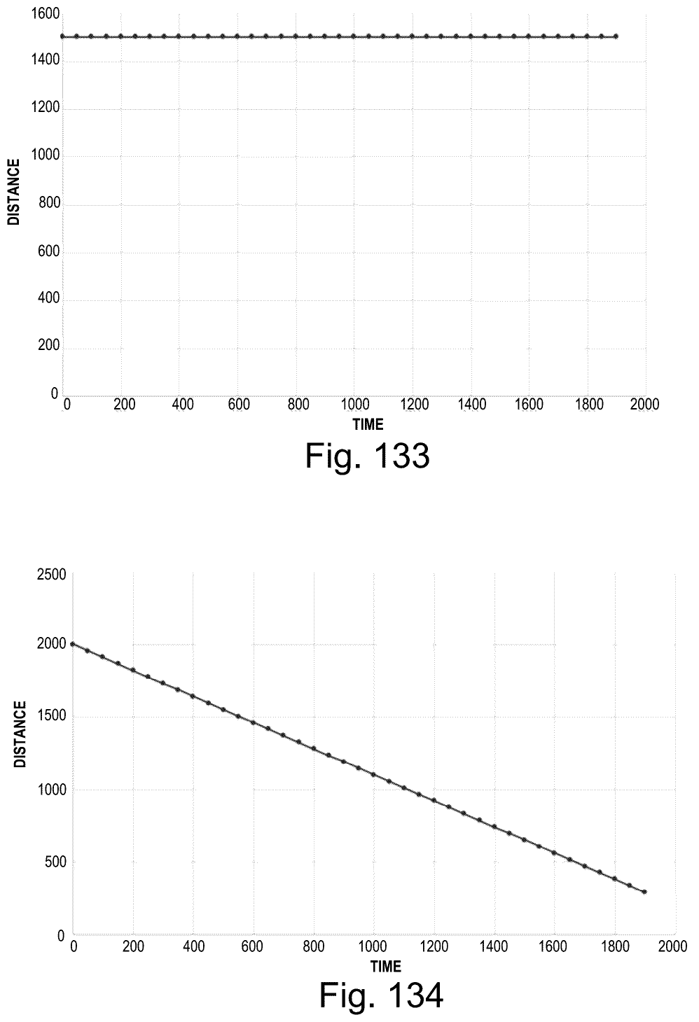

FIG. 133 shows a second example graph of Germpass Unit sensor data measuring a person's movements as a function of distance versus time measurements, where the person is standing still.

FIG. 134 shows a third example graph of Germpass Unit sensor data measuring a person's movements as a function of distance versus time measurements, where the person is moving in a third direction toward Germpass Unit.

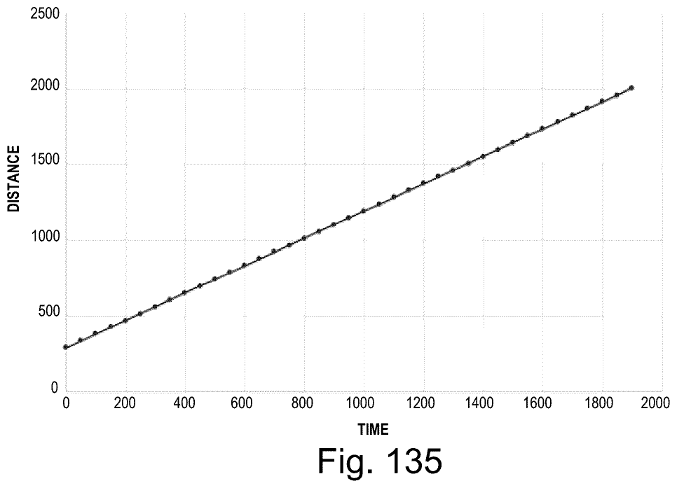

FIG. 135 shows a fourth example graph of Germpass Unit sensor data measuring a person's movements as a function of distance versus time measurements, where the person is moving in a fourth direction away from the Germpass Unit.

The Germpass Unit User Intent Interpretation Procedure (FIG. 130) represents a sophisticated, real-time approach to pathogen control at public touch points. This procedure outlines how a Germpass Unit interacts with users to detect, interpret, and respond to their actions, providing access to germ-protected fomites such as door handles and buttons while preventing contamination. By employing advanced sensor technology and intelligent decontamination algorithms, the system ensures that fomites are protected from contamination between user interactions.

In at least one embodiment, Germpass Units exemplify advanced hygienic technology by integrating adaptive sensor systems, intelligent control algorithms, and user-friendly interfaces. Its design prioritizes safety, efficiency, and sustainability, making it suitable for a wide range of applications—from healthcare facilities to public transportation hubs. By dynamically adjusting to environmental conditions and user behaviors, the Germpass Unit(s) maintain optimal performance while providing a reliable barrier against pathogen transmission. Through detailed error handling, robust maintenance protocols, and considerations for security and privacy, the system(s) ensure continuous functionality and user trust.

### Detection and Interpretation of User Intent

1. Presence Detection: The Germpass Unit uses a combination of sensor inputs to detect the presence of individuals approaching the fomite. Sensors monitor the environment to identify when a person is within a certain proximity.
2. Motion Calculation: Once presence is detected, the system calculates the user's proximity, velocity, and direction of movement. This involves analyzing data from the sensors to determine the user's movement patterns.
3. Intent Interpretation: The system interprets the user's intent based on the calculated motion parameters. If the user's movement and proximity suggest an intention to interact with the fomite, the system proceeds to grant access.

### Control of Access to Fomites

4. Access Door Operation: If access is granted, the Germpass Unit opens an access door or panel, exposing the fomite for user interaction. This ensures that the fomite is only exposed when necessary, reducing the risk of contamination.
5. User Interaction: The user interacts with the fomite, such as turning a door handle or pressing a button, while the system continues to monitor for the completion of the interaction.

### Decontamination Cycle

6. Initiation of Decontamination: After the user completes the interaction and moves away, the Germpass Unit automatically initiates a decontamination cycle using ultraviolet germicidal irradiation (UVGI), specifically UV-C light. This rapid decontamination process ensures the fomite is sanitized before the next user approaches.
7. Completion and Reset: Upon completion of the decontamination cycle, the access door closes, and the system resets, ready to detect the next user.

This procedural flow is structured to minimize unnecessary decontamination cycles, conserve power, and efficiently manage the interaction with the fomite. By accurately interpreting user intent while minimizing unnecessary operations, the system enhances user safety and convenience. It reduces contamination risk by ensuring the fomite is only exposed when necessary and enables efficient, contactless decontamination.

The Germpass Unit operates in both embedded and retrofit embodiments, making it suitable for high-traffic public spaces such as doors, restroom latches, and elevator buttons. Its novel methods for detecting user intent, managing energy-efficient decontamination cycles, and automating fomite protection represent a significant advancement in public health technology. By ensuring cleanliness without requiring constant manual intervention, the Germpass Unit sets itself apart from traditional decontamination systems, making it an ideal solution for enhancing public safety.

The Germpass Unit integrates various types of hardware and software components to ensure efficient and safe interactions in public environments. Central to its operation are the ultrasonic sensor (e.g., TDK CH201), microcontroller processing units, and UV-C decontamination systems. These elements work in harmony to interpret user intent through real-time distance and velocity measurements. This capability ensures smooth operation and enhances user experience. The inclusion of motorized drive assemblies, coupled with UV-C decontamination, enhances the unit's functionality, making it well-suited for high-traffic public areas.

Implementing the Germpass Unit across various public settings may require robust hardware and software integration. Alongside the TDK CH201 ultrasonic sensor, infrared detectors are employed to ensure accurate user detection. For precise motion detection and direction calculations, the system applies a combination of filtering techniques to the raw sensor data. Specifically, median and moving average filters are used to smooth out noise, guaranteeing an accurate interpretation of user intent.

The unit's Ultraviolet Germicidal Irradiation (UVGI) system is powered by 265 nm UV-C LEDS, providing rapid and effective sterilization. To optimize energy efficiency, the system incorporates proximity sensors inside the enclosure to detect hand interactions. This feature prevents unnecessary sterilization cycles when the fomite is not touched, significantly extending battery life and enhancing the system's overall efficiency.

### Enhanced Access Security Features

In at least some embodiments, the Germpass Unit incorporates advanced authentication and access control capabilities through integration with various remote identification technologies. The system includes receivers and decoders for detecting and processing remote signals from fobs, RFID tags, and NFC devices. When a user approaches with an authorized remote device, the system's integrated receiver detects the unique identifier signal broadcast by the device. The signal is decoded and validated against a local or remotely accessed database of authorized credentials. Upon successful validation, the system's controller initiates the access panel opening sequence, providing touchless access to the protected fomite. This capability is particularly valuable in secure facilities where access control is paramount, such as healthcare environments, research laboratories, or restricted corporate areas. The remote signal detection system operates concurrently with the unit's proximity and motion sensors, providing redundant activation methods to accommodate different user preferences and security requirements.

In some embodiments, the facial recognition functionality is implemented through integration of compact high-resolution cameras and specialized image processing hardware within the Germpass Unit. The system captures facial images when users approach, processes these images using advanced facial recognition algorithms, and compares the processed data against an authorized user database. The facial recognition system is trained to operate effectively under various lighting conditions and angles of approach. When an authorized face is detected and validated, the access panel opens automatically. This touchless activation method enhances both security and hygiene by eliminating the need for physical credential presentation. The facial recognition system maintains user privacy by processing biometric data locally within the unit, with only encrypted authentication results being transmitted to external systems when required.

In some embodiments, for enhanced security applications, the Germpass Unit incorporates biometric identification capabilities including fingerprint scanning and iris recognition. The biometric sensors are strategically positioned to capture user data while maintaining the unit's streamlined form factor. Fingerprint scanning is implemented through integration of capacitive or optical sensors in easily accessible locations on the unit's exterior. The iris recognition system utilizes specialized near-infrared cameras and illuminators to capture detailed iris patterns even in varying ambient light conditions. Biometric data is processed using dedicated hardware acceleration to ensure rapid authentication while maintaining system security.

In at least some embodiments, Germpass Units may also be configured or designed to provide comprehensive logging functionality to maintain detailed records of all user interactions and system operations. The logging system captures multiple data points for each interaction event, including unique user identifiers (when available through remote or biometric authentication), precise timestamps of access initiation and completion, duration of fomite exposure, and completion status of subsequent decontamination cycles. The logging system utilizes non-volatile memory to ensure data persistence even during power interruptions. The stored interaction data includes encrypted user identification information when available through authenticated access methods, while maintaining appropriate privacy controls and compliance with data protection regulations.

The system incorporates wireless communication capabilities through integration of low-power wireless transceivers supporting multiple protocols including WiFi, Bluetooth Low Energy, and cellular data transmission. The wireless subsystem enables real-time transmission of interaction logs to remote monitoring and management systems. The transmission protocol implements robust encryption to protect sensitive data during transit. The wireless system operates in both real-time streaming mode for immediate data transmission and batch mode for periodic uploads, adapting automatically based on network availability and power conditions. When operating in batch mode, the system accumulates interaction records in local storage and transmits them during configured upload windows or when triggered by specific events such as storage capacity thresholds.

The logging and wireless transmission capabilities enable advanced analytics and maintenance operations. Facility managers may analyze usage patterns to optimize cleaning cycles and maintenance schedules. The real-time transmission capability supports immediate notification of system status changes or maintenance requirements. The wireless interface also enables remote configuration updates and system monitoring, reducing the need for physical access to individual units. In at least some embodiments, the logging system maintains detailed records of all maintenance activities, including UV-C component replacements, sensor calibrations, and system updates, providing a comprehensive audit trail of the unit's operational history. Component Interactions and Procedural Steps

Step 1301—Standby Mode: In the initial stage, the Germpass Unit operates in a low-power standby mode with its access panel securely closed. This closed configuration protects the fomite (the common touchpoint) from airborne pathogens and environmental contaminants. During this mode, the system's array of sensors—including ultrasonic sensors like the TDK CH201, infrared presence detectors, and motion sensors—remains active while consuming minimal power. The microcontroller manages periodic sensor checks, maintaining system readiness while optimizing energy consumption.

Implementation Example: In both retrofit and embedded embodiments, such as installations on doors, elevator buttons, or gas pump handles, the Germpass Unit utilizes the TDK CH201 ultrasonic sensor operating in free-run mode. This sensor continuously captures environmental measurements at intervals, typically every 50 milliseconds, without requiring external trigger signals. The microcontroller employs low-power modes during standby, waking only to process sensor data. For instance, in a gas station pump handle application, the system adjusts its sensors to account for outdoor conditions like temperature fluctuations and varying light levels, ensuring accurate detection without unnecessary power consumption.

Adjustable Parameters and Calibration: The system may dynamically adjust parameters like the distance threshold (D1) and sensor sensitivity based on the environment. In high-traffic areas, such as busy elevators or public restrooms, the distance threshold may be reduced to allow quicker activation. In low-traffic areas, it may be increased to prevent false triggers from passersby. The microcontroller calibrates sensor sensitivity in real-time, accounting for factors like ambient noise and temperature variations, ensuring reliable operation across different settings.

Power Management and Sustainability: During standby mode, some components may be powered down to conserve energy. The system utilizes low-power microcontrollers and energy-efficient sensors. Options for solar panels or energy harvesting from mechanical movements (like door swings) may supplement power, extending battery life or reducing dependence on AC power. In battery-powered configurations, efficient power management extends battery life expectancy, reducing maintenance requirements. Step 1302—Presence of Person Detected? At this stage, the Germpass Unit continuously monitors for the presence of a person within its detection range. If no individual is detected, the system remains in standby mode. Upon confirming the presence of a person, the system proceeds to the next step. Detection is based on signals from the sensor array, which analyzes changes in the environment to identify a potential user.

Implementation Example: The Germpass Unit employs the TDK CH201 ultrasonic sensor in free-run mode to autonomously measure distance at regular intervals. The sensor's built-in algorithms, such as the static target rejection algorithm, detect changes in the environment by calculating running average echo amplitudes for each distance measurement. This enables the sensor to identify the approach of a person. The microcontroller processes the sensor data using filtering algorithms to distinguish actual human presence from environmental noise or false triggers. In embedded systems like elevator buttons or restroom latches, sensors may detect both vertical motion and proximity, ensuring interaction only when necessary.

Sensor Calibration and Synchronization: Sensors are calibrated during installation and periodically recalibrated to maintain accuracy. The system may perform self-diagnostics to adjust for sensor drift due to environmental factors. By utilizing multi-sensor fusion—combining data from ultrasonic and infrared sensors—the system improves detection accuracy and reduces false positives. For example, in an elevator control panel, the sensors are calibrated to ignore reflections from metallic surfaces or interference from other electronic devices.

Step 1304—Measure Distance of Person Over Time: Once a person is detected, the system begins continuously measuring their distance from the Germpass Unit. This ongoing measurement helps determine if the person is moving closer to or away from the fomite. The system maintains a rolling buffer of distance measurements to enable velocity calculations and trajectory analysis.

Implementation Example: The CH201 ultrasonic sensor measures distance by emitting ultrasonic pulses and receiving echoes. The microcontroller calculates the distance by analyzing the time delay between the sent pulse and the received echo. Raw distance measurements are filtered using techniques like median filtering or moving average filters to reduce noise and smooth out extreme values or measurement spikes. The measurement subsystem accounts for potential interference from multiple individuals or objects by focusing on the strongest return signal. For instance, in a public restroom latch, the system measures the distance of an approaching hand rather than the whole person, requiring higher sensitivity and precision due to the smaller detection area.

Adjustable Parameters: The system may adjust how frequently it takes distance measurements based on traffic patterns. During peak hours, measurements may be more frequent to accommodate rapid user interactions. Advanced algorithms filter out background noise and transient events, ensuring only relevant distance changes are considered.

Step 1306-Measure Velocity Over Time: Simultaneously, the system calculates the person's velocity by comparing sequential distance measurements. Understanding the user's movement speed and direction allows the system to predict intent and respond appropriately.

Implementation Example: Velocity is computed using the formula:

$$Velocity ( V ) = \frac{Δ d}{Δ t} = \frac{d 2 - d 1}{t 2 - t 1} ,$$

where d1 and d2 are consecutive distance readings, and t1 and t2 are their respective timestamps. A negative velocity indicates the person is moving toward the fomite, while a positive value indicates movement away. Filtering techniques like a moving average filter smooth out the velocity data, ensuring that noise or minor fluctuations do not affect the calculations. For example, in a gas station pump handle application, the system distinguishes between a customer approaching to use the pump and someone walking past, adjusting its response to open the access panel only when appropriate.

Error Handling and Fail-Safes: If velocity calculations are inconsistent or suggest improbable movement (such as sudden large jumps in distance), the system flags a potential sensor error. Multiple sensors provide overlapping data to ensure that a single sensor failure doesn't compromise velocity calculations. The system may initiate self-diagnostic procedures to identify and address sensor malfunctions.

Step 1308—Determine Direction of Motion: The system determines whether the person is moving toward or away from the Germpass Unit. This information is useful for deciding whether to initiate interaction procedures.

Implementation Example: The direction of motion is derived from the sign of the velocity. A negative velocity indicates the person is moving toward the unit, while a positive value indicates movement away. The microcontroller applies additional filtering and state logic to prevent rapid direction classification changes due to minor user movements or measurement noise. In an office building's main entrance door handle, the system accounts for people congregating nearby by requiring a consistent approach direction before opening the access panel.

Adjustable Parameters: The threshold for determining approach may be adjusted. In environments where users may move erratically, such as crowded venues, the system may require more consistent directional data before acting. This helps prevent false activations from users moving parallel to or past the unit.

Step 1310—Is Distance Less Than D1? In at least one embodiment, the system checks if the person is within the predefined distance threshold (D1). If the person is closer than D1, the system prepares to open the access panel. If not, it returns to standby mode.

Implementation Example: The CH201 sensor continuously measures and updates the distance between the user and the unit. The microcontroller compares the most recent filtered distance value against the D1 parameter, accounting for measurement uncertainty through hysteresis in the comparison logic. In public environments, this predefined distance ensures that the Germpass Unit remains sealed unless someone is actively approaching. For example, in a hospital setting, the Germpass Unit at a staff-only door may have a shorter D1 to allow rapid access for medical personnel.

Adjustable Parameters and Calibration: D1 may be adjusted based on the environment. In high-traffic areas, D1 may be reduced to prevent the system from being overwhelmed by high user volume. In low-traffic areas, it may be increased to avoid false triggers. The system may also adapt DI dynamically based on time of day or specific events, learning typical user behavior over time to optimize responsiveness and efficiency. Step 1312—Is Motion Toward the Unit? In at least one embodiment, the system confirms whether the detected person is moving toward the fomite. If so, it proceeds to open the access panel. If the motion is determined to be in another direction, the system returns to standby mode, preserving resources. Implementation Example: The system evaluates both proximity within the D1 threshold and consistent negative velocity to confirm user approach intent. The microcontroller implements a state machine that tracks approach confidence based on multiple consecutive measurements indicating a maintained approach vector. In an automated ticket kiosk, the system ensures that only users intending to interact with the screen trigger the panel opening, conserving energy and reducing wear on mechanical parts.

Error Handling and Fail-Safes: The system may require multiple consistent readings before acting, reducing the chance of false activation due to sensor glitches. If sensors disagree or if data is inconsistent, the system defaults to the safest state-keeping the panel closed-and logs the event for maintenance review. The system may initiate self-diagnostic procedures to check sensor functionality and recalibrate if necessary.

Step 1314—Open Access Panel: If all conditions are met, the Germpass Unit opens its access panel to allow the user to interact with the protected fomite. This ensures that users may interact with the fomite only when necessary, reducing unnecessary panel cycles and maintaining hygiene.

Implementation Example: A motorized drive assembly, powered by stepper motors, moves the access panel to an open configuration. For multi-panel configurations, the drive motor engages the drive chain connected to the drive panel, initiating the synchronized retraction sequence of the stacked access panels. The assembly includes safety features such as embedded obstruction sensors to detect any objects that may interfere with panel movement. The panel movement mechanism utilizes channel guides and support arms to maintain precise alignment during the retraction process. The drive assembly's pulley system ensures smooth panel movement, while UV-reflective coatings on panel surfaces maintain the system's germicidal effectiveness. In an elevator, the panel covering the buttons slides open accompanied by a green light and a gentle tone, indicating to the user that they may select their floor.

User Feedback Mechanisms: The system provides visual indicators, such as LEDs changing color or illuminating, to signal that the panel is opening. Auditory signals, like soft chimes or beeps, indicate the system's status, aiding users with visual impairments. These feedback mechanisms enhance user experience and ensure clear communication of the system's state.

Step 1315—Access Panel Open as Long as User Is Approaching: The panel remains open while the system detects the person within range and moving toward the unit. Continuous monitoring ensures that the access panel stays open only as long as necessary. If the system detects that the person has moved away or is no longer within range, it prepares to close the panel.

Implementation Example: The system continues to monitor velocity and direction. If the user remains within the active interaction zone and continues approaching, the panel stays open. If the system detects user departure—-either through distance exceeding a threshold or consistent positive velocity indicating movement away—it initiates panel closure. At a shopping mall entrance, the Germpass Unit detects a group of people approaching and keeps the panel open longer to allow all users to interact with the door handle before initiating closure.

Adjustable Parameters: The system may adjust how long the panel remains open based on user interaction patterns. In high-traffic areas, the panel may close more quickly to initiate decontamination sooner. The system may detect multiple users approaching and adjust timing to accommodate them without unnecessary panel cycling.

Error Handling and Fail-Safes: Obstruction sensors ensure that if an object blocks the panel's path, movement is halted to prevent damage or injury. Emergency protocols allow the panel to remain open or closed based on security needs or maintenance activities.

Step 1316—Close Access Panel: Once the system determines that the user has completed their interaction or moved away, it initiates the closure of the access panel. Safety mechanisms ensure that no obstruction blocks the panel's path.

Implementation Example: The drive motor reverses direction, engaging the chain drive to extend the stacked panels from their retracted position in the panel bay. The system maintains active monitoring through obstruction sensors to prevent panel closure if objects are detected in the panel path. The microcontroller coordinates the synchronized movement of multiple panels through the channel guide system, ensuring proper panel alignment and complete enclosure sealing. In a corporate office, the panel begins to close after the employee releases the door handle, with lights indicating the process. If someone tries to grasp the handle during closure, the system detects the obstruction and reopens the panel.

User Feedback Mechanisms: Visual cues, such as LEDs changing color to yellow during closure, signal that the panel is closing and caution users to keep clear. Auditory alerts, like a soft warning tone, inform nearby users that the panel is in motion.

Error Handling and Fail-Safes: The system employs infrared or pressure sensors to detect obstructions, halting the panel if necessary. If the panel fails to close properly, the system alerts maintenance personnel and may lock the panel in a safe position to prevent misuse. The system may initiate diagnostic procedures to identify mechanical issues and schedule repairs.

Step 1318—Initiate Decontamination Cycle: After closing the access panel, the system begins a UV-C decontamination cycle to eliminate pathogens on the fomite's surface. This process ensures that the fomite is hygienic for the next user.

Implementation Example: UV-C LEDs emitting at an optimal wavelength of 265 nm are activated inside the chamber, irradiating the fomite. Multiple LED sources mounted at precisely calculated angles ensure complete coverage of the fomite surface. UV-reflective coatings on interior surfaces enhance decontamination effectiveness through controlled light reflection. The microcontroller manages LED activation timing and intensity to deliver the required UV dose for 99.9% pathogen reduction. In a hospital door handle application, the decontamination cycle is set for a longer duration with higher intensity to meet stringent cleanliness standards, whereas in a retail store, a shorter cycle suffices.

Adjustable Parameters and Calibration: The UV-C exposure time and intensity are adjustable based on the level of disinfection required, which may vary between environments. The system may calibrate UV-C output to balance energy consumption with decontamination effectiveness. Sensors monitor UV-C LED performance, adjusting usage to extend their lifespan and scheduling maintenance when degradation is detected.

Power Management and Sustainability: UV-C LEDs are energy-efficient and only activate when necessary, conserving power. The system ensures that energy consumption during decontamination is optimized, contributing to overall sustainability.

Step 1320—Decontamination Cycle Completed? Upon completing the UV-C decontamination cycle, the system verifies the process's success and returns to standby mode, ready for the next user interaction.

Implementation Example: Sensors verify the status of the UV-C cycle, and the microcontroller confirms successful completion. In an airport security checkpoint, the Germpass Unit logs each decontamination cycle's completion and alerts staff if any anomalies are detected, ensuring a consistent level of hygiene. The system transitions back to its default standby state while maintaining the sealed panel configuration.

User Feedback Mechanisms: Status indicators, such as LEDs illuminating blue during decontamination and turning green when complete, communicate the system's readiness to users. In some settings, the system may send a signal to a central monitoring station confirming successful decontamination.

Error Handling and Fail-Safes: The system checks UV-C LED functionality and exposure duration to ensure effectiveness. If the cycle fails (due to a faulty LED or other issues), the system may lock the panel and notify maintenance personnel. The decontamination cycle management subsystem ensures reliable pathogen reduction while optimizing system availability for subsequent users.

The Germpass Unit incorporates robust error handling and fail-safe mechanisms to ensure continuous functionality and user safety. The system performs regular self-diagnostics on sensors, mechanical components, and electrical systems. If anomalies are detected, it enters a safe mode and alerts maintenance personnel. Various components like sensors and motors have redundancies to ensure continued operation even if one component fails. In case of power failure, the system may revert to a mechanical default state, allowing manual use of the fomite. The system logs errors and events for maintenance review, facilitating timely repairs and system improvements.

Routine maintenance may be desirable for optimal performance of the Germpass Unit. Scheduled maintenance includes cleaning sensors, lubricating mechanical parts, and verifying UV-C LED output. UV-C LEDs and batteries (if applicable) have specified lifespans and are replaced as part of preventive maintenance. The system logs performance data to predict when components will fail, allowing for timely replacements. In environments with heavy use, maintenance schedules may be more frequent to ensure reliability. The system's design emphasizes durability and case of maintenance, reducing downtime and operational costs.

While the Germpass Unit collects data on user proximity and movement, it handles data security with utmost importance. The system processes proximity and movement data in real-time without storing personal information, ensuring user privacy. Data transmissions, if networked, are encrypted to prevent unauthorized access. The system adheres to relevant data protection regulations, such as GDPR, ensuring legal compliance. By anonymizing data and limiting retention, the system mitigates privacy concerns associated with sensor data collection.

The Germpass Unit is designed with energy efficiency and sustainability in mind. Components are selected for low power consumption, and the system intelligently manages power based on usage patterns. During idle periods, non-desirable components are powered down to conserve energy. Alternative energy sources, such as solar panels or kinetic energy harvesters, may supplement power, particularly in remote or outdoor installations. Materials are chosen for durability and recyclability, minimizing environmental footprint. Efficient power management extends battery life in battery-powered configurations, reducing maintenance and environmental impact.

The system ensures accurate sensor performance through automated calibration and synchronization. Sensors are calibrated during installation and periodically recalibrated using known reference points or during low-traffic periods. The system adjusts sensor sensitivity based on ambient conditions, such as temperature or humidity, to maintain accuracy. In large facilities, multiple Germpass Units may share data to improve overall system performance, utilizing integrated sensor networks for enhanced detection accuracy.

Each procedural step incorporates decision-making logic that considers multiple factors, ensuring seamless transitions. From standby to presence detection, activation occurs only when sensors confirm a valid presence, reducing false triggers. The transition from detection to panel opening may require both proximity within D1 and consistent approach direction, verified through multiple sensor readings. The system maintains a state machine that tracks conditions and adjusts actions accordingly. By incorporating adjustable parameters and adaptive algorithms, the system optimizes responsiveness while preventing unnecessary activations.

### Example Germpass Door Handle Unit Interaction Scenarios

The Germpass Door Handle Unit is an innovative device designed to reduce the transmission of germs through door handles, which are common fomites—objects to carry infection. Installed over existing door handles, the Germpass Unit utilizes embedded ultrasonic sensors to detect user presence and intent, an access panel to control physical interaction, and ultraviolet germicidal irradiation (UVGI decontamination) to sanitize the handle after each use. In at least some embodiments, the Germpass Door Handle Unit intelligently responds to user behavior to maintain hygiene and safety standards. By accurately detecting presence, velocity, and direction, it ensures the access panel opens only when necessary, and decontamination cycles are initiated appropriately. This optimizes energy use, extends component lifespan, and reduces the spread of pathogens.

### Example Scenario 1: Typical Use Case—User Interacts With the Door Handle

In a busy office environment, a Germpass Unit is installed over a door handle. A person approaches the door intending to open it. The system detects their presence, grants access, and ensures decontamination afterward.
Step 1: Standby Mode (Step 1301): The Germpass Unit is initially in standby mode. The access panel is closed, and the system actively scans for the presence of a person using embedded ultrasonic motion sensors. These sensors emit ultrasonic waves and listen for their echoes to detect movement and presence within a specific range.
Step 2: Presence Detected (Step 1302): As the person walks toward the door, the ultrasonic sensors detect their presence within a 4-foot proximity threshold. The system begins taking periodic distance measurements by timing the delay between emitted ultrasonic pulses and their echoes.
Step 3: Distance Measurement (Step 1304): The system continuously measures the user's distance from the door handle. By calculating the time it takes for the ultrasonic waves to return, the system determines the precise distance of the user at regular intervals.
Step 4: Velocity Calculation (Step 1306): Using the changes in distance over time, the system calculates the user's velocity. Velocity is determined by the rate at which the distance measurements decrease as the user approaches the door handle, using the formula:

$$Velocity ( V ) = \frac{Δ d}{Δ t} = \frac{d 2 - d 1}{t 2 - t 1}$$

Step 5: Direction Determination (Step 1308): By analyzing the sequence of distance measurements and the calculated velocity, the system determines the user's direction of motion. If the distance to the user is decreasing and their velocity vector is directed toward the unit, the system confirms they are approaching the door handle.
Step 6: Intent Confirmation (Step 1310): The system evaluates whether the user intends to interact with the door handle. If the user is within the predetermined threshold distance (e.g., less than 4 feet) and moving toward the handle, the system confirms the user's intent to interact with the fomite.
Step 7: Threshold Confirmation (Step 1312): Upon confirming the user's intent, the system verifies that all conditions are met to proceed with granting access. This includes ensuring the user's proximity and direction align with interaction criteria.
Step 8: Access Panel Opens (Step 1314): The system automatically opens the access panel. The panel retracts smoothly to expose the door handle, allowing the user to grasp it. The access panel serves not only as a barrier to prevent unintended contact but also as a protective shield during decontamination cycles.
Step 9: User Interaction: The user grabs the door handle, opens the door, and walks through. The system continues to monitor the user's presence, ensuring the access panel remains open for the duration of the interaction.
Step 10: Continuous Monitoring (Step 1315): While the user is interacting with the door handle, the system continuously checks their distance and direction. This ensures that the access panel stays open as long as the user is within the fomite region, preventing premature closure.
Step 11: User Moves Away: After opening the door, the system detects that the user is moving away from the Germpass Unit. The distance measurements increase, and the velocity indicates movement away from the unit.
Step 12: Access Panel Closes (Step 1316): Recognizing that the user has left the immediate vicinity, the system closes the access panel. The panel returns to its closed position, sealing off the door handle from further contact.
Step 13: Initiating Decontamination Cycle (Step 1318): With the access panel closed, the Germpass Unit initiates a UVGI decontamination cycle. Ultraviolet-C (UV-C) light is emitted inside the sealed chamber to irradiate the door handle for a predetermined duration, effectively killing germs and pathogens.
Step 14: Safety Measures: The access panel acts as a barrier to contain the UV-C light, ensuring that it does not escape and pose a risk to users. The interior surfaces are designed to reflect UV-C light efficiently, maximizing the effectiveness of the decontamination while maintaining user safety.
Step 15: Decontamination Cycle Completed (Step 1320): Upon completing the decontamination cycle, the system verifies that the UV-C exposure time meets the necessary criteria for effective germicidal action. The system then turns off the UV-C light.
Step 16: Return to Standby Mode: The Germpass Unit returns to standby mode, with the access panel closed and sensors active, ready to detect the next user.

By initiating the decontamination cycle only when necessary, e.g., after confirmed contact with the door handle, or after confirmation that the presence of a foreign object (e.g., user's hand) has been detected in the interior chamber of the Germpass Unit, the system may conserve energy and extend the lifespan of the UV-C components and battery power source(s).

Throughout the interaction, the system may display visual indicators, such as LED lights, to signal the status of the access panel and decontamination cycle. For example, a green light may illuminate when the access panel opens, indicating the door handle is available for use, while a blue light may activate during the decontamination cycle.

### Example Scenario 2: User Passes by the Door Without Interaction

A person walks near the door but does not intend to interact with the door handle. The system detects this behavior and avoids unnecessary panel operations and decontamination cycles.
Step 1: Standby Mode (Step 1301): The Germpass Unit is in standby mode, with the access panel closed and sensors actively scanning the environment.
Step 2: Presence Detected (Step 1302): The ultrasonic sensors detect the user's presence within the 4-foot proximity threshold.
Step 3: Distance Measurement (Step 1304): The system begins taking periodic distance measurements to track the user's proximity to the door handle.
Step 4: Velocity Calculation (Step 1306): Using the changes in distance over time, the system calculates the user's velocity.
Step 5: Direction Determination (Step 1308): Analyzing the distance and velocity data, the system determines the user's direction of movement. In this case, the user is moving parallel to the door, not toward the handle.
Step 6: Intent Evaluation (Step 1310): Since the user is not approaching the door handle within the threshold distance and their direction is not toward the unit, the system concludes there is no intent to interact with the fomite.
Step 7: Threshold Confirmation (Step 1312): The system verifies that conditions for granting access are not met, as the user does not intend to interact with the handle.
Step 8: Access Panel Remains Closed: The system decides not to open the access panel, conserving energy and reducing wear on mechanical components.
Step 9: Continuous Monitoring: The system continues to monitor the user's movement until they are out of detection range.
Step 10: Return to Standby Mode: After the user moves away, the system remains in standby mode, ready for the next detection.

By avoiding unnecessary activation of the access panel and decontamination cycle, the system conserves energy and extends component life.

### Example Scenario 3: False Alarm—User Changes Direction Without Interaction

A person approaches the door as if to open it but changes direction before touching the handle. The system adjusts its response to this change in behavior.
Step 1: Standby Mode (Step 1301): The system is in standby mode, scanning for user presence.
Step 2: Presence Detected (Step 1302): The ultrasonic sensors detect the user's presence within the 4-foot threshold.
Step 3: Distance Measurement (Step 1304): The system begins measuring the user's distance from the door handle at regular intervals.
Step 4: Velocity Calculation (Step 1306): The user's velocity is calculated based on the rate of change in distance.
Step 5: Direction Determination (Step 1308): The system determines that the user is moving toward the door handle.
Step 6: Intent Confirmation (Step 1310): The system confirms the user's intent to interact with the fomite, as they are within the threshold distance and approaching the handle.
Step 7: Threshold Confirmation (Step 1312): All conditions for granting access are met, and the system proceeds accordingly.
Step 8: Access Panel Opens (Step 1314): The access panel opens to provide access to the door handle.
Step 9: Continuous Monitoring (Step 1315): The system continuously monitors the user's distance and direction.
Step 10: User Changes Direction: Before touching the handle, the user turns around and walks away. The system detects an increase in distance and a reversal in direction.
Step 11: Access Panel Closes (Step 1316): Recognizing that the user has moved away without interacting with the handle, the system closes the access panel.
Step 12: Skipping Decontamination Cycle: Since the user did not touch the door handle, the system skips the UVGI decontamination cycle. This conserves energy and extends the life of the UV-C components.
Step 13: Return to Standby Mode: The system returns to standby mode, ready to detect the next user. By accurately detecting changes in user behavior, the system ensures safety and operational efficiency, avoiding unnecessary decontamination processes.

### Example Walk-Through Scenario: Application in a Hospital Setting

In a hospital, a Germpass Unit is installed on a door leading to a patient ward. A healthcare worker approaches the door intending to enter.
Step 1: Standby Mode: The system is in standby mode, with the access panel closed and sensors active.
Step 2: Presence Detected (Step 1302): The ultrasonic sensors detect the healthcare worker within the 4-foot range.
Step 3: Distance Measurement (Step 1304): Distance measurements begin to track the worker's approach.
Step 4: Velocity Calculation (Step 1306): The system calculates the worker's velocity toward the door handle.
Step 5: Direction Determination (Step 1308): The system confirms the worker is moving toward the unit.
Step 6: Intent Confirmation (Step 1310): The system determines the worker intends to interact with the door handle.
Step 7: Threshold Confirmation (Step 1312): All conditions for access are met, and the system proceeds to grant access.
Step 8: Access Panel Opens (Step 1314): The access panel opens, granting access to the door handle.
Step 9: User Interaction: The healthcare worker opens the door and enters the ward.
Step 10: Continuous Monitoring (Step 1315): The system ensures the access panel remains open during the interaction.
Step 11: User Moves Away: After passing through, the system detects the worker moving away.
Step 12: Access Panel Closes (Step 1316): The access panel closes, sealing off the handle.
Step 13: Initiating Decontamination Cycle (Step 1318): Given the high-risk environment, the system promptly initiates the UVGI decontamination cycle.
Step 14: Decontamination Cycle Completed (Step 1320): After decontamination, the system returns to standby mode.

### Example Walk-Through Scenario: Delayed Access Door Closing for Multi-Person Access

In this scenario, a Germpass Door Handle Unit is installed at a busy office building entrance where multiple people frequently enter/exit in groups. The system implements an intelligent delay mechanism to accommodate sequential users while maintaining optimal sanitization.

When a user approaches the door handle, the ultrasonic sensors detect their presence within the 4-foot proximity threshold. The system calculates the user's velocity and direction using the formula V=Δd/Δt, where d represents distance measurements taken at 50 ms intervals. Upon confirming approach intent, the access panel opens smoothly via the motorized drive assembly to expose the sanitized door handle.

After the first user interacts with the handle, instead of immediately closing, the system initiates a 20-second (T3) delay timer. During this interval, the system maintains active monitoring through its sensor array to detect additional approaching users. The microcontroller processes sensor data through noise rejection and validation filters to accurately identify subsequent users.

For example, when a group of colleagues exits the office, the first person opens the door. The system detects their completed interaction but keeps the access panel open. As the second and third colleagues approach within the next few seconds, the system recognizes their presence and maintains the open configuration. The T3 timer resets with each new valid user detection, ensuring adequate access time for the entire group.

The Germpass Unit implements an adaptable delayed closing mechanism through its T3 delay parameter, which may be configured across a broad range from 5 seconds to greater than 30 seconds based on multiple environmental and operational factors. In high-traffic environments such as busy office building entrances or shopping centers, the T3 delay may be set to longer durations (20-30+seconds) to accommodate groups of people entering in succession, reducing unnecessary open/close cycles and conserving system resources. In lower-traffic locations like individual restroom stalls or private offices, shorter T3 delays (5-15 seconds) may be utilized to minimize fomite exposure while still providing adequate access time. The delay duration may also be dynamically adjusted based on time of day, with longer delays during peak usage hours and shorter delays during off-peak periods. Additional factors influencing the T3 parameter include the specific type of fomite being protected, local security requirements, and facility operational policies. For example, hospital environments may implement shorter delays to minimize potential contamination exposure, while retail environments may use longer delays to enhance customer convenience. The system also considers the physical characteristics of the installation location, such as door swing paths or space constraints, when determining optimal delay timing. This delayed closing mechanism provides several advantages:
Reduces mechanical wear by avoiding rapid open/close cycles
Conserves energy by preventing multiple UV-C decontamination cycles
Improves user experience for groups entering/exiting together
Maintains efficient traffic flow in high-volume areas

The system continues monitoring proximity and direction during the delay period. If no additional users are detected for the full 20-second interval, the access panel closes via the drive assembly. The system then initiates a single UV-C decontamination cycle at 265 nm wavelength to sanitize the handle, preparing it for the next user group.

The T3 delay parameter may be adjusted based on usage patterns and traffic volumes. For example, during peak hours the delay may be extended to 30 seconds, while in low-traffic periods it may be reduced to 10 seconds. This adaptive timing optimizes both user convenience and system efficiency.

Throughout the delay period, the system maintains its safety protocols. The obstruction sensors remain active to prevent panel closure if any objects are detected in the panel path. Visual indicators, such as LED lights, signal the system's status to users. The microcontroller continues high-frequency sampling of all sensor inputs while managing the delay timer, ensuring responsive and safe operation.

### Example Walk-Through Scenario: Interior Object Detection for Optimized Decontamination

In this scenario, a Germpass Door Handle Unit incorporates an advanced interior detection system utilizing infrared proximity sensors positioned within the enclosure to detect physical interaction with the door handle. This implementation demonstrates how the system optimizes decontamination cycles based on confirmed contact events.

The system employs multiple sensor technologies working in concert. The primary ultrasonic sensor (TDK CH201) monitors external approach vectors, while the internal infrared proximity sensors focus specifically on detecting objects within the enclosure space surrounding the door handle. These interior sensors are strategically placed to create detection zones that encompass the handle's interaction area.

Consider a typical interaction sequence: A person approaches the door, triggering the external ultrasonic sensor which detects their presence within the 4-foot threshold. The system calculates their velocity and direction using sequential distance measurements taken at 50 ms intervals. Upon confirming approach intent through negative velocity values (indicating movement toward the unit), the microcontroller activates the drive assembly to open the access panel.

The internal proximity sensors now actively monitor the space around the door handle. These sensors operate at a higher sampling rate (e.g., 100Hz) to ensure precise detection of hand movements within the enclosure. The sensors utilize infrared beams to create an invisible detection field around the handle. When a hand breaks this field, the system registers a potential interaction event.

However, in this scenario, the person receives a phone call just as they reach the door. They stop, turn around, and walk away without touching the handle. The internal proximity sensors detect no breach of the detection field around the handle. The microcontroller processes this data and determines that although the access panel was opened, no physical contact occurred with the fomite. The system's decision-making algorithm evaluates two notable criteria:
1. Confirmation from external sensors that the user has moved away (positive velocity values)
2. Verification from internal sensors that no object entered the handle's detection zone Based on this data, the microcontroller executes an optimized response sequence:
Initiates closure of the access panel via the drive assembly
Bypasses the UV-C decontamination cycle
Returns to standby mode while maintaining active sensor monitoring

This intelligent response mechanism provides several advantages:
Extends battery life by avoiding unnecessary UV-C activation
Reduces wear on UV-C LED components
Optimizes system efficiency through conditional decontamination
Maintains rapid response capability for subsequent users

The interior detection feature represents a significant advancement over basic motion-sensing systems.

By confirming actual contact events rather than merely detecting presence, the system achieves superior operational efficiency while maintaining its primary function of pathogen control. This selective activation approach substantially extends the operational life of both the battery and UV-C components, making the system more practical for high-traffic installations.

The microcontroller maintains detailed logs of both activated and bypassed decontamination cycles, providing valuable usage analytics while helping to optimize system performance and maintenance schedules. This data-driven approach enables continuous refinement of detection thresholds and timing parameters, further enhancing the system's efficiency and effectiveness.

### Example Walk-Through Scenario: Restroom Stall Latch Unit Operation

The Germpass Restroom Stall Latch Unit implements a specialized operational flow designed for the unique requirements of restroom environments. This scenario demonstrates how the system manages access and decontamination based on stall occupancy detection.

The system utilizes multiple sensor types to ensure accurate occupancy detection. A primary ultrasonic sensor (TDK CH201) monitors the stall entrance, while additional infrared sensors track movement within the stall space. These sensors work in concert to provide reliable occupancy data to the microcontroller.

When a user approaches the stall, the ultrasonic sensor detects their presence and tracks their movement. The system calculates velocity and direction using sequential distance measurements at 50 ms intervals. As the user enters the stall, the sensors detect their movement through the entrance threshold. The microcontroller processes this data to confirm stall entry, triggering the motorized drive assembly to open the access panel covering the latch mechanism.

The system maintains continuous monitoring of the stall interior through its sensor array. For example, when a user enters a stall at a busy airport restroom, the system:
1. Detects initial approach through ultrasonic sensing
2. Confirms stall entry through sequential sensor triggers
3. Opens the access panel via the drive assembly
4. Maintains panel open state while occupancy is detected
5. Monitors for exit events through sensor data analysis

During occupancy, the system processes sensor data through filtering algorithms to maintain accurate presence detection while eliminating false readings from normal user movement within the stall. The microcontroller employs adaptive thresholds to account for various user behaviors and movement patterns.

When the user exits the stall, the sensor array detects this transition through:
Loss of occupancy signals from interior sensors
Movement detection through the stall threshold
Velocity calculations indicating exit trajectory

Upon confirming the stall is vacant, the system initiates its closure sequence:
1. Activates the drive assembly to close the access panel
2. Verifies complete panel closure through position sensors
3. Initiates the UV-C decontamination cycle at 265nm wavelength
4. Returns to standby mode after cycle completion

This implementation provides several advantages, including, for example:
Hands-free operation enhances hygiene
Automatic panel management reduces touch points
Immediate access to latch during occupancy
Efficient decontamination after each use

The system includes safety features to prevent panel closure while the stall is occupied. If the occupancy sensors detect any presence during the closure sequence, the system immediately halts panel movement and returns to the open state. This failsafe mechanism ensures user safety and prevents mechanical issues.

The UV-C decontamination cycle activates only after confirming both stall vacancy and panel closure. The system's reflective interior surfaces optimize UV-C coverage of the latch mechanism, ensuring thorough sanitization between users. This targeted approach maximizes pathogen reduction while minimizing energy consumption.

Throughout operation, the system maintains active monitoring of its battery status, UV-C LED performance, and mechanical components. Visual indicators on the unit display system status, while the microcontroller logs usage patterns and operational data for maintenance purposes. This comprehensive approach ensures reliable performance in high-traffic restroom environments while optimizing resource utilization and maintaining high standards of hygiene.

In at least some embodiments, the system may use extended UV-C exposure times to ensure higher levels of decontamination. Additional sensors may verify the effectiveness of the decontamination process.

In some embodiments, ultrasonic sensors operate by emitting high-frequency sound waves, measuring the time taken for these waves to reflect back after striking an object. This approach enables the system to accurately detect the presence and movement of objects, such as human users, without any physical contact. The detection system may calculate the velocity by analyzing the rate of change in distance over time.

To determine the direction of movement, the system evaluates the user's velocity. A positive velocity, indicating an increasing distance from the unit, suggests the user is moving away. Conversely, a negative velocity, representing a decreasing distance, indicates the user is approaching the fomite. If the distance remains relatively constant over time, the system may interpret the motion as parallel to the unit, implying that the user does not intend to interact.

In at least some embodiments, ultraviolet germicidal irradiation (UVGI) is used for decontamination. where UV-C light is employed to disrupt the DNA and RNA of microorganisms. This process prevents the microorganisms from replicating. effectively neutralizing potential pathogens on the surface of the fomite. The exposure time required for decontamination is calculated based on the intensity of the UV-C light and the specific types of pathogens targeted, ensuring sufficient exposure for optimal decontamination.

Safety mechanisms are integral to the system, ensuring that users are not exposed to harmful UV-C light during operation. The access panel and the enclosing structure are designed to fully contain the UV light during the decontamination cycle. The system only activates the UV-C light when the access panel is securely closed, safeguarding users during the process.

The access panel of the apparatus serves multiple functions. It acts as a physical barrier to prevent unintended contact with the fomite when not in use. During the decontamination cycle, the panel functions as a safety shield to contain the UV-C light. Additionally, the system is equipped with user feedback mechanisms, such as visual indicators or auditory signals, to inform the user of the system's readiness and guide them during interaction. The access panel is designed to signal availability when open, and integrates with sensors that trigger user-specific responses to optimize interaction and safety.

Notable takeaways from this system's design include its focus on energy efficiency, safety, adaptability. and user experience. For example, efficiency is a noteworthy feature, as the system conserves energy by activating only when user interaction is detected. The decontamination cycle is initiated only when necessary, preventing unnecessary cycles and reducing power consumption. By accurately interpreting user intent and monitoring usage patterns, the system minimizes idle operation and ensures optimal performance. Safety is ensured through controlled access and UV-C shielding. The system protects users from harmful UV-C exposure by containing the light within a sealed enclosure and only activating the decontamination process when the access panel is securely closed. Additionally, by decontaminating fomites after each interaction, it significantly reduces the risk of germ transmission in high-traffic environments. Adaptability is another notable strength, as the system adjusts to various user behaviors and environments. Whether deployed in offices, hospitals, or public spaces, the system may be configured to suit different traffic levels and interaction patterns. Adjustable parameters, such as detection thresholds and decontamination timing, allow for optimal operation across different settings. User experience is enhanced by providing clear feedback through visual or auditory signals. The system ensures seamless interaction by guiding users when the fomite is ready for use and signaling when the decontamination process is complete. This improves user confidence and encourages compliance with hygiene protocols, making the system intuitive and user-friendly in various public and private environments.

In at least some embodiments, the Germpass Unit is designed to operate autonomously, requiring minimal direct interaction from users beyond the physical use of the protected fomite, such as a door handle or restroom latch. The interaction between users and the Germpass Unit is seamless, contactless, and highly automated. Users need only to approach and interact with the fomite; all other processes-including opening the access panel and initiating UV-C decontamination-are managed autonomously by the unit.

As a user approaches the fomite, the system's sensors detect their presence, motion, proximity, and direction. The Germpass Unit intelligently assesses the user's intent to interact, minimizing unnecessary actions like opening the access panel for passersby or initiating decontamination when no interaction occurs. If the system confirms the user's intent, it automatically opens the access panel, providing immediate access to the fomite without requiring any manual input.

After the user engages with the fomite, the system closes the access panel and initiates the UV-C decontamination cycle. This process ensures that the touchpoint remains germ-free for subsequent users, minimizing their exposure to pathogens and maintaining a high level of hygiene. In situations where a user approaches but does not interact with the fomite, the Germpass Unit conserves energy by closing the panel without initiating the decontamination cycle. This approach balances user convenience with effective decontamination, reducing unnecessary UVGI cycles and conserving energy.

For embedded Germpass Units, such as those integrated directly into doors or ATM machines, the user experience remains identical. The sensor-driven procedures ensure that access to the fomite is provided only when needed, with users experiencing no delay while benefiting from enhanced germ protection. All processes—including panel opening, closing, and decontamination—occur seamlessly and automatically, maintaining a consistent and efficient user interaction across different implementations of the Germpass Unit.

The Germpass Unit represents a significant advancement in decontamination technology, integrating multiple innovative features that set it apart from prior systems. Each distinguishing aspect contributes to its effectiveness in maintaining pathogen-free fomites in high-traffic public environments.

### Noteworthy Germpass Unit Features

1. Real-Time User Intent Detection: The Germpass Unit employs advanced ultrasonic sensors to measure the proximity, velocity, and direction of users in real time. Unlike traditional systems that rely solely on basic motion detection, this sophisticated sensing technology interprets user intent with high precision. The ultrasonic sensors operate autonomously in free-run mode, providing continuous updates without external triggers. By calculating the rate of change in distance (velocity) and assessing whether a user is approaching, stationary, or moving away, the system determines if there is intent to interact with the fomite. This real-time analysis ensures appropriate responses, significantly reducing unnecessary operations and enhancing overall efficiency.
2. User-Intent-Based Access Control: Building upon its advanced sensing capabilities, the Germpass Unit features intelligent access control mechanisms. The system analyzes user movement patterns to determine the intention to interact with the fomite. Only when a user's intent is confirmed does the motorized access panel open, reducing unnecessary exposure to potential contaminants. This selective access minimizes the likelihood of contamination spread and decreases wear on mechanical components, enhancing both safety and the unit's lifespan.
3. Seamless, Automated Decontamination: After a user completes interaction with the fomite, the Germpass Unit automatically initiates a decontamination cycle using ultraviolet germicidal irradiation (UVGI). The system incorporates UV-C LED chips that emit light at the optimal germicidal wavelength of 265 nm, known for its effectiveness in destroying a wide range of pathogens. This automated process may require no additional user input, providing a seamless experience while maintaining high hygiene standards. By ensuring the decontamination cycle is activated only after actual contact, the unit optimizes resource utilization and upholds effective sterilization protocols.
4. Optimized UV-C Exposure: The Germpass Unit is designed to maximize the effectiveness of the UV-C decontamination process. The interior surfaces of the enclosure are constructed with highly reflective materials such as aluminum or polytetrafluoroethylene (PTFE). These materials enhance the reflection of UV-C light within the chamber, ensuring even and comprehensive exposure of the fomite surface to the germicidal wavelength. This design minimizes the time required for effective pathogen destruction, ensuring rapid and efficient decontamination.
5. Efficient Energy Management: Energy efficiency is a notable aspect of the Germpass Unit's design. By utilizing sensors capable of interpreting user intent, the system limits the activation of motorized components and UV-C decontamination cycles to when they are genuinely needed. Decision-making algorithms reduce the frequency of mechanical operations like panel opening and closing and prevent unnecessary UV-C cycles when no contact has occurred. This strategic approach conserves battery life, reduces energy consumption, and extends the operational lifespan of both mechanical parts and the UV-C light source.
6. Energy Efficiency and Component Longevity: The intelligent decision-making process not only conserves energy but also contributes to the longevity of the unit's components. By minimizing unnecessary mechanical movements and UV-C activations, the system reduces wear and tear on mechanical parts and prevents premature degradation of the UV-C LEDs. Skipping the decontamination cycle when no contact is detected is a novel method for preserving the lifespan of notable components, ensuring the unit remains reliable and effective over an extended period.
7. Real-Time Motion Detection: Continuous measurement of user distance and velocity enables precise motion detection capabilities. The ultrasonic sensors provide real-time data that allow the system to identify user behaviors accurately. By determining whether a person is approaching, stationary, or moving away, the Germpass Unit eliminates unnecessary access panel operations. This precision saves battery life, reduces mechanical strain, and increases operational efficiency by preventing superfluous movements that do not contribute to the unit's primary function.
8. Selective Decontamination: Engineered to initiate UVGI decontamination cycles exclusively when a user interacts with the fomite, the Germpass Unit refrains from activating the UVGI cycle if a person approaches but does not make contact. This selective decontamination strategy preserves the lifespan of the UV-C LEDs, minimizes power consumption, and ensures resources are utilized only when necessary. By avoiding unnecessary sterilization cycles, the unit maintains optimal performance while conserving energy and reducing maintenance requirements.
9. Intelligent Sensor Integration: The integration of multiple sensing technologies is a hallmark of the Germpass Unit's innovative design. In addition to ultrasonic sensors for distance and velocity detection, the system incorporates proximity and motion sensors within the enclosure. This network of sensors ensures precise monitoring of user intent and fomite interaction. The ultrasonic sensors operate autonomously in free-run mode, providing continuous updates without the need for external triggers. This intelligent sensor integration enables the unit to respond accurately to a wide range of user behaviors, enhancing both efficiency and effectiveness.

The advanced features of the Germpass Unit make it particularly suitable for deployment in high-traffic public environments such as airports, hospitals, schools, and commercial buildings. By maintaining pathogen-free fomites, the unit plays a notable role in preventing the spread of infectious diseases. The intelligent sensing and energy management systems ensure efficient operation, reducing operational costs and maintenance requirements. Enhanced safety features protect users and maintenance personnel by minimizing unnecessary exposure to UV-C light and potential contaminants.

The Germpass Unit's innovative integration of real-time user intent detection. optimized UV-C exposure, efficient energy management, and intelligent sensor integration distinguishes it from prior decontamination technologies. Each aspect is carefully designed to enhance performance, conserve resources, and extend component longevity. By addressing the challenges associated with maintaining hygiene in public spaces, the Germpass Unit represents a significant advancement in decontamination technology, offering practical benefits and improved public health outcomes.

### Noteworthy Features of the Germpass Unit User Intent Interpretation Procedure

1. Proximity and Motion-Based Access Panel Operation: The Germpass Unit introduces a novel approach to access panel management by utilizing proximity and motion-based calculations. Unlike traditional systems that rely solely on simple user presence to trigger actions, this unit employs advanced sensors to detect not only the presence but also the velocity and direction of a user's movement. By intelligently determining the optimal time to open the access panel based on detailed motion and directional analysis, the system minimizes unnecessary exposure of the fomite. This method reduces mechanical wear on the access panel mechanism and enhances the overall efficiency and durability of the system. The intelligent timing ensures that the panel opens precisely when needed, providing a seamless user experience while maintaining the integrity of the decontamination process.
2. Skipping Unnecessary Decontamination Cycles: An innovative energy-saving mechanism is integrated into the Germpass Unit through its ability to skip unnecessary UV-C decontamination cycles. If the system detects that no physical interaction with the fomite has occurred-for instance, when a user approaches but then walks away-it intelligently decides not to initiate the decontamination process. This selective approach reduces energy consumption and extends the operational life of the UV-C components by preventing unnecessary use. By ensuring that resources are only utilized when necessary, the unit optimizes its functionality for high-use environments, making it more efficient and cost-effective. This feature also contributes to reduced maintenance requirements and operational costs over time.
3. Adaptive Decontamination Timing: The Germpass Unit features adaptive decontamination timing. allowing it to vary the duration of the decontamination cycle based on environmental factors such as battery life, UV-C component wear, or current traffic levels. For example, during periods of high usage, the system may adjust to shorter decontamination cycles to maintain throughput, whereas during low-traffic periods, it may extend the cycle for more thorough decontamination. This flexibility increases the system's efficiency and durability in various scenarios. By adjusting the decontamination process according to real-time conditions, the system becomes more adaptable to public settings like restrooms, elevators, and gas pumps. This adaptability ensures consistent performance while optimizing energy consumption and prolonging component life.
4. Advanced Motion and Intent Recognition: Employing sophisticated ultrasonic sensors, the Germpass Unit measures user proximity, velocity, and direction of movement to achieve advanced motion and intent recognition. By continuously capturing these metrics, the system may accurately predict a user's intent to interact with the fomite. For instance, if a user is approaching directly towards the unit at a certain speed, the system interprets this as an intention to engage. This feature ensures that the access panel opens only when the user is approaching with the intent to interact, thereby reducing unnecessary exposure of the fomite and enhancing security. The system's ability to discern user intent enhances operational efficiency and contributes to a more intuitive and user-friendly experience.
5. Energy-Saving Decontamination Control: The Germpass Unit introduces energy-saving decontamination control by intelligently deciding when to initiate the UV-C decontamination process. If no physical contact with the fomite is detected-such as when a user approaches but does not touch the fomite-the system skips the UV-C decontamination cycle. This decision reduces energy consumption and extends the lifespan of the UV-C components by avoiding unnecessary use. Unlike conventional systems that initiate cleaning cycles after every user interaction regardless of contact, this conditional decontamination triggering optimizes UVGI (Ultraviolet Germicidal Irradiation) usage. By ensuring that decontamination occurs only when necessary, the system becomes more efficient and suitable for high-use environments, reducing operational costs and environmental impact.
6. Automated, Real-Time Decontamination with UV-C: The unit incorporates automated, real-time decontamination using UV-C LEDs with an optimized wavelength, such as 265 nm, which is known for its peak germicidal effectiveness. This ensures rapid and efficient inactivation of microorganisms on the fomite surface. Reflective materials, such as polished aluminum or specialized coatings inside the unit, enhance

UV-C light distribution by reflecting the radiation uniformly across the fomite. This design provides comprehensive coverage, ensuring that all surfaces are effectively disinfected. This real-time, automated decontamination process is faster and more effective than traditional methods like chemical disinfectants, which may require manual application and have longer contact times. The elimination of manual intervention not only enhances user safety by reducing exposure to harmful chemicals but also increases operational efficiency.
7. Dynamic Motion Sensing: Dynamic motion sensing is a notable inventive feature wherein the Germpass Unit uses continuous velocity measurements to distinguish itself from other systems. The unit doesn't merely detect the presence of a user; it calculates the speed and direction of their movement in real-time. This capability allows the system to respond in a nuanced manner based on the user's intent to engage with the fomite. For example, if a user is moving quickly past the unit without slowing down, the system interprets this as a lack of intent to interact and keeps the access panel closed. Conversely, if a user slows down or stops in front of the unit, the system prepares to open the panel. By understanding whether a user is approaching, standing still, or moving away, the system may adjust its actions accordingly, enhancing both efficiency and user experience while reducing unnecessary wear on mechanical components.
8. Panel Control Based on Motion Direction: The system employs intelligent panel control based on the detected direction of motion. If the user's movement is parallel to or away from the unit-indicating that they are walking past or moving away-the access panel remains closed. This feature conserves energy by preventing the unnecessary operation of mechanical components and prolongs the lifespan of the hardware by reducing mechanical wear on the access panel mechanism. By ensuring that the panel only opens when a user is directly approaching with the intent to interact, the system enhances security and operational longevity. This approach also minimizes the potential for contamination by limiting exposure of the fomite to the environment.
9. Conditional Decontamination Triggering: Conditional decontamination triggering is an innovative feature where the Germpass Unit initiates the UV-C decontamination cycle only after confirmed physical contact with the fomite. Unlike conventional systems that initiate cleaning cycles after every user interaction, regardless of whether contact occurred, this selective approach optimizes UVGI usage. By reducing unnecessary decontamination cycles, the system extends the operational life of the UV-C components and reduces energy consumption. This makes the unit more efficient and suitable for high-use environments where constant decontamination after every non-contact interaction would be impractical and wasteful. This intelligent control ensures that decontamination resources are allocated effectively, maintaining high hygiene standards without unnecessary expenditure.

Collectively, these features ensure that the Germpass Unit addresses real-world needs for rapid, user-focused decontamination while extending operational longevity and reducing unnecessary energy expenditure. By incorporating advanced motion sensing, intelligent access panel control, adaptive decontamination timing, and efficient UV-C decontamination methods, the system provides a comprehensive solution that is both effective and efficient for public use settings. The integration of these features enhances user safety, optimizes resource utilization, and offers a significant advancement over traditional decontamination systems.

Although several example embodiments of one or more aspects and/or features have been described in detail herein with reference to the accompanying drawings, it is to be understood that aspects and/or features are not limited to these precise embodiments, and that various changes and modifications may be effected therein by one skilled in the art without departing from the scope of spirit of the invention(s) as defined, for example, in the appended claims.

1. A germ decontamination apparatus adapted to decontaminate at least one fomite region of an object fixedly attached to a support structure, the apparatus comprising:
a housing defining an interior enclosure;
the housing including a rear housing portion and a front housing portion;
the rear housing portion including a rear opening disposed therein, the rear opening being configured or designed to provide access to the interior enclosure;
the housing being configured or designed to be attachable to the support structure in a first configuration which permits the entirety of the at least one fomite region to be exposed to the interior enclosure;
the front housing portion including a front cover plate and a front opening configured or designed to provide access to the interior enclosure;
a movable access door movably attached to the housing for preventing access to the interior enclosure via the front opening, the access door being configurable in a closed configuration which prevents access to the interior enclosure, the access door being further configurable in an open configuration which permits access to the interior enclosure;
wherein the access door comprises a first movable, stackable access panel, the first access panel being movable into a first stacked configuration with the front cover plate such that the first access panel is stacked either behind or in front of the front cover plate when the access door is configured in the open configuration;
the first access panel including at least one first edge portion and a first body portion, the at least one first edge portion being different from the first body portion, the first body portion including a first curved body portion;
a drive mechanism for causing the access door to open or close in response to at least one triggering event;
one or more ultraviolet light sources disposed at the interior enclosure and configured to decontaminate the at least one fomite region;
one or more sensors configured to detect the at least one triggering event; and
a controller configured to control the one or more sensors, the drive mechanism, and/or the one or more light sources.

2. The apparatus of claim 1 wherein a cross-section of the first access panel body is substantially U-shaped or substantially C-shaped.

3. The apparatus of claim 1 wherein the access door further comprises a second movable access panel, the second access panel being movable into a second stacked configuration such that the second access panel is stacked either behind or in front of the first access panel when the access door is configured in the open configuration.

4. The apparatus of claim 1:
wherein the object is a door knob or door handle;
wherein the support structure is a door;
wherein the controller is configured or designed to execute instructions for:
detecting a presence of a first person;
configuring the access door in the open configuration to enable the detected first person to access the object in the interior enclosure;
detecting an absence of the first person while the access door is configured in the open configuration; and
delaying closing of the access door for at least 5 seconds after detecting the absence of the first person.

5. The apparatus of claim 1 wherein the controller is configured or designed to execute instructions for:
detecting a presence of a first person within a first predetermined distance from the apparatus;
determining a current direction of motion of the first person; and
configuring the access door in the open configuration in response to determining that: (i) the first person is within a first predetermined distance from the apparatus, and (ii) the current direction of movement of first person is toward the apparatus.

6. The apparatus of claim 1 wherein the controller is configured or designed to execute instructions for:
detecting a presence of a first person within a first predetermined distance from the apparatus;
determining a current direction of motion of the first person;
configuring the access door in the open configuration in response to determining that: (i) the first person is within a first predetermined distance from the apparatus, and (ii) the current direction of movement of first person is toward the apparatus;
causing the access door to be or to remain in a closed configuration in response to determining that the first person is greater than the first predetermined distance from the apparatus; and
causing the access door to be or to remain in a closed configuration in response to determining that the current direction of movement of first person is not toward the apparatus.

7. The apparatus of claim 1:
wherein the object is a latch or locking mechanism for a door of a restroom stall;
wherein the controller is configured or designed to execute instructions for:
detecting a presence of a first person in the restroom stall;
configuring the access door in the open configuration while the first person is detected in the restroom stall;
detecting an absence of the first person in the restroom stall;
configuring the access door in the closed configuration in response to detecting the absence of the first person in the restroom stall; and
initiating a first decontamination operation for decontaminating the at least one fomite region in response to: (i) detecting the absence of the first person in the restroom stall, and (ii) determining that the access door in the closed configuration.

8. The apparatus of claim 1 further comprising a first sensor configured or designed to monitor the interior enclosure and to detect a presence of an external object within the interior enclosure;
wherein the controller is configured or designed to execute instructions for:
configuring the access door in the open configuration;
determining if the external object has been detected within the interior enclosure;
initiating a first decontamination operation for decontaminating the at least one fomite region in response to determining that the external object has been detected within the interior enclosure; and
preventing initiation of the first decontamination operation in response to determining that the external object has not been detected within the interior enclosure.

9. The apparatus of claim 1 further comprising a first sensor configured or designed to monitor the interior enclosure and to detect if an external object has made contact with the object within the interior enclosure;
wherein the controller is configured or designed to execute instructions for:
configuring the access door in the open configuration;
determining if the external object has made contact with the object within the interior enclosure;
initiating a first decontamination operation for decontaminating the at least one fomite region in response to determining that the external object made contact with the object; and
preventing initiation of the first decontamination operation in response to determining that the external object did not make contact with the object.

10. The apparatus of claim 1:
wherein the object is an elevator button panel of an elevator cab;
wherein the controller is configured or designed to execute instructions for:
detecting a presence of at least one person in elevator cab;
configuring the access door in the open configuration in response to detecting the presence of the at least one person in elevator cab;
detecting current vertical movement of the elevator cab;
configuring the access door in the closed configuration in response to detecting the vertical movement of the elevator cab; and
initiating a first decontamination operation for decontaminating the at least one fomite region in response to: (i) detecting the vertical movement of the elevator cab, and (ii) determining that the access door in the closed configuration.

11. The apparatus of claim 1:
wherein the object is an elevator button panel of an elevator cab;
wherein the controller is configured or designed to execute instructions for:
detecting a presence of at least one person in elevator cab;
detecting that the elevator cab is currently not moving; and
configuring the access door in the open configuration in response to: (i) detecting the presence of the at least one person in elevator cab, and (ii) in response to detecting that the elevator cab is currently not moving detecting current vertical movement of the elevator cab;
configuring the access door in the closed configuration in response to detecting the vertical movement of the elevator cab; and
initiating a first decontamination operation for decontaminating the at least one fomite region in response to: (i) detecting the vertical movement of the elevator cab, and (ii) determining that the access door in the closed configuration.

12. The apparatus of claim 1:
wherein the first panel and the front cover plate are configured in the first stacked configuration while the access door is configured in the open configuration; and
wherein the first panel and the front cover plate are configured in a non-planar, stepped configuration while the access door is configured in the closed configuration.

13. The apparatus of claim 1, wherein the housing is configured or designed to envelope the at least one fomite region while the housing is attached to the support structure according to the first configuration, and while the access door is configured in the closed configuration.

14. The apparatus of claim 1, wherein the rear opening is configured or designed to enable the at least one fomite region to pass therethrough.

15. The apparatus of claim 1:
wherein the object is a fixture fixedly attached to the support structure, and
wherein the housing is configured or designed to be fixedly attachable to the support structure in a manner which causes the entirety of the at least one fomite region to be exposed to the interior enclosure via the rear opening.

16. The apparatus of claim 1 further comprising at least one portable power source for providing power to at least one electronic component of the portable germ decontamination apparatus.

17. The apparatus of claim 1, wherein the one or more sensors comprise an obstruction sensor, a motion sensor or detector, a light sensor, a sound sensor, and/or a heat or infrared sensor.

18. The apparatus of claim 1, wherein the controller is configured or designed to execute a plurality of instructions for:
implementing the decontamination of the at least one fomite region in a manner which causes inactivation of germ populations of the at least one fomite region by at least 99%.

19. The apparatus of claim 1, wherein the one or more ultraviolet light sources are configured or designed to produce UV-C radiation with a wavelength in the range of 200-280 nm.

20. The apparatus of claim 1, wherein the housing is configured or designed to provide a substantially airtight interior enclosure while the housing is attached to the support structure via the first configuration, and while the access door is in the closed configuration.

21. The apparatus of claim 1 being further configured or designed to decontaminate all exposed fomite regions of the object while the housing is attached to the support structure via the first configuration, and while the access door is in the closed configuration.

22. The apparatus of claim 1 further comprising a wireless transceiver configured or designed to facilitate wireless communication with a second germ decontamination apparatus comprising a second movable access door; and
wherein the controller is configured or designed to execute a plurality of instructions for transmitting a first control signal to the second germ decontamination apparatus to cause the second germ decontamination apparatus to open the second access door.

23. A germ decontamination apparatus adapted to decontaminate at least one fomite region of an object fixedly attached to a support structure, the apparatus comprising:
a housing defining an interior enclosure;
the housing including a rear housing portion and a front housing portion;
the rear housing portion including a rear opening disposed therein, the rear opening being configured or designed to provide access to the interior enclosure;
the housing being configured or designed to be attachable to the support structure in a first configuration which permits the entirety of the at least one fomite region to be exposed to the interior enclosure;
the front housing portion including a front cover plate and a front opening configured or designed to provide access to the interior enclosure;
a movable access door movably attached to the housing for preventing access to the interior enclosure via the front opening, the access door being configurable in a closed configuration which prevents access to the interior enclosure, the access door being further configurable in an open configuration which permits access to the interior enclosure;
wherein the access door comprises a first movable, stackable access panel, the first access panel being movable into a first stacked configuration with the front cover plate such that the first access panel is stacked either behind or in front of the front cover plate when the access door is configured in the open configuration;
the first access panel including at least one first edge portion and a first body portion, the at least one first edge portion being different from the first body portion, the first body portion including a first curved body portion;
a drive mechanism for causing the access door to open and close;
one or more sensors;
a first decontamination mechanism disposed at the interior enclosure and configured or designed to decontaminate the at least one fomite region; and
a controller configured to control the one or more sensors, the drive mechanism, and/or first decontamination mechanism.

24. The apparatus of claim 23 wherein the access door further comprises a second movable access panel, the second access panel being movable into a second stacked configuration such that the second access panel is stacked either behind or in front of the first access panel when the access door is configured in the open configuration.

25. The apparatus of claim 23:
further comprising a first sensor configured or designed to monitor the interior enclosure, and to detect a presence of an external object being placed within the interior enclosure.

26. The apparatus of claim 23 further comprising a first sensor configured or designed to monitor the interior enclosure, and to detect if an external object has made contact with the object within the interior enclosure.

27. The apparatus of claim 23 wherein the controller is configured or designed to execute instructions for:
detecting a presence of a first person within a first predetermined distance from the apparatus;
determining a current direction of motion of the first person; and
configuring the access door in the open configuration in response to determining that: (i) the first person is within a first predetermined distance from the apparatus, and (ii) the current direction of movement of first person is toward the apparatus.

28. The apparatus of claim 23 wherein the controller is configured or designed to execute instructions for:
detecting a presence of at least one person;
configuring the access door in the open configuration in response to detecting the presence of the at least one person;
detecting movement of the apparatus; and
configuring the access door in the closed configuration in response to detecting movement of the apparatus.

29. The apparatus of claim 23 further comprising a wireless transceiver configured or designed to facilitate wireless communication with a second germ decontamination apparatus comprising a second movable access door; and
wherein the controller is configured or designed to execute a plurality of instructions for transmitting a first set of control signals to the second germ decontamination apparatus for controlling movement of the second access door.
